(19) United States
(12) Patent Application Publication
(10) Pub. No.: US 2024/0254398 A1
(43) Pub. Date: Aug. 1, 2024

(54) HIGH PRODUCTIVITY BIOPROCESSES FOR THE MASSIVELY SCALABLE AND ULTRA-HIGH THROUGHPUT CONVERSION OF CO2 INTO VALUABLE PRODUCTS

(71) Applicant: Kiverdi, Inc., Pleasanton, CA (US)

(72) Inventors: John Reed, Pleasanton, CA (US); David Stout, Menlo Park, CA (US); Elisabeth Perea, Menlo Park, CA (US); Jin-Ping Lim, Pleasanton, CA (US); Jil Geller, Pleasanton, CA (US); Dan Robertson, Pleasanton, CA (US); Robert Wilson, Menlo Park, CA (US); Ripudaman Malhotra, Menlo Park, CA (US)

(21) Appl. No.: 18/560,290

(22) PCT Filed: May 17, 2022
(86) PCT No.: PCT/US2022/029657

§ 371 Date: Nov. 10, 2023

Related U.S. Application Data
(60) Provisional application No. 63/189,615, filed on May 17, 2021.

(51) Int. Cl. C10G 2/00; C12M 1/06; C12M 1/34; C12N 1/20; C12P 1/04; C12R 1/01

(52) U.S. Cl. CPC C10G 2/50; C12M 27/02; C12M 41/26; C12M 41/34; C12M 41/40; C12N 1/20; C12P 1/04; C12R 2001/01

(57) ABSTRACT

Bioreactors and methods for growing a chemoautotrophic culture of a microorganism, such as a hydrogen-oxidizing or carbon monoxide-oxidizing microorganism, are provided. The bioreactors and methods provide for enhanced growth and productivity of microorganisms that use gaseous sources of carbon and energy and provide an environment for carbon fixing to produce organic molecules of interest and/or biomass. The present bioreactors and methods achieve enhanced growth with respect to cell density, culture duration and/or growth rate, while maintaining safe operating conditions.

### CROSS-REFERENCE TO RELATED APPLICATIONS

This application claims the benefit of U.S. Provisional Application No. 63/189,615, filed on May 17, 2021, which is incorporated by reference herein in its entirety.

### FIELD OF THE INVENTION

The present disclosure relates to bioreactor designs for high productivity growth of microorganism cultures, in particular high productivity growth of microorganisms on a gaseous substrate as a carbon and energy source. The present disclosure also relates to the field of single cell protein, and the production of protein concentrates, protein isolates, and protein hydrolysates produced from biological sources, and methods of making the same. In particular, the present disclosure relates to producing biobased products from renewable sources, such as biological processes designed to capture carbon dioxide emissions and other waste carbon conversion or diversion processes.

### BACKGROUND

By 2050 the world's population is expected to reach 10 billion people, resulting in a 70% increase in food demand. Current food production generates over 20% of total greenhouse gases (GHG) and uses >37% of the planet's land. A major contributor to these emissions is the depletion and release of soil carbon through current agricultural practices, which results in a loss of soil fertility in addition to large GHG emissions.

Single cell protein (SCP) is a very promising source of protein and other nutrients that does not rely on agricultural land or inputs, with dramatically lower GHG, land, and water footprint than current plant and animal sources of food.

However, existing processes and reactor designs to produce SCP, as well as other bio-based products, have numerous limitations. One promising approach to producing SCP is using a gas bioprocess, and in particular gas bioprocesses that consume CO₂ as the carbon source to produce protein and other value-added products. However, there are currently a number of limitations with gas bioprocesses. High mass transfer rates often can only be achieved with energy-intensive mixing that is not practical. Gas bioprocesses often have excess waste of gaseous feedstocks. Existing reactor designs for gas fermentation do not achieve the required mass transfer of gases into solution for gas bioprocesses, or else they do not meet acceptable safety criteria for utilizing potential flammable or explosive gas mixes.

In Stirred Tank Reactors (STRs), gases are introduced in the bottom of the reactor and agitation with a range of impeller designs breaks up gas bubbles and increases gas/liquid contact time due to mixing before gases leave the water-column. Energy requirements for high agitation rates necessary to enhance mass transfer are high. STRs generally operate with gases flowing through in a single pass, whereby a significant portion of the gases is wasted due to inadequate residence time for full absorption by the liquid.

Chemoautotrophic microorganisms are generally microbes that can perform CO₂ fixation, obtaining the reducing equivalents needed for CO₂ fixation from an inorganic external source. Such organisms may be employed in hybrid chemical/biological processes where the biological step is limited to the fixation of C1 compounds, such as CO₂, alone, which corresponds to the dark reaction of photosynthesis, while reducing equivalents needed for carbon fixation are generated through an abiotic process. A wide array of abiotic technologies may be used to power the process, such as solar photovoltaic (PV), solar thermal, wind, geothermal, hydroelectric, or nuclear. Such technologies can be used to generate reducing equivalents needed for carbon fixation, and particularly hydrogen gas or reduced hydrogen atoms or hydride, from abundant water resources including and particularly non-potable water, salt water, and brine sources, through the use of established electrolysis technologies, Thus, chemoautotrophic microorganisms show promise in the capture and conversion of CO₂ gas to fixed carbon as well as in the biological conversion of syngas or producer gas to fixed-carbon products.

Bioreactors that are configured for enhanced growth of such microorganisms would be desirable.

### BRIEF SUMMARY OF THE INVENTION

Bioreactors and methods for culturing chemoautotrophic and other types of microorganisms are provided.

Provided herein are novel, ultra-high pressure, knallgas (CO₂/H₂/O₂) and other bioprocesses with the goals of achieving unprecedented productivities, yields and increasingly favorable techno-economic and life cycle outcomes. In addition to process engineering for high pressure reactor performance, the present invention describes the use of adaptive evolution to select pressure tolerant phenotypes of knallgas and other production organisms. The combined reactor and organism platform technology is applicable to production of protein directly from CO₂, as well as to other valuable products such as nutritionals, oils, chemicals, and fuels.

Certain embodiments of the present invention comprise a bioreactor design and operation that addresses the need to attain at large scale a targeted mass transfer of gases (determined by k_{L}a (volumetric mass transfer coefficient), and pressure) into aqueous solution, along with other with other conditions (e.g., temperature (T), pH, dissolved oxygen (DO), dissolved nutrients, shear stresses) for optimal biomass productivity and H₂ and CO₂ conversion in a gas-fermentation process.

The high pressure processes described herein are applicable to the production of high-protein, microbial-based products, such as nutritionals, food (e.g., meat analogues), and feed products. The present invention enables the ability to achieve at least about or greater than about 80% protein composition by weight. In certain such embodiments the at least about or greater than about 80% protein composition by weight is achieved using a Cupriavidus microorganism, such as the knallgas species Cupriavidus necator or a mutant thereof, with a well-balanced amino acid profile, high vitamin content, and bioavailable minerals essential for human metabolism and health.

With the enhancements of the knallgas bioprocess and other gas bioprocesses described in this invention, one or more outcomes may follow: driving down production costs; improving carbon utilization efficiency; and/or further increasing the lifecycle assessment (LCA) advantage over conventional protein sources. These are key steps to achieving production metrics that enhance cost-advantaged commercial scale production.

The present invention addresses the utilization of chemoautotrophic microorganisms for CO₂ fixation, and processes for the provision of reducing equivalents needed for CO₂ fixation from an inorganic external source. In certain embodiments of the present invention, chemoautotrophic microorganisms, and in particular knallgas microorganisms, are employed in hybrid chemical/biological processes where the biological step is limited to just the fixation of C1 compounds, such as but not limited to CO₂, which corresponds to the dark reaction of photosynthesis, while reducing equivalents needed for carbon fixation are generated through an abiotic process. The present invention addresses the use of a wide array of abiotic technologies to power the process, including but not limited to one or more of the following: solar photovoltaic (PV), solar thermal, wind, geothermal, hydroelectric, or nuclear power. In certain embodiments of the present invention, these abiotic technologies provide the power and/or heat required to generate the reducing equivalents needed for carbon fixation, In certain such embodiments, the reducing equivalents comprise hydrogen gas or reduced hydrogen atoms or hydride, that have been generated from water through the use of established electrolysis technologies, In certain embodiments chemoautotrophic microorganisms, and particularly knallgas microorganism are used in the capture and conversion of CO₂ gas to fixed carbon. In as well as in the biological conversion of syngas or producer gas to fixed-carbon products.

The present invention addresses the efficient delivery of reducing equivalents via electrolytically generated H₂, increasing H₂ and/or CO₂ utilization efficiency, and producing a high impact product in a scalable process while reducing or eliminating CO₂ emissions. In certain non-limiting embodiments the present invention uses the CO₂ emitted from an ethanol plant as a carbon source for the production of biochemicals. In certain such embodiments, the said CO₂ captured from an ethanol plant is purified to food grade prior to introduction to a bioreactor of the present invention. Certain embodiments of the present invention represent an advanced high pressure continuous bioprocess for sustainable and economic CO₂/H₂ conversion to a high-protein product, such as a nutritional, food, or feed product. In certain embodiments using waste CO₂ from an ethanol plant and electrolytically generated H₂ gas fermentation, engineering innovations and production strain developments described in the present invention dramatically increase culture productivity with concomitant life cycle and techno-economic gains. In certain embodiments of the present invention the biomass productivity in a continuous process on CO₂ as the sole carbon source is at least about ≥1 grams per liter per hour (g/L/h) or about ≥2 g/L/h or about ≥3 g/L/h or about ≥5 g/L/h or about ≥8 g/L/h or about ≥10 g/L/h with biomass protein content at least about ≥60% or about ≥65% or about ≥70% or about ≥75% about ≥80% by weight (wt). In certain embodiments the H₂ yield is at least about ≥2.5 grams biomass per gram H₂ or about ≥3 g biomass/g H₂ or about ≥3.35 g biomass/g H₂ or about ≥3.45 g biomass/g H₂.

Knallgas bioprocesses as production platforms described in certain aspects of the present invention are unique in that pressure can be effectively applied as an intensive variable for the optimization of an efficient scaled process. In certain embodiments, the knallgas bioprocess has gaseous reactants, but no or substantially no gaseous products. In certain such embodiments, the said knallgas bioprocess has only, or substantially only, solid and liquid products (e.g., biomass and water). Therefore, in certain embodiments of the present invention, high pressure is used to drive the biosynthetic reaction, both kinetically and thermodynamically, from reactants towards products. This feature stands in marked contrast to most other bioprocesses, such as heterotrophic processes, other gas bioprocesses (e.g., methanotrophic or carboxydotrophic), or photosynthetic processes, which have a CO₂ or O₂ waste product that must be degassed. In these cases, high pressure provides little benefit, or is detrimental. Using high pressure to drive a reaction towards products is well-established in large scale, commercial gas-to-liquid (GTL) processes such as Haber-Bosch, methanol synthesis, Fischer-Tropsch (F-T), and oil hydrogenation. But the approach of using high pressure to kinetically and thermodynamically drive reactants towards products has found little or no application to date in commercial biological processes and fermentations.

Certain embodiments of the present invention provide for significant improvements in productivity of cultures of chemoautotrophic microorganisms, and particularly knallgas microorganisms, such as but not limited to Cupriavidus microorganisms, e.g., Cupriavidus necator. Certain embodiments use strain and/or process engineering to extend observed effects of pressure to attain an order of magnitude improvement in productivity (g/L/h) on CO₂ and refine a robust, economic, and sustainable process.

Certain embodiments significantly improve the utilization of CO₂. Certain embodiments of the present invention comprise a H₂ and CO₂ based process that only captures CO₂ and incorporates it into the product, with no off-gases created in the process. In certain embodiments, the fermentation is operated using three main gas feedstocks: hydrogen, carbon dioxide, and oxygen. In certain such embodiments, no gaseous products are generated by the fermentation. In certain such embodiments, protein-rich biomass and water are the main products created during the fermentation when utilizing gases. Certain embodiments can readily pair up with traditional carbon emitters, such as an ethanol fermentation process, and consume all of the CO₂ effluent. In certain embodiments of the present invention, atmospheric CO₂ capture units are utilized as a carbon source (e.g., CO₂ source) for bioprocesses described herein. In certain embodiments of the present invention, a biogenic source of CO₂ is utilized as a carbon source and/or carbon is utilized that is drawn from the biogenic carbon cycle. In certain embodiments, the CO₂ and/or carbon sources is derived from the so-called the fast domain of the carbon cycle (the atmosphere, ocean, vegetation and soil), with carbon turnover times in the range of 1 to 100 years. In contrast, to the biogenic and/or fast domain of the carbon cycle is the geologic cycle or slow domain of the carbon cycle where carbon turnover times typically exceed 10,000 years. In certain embodiments of the present invention, no carbon is transferred from the slow domain of the carbon cycle or the geological carbon cycle to the fast domain of the carbon cycle or the biogenic carbon cycle. Certain embodiments and life cycles of embodiments of the present invention operate within the fast domain carbon cycle. Certain aspects of the present invention are aimed at further accelerating the biogenic carbon cycle such that biogenic carbon is more rapidly directed into organic molecules and/or biochemicals and/or biobased products of use for human, animal, plant, and/or microbial nutrition and/or for other useful or valuable applications of said organic molecules and/or biochemicals and/or biobased products.

In certain embodiments of the present invention, biomass is produced from CO₂ that comprises Single Cell Protein (SCP). Certain aspects of the invention cover applications of the SCP such as use as a protein-rich animal feedstuff, or as a protein source or source of other nutrients for another organism (e.g., microorganism, fungi, plants, animals, humans).

In one aspect, a method is provided for culturing a microorganism, including: delivering oxygen gas into a culture of a hydrogen-oxidizing or carbon monoxide-oxidizing microorganism contained in a reactor vessel, wherein a gas headspace overlies the culture; measuring a level of oxygen gas in the headspace or a level of dissolved oxygen in the culture; regulating a rate of delivery of oxygen gas into the culture based on the measured level of oxygen gas; measuring a level of pH in the culture; and regulating a rate of delivery of a base and a nutrient amendment based on the measured level of pH, wherein the rate of delivery of the nutrient amendment is proportional to the rate of delivery of the base, to thereby continuously culture the microorganism.

In another aspect, a biological and chemical method is provided for the biological conversion of inorganic and/or organic molecules comprising one or more carbon atoms into organic molecules, said method comprising: introducing chemical reactants comprising inorganic and/or organic molecules comprising one or more carbon atom and comprising a gaseous substrate into an enclosed environment within a bioreactor that is held at an elevated pressure compared to an ambient pressure outside of the bioreactor, wherein the enclosed environment comprises microorganism cells in a culture medium under conditions that are suitable for growing the microorganism cells and using the microorganism cells as a biocatalyst, wherein the inorganic and/or organic molecules comprising one or more carbon atom are utilized as a carbon source by the microorganism cells for growth and/or biosynthesis of organic molecule products along with production of inorganic co-products (such as water); and converting the inorganic and/or organic molecules comprising one or more carbon atoms into the organic molecule products within the environment via at least one carbon-fixing reaction and/or at least one anabolic biosynthetic pathway contained within the microorganism cells, wherein the carbon fixing reaction and/or anabolic biosynthetic pathway is at least partially driven by chemical and/or electrochemical energy provided by electron donors and/or electron acceptors contained within the gaseous substrate, which have been generated chemically and/or electrochemically and/or thermochemically and/or are introduced into the environment from at least one source external to the environment, and are reacted by the microorganism cells within the environment, wherein the chemical reactants introduced into the environment comprise gaseous reactants, and wherein the organic products resulting from conversion of the carbon source and co-products of said conversion, and the products from the reaction of the electron donors and electron acceptors within the environment are all solids and/or liquids and/or dissolved solutes, and wherein none of the products or co-products from the conversion of the carbon source, and none of the products from the reaction of electron donors and electron acceptors, thermodynamically favor the gas phase, and wherein increased partial pressures of the gaseous reactants contained within the environment increase the thermodynamic driving force and kinetic rates for the conversion of the carbon source and/or the reaction of electron donors and electron acceptors.

In another aspect, a method is provided for culturing a microorganism, including: delivering a gas mixture including oxygen gas into a culture of a hydrogen-oxidizing or carbon monoxide-oxidizing microorganism in a vessel of a bioreactor, wherein the gas mixture is delivered under an amount of pressure; measuring a level of dissolved oxygen in the culture; and regulating the amount of pressure based on the measured level of dissolved oxygen, to thereby continuously culture the microorganism. In another aspect, a method is provided for culturing a microorganism, including: delivering a gas mixture including oxygen gas into a culture of a hydrogen-oxidizing or carbon monoxide-oxidizing microorganism in a vessel of a bioreactor, wherein the gas mixture is delivered under elevated pressure; measuring a level of oxygen in the headspace; and regulating the flow on delivered oxygen gas based on the mol fraction of oxygen gas in the headspace.

### BRIEF DESCRIPTION OF THE DRAWINGS

FIG. 1A is a schematic diagram showing a bioreactor, according to embodiments of the present disclosure.

FIG. 1B is a schematic diagram showing a bioreactor, according to embodiments of the present disclosure.

FIG. 2 is a schematic diagram showing a bioreactor, according to embodiments of the present disclosure.

FIG. 3 is a schematic diagram showing a bioreactor, according to embodiments of the present disclosure.

FIG. 4A shows a basket impeller for use in a bioreactor, according to embodiments of the present disclosure.

FIG. 4B is a schematic diagram showing operation of a basket impeller for use in a bioreactor, according to embodiments of the present disclosure.

FIG. 5 shows a combination of a hollow gas entrainment impeller and a basket impeller for use in a bioreactor, according to embodiments of the present disclosure.

FIG. 6 shows gas entrainment impellers for use in a bioreactor, according to embodiments of the present disclosure.

FIG. 7 shows a schematic representation of oxygen desorption-absorption in a bioreactor.

FIG. 8 is a schematic diagram showing a bioreactor equipped with a membrane oxygenator, according to embodiments of the present disclosure.

FIG. 9 is a schematic diagram showing a membrane oxygenator for use with a bioreactor, according to embodiments of the present disclosure.

FIG. 10 shows a membrane oxygenator for use with a bioreactor, according to embodiments of the present disclosure.

FIG. 11 is a schematic diagram showing a bioreactor equipped with a membrane oxygenator, according to embodiments of the present disclosure.

FIG. 12 is a schematic diagram showing a bioreactor equipped with a membrane oxygenator, according to embodiments of the present disclosure.

FIG. 13 is a schematic diagram showing a bioreactor equipped with a membrane oxygenator, according to embodiments of the present disclosure.

FIG. 14 is a schematic diagram showing a bioreactor configured with a headspace reactor for control of the headspace mol % O2 in reactor 1, according to embodiments of the present disclosure.

FIG. 15 is a graph showing optical density (OD) of a culture as a function of time of operation, according to embodiments of the present disclosure, where the culture was grown on CO₂ in a fed-batch system, and where after reaching a certain OD the bulk of the culture was harvested, with a residual of broth left over to provide an inoculum for a following fed-batch run where the culture was grown up again on CO₂. This cycle can be done repeatedly and the mode of operation is called “draw and fill”.

FIG. 16 is a graph showing changes in a bioreactor as a function of operation time, according to embodiments of the present disclosure, where an initial inoculum is grown up to a certain OD in a fed-batch mode, and then the bioreactor is maintained around a certain target OD (e.g., around OD˜70) by continuous harvesting of culture broth and replenishment with fresh nutrient media along with continuously added CO₂ carbon source.

FIG. 17 is a graph showing changes in a bioreactor as a function of operation time, according to embodiments of the present disclosure, where an initial inoculum is grown up to a certain OD in a fed-batch mode, and then the bioreactor is maintained around a certain target OD by continuous harvesting of culture broth and replenishment with fresh nutrient media. During this continuous run an average dry cell weight (DCW) productivity of around 3 g/L/h grown on CO₂ as sole carbon source was maintained for around 100 hours.

FIG. 18 is a graph showing optical density of a culture as a function of time of operation, according to embodiments of the present disclosure, where the culture was grown on CO₂ in a fed-batch system up to over OD=300, which corresponds to a DCW titer of around 75 to 100 g/L.

FIG. 19 is a graph plotting the ratio DCW to OD against optical density of cultures, according to embodiments of the present disclosure.

FIG. 20 is a graph plotting estimated protein content (wt %) against optical density of cultures, according to embodiments of the present disclosure.

FIG. 21 is a schematic diagram showing a bioreactor configured with a headspace reactor for headspace % O₂ control and a split gas feed, according to embodiments of the present disclosure.

FIG. 22 is a graph showing changes in a bioreactor as a function of operation time, according to embodiments of the present disclosure, where an initial inoculum is grown up to a certain OD in a fed-batch mode, and then the bioreactor is maintained around a certain target OD (e.g., around OD˜70) by continuous harvesting of culture broth and replenishment with fresh nutrient media along with continuously added CO₂ carbon source.

FIG. 23 is a graph showing changes in a bioreactor as a function of operation time, according to embodiments of the present disclosure where an initial inoculum is grown up to a certain OD in a fed-batch mode, and then the bioreactor is maintained around a certain target OD (e.g., around OD˜60) by continuous harvesting of culture broth and replenishment with fresh nutrient media along with continuously added CO₂ carbon source.

FIG. 24 schematically shows an embodiment of a single cell protein production process.

FIG. 25 shows experimental correlation between continuous stirred tank reactor (CSTR) productivity and the headspace gas pressure inside the bioreactor.

FIG. 26 shows extrapolation of productivity vs. pressure trend shown in FIG. 19 out to commercial gas-to-liquid (GTL) process pressures.

FIG. 27 shows raw data from pressure ramp up experiment in a CSTR from 2 to 5 bar.

FIG. 28 shows Productivity versus Total Pressure trend for different CSTR set-ups—all conditions have mol % O₂≤5% in the headspace.

FIG. 29 shows linear correlation between inverse dilution rate (1/p) (equals inverse specific growth rate (g biomass produced/h/g standing biomass) in a turbidostat) and inverse H₂ yield (1/Y).

FIG. 30 shows a traditional high-P reactor (Parr Instruments) and description of its drawbacks for use in bioprocesses as a bioreactor.

FIG. 31 schematically shows a gas recirculation loop.

FIG. 32 schematically shows production and extraction of nutrients, and provision of nutrients to a second organism.

FIG. 33 schematically shows production of organic nutrients from CO₂, H₂, and other inorganic inputs, and extraction of nutrients and provision to a second organism.

### DETAILED DESCRIPTION

Provided herein are bioreactors and methods for growing cultures of microorganisms. The bioreactors and methods of the present disclosure provide for high productivity growth of microorganisms that use a gaseous substrate, such as synthesis gas or producer gas or pyrolysis gas or H₂ and CO₂ gas mixtures, as a carbon and energy source. The present bioreactors and methods provide gaseous substrates that serve as electron donors and/or electron acceptors and/or carbon sources to microorganisms, such as a hydrogen-oxidizing and/or carbon monoxide-oxidizing and/or knallgas microorganisms, to sustain chemoautotrophic growth. A non-limiting example of a gaseous electron donor includes hydrogen gas, and a non-limiting example of a gaseous electron acceptor includes oxygen gas, and a non-limiting example of a gaseous carbon source includes carbon dioxide gas.

Provided herein are biological and chemical methods for the biological conversion of inorganic and/or organic molecules containing one or more carbon atoms, into organic molecules comprising: introducing inorganic and/or organic molecules consisting of one or more carbon atom, into an enclosed environment within a bioreactor that is held at an elevated pressure compared to the ambient pressure outside of the bioreactor; wherein the enclosed environment contains microorganism cells in a culture medium under conditions that are suitable for growing the microorganism cells and using them as a biocatalyst; introducing a gaseous substrate into the enclosed environment; wherein the inorganic and/or organic molecules containing one or more carbon atom are used as a carbon source by the microorganism cells for growth and/or biosynthesis; converting the inorganic and/or organic molecules containing one or more carbon atoms into the organic molecule products of cell growth and/or biosynthesis within the environment via at least one carbon-fixing reaction and/or at least one anabolic biosynthetic pathway contained within the microorganism cells; wherein the carbon fixing reaction or anabolic biosynthetic pathway is at least partially driven by chemical and/or electrochemical energy provided by electron donors and/or electron acceptors contained within the gaseous substrate, which have been generated chemically and/or electrochemically and/or thermochemically and/or are introduced into the environment from at least one source external to the environment, and reacted by the microorganism cells within the environment; wherein the chemical reactants introduced into the environment comprise gaseous reactants, and wherein the organic products resulting from conversion of the carbon source and co-products of said conversion, and the products from the reaction of electron donors and electron acceptors within the environment are all solids and/or liquids and/or dissolved solutes, and wherein none of the said organic products or co-products from the conversion of the carbon source, or the products from the reaction of electron donors and electron acceptors thermodynamically favor the gas phase, and; wherein increased partial pressures of the gaseous reactants contained within the environment increase the thermodynamic driving force and kinetic rates for the conversion of the carbon source and/or the reaction of electron donors and electron acceptors. In certain embodiments of the present invention, the said elevated pressure is at least 1 bar gauge higher pressure than the ambient pressure outside of the bioreactor. In certain such embodiments, the said gaseous substrate comprises the said carbon source. In certain embodiments the said microorganism cells are chemoautotrophic. In certain embodiment the said carbon source is CO₂, the said electron donor is H₂, the said electron acceptor is O₂, and the said microorganism cells comprise knallgas microorganisms. In certain embodiments the said knallgas microorganisms comprise Cupriavidus necator. In certain embodiments the said knallgas microorganisms comprise microorganisms selected from one or more of the following genera: Cupriavidus sp., Rhodococcus sp., Hydrogenovibrio sp., Rhodopseudomonas sp., Hydrogenobacter sp., Gordonia sp., Arthrobacter sp., Streptomycetes sp. Rhodobacter sp., and/or Xanthobacter sp. In certain embodiments the said bioreactor is run in a continuous process wherein fresh, cell-free culture medium is continually flowed into the environment, and culture broth containing cells and/or the products of biosynthesis are continually removed from the environment. In certain such the said bioreactor is run as a turbidostat and/or a chemostat. In certain embodiments the said bioreactor is connected to an external gas recirculation loop. In certain embodiments the said electron donor is hydrogen generated by the electrolysis of water performed using one or more of: Proton Exchange Membranes (PEM); liquid electrolytes such as KOH; alkaline electrolysis; Solid Polymer Electrolyte electrolysis; high-pressure electrolysis; and high temperature electrolysis of steam (HTES). In certain embodiments the said electron acceptor is oxygen that is also generated by the electrolysis of water. In certain embodiments the said electron donors and/or electron acceptors are generated or recycled using renewable, alternative, or conventional sources of power that are low in greenhouse gas emissions, and wherein said sources of power are selected from at least one of photovoltaics, solar thermal, wind power, hydroelectric, nuclear, geothermal, enhanced geothermal, ocean thermal, ocean wave power, and tidal power. In certain embodiments the said electron donors and/or electron acceptors are generated using grid electricity during periods when electrical grid supply exceeds electrical grid demand, and wherein storage tanks buffer the generation of said electron donors and/or electron acceptor, and their consumption in the said carbon-fixing reaction. In certain embodiments the bioreactor or bioreactors of the present invention comprise but are not limited to one or more of the following: a stirred tank reactor (STR); a stirred tank reactor (STR) with a hollow gas entrainment impeller utilized to re-entrain headspace gases; a bubble column; a gas lift bioreactor; a trickle bed bioreactor; a pressure cycle loop bioreactor; a mechanically stirred loop; an ejector loop reactor or a venturi bioreactor; a membrane bioreactor.

In certain embodiments of the present invention, microorganism strains are grown chemoautotrophically. Certain embodiments of the present invention comprise a gas bioprocess that entails the feeding of relatively insoluble gases into an aqueous nutrient medium to provide the microorganisms energy and carbon-source required for growth. Efficient gas transfer into solution, and complete or substantially complete gas utilization, are key to achieving an economically viable and safe operation. Certain embodiments of the present invention increase the efficiency of gas transfer into solution and/or increase gas utilization and/or reduce any waste of gaseous feedstocks.

Operation at high pressures. In certain embodiments of the present invention, a method for growth of a microorganism culture (bioprocess) is run at high pressures, i.e., pressures greater than atmospheric pressures. In certain embodiments of the present invention, a bioreactor contains a high-pressure environment, i.e., an environment with a pressure greater than atmospheric pressure. In certain embodiments of the present invention, the bioprocess is run at a nearly unprecedented pressure or an unprecedented pressure for a bioprocess. In certain embodiments of the present invention, H₂ and/or O₂ and/or CO₂ gases are used as feedstocks. In certain embodiments using H₂ and O₂ feedstocks, any H₂ and O₂ gases that are present within the bioreactor headspace and/or gas recirculation loops, and/or any other accumulated gas phase within the bioreactor system excluding gas bubbles in liquid suspension, are in a non-flammable mixture composition. In certain embodiments, H₂ and O₂ gases are present in a flammable mixture composition within certain gas bubbles held in liquid suspension, but by the time the bubbles rise and coalesce into a headspace and/or any other accumulated gas filled space or pipe, the mixture has a non-flammable composition. In certain such embodiments, this is due to the microbial culture consuming down H₂ and O₂ to the point where the gas mixture lies outside of the flammability range.

Certain embodiments of the present invention comprise a bioprocess that uses mixtures of, CO₂, O₂, and H₂. In certain embodiments, gases are only consumed in the bioprocess, they are not produced, e.g., a very important aspect that highly impacts the bioreactor and gas recirculation design is that gases are only consumed in the bioprocess, they are not produced. In certain such embodiments, only liquids and/or solids are produced. Thus, in such embodiments the degassing features used to remove metabolic waste gases, and particularly CO₂, which are found in many aerobic and anaerobic bioreactors used for sugar-based, methane-based, and carboxydotrophic bioprocesses, are not a requirement in a bioreactor as described herein. In such embodiments, the degassing features used to remove O₂ from bioprocesses based on the photosynthetic conversion of CO₂ (e.g., algal), are also not required. In certain such embodiments, degassing features are not included in the bioreactor or associated systems of the present invention.

In certain embodiments of the present invention, the design and operation of bioreactors operating at pressures of up to 50 bar, and/or above 50 bar, uses and/or adopts proven designs and best practices developed over the past century in high pressure chemical GTL processes. In certain such embodiments, proven designs and practices drawn from chemical GTL processes, which are utilized in the present invention, are unprecedented and/or novel in their application to a biological culture as opposed to a chemical reaction.

An increase in productivity with applied pressure (P) has been observed for Cupriavidus in knallgas bioprocesses as described in the experimental examples section below. Not intending for the present invention to be limited by theory, the observed effect of P on productivity may be explained using the equation for O₂ transfer rate (OTR g O₂/L/h) from gas phase to aqueous solution:

$$OTR = {k}_{L} a ( {k}_{H} {pO}_{2} - DO )$$

where k_{L}a is the mass transfer coefficient, k_{H} is Henry's constant, pO₂ is the partial pressure of O₂, and DO is the dissolved O₂ in the bulk solution. The partial pressure of O₂ is equal to the mol fraction of O₂ (f_{O2}) multiplied by the total pressure i.e., pO₂=f_{O2} P.

The equation relating the OTR and the productivity (p) of Alcaligenes eutrophus (old name for Cupriavidus necator) on H₂/CO₂/O₂, as described by Tanaka et al (1995) [Tanaka, K., Ishizaki, A., Kanamaru, T., & Kawano, T. (1995). Production of poly(D-3-hydroxybutyrate) from CO₂, H₂, and O₂ by high cell density autotrophic cultivation of Alcaligenes eutrophus. Biotechnol Bioeng. https://doi.org/10.1002/bit.260450312 is incorporated herein by reference in its entirety]:

$$p = {Y}_{{O}_{2}} \times OTR$$

where Y_{O2} is the O₂ yield (g biomass/g O₂)

Combining the equations, and given that the culture is generally O₂-limited in a CSTR running on non-flammable, H₂-rich/O₂-lean gas mixtures, with the DO able to be approximated as roughly ˜0, (except at extremely high dilution rates/low optical density (OD)), the productivity and P are related as follows:

$$p = {Y}_{{O}_{2}} \times {k}_{L} a ( {k}_{H} {pO}_{2} - DO ) \approx {Y}_{{O}_{2}} {k}_{L} a {k}_{H} {pO}_{2} = {Y}_{{O}_{2}} {k}_{L} a {k}_{H} {f}_{{O}_{2}} P$$

From this equation it can be seen that the productivity is expected to increase with the pressure, provided the product of the pre-factor terms (Y_{O2}k_{L}ak_{H}f_{O2}) does not decrease in a way that counteracts or negates the increase provided by increasing P.

Fermentations with non-barophilic yeast and bacteria have been run successfully at pressures up to 130 bar. Moreover, pressure-tolerant phenotypes have been selected by adaptive evolution of non-barophilic microorganisms [Bartlett, D. H. (1992) Microbial life at high pressures. Sci Prog Oxford 76: 479-496]. H₂-oxidizing bacteria are known to be dominant primary producers at the base of the food chain in deep sea hydrothermal vent communities where the ambient pressures often exceed 50 bar [Adam, N., and Perner, M. (2018). Microbially mediated hydrogen cycling in deep-sea hydrothermal vents. Frontiers in Microbiology. https://doi.org/10.3389/fmicb.2018.02873]. This includes species that appear to be related to Cupriavidus. For example, [NiFe]-hydrogenase genes identified in the metagenome of a chimney sample from the hydrogen-rich, ultramafic Lost City hydrothermal field reportedly show the greatest resemblance to Ralstonia eutropha hydrogenase (R. eutropha is a species synonym for Cupriavidus necator) [Brazelton, W. J., Nelson, B., & Schrenk, M. O. (2012). Metagenomic evidence for H₂ oxidation and H₂ production by serpentinite-hosted subsurface microbial communities. Frontiers in Microbiology. https://doi.org/10.3389/fmicb.2011.00268]. The Lost City vents are reportedly at water depths from 700 to 800 meters [Connelly, D. P., Copley, J. T., Murton, B. J., Stansfield, K., Tyler, P. A., Wilcox, S. (2012). Hydrothermal vent fields and chemosynthetic biota on the world's deepest seafloor spreading center. Nature Communications. https://doi.org/10.1038/ncomms1636], which corresponds to hydrostatic pressures of from 70 to 80 bar. So, while some Cupriavidus strains are isolated from an environment at close to ambient pressures (i.e., ˜ 1 bar), it appears that closely related species, perhaps even members of Cupriavidus sp./Cupriavidus necator itself, may have evolved to live at pressures that exceed 50 bar. In certain embodiments of the present invention, a hydrogen-oxidizing organism is utilized that has been isolated from a natural environment, such as but not limited to a deep-sea hydrothermal vent, where the ambient pressure is greater than atmospheric pressure, or is around 50 bar, or exceeds 50 bar. In certain such embodiments, the microorganism (e.g., isolated microorganism) is a member of Cupriavidus or Ralstonia. In certain such embodiments, the microorganism (e.g., isolated microorganism) has a [NiFe]-hydrogenase gene closely related to Ralstonia eutropha hydrogenase.

Compression of synthesis gas, e.g., H₂ and N₂, up to the pressure level of the synthesis loop in the Haber-Bosch process, has in the past used reciprocating compressors. The H₂ and N₂ was typically compressed to around 300 bar in the majority of the plants up to the mid-20^{th} century. Even higher pressures were used in a few installations, for example, in Claude and Casale units.

In certain embodiments of the present invention, reciprocal compressors are utilized to compress a gas stream that is used in high pressure bioreactors of the present invention. In certain such embodiments, the said gas stream comprises one or more of H₂, CO₂, O₂, CO, and/or CH₄. In certain embodiments, one or more gas streams entering a bioreactor is compressed using reciprocal compressors. In certain such embodiments each of these said gas streams comprises one or more of H₂, CO₂, O₂, CO, and/or CH₄. In certain such embodiments, reciprocal compressors are utilized to compress the said gas stream to at least about any of 5 bar, 10 bar, 20 bar, 40 bar, 80 bar, 160 bar, 320 bar or greater than 320 bar. In certain embodiments of the present invention, a gas generation process (e.g., generation of H₂, CO₂, and/or CO) and/or water gas shift conversion is utilized that operates at close to atmospheric pressure. In certain embodiments of the present invention a gas stream is first compressed to around 25 bar, at which pressure, in certain embodiments a CO₂ removal step is performed, and afterwards to around 50 bar or less or around 100 bar or less or around 200 bar or less or around 300 bar or less. In certain such embodiments, an additional purification step is performed at the higher pressure. In certain such embodiments, the said CO₂ removed at the CO₂ removal step and/or the said purified gas stream is fed into a high pressure bioreactor of the present invention. In certain embodiments, steam reforming and/or partial oxidation generate synthesis gas at a pressure level sufficient to flow directly into a CO₂ removal operation without any additional compression. As gasification proceeds with a considerable volume increase and feedstocks such as natural gas are usually already available under pressure at battery limits, considerable savings in compression energy can be achieved in this way. In certain embodiments of the present invention, reciprocating compressors with as many as seven stages (e.g., 1, 2, 3, 4, 5, 6, or 7 stages) in linear arrangement are utilized. In certain such embodiments, intermediate cooling is used. In certain embodiments, a CO₂ removal section is installed between two stages, such as between the 3rd and 4th stages of reciprocal compressors. In certain embodiments of the present invention, a reciprocating compressor with a suction volume of up to 15,000 m³ at standard temperature and pressure (STP) or at least 15,000 m³ (STP) or greater than 15,000 m³ (STP) is used in the first stage of compressors. In certain embodiments, flywheels are used as the rotors of synchronous motors with rotational frequencies of around 125 rpm and with two crankshafts on both sides connected over crossheads with the piston rod for horizontally arranged stages. In certain embodiments of the present invention, gas engines are used as drivers of reciprocating compressors. In certain embodiments, electric motors are used as drivers of reciprocating compressors. In certain embodiments, horizontally balanced compressors are utilized in which the cylinders are in parallel configuration on both sides of a common crankshaft. In certain embodiments, asynchronous motors are used to drive compressors. In certain embodiments, gas engine drivers are used to drive compressors. In certain such embodiments, the said gas engine drive is a two-stroke type. In certain gas engine drivers used in the present invention, a common crankshaft is used for the piston rods of the gas machine cylinders and the compressor cylinders.

In certain embodiments, steam turbines with speed reduction gears are used to drive compressors. In certain embodiments, the various compression services, e.g., fuel gas, process air or oxygen, and synthesis gas (e.g., H₂ and CO₂) compression, are apportioned among the crankshaft throws in such a manner that a single compressor can perform multiple or all compression duties.

Centrifugal compressors are typically used in modern single-train ammonia plants. The first of these type of plants, having capacities of around 600 t/d, were built in the mid-1960s. In more modern plants with sizes in the range of 1200-2000 t/d plants centrifugal compressors can produce operating pressures in the range of 170-190 bar.

In certain embodiments of the present invention, centrifugal compressors are used for the compression duties of gases, such as but not limited to H₂, CO₂, and/or O₂, and gas recycle, process air, and refrigeration. In certain embodiments of the present invention, the centrifugal compressors are directly driven by steam turbines. This avoids the losses associated with generation and transmission of electric power. In certain embodiments of the present invention, the gas compressors, including recycle, are almost or are exclusively driven by steam turbines. In certain such embodiments, the steam turbines are extraction turbines with a condensing section. In certain such embodiments, steam is extracted at suitable pressure levels (e.g., about 45-about 55 bar) to provide, for example, the process steam in steam reforming plants, and for other drivers, e.g., air compressor, refrigeration compressor, boiler feed water pumps, and blowers. In certain embodiments the extracted steam is utilized for heat treatment in downstream processing (DSP) of biomass produced according to the present invention. In certain embodiments of the present invention, gas turbines are used as drivers for compressors. In certain such embodiments, the exhaust may be used for steam production, for preheating duties, or as combustion air in the primary reformer. In certain embodiments, the carbon dioxide is used as a carbon source in the bioreactor.

In certain embodiments, the passage width at the outer circumference of a centrifugal compressor impeller is around 2.8 mm. In certain embodiments of the present invention a centrifugal compressor is used to produce pressures of at least 5 bar or at least 10 bar or at least 20 bar or at least 40 bar or at least 80 bar or around 145-150 bar or over 150 bar including in a range of around 170-190 bar. In certain embodiments, the minimum gas flow from the last wheel of the centrifugal compressor is 350 m³ (STP) or less. In certain embodiments, the maximum wheel tip speed in the centrifugal compressor is around 330 m/s or higher. In certain embodiments of the present invention the centrifugal compressor has at least one impeller or at least 2 impellers or at least 4 impellers or at least 8 impellers or at least 16 impellers or around 18 to 20 impellers or less. In certain embodiments, the pressure is increased in a gas stream, for example, from about 25 to about 200 bar, using a centrifugal compressor with around 18-20 impellers. A compressor shaft must have sufficient rigidity to avoid excessive vibration, and this limits the possible length. In certain embodiments the compressor shaft has at least one impeller or at least 2 impellers or at least 4 impellers or at least 8 impellers or around eight or nine impellers. In certain embodiments, several compressor casings are arranged in series. In certain embodiments, each compressor casing has a compression ratio of around 1.8 to 3.2. In certain embodiments, the number of compressor casings is one or two. In certain embodiments, geared or metal diaphragm couplings are used to connect the shafts of the individual casings.

In certain embodiments of the present invention, other compression duties in the plant, such as but not limited to process air in steam reforming plants and air, oxygen, and nitrogen compression in partial oxidation plants, are also performed by centrifugal compressors. In certain embodiments compression in the refrigeration section is performed using centrifugal compressors. In certain embodiments, screw compressors are utilized.

In certain embodiments, the bearing temperatures and/or the axial position of the rotor and/or the radial vibrational deflections are continuously monitored by sensors.

In certain embodiments, a rotating shaft is sealed against the atmosphere. Certain embodiments do not use mechanical contact shaft seals. In certain embodiments, liquid-film shaft seals with cylindrical bushings (floating rings) are applied. In certain embodiments, an oil film between the shaft and a floating ring, capable of rotation, provides the sealing. In certain embodiments, a floating ring is sealed to a compressor casing by O-rings. In certain embodiments, seal oil flows between both halves of a floating ring. In certain such embodiments, part of the oil returns to a reservoir, while the remainder flows against the gas pressure into a small chamber from which, together with a small quantity of gas, it is withdrawn through a reduction valve. In certain embodiments the seal oil pressure in the floating ring cavity is slightly higher than the gas pressure within the casing. In certain such embodiments the higher pressure is provided by a static height difference of the oil level in the elevated oil buffer vessel. In certain embodiments, no oil contamination enters the gas stream. In certain embodiments, a seal oil supply is combined with a lubricating oil system. In certain such embodiments, the oil reservoir, filters and/or pumps are shared.

In certain embodiments of the present invention, centrifugal compressors are used with dry, oil-less gas seals. In certain embodiments nitrogen or carbon dioxide is used as an inert fluid for the seal, which is achieved at the radial interface of rotating and stationary rings. During operation, the seal is not completely tight; some of the seal gas flows back to the suction side to be re-compressed, and a small amount from the suction side may go to the atmospheric side. During stops, when the shaft is not rotating, the seal ring is pressed tight against the seat by means of a spring and the differential gas pressure. In certain embodiments, a dry gas seal is used in combination with oil-lubricated bearings (dry/wet). In certain embodiments, there is no contact between oil and gas.

In certain embodiments of the present invention, magnetic bearings are used in centrifugal compressors. In certain embodiments, a combination of magnetic bearings and dry seals (dry/dry) are utilized, which totally replace the oil system.

In certain embodiments of the present invention integrated geared centrifugal compressors are utilized. In certain such embodiments, the driver (e.g., a steam turbine) drives a common gear to which the individual compression stages are connected. In certain embodiments, each stage has a single impeller which runs at a speed of around 25,000 rpm or higher. In certain embodiments, the compressors have three or four stages. In certain such embodiments the gas flow is around 75,000 m³/h at a pressure of around 75 bar.

In certain embodiments of the present invention, compressor control is achieved by controlling the rotational speed of the driver. In certain such embodiments, the rotational speed of the driver is controlled using a distributed control system (DCS). If the volumetric flows through the machine at start-up or during reduced load operation deviate too far from the design values, it may be necessary to re-circulate gas through individual stages or through the whole machine. Otherwise, the compressor can enter a state of pulsating flow, called surge, which could cause damage. In certain embodiments, an anti-surge control (minimum by-pass control, kickback control) is utilized to prevent this condition, as well as to minimize the incidence and degree of uneconomical recirculation.

Certain aspects of the present invention relate to bioreactor and bioprocess designs and methods that extend the range of possible operating pressures in a microbial bioprocess out to around at least 5 bar pressure, or at least 10 bar, or at least around 20 bar, or at least around 30 bar, or at least around 50 bar, and/or above 50 bar (i.e., >50 bar). In certain embodiments of the present invention, input gases (e.g., CO₂, H₂, and/or O₂) are compressed and pumped into a bioreactor that contains a microbial culture (e.g., a knallgas culture) maintained at an elevated pressure inside the bioreactor vessel. In certain embodiments the elevated pressure is at least around 3 bar, or at least around 5 bar, or at least around 10 bar, or at least around 20 bar, or at least around 40 bar, or at least around 50 bar, or at least around 80 bar, or at least around 160 bar, or at least around 320 bar, or at least around 400 bar pressure. In certain such embodiments, the culture growth in the bioreactor held at elevated pressure exhibits increased productivity and/or yields compared to the culture growth at close to ambient pressures. In certain such embodiments the culture comprises one or more knallgas species. In certain such embodiments the culture comprises a Cupriavidus species. In certain embodiments, the type of bioreactor maintained at elevated pressure is a stirred tank reactor (STR). In certain embodiments the STR maintained at elevated pressure is operated as a continuous stirred tank reactor (CSTR). In certain non-limiting embodiments a CSTR held at a pressure falling within a range of from around 3 bar to around 5 bar is used to produce Cupriavidus necator biomass from CO₂ carbon source at a productivity of greater than around 1 g/L/h or greater than around 1.5 g/L/h or greater than around 2 g/L/h or greater than around 2.5 g/L/h or greater or equal to around 2.85 g/L/h or greater or equal to around 3 g/L/h. In certain embodiments, a CSTR bioprocess run as a turbidostat at around 4 bar pressure, on CO₂ as the sole carbon source to grow C. necator, and produce biomass and biochemicals, has an average biomass productivity of around 2.85 g/L/h. In certain such embodiments an average productivity of around 2.85 g/L/h is maintained and calculated over a runtime period of around 24 hours or longer, or around 48 hours or longer, or around 72 hours or longer, or around a week or longer, or around a month or longer, or around 100 days or longer, or around 200 days or longer, or around 300 days or longer. Certain embodiments of the present invention comprise a bioprocess involving CO₂, H₂, or O₂ gaseous inputs and inputs of dissolved inorganic mineral nutrients to a culture comprising C. necator contained within a CSTR held at a target pressure where there is a strong positive correlation between pressure and the productivity (g/L/h) of biomass and/or biochemicals from CO₂. In certain embodiments, the positive correlation in the said bioprocess between reactor pressure and productivity on CO₂ continues up to at least around 5 bar, or at least around 10 bar, or at least around 20 bar, or at least around 50 bar, or at least around 100 bar. In certain embodiments of the present invention, part of the pressure applied to the knallgas culture (e.g., C. necator culture) is due to hydrostatic pressure that results from scaling-up the bioreactor in the vertical direction and thus deepening the water column in the bioreactor. In certain embodiments of the present invention, scaling-up the bioreactor in the vertical direction results in an increase in the average hydrostatic pressure experienced by a knallgas culture (e.g., a C. necator culture), which in turn results in an increased average productivity of the culture on CO₂.

In certain embodiments, a bioreactor of the present invention is held at a certain target pressure of at least around 2 bar, or at least around 3 bar, or at least around 4 bar, or at least around 5 bar, or at least around 10 bar, or at least around 20 bar, or at least around 50 bar, or at least around 100 bar, or at least around 200 bar, while other process parameters are also measured and controlled such as but not limited to one or more of the following: optical density of the culture measured at 600 nm (OD₆₀₀); reactor temperature (T ° C.); pH; base delivery (L) required to maintain a target pH; the dissolved O₂ in the culture broth (DO); and the rate of the continuous feed of fresh aqueous inorganic mineral media (L/h) during continuous operation. In certain such embodiments, the targeted pH is around pH ˜7; In certain embodiments comprising a bioreactor held at an elevated pressure of at least around 2 bar, or at least around 3 bar, or at least around 4 bar, or at least around 5 bar, or at least around 10 bar, or at least around 20 bar, or at least around 50 bar, or at least around 100 bar, or at least around 200 bar, input of aqueous mineral media is balanced by a continuous withdrawal of culture broth such that a roughly constant OD₆₀₀ is maintained within the bioreactor overtime. In certain such embodiments, the said roughly constant OD₆₀₀ falls within a range of OD₆₀₀ covering from around OD₆₀₀˜5 to around ˜200, or around OD₆₀₀˜10 to 100, or around OD₆₀₀˜20 to 80, or around OD₆₀₀˜40 to 70, or around OD₆₀₀˜50 to 60. In certain embodiments, the said continuously harvested culture broth is subjected to downstream processing (DSP) which includes but is not limited to dewatering and/or drying and/or further protein purification operations. In certain embodiments the harvested culture broth goes directly into DSP, while in other embodiments the harvested maybe stored in a cooled buffer tank (e.g., around 4° C.) for up to around a day, from which it is withdrawn and then subjected to DSP. In certain embodiments, a bioreactor held at a certain target pressure of at least around 2 bar, or at least around 3 bar, or at least around 4 bar, or at least around 5 bar, or at least around 10 bar, or at least around 20 bar, or at least around 50 bar, or at least around 100 bar, or at least around 200 bar, is run as a turbidostat. In certain embodiments, a bioreactor held at a certain target pressure of at least around 2 bar, or at least around 3 bar, or at least around 4 bar, or at least around 5 bar, or at least around 10 bar, or at least around 20 bar, or at least around 50 bar, or at least around 100 bar, or at least around 200 bar, has a roughly constant liquid volume in the bioreactor (i.e., working volume) over the duration of the continuous runtime via the balancing of the input of aqueous mineral media into the bioreactor, water production by the culture inside the bioreactor (e.g., knallgas culture), and the continuous withdrawal of culture broth out of the bioreactor. In certain embodiments of the present invention, the bioreactor is run at elevated pressure as a chemostat. In certain embodiments of the present invention, the bioreactor is run at elevated pressure as both a turbidostat and a chemostat simultaneously. In certain embodiments, a bioreactor of the present invention is held at a certain target pressure of at least around 2 bar, or at least around 3 bar, or at least around 4 bar, or at least around 5 bar, or at least around 10 bar, or at least around 20 bar, or at least around 50 bar, or at least around 100 bar, or at least around 200 bar, and operated continuously for at least around 3 days, or at least around 7 days, or at least around 10 days, or at least around 16 days, or at least around 20 days, or at least around 30 days, or at least around 100 days, or at least around 200 days, or at least around 300 days.

In certain embodiments, a bioreactor of the present invention under continuous operation is gradually ramped up in pressure overtime. In certain such embodiments, the ramping up in pressure occurs over around at least 3 days or at least around 7 days, or at least around 10 days, or at least around 16 days, or at least around 20 days, or at least around 30 days, or at least around 100 days, or at least around 200 days, or at least around 300 days. In certain embodiments, the bioreactor pressure is incremented up around one bar pressure every few days while the bioreactor is continuously operating. In certain embodiments, the bioreactor pressure is incremented up more than one bar pressure every few days while the bioreactor is continuously operating. In certain non-limiting embodiments, the bioreactor is incremented from close to ambient pressure up to around 2 bar, and then to around 3 bar, and then to around 4 bar, and then to around 5 bar pressure. In certain such embodiments, the bioreactor is held for several days at each pressure (e.g., around 48 to 72 hours). In certain embodiments, other run parameters including but not limited to one or more of the following: agitation rate; pH; T; OD₆₀₀; input molar gas flows; and % composition of H₂, CO₂, and O₂; are targeted at constant values while the pressure is varied (e.g., ramped up) and/or incremented up or down. In certain embodiments, the pressure is continuously increased or decreased (e.g., at a constant bar per time rate) during a run until a target pressure is reached and then maintained. In certain embodiments, the dilution rate (μh⁻¹) of the bioreactor is allowed to vary as the pressure is ramped-up or down in order to maintain a targeted OD₆₀₀ (i.e., turbidostatic operation). In certain embodiments of the present invention, process parameters including but not limited to one or more of the following: agitation rate; pH; T; OD₆₀₀; input molar gas flows and % gas composition of H₂, CO₂, and O₂; mineral nutrient medium composition; are measured, monitored, and controlled (e.g., optimized) while the pressure is increased or decreased and/or incremented upwards or downwards. In certain such embodiments, process parameters including but not limited to one or more of the following: agitation rate; pH; T; OD₆₀₀; input molar gas flows and % gas composition of H₂, CO₂, and O₂; mineral nutrient medium composition; continue to be measured, monitored, and controlled (e.g., optimized) once a target pressure (e.g., optimal pressure) has been reached and maintained. In certain embodiments, the data on process parameters is analyzed and subjected to methods known in the field of design of experiments, data science, and/or artificial intelligence to further enhance productivity gains with increasing reactor pressure and/or to improve (e.g., optimize) other run metrics such as but not limited to one or more of the following: H₂ yield (e.g., g biomass/g H₂ consumed); O₂ yield (e.g., g biomass/g O₂ consumed); CO₂ yield (e.g., g targeted organic molecule/g CO₂ consumed where the targeted organic molecule may be a specific protein, amino acid, lipid, polysaccharide, biopolymer, etc.); weight % protein; weight % targeted organic compound (e.g., amino acid, peptide, lipid, PHB, etc.), H₂ conversion (i.e., g H₂ consumed/g H₂ input), CO₂ conversion (i.e., g CO₂ consumed/g CO₂ input), O₂ conversion (i.e., g O₂ consumed/g O₂ input), OPEX (e.g., $/kg product), CAPEX (e.g., $/kg product), cost of goods sold (e.g., $/kg product),

It has been observed that while a positive correlation generally exists between total P and productivity for CO₂ conversion bioprocesses run using a C. necator culture according to the present invention, the rate at which the productivity increases with pressure varies depending upon other process parameters including but not limited to: agitation rate; pH; T; OD₆₀₀; input molar gas flows (e.g., VVM and specific gas velocity) and % composition of H₂, CO₂, and O₂; and mineral nutrient medium composition. Certain aspects of the present invention relate to measuring, monitoring, and controlling (e.g., optimizing) parameters including but not limited to one or more of the following: agitation rate; pH; T; OD₆₀₀; input molar gas flows (e.g., VVM and specific gas velocity) and % gas composition of H₂, CO₂, and O₂; mineral nutrient medium composition, so as to improve, increase, and/or maximize the rate of increase in bioprocess productivity with increasing operating pressure (i.e., g/L/h increase per bar increase). An improvement in the productivity response by the system (i.e., bioprocess) to increasing operational pressure can be confirmed by means including an observed to increase in the slope of the power law or linear trendline fit to the productivity versus pressure trend. In certain embodiments, the slope of the power law and/or linear trendline fit to the productivity versus pressure trend of bioprocesses run according to the present invention and increased through selecting improved or optimal set point values for the various run parameters including but not limited to one or more of the following: agitation rate, pH, T, OD₆₀₀, input molar gas flows (e.g., standard VVM and/or specific gas velocities); % composition of H₂, CO₂, and O₂; and/or mineral nutrient medium composition. In certain such embodiments, the judicious selection (e.g., optimization) of the said run parameters can increase the productivity achieved at a given pressure by at least 50%, or at least 100%, or at least 150%, or at least 200%, or at least 300%. In certain such embodiments, the said increases in productivity achieved at a given pressure are also realized while simultaneously limiting the mol % of O₂ in the reactor headspace to mol % O₂≤5%, or mol % O₂≤4%, or mol % O₂≤3%, or mol % O₂≤3%, or mol % O₂≤2%, or mol % O₂≤1%. In a specific embodiment of the present invention, the selection of an improved group of set point values for various run parameters including but not limited to one or more of the following: agitation rate, pH, T, OD₆₀₀, input molar gas flows (e.g., standard VVM and/or specific gas velocities); % composition of H₂, CO₂, and O₂; and/or mineral nutrient medium composition, improve the productivity in a CSTR run at 4 bar pressure from around 1 g/L/h to around 1.5 g/L/h to around 2.5 g/L/h to 3 g/L/h, while in all cases limiting the mol % of O₂ in the reactor headspace to mol % O₂≤5%.

In certain embodiments of the present invention, the bioreactor pressure is ramped overtime to at most a pressure of around 50 bar, and then held at that end point pressure and operated continuously. In other embodiments of the present invention, the bioreactor pressure is ramped over time to a pressure of at least around 50 bar, and then held at that end point pressure and operated continuously. In certain embodiments of the present invention, the bioreactor productivity on CO₂ increases as the pressure increases. In certain such embodiments, the increase in productivity with pressure follows a power law fit. In certain embodiments, the power law fit of the bioprocess productivity to bioreactor pressure has an exponent in the range of around 0.5 to 1, or around 0.6 to 0.9, or around 0.65 to 0.85. In certain embodiments, the power law fit of the bioprocess productivity to bioreactor pressure has an exponent of around 0.7-0.8, and a constant pre-factor coefficient of around one. In certain embodiments, the increase in productivity with pressure follows a linear trend. In certain embodiments, the power law relationship or linear trend between productivity on CO₂ and pressure may be used to target a particular productivity by targeting a particular operating pressure. In certain embodiments, a power law relationship or a linear trend between productivity on CO₂ and pressure extends out to pressures used in established chemical GTL processes, but which are unprecedented in commercial bioprocesses and fermentations. In certain embodiments, the trend of increasing productivities on CO₂ carbon source with increasing operating pressure is continued by a combination of elevating bioreactor pressure, and control (e.g., optimization) of other run parameters, out to pressure ranges used in established chemical GTL processes. In certain embodiments, by extending the trend of increasing productivity with increasing operating pressure out to pressure ranges used in established chemical GTL processes ultra-high productivities are reached on CO₂ that are unprecedented for any biological process on any substrate: heterotrophic, chemotrophic, or photosynthetic. In certain embodiments, ultra-high biomass productivities on CO₂ are attained in a bioprocess of the present invention by operating at pressures similar to those used in established chemical GTL processes, where the said biomass productivities are unprecedented for any biological process on any substrate; heterotrophic, chemotrophic, or photosynthetic. In certain embodiments, the bioreactor of the present invention is operated at a target pressure that falls within the range of 10 to 45 bar, which is the pressure range that covers pressures commonly used in the Fischer-Tropsch process [Botes, F. G., Dancuart, L. P., Nel, H. G., Steynberg, A. P., Vogel, A. P., Breman, B. B., & Font Freide, J. H. M. (2011). Middle distillate fuel production from synthesis gas via the Fischer-Tropsch process. In Advances in Clean Hydrocarbon Fuel Processing: Science and Technology. https://doi.org/10.1533/9780857093783.4.329]. In certain embodiments, the bioreactor of the present invention is operated at a target pressure that falls within the range of 40 to 120 bar, which is the pressure range that covers pressures commonly used in methanol synthesis [https://www.netl.doe.gov/research/coal/energy-systems/gasification/gasifipedia/methanol]. In certain embodiments, the bioreactor of the present invention is operated at a target pressure that falls within the range of 150 to 400 bar, which is the pressure range that covers pressures commonly used in the Haber-Bosch process [Appl, M. (2011). Ammonia, 2. Production Processes. In Ullmann's Encyclopedia of Industrial Chemistry. https://doi.org/10.1002/14356007.o02_o11]. In certain embodiments of the present invention, methods and equipment that have developed in the chemical industry for setting up and operating high pressure reactions, and in particular high pressure reactions involving H₂ reactant, and more particularly GTL processes where gaseous reactants comprising H₂ are converted to liquid and/or non-gaseous products in a process that is thermodynamically and/or kinetically driven by elevated pressure for the production of low cost, high volume commodities such as ammonia, methanol, and FT-diesel, are adopted, adapted, and/or repurposed for the high pressure bioprocesses of the present invention. In certain embodiments of the present invention strain and/or process engineering is utilized to extend the observed trend of increasing productivity with pressure out to GTL-type pressures. In certain embodiments of the present invention, elevated pressure in combination with control (e.g., optimization) of other bioprocess parameters, is used to attain biomass productivities of at least around ≥2 g/L/hr, or ≥3 g/L/hr, or ≥5 g/L/hr, or ≥10 g/L/hr, or ≥20 g/L/hr, or ≥30 g/L/hr, or ≥40 g/L/hr, or ≥50 g/L/hr, or ≥70 g/L/hr, or ≥90 g/L/hr, or ≥100 g/L/hr. In certain such embodiments, the highest bioprocess productivities ever recorded on any substrate—heterotrophic, chemotrophic, or photosynthetic—are attained. In certain such embodiments, the said high productivities are maintained for extended periods of times (e.g., at least 24 hours, or at least 48 hours, or at least 72 hours, or at least 1 week, or at least 1 month, or at least 100 days, or at least 200 days, or at least 300 days), by continuous operation of the said high pressure bioprocess (e.g., as a CSTR).

While not intending for the present invention to be limited by theory, if the O₂ yield, k_{L}a, k_{H}, and f_{O2} terms remained constant with increasing P, then one would expect a roughly linear increase in productivity with pressure by the aforementioned relationship:

$$p = {Y}_{{O}_{2}} \times {k}_{L} a ( {k}_{H} {pO}_{2} - DO ) \approx {Y}_{{O}_{2}} {k}_{L} a {k}_{H} {pO}_{2} = {Y}_{{O}_{2}} {k}_{L} a {k}_{H} {f}_{{O}_{2}} P$$

However, it has been observed that the increase in productivity with P often appears to be less than linear, and better fit with a power law that generally has an exponent less than 1 i.e., sublinear. While not intending for the present invention to be limited by theory, in cases where f_{O2} is held fixed, it suggests that one or more of Y_{O2}, k_{L}a, or k_{H} can decrease as P is increased (and f_{O2} held fixed). Certain aspects of the present invention relate to designs and measures to prevent or mitigate any decreases in Y_{O2}, k_{L}a, k_{H}, or f_{O2} as P is increased. Certain aspects of the present invention relate to increasing one or more of the following terms: Y_{O2}, k_{L}a, k_{H}, or f_{O2}, as P is increased, while avoiding or minimizing a decrease in the other terms. In certain embodiments, the f_{O2} term is increased, while simultaneously keeping the mol % of O₂ in the bioreactor headspace and/or any other accumulated gaseous phases within the bioreactor system (e.g., gas recirculation loops, vent lines, etc.) below the MOC. In certain embodiments, designs, equipment, and/or methods drawn from the known art and science of aerobic bioprocessing, gas fermentation, and/or chemical GTL processes are applied to prevent k_{L}a from decreasing with increasing P and/or else at least minimizing any reduction that occurs with increasing P.

In certain embodiments of the present invention, the dilution rate (μh⁻¹) of a CSTR, and/or another continuous bioreactor design, is varied while maintaining the said bioreactor at a targeted pressure (P) and other run parameters (e.g., agitation rate, pH, T, input molar gas flows and % composition of H₂, CO₂, and O₂, mineral nutrient medium composition etc.). In certain such embodiments, the OD₆₀₀ is allowed to vary in response the change in dilution rate. OD₆₀₀ generally decreases as the dilution rate increases for a given set of other run parameters (e.g., P, agitation rate, pH, T, input molar gas flows and % composition of H₂, CO₂, and O₂, mineral nutrient medium composition etc.). When run parameters including but not limited to P, agitation rate, pH, T, input molar gas flows and % composition of H₂, CO₂, and O₂, mineral nutrient medium composition, etc. are held constant, a positive correlation is generally observed between the inverse H₂ yield (1/Y_{H2}) and the inverse of the CSTR dilution rate (1/μ). In a turbidostat the dilution rate μ equals the specific growth rate of the culture (g biomass produced/h/g standing biomass).

Not intending for the present invention to be limited by theory, the equation predicting this linear relationship:

$$\frac{1}{Y} = \frac{m}{μ} + \frac{1}{{Y}_{μ \_ inf}}$$

has been reported before by Bongers [Bongers, L. Energy generation and utilization in hydrogen bacteria. J. Bacteriol. (1970) doi:10.1128/jb.104.1.145-151.1970 is incorporated herein by reference in its entirety] and many other authors, where:
Y=observed yield (g biomass/mol H₂).
m=slope=maintenance energy (mol H₂/g biomass/h).
μ=specific growth rate (h⁻¹)=dilution rate in turbidostat CSTR.
Y_{μ_inf}=inverse intercept=ideal yield (g biomass/mol H₂) of cell synthesis in limit of the absence of any cell maintenance costs.

While for a given set of run parameters, a good linear fit is generally observed between 1/p and 1/Y for a turbidostatic CSTR operated according to the present invention, a different set of run (e.g., fixed) conditions (e.g., P, agitation rate, pH, T, input molar gas flows (standard VVM) and % composition of H₂, CO₂, and O₂, mineral nutrient medium composition etc.) can produce a different slope (m) an/or vertical axis intercept (1/Y_{μ_inf}). Nonetheless, the an increase in Y_{H2} with increasing p is generally observed. In certain embodiments of the present invention, the H₂ yield is increased to a targeted value by increasing the dilution rate in a CSTR of the present invention, and/or another continuous bioreactor-type of the present invention. In certain non-limiting embodiments of the present invention the H₂ yield is any of around or at least around any of Y_{H2}≥2 g biomass/g H₂, Y_{H2}≥2.5 g biomass/g H₂, Y_{H2}≥3 g biomass/g H₂, Y_{H2}≥3.35 g biomass/g H₂, or Y_{H2}≥3.45 g biomass/g H₂.

It has generally been observed that Y_{H2} increases in turbidostatic runs as P is increased. Not intending for the present invention to be limited by theory, one explanation for this is that as the productivity increases with P, the μ is increased to maintain a constant OD in a turbidostat. Therefore, by the above relationship between Y and p, one would expect Y_{H2} to increase due to the increasing p. In certain embodiments of the present invention, a targeted combination of Y_{H2} and productivity (i.e., increased or improved Y_{H2} and/or productivity) is attached in a CSTR and/or another continuous bioreactor according to the present invention by operating the said continuous bioreactor as a turbidostat as P is increased, which produces an increase in the μ and Y_{H2} as well as an increase in productivity, with increasing P. In certain embodiments of the present invention a H₂ yield of any of around or at least around Y_{H2}≥3 g biomass/g H₂, Y_{H2}≥3.35 g biomass/g H₂, Y_{H2}≥3.45 g biomass/g H₂, Y_{H2}≥3.5 g biomass/g H₂, or Y_{H2}≥3.7 g biomass/g H₂, is combined with a productivity of any of around or at least around ≥1 g/L/hr, ≥2 g/L/hr, ≥3 g/L/hr, ≥5 g/L/hr, ≥10 g/L/hr, ≥20 g/L/hr, ≥30 g/L/hr, ≥40 g/L/hr, ≥50 g/L/hr, ≥70 g/L/hr, ≥90 g/L/hr, ≥100 g/L/hr.

Not intending to be limited by theory, another factor to consider in addition to the aforementioned effect of dilution rate on increasing Y_{H2} with increasing P, is that the thermodynamic driving force for both the knallgas respiration reaction (i.e., H₂(g)+½O₂(g)→H2O(l)), as well as the biomass synthesis reactions (average empirical biomass reaction for C. necator found to be: 8.45H₂(g)+4.1CO₂(g)+NH₃(aq)→C_{4.1}H_{6.9}O_{1.7}N(s)+6.5H₂O(l)), will increase as the total P, and pH₂, pCO₂, and pO₂ increase. Since all the main reactants are gases, while all of the products are solids or liquids, increasing P and the partial pressures of the reactant gases will make both the CO₂-fixing and knallgas respiration reactions more thermodynamically favorable since they convert gases into liquids and solids. In certain embodiments of the present invention, the H₂ yield is increased by increasing P, pH₂, pCO₂, and pO₂.

In knallgas bioprocesses, the O₂ yield (Y_{O2} g biomass/g O₂) is highly positively correlated stoichiometrically with the H₂ yield (Y_{H2} g biomass/g H₂). In certain embodiments, methods of the present invention that are used to increase Y_{H2} simultaneously serve to increase Y_{O2}, and consequently positively impact productivity (i.e., increase productivity). Without intending to be limited by theory, in increase in productivity due to the simultaneous, correlated increase in Y_{H2} and Y_{O2} may be explained by the aforementioned equation p=Y_{O}_{₂}×k_{L}a(k_{H}pO₂−DO)≈Y_{O}_{₂}k_{L}ak_{H}pO₂=Y_{O}_{₂}k_{L}ak_{H}f_{O}_{₂}P. In certain embodiments of the present invention the productivity is increased via simultaneously increasing Y_{O2} and P. In certain such embodiments the increase in Y_{O2} is associated with an increase in Y_{H2}. In certain such embodiments a H₂ yield of any of around or at least around Y_{H2}≥3 g biomass/g H₂, Y_{H2}≥3.35 g biomass/g H₂, Y_{H2}≥3.45 g biomass/g H₂, Y_{H2}≥3.5 g biomass/g H₂, or Y_{H2}≥3.7 g biomass/g H₂, is combined with a productivity of any of around or at least around 2 g/L/hr, ≥3 g/L/hr, ≥5 g/L/hr, ≥10 g/L/hr, ≥20 g/L/hr, ≥30 g/L/hr, ≥40 g/L/hr, ≥50 g/L/hr, ≥70 g/L/hr, ≥90 g/L/hr, or ≥100 g/L/hr.

In knallgas bioprocesses, the O₂ yield (Y_{O2} g biomass/g O₂) is highly correlated stoichiometrically with the H₂ yield (Y_{H2} g biomass/g H₂). In certain embodiments of the present invention, methods used to increase Y_{H2} simultaneously serve to maintain or improve Y_{O2}, and consequently positively impact productivity. as P is increased should, in certain embodiments of the present invention.

In certain embodiments of the present invention, strain and/or process engineering is utilized to extend the observed trend of increasing productivity with P out to GTL-type pressures (e.g., up to any of around or at least around ≥10 bar, ≥20 bar, ≥30 bar, ≥40 bar, ≥50 bar, ≥100 bar, ≥200 bar, ≥300 bar, and/or ≥400 bar). In certain embodiments of the present invention, high pressure (P) bioreactors are utilized in a continuous knallgas bioprocess. Certain such embodiments operate in high P regimes that are unprecedented in bioprocesses, but which are commonplace in chemical GTL processes. Certain aspects of the present invention relate to leveraging extensive experience in running continuous knallgas bioprocesses at elevated pressures and experience and expertise in the art and science of designing, modeling, and constructing high P chemical reactors and in the chemical engineering of reactions involving flammable gases such as H₂ and/or syngas.

In certain embodiments of the present invention, the gas headspace of the bioreactor and/or any other significant accumulations of gaseous phase within the bioprocess system (e.g., gas recirculation loops, vent lines, etc.) is maintained with an O₂ concentration below the MOC (e.g., mol fraction of O₂ (f_{O2}%) constrained to f_{O2}%≤5%). In certain cases of such embodiments, the bubbles of gas held in liquid suspension within the bioreactor are not considered “significant accumulations” of gaseous phase. With the bioreactor headspace mol fraction of O₂ (f_{O2}%) constrained to below the MOC (e.g., f_{O2}%≤5%), elevating the total pressure (P) results in higher pO₂ at a fixed mol % given that pO₂=f_{O2}%*P (e.g., 0.05 bar O₂ at P=1 bar, 0.1 bar O₂ at P=2 bar, 0.15 bar O₂ at P=3 bar etc.). Not intending for the present invention to be limited by theory, it is believed this relaxes the O₂-limitation on growth of a knallgas culture that often exists when a bioreactor headspace is maintained below the MOC. The relaxed O₂-limitation on growth and/or removal of O₂-limitation on growth, in turn enables increased productivity for the knallgas culture used in certain embodiments of the present invention.

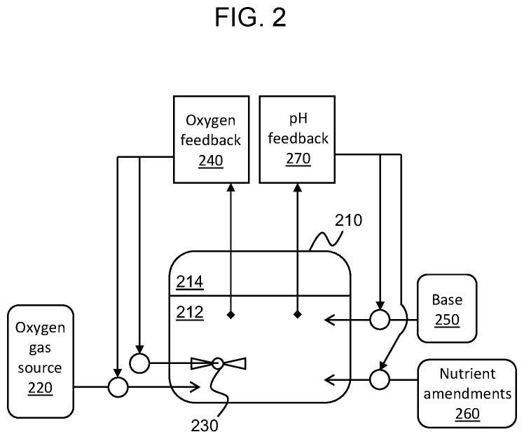

According to Schroeder et al 2005 [Schroder, V and Holtappels, K. (2005) “Explosion characteristics of hydrogen-air and hydrogen-oxygen mixtures at elevated pressures,” in International Conference on Hydrogen Safety, Congress Palace, Pisa, Italy, 2005, is incorporated herein by reference in its entirety], the flammability range of H₂-air gas compositions narrows as the pressure increases above ambient up to around 20 bar. Specifically, lower mol fractions of H₂ lie at and above the upper explosivity limit (UEL), i.e., in the fuel-rich/O₂-lean region of the flammability diagram, as P increases. Consequently, higher mol fractions of O₂ than that which lies at the UEL boundary under ambient pressure (e.g., ˜5% mol % O₂) lie outside the flammability range at elevated P (i.e., on the fuel-rich/O₂-lean non-flammable side of the flammability diagram). Thus, non-flammable fuel-rich gas mixtures can contain a higher f_{O2}% at elevated pressures, than at atmospheric pressure (see FIG. 2 in Schroeder et al). This trend of increasing f_{O2}% reverses above 20 bar, however, even at 50 bar the mol fraction of O₂ demarking the boundary between flammable and non-flammable H₂-air mixtures remains higher than at ambient pressures (i.e., >5% O₂). CO₂ as an inerting gas, behaves similar to N₂ in its impact on the flammability diagram of gas mixtures comprising H₂ and O₂. Thus, a similar narrowing of the flammability range occurs in H₂—CO₂—O₂ gas mixtures with increasing P. While not intending for the present invention to be limited by theory, increasing P can not only increase pO₂ in a safe H₂—CO₂—O₂ gas mixture by increasing the P term in pO₂=f_{O2}%*P, but also by enabling a safe increase in f_{O2}% (i.e., an increase in f_{O2}% while remaining below the MOC at a given P in the bioreactor headspace). In certain embodiments of the present invention, the productivity of a knallgas bioprocess is increased not only by increasing P, but also by increasing f_{O2}%, while still maintaining the mol % O₂ of the gas mixture in the bioreactor headspace and/or any other significant accumulations of gaseous phase within the bioprocess system (e.g., gas recirculation loops, vent lines, etc.) below the MOC at a given P. In other embodiments, the productivity is only increased by increasing P, while the f_{O2}% is kept fixed, providing an additional margin of safety since the difference between the f_{O2}% and the MOC will increase compared to the case at ambient P (e.g., atmospheric P), as P is elevated. In certain embodiments of the present invention the productivity is increased via simultaneously increasing Y_{O2}, f_{O2}, and P. In certain such embodiments a biomass productivity is attained of at least around ≥2 g/L/hr, or ≥3 g/L/hr, or ≥5 g/L/hr, or ≥10 g/L/hr, or ≥20 g/L/hr, or ≥30 g/L/hr, or ≥40 g/L/hr, or ≥50 g/L/hr, or ≥70 g/L/hr, or 90 g/L/hr, or ≥100 g/L/hr.

Adaptive laboratory evolution (ALE) is a powerful technology particularly amenable to evolving industrially relevant phenotypes and has been used to select for nutrient adaptation and environmental stress resistance, e.g., temperature, high salt, and P [Dragosits, M. and Mattanovich, D (2013) Adaptive laboratory evolution-principles and applications for biotechnology. Microbial Cell Fact 12:64], [Marietou, A., et al. (2015) Adaptive laboratory evolution of Escherichia coli K-12 MG1655 for growth at high hydrostatic pressure. Front Microbiol 5:749.], [Lee, S and Kim, P (2020) Current status and applications of adaptive laboratory evolution in industrial microorganisms. J Microbiol Biotechnol 30:793-803. https://doi.org/10.4014/imb.2003.03072], [González-Villanueva, M.; Galaiya, H.; Staniland, P.; Staniland, J.; Savill, I.; Wong, T. S.; Tee, K. L. Adaptive Laboratory Evolution of Cupriavidus necator H16 for Carbon Co-Utilization with Glycerol. Int. J. Mol. Sci. 2019, 20, 5737. https://doi.org/10.3390/ijms20225737]. ALE of E. coli under pressure selection conditions has evolved strains and identified gene mutations conferring tolerance to pressures up to 50 Mpa (500 bar) [Hauben, K J., et al. (1997) Escherichia coli mutants resistant to inactivation by high hydrostatic pressure. Appl. Environ. Microbiol. 63: 945-950.]. ALE does not require prior knowledge of genotype-phenotype relationships. Unlike directed mutagenesis that improves a phenotype but can also accumulate non-beneficial mutations, ALE non-intuitively finds genome-wide adaptive mutations that contribute to fitness. In certain embodiments of the present invention using the the consistent environmental conditions of a continuous culture, a lineage of mutations will be developed in response to selection at elevated P resulting in the selected phenotype i.e., increase tolerance and performance at elevated pressure.

In certain embodiments of the present invention, adaptive evolution is utilized to evolve improved performance at high P by knallgas organisms. In certain such embodiments, adaptive laboratory evolution (ALE) is performed on C. necator for increased productivity and tolerance of elevated P (e.g., P around of at least at least around 7 bar, or at least around 10 bar, or at least around 20 bar, or at least around 50 bar, or at least around 100 bar, or at least around 200 bar). In certain embodiments, knallgas strains have been selected that have evolved under elevated P for robust performance phenotypes as measured by one or more of: enhanced biomass productivity, yield, and stress tolerance. In certain embodiments, ALE is used in continuous culture format to accelerate the generation of genome-wide mutations that confer P-tolerant phenotypes. In certain embodiments, the reactor is operated at a high μ, e.g., an exponential growth regime, to select for the fastest growing mutants at elevated P while washing out slow growers.

In certain embodiments of the present invention, only partial conversion of gaseous feedstocks can be attained on its passage through the working volume of the bioreactor due to thermodynamic, kinetic, and/or stoichiometric limits. In certain such embodiments, the gaseous feedstocks comprise hydrogen and the limitation to complete conversion is kinetic. In certain embodiments, the gaseous feedstocks comprise hydrogen and the limitation is due to oxygen transfer rate into solution that is required for the reactions with CO₂ and O₂ that consume H₂. In certain embodiments, the gaseous feedstocks comprise hydrogen and the limitation to complete conversion is due to insufficient hydrogen transfer rate into solution. In certain embodiments, the gaseous feedstocks comprise hydrogen and the limitation is due to oxygen depletion and/or excess hydrogen. In certain embodiments, the amount of hydrogen reacted in a given passage through the working volume of the bioreactor is ≤5%, or ≤10%, or ≤20%, or 25-35%, or ≤30%, or ≤40%, or ≤50%, or ≤60%, or ≤70%, or ≤80%, or ≤90%, or ≤95%. In other embodiments, the amount of hydrogen reacted in a given passage through the working volume of the bioreactor is greater than or equal to 95%.

In certain embodiments of the present invention, gas is recirculated from the bioreactor headspace back to the bottom of the working volume (i.e., liquid contained in the bioreactor) where the gas is then sparged and/or diffused into the liquid at the base of the working volume and/or at various points in the working volume. In certain such embodiments there is a pressure drop in the gas recirculation loop associated with the flow of gases through the working volume. In certain embodiments, recompression of the recycle gas is required to overcome the pressure drops in the gas recirculation loop. In certain embodiments, the shaft of the final casing in the centrifugal compressor also bears the impeller for the compression of the recycle gas. In certain embodiments, the mixing of make-up gas and recycle gas is performed inside the casing of the centrifugal compressor, and in other embodiments it is performed outside the casing of the centrifugal compressor

It is readily recognized that there are a number of different configurations possible for the gas recirculation loop of the present invention. FIG. 31 shows one non-limiting possibility for the purpose of illustration. Gas recirculation loops in the present invention can vary according to the presence and/or location of condensation and the point at which the make-up gas is introduced, which may be more than a single point, and which may involve different gas mixtures at different points. In certain embodiments, condensable gases or vapors are separated from the unreacted non-condensable gases by condensation. In certain such embodiments, the condensable vapors include water and/or the non-condensable gases include H₂. In certain embodiments, the unconverted gas is supplemented with fresh gas (e.g., H₂, CO₂, and/or O₂) and recycled to the bioreactor. In certain embodiments of the present invention, the concentration of the inert gases (e.g., N₂, methane, and argon) in the gas recirculation loop is controlled by withdrawing a small continuous purge gas stream. After the gas leaves the working volume of the bioreactor, in certain embodiments water is condensed by cooling and the dried recycle gas flows to the recycle compressor. In certain embodiments, the make-up gas contains water. In certain embodiments, the condensation stage is located partially or wholly between the make-up gas supply point and the working volume of the bioreactor. In certain embodiments, recycle compression follows directly after condensing and separating water. In this configuration, it is possible to cool the recycle gas using cooling water or air or glycol immediately before admixing the make-up gas (i.e., before diluting the recycle gas) and thereby to reduce the energy expenditure for refrigerated cooling. In certain embodiments the gas is cooled by adiabatic expansion prior to compression. In certain embodiments the cooling is provided by expansion of the gas over a turbo-expander. In certain embodiments, water vapor and/or other condensable gases are liquified and separated from the non-condensable gases (e.g., H₂) using a turbo-expander.

In certain embodiments of the present invention, a reciprocating compressor is used for the recycle of gases. In certain such embodiments, a recycle cylinder is mounted together with the other cylinders on a reciprocating frame. In certain embodiments a rotary compressor known as a mole pump is used where the compressor and electric driver are completely enclosed in a common high-pressure shell.

In certain embodiments, the make-up gas is introduced into a high pressure recycle loop and acts as the driving fluid of an injector, which compresses the recycle gas.

A major technoeconomic (TEA) challenge to overcome in operating at elevated pressures is the tradeoff between increased capital expenditure (CAPEX)/operating expenses (OPEX) imparted by higher pressure operation and the decrease in CAPEX from higher productivities and OPEX from higher yields. In certain embodiments of the present invention an optimal compromise between the increased cost in terms of CAPEX and/or OPEX and the increased benefit in terms of CAPEX from increased productivity and/or OPEX from higher yields is utilized in designing plants based according to the present invention. Evaluation criteria include energy consumption, investment, and reliability.

In certain non-limiting embodiments of the present invention, electrolysis, e.g., electrolysis of water, is utilized to produce H₂ that serves as an electron donor for the fixation and reduction of CO₂. In certain such embodiments, surplus O₂ produced by electrolysis of water, beyond what is required in the bioprocess, (e.g., the knallgas bioprocess, O₂ requirements), is the major co-product to the biomass and/or biochemicals produced from CO₂, via the chemoautotrophic bioprocess of the present invention. In certain such embodiments, the said biomass and/or biochemicals produced chemoautotrophically from CO₂ comprise proteins, lipids, and/or polysaccharides. In certain such embodiments, the surplus O₂ produced by electrolysis can range from about 50% to about 75% of the total O₂ generated from electrolysis, while all the H₂ produced by electrolysis is consumed in the bioprocess.

Certain embodiments of the present invention comprise one or more bioreactors and one or more gas and/or or liquid recirculation loops. Certain non-limiting embodiments of the present invention are “headspace-free”, meaning that during operation, the liquid and gas suspension (e.g., liquid containing gas bubbles) fills the entire reactor volume, or at least or at least about 95%, or at least or at least about 90%, or at least or at least about 85% of the reactor volume, such that macroscopic pockets of gas phase within the reactor system are eliminated or minimized. In certain embodiments with a headspace-free reactor, the potential for hazardous conditions arising from the accumulation of large gas volumes is greatly reduced.

In certain embodiments of the present invention the input gases, i.e., gaseous feedstocks are fully utilized i.e., at least around 95% of the gaseous feedstock is converted, or at least around 97%, or at least around 90% is converted. In certain embodiments, hydrogen is one of the input gases, and it is fully utilized i.e., at least around 90% of the input H₂ is converted, or at least around 95%, or at least around 97%, or at least around 98%, or at least around 99% of the input H₂ is converted, either in a single pass through the working volume, or through multiple passes through the working volume enabled by internal and/or external gas recirculation.

Further aspects of the bioreactors and methods of the present disclosure are described herein.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The methods and techniques of the present disclosure are generally performed according to conventional methods well-known in the art. Generally, nomenclatures used in connection with, and techniques of biochemistry, enzymology, molecular and cellular biology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated.

Singleton, et al., Dictionary of Microbiology and Molecular Biology, second ed., John Wiley and Sons, New York (1994), and Hale & Markham, The Harper Collins Dictionary of Biology, Harper Perennial, NY (1991) provide one of skill with a general dictionary of many of the terms used in this invention. Any methods and materials similar or equivalent to those described herein can be used in the practice or testing of the methods, systems, and compositions described herein.

The practice of the present invention will employ, unless otherwise indicated, conventional techniques of molecular biology (including recombinant techniques), microbiology, cell biology, and biochemistry, which are within the skill of the art. Such techniques are explained fully in the literature, for example, Molecular Cloning: A Laboratory Manual, second edition (Sambrook et al., 1989); Oligonucleotide Synthesis (M. J. Gait, ed., 1984; Current Protocols in Molecular Biology (F. M. Ausubel et al., eds., 1994); PCR: The Polymerase Chain Reaction (Mullis et al., eds., 1994); and Gene Transfer and Expression: A Laboratory Manual (Kriegler, 1990).

Numeric ranges provided herein are inclusive of the numbers defining the range.

Unless otherwise indicated, nucleic acids are written left to right in 5′ to 3′ orientation; amino acid sequences are written left to right in amino to carboxy orientation, respectively.

### Definitions

“A,” “an” and “the” include plural references unless the context clearly dictates, thus the indefinite articles “a”, “an,”, and “the” as used herein in the specification and in the claims, unless clearly indicated to the contrary, should be understood to mean “at least one.”

The term “about” or “around” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods or in connection with a disclosed composition.

“Acetogen” refers to a microorganism that generates acetate and/or other short chain organic acids up to C4 chain length as a product of anaerobic respiration.

“Acidophile” refers to a type of extremophile that thrives under highly acidic conditions (usually at pH 2.0 or below).

The term “amino acid” refers to a molecule containing both an amine group and a carboxyl group that are bound to a carbon, which is designated the alpha-carbon. Suitable amino acids include, without limitation, both the D- and L-isomers of the naturally occurring amino acids, as well as non-naturally occurring amino acids prepared by organic synthesis or other metabolic routes. In some embodiments, a single “amino acid” might have multiple sidechain moieties, as available per an extended aliphatic or aromatic backbone scaffold. Unless the context specifically indicates otherwise, the term amino acid, as used herein, is intended to include amino acid analogs. Standard three-letter abbreviations for amino acids are used herein, for example: Cys Cysteine; Gin Glutamine; Glu Glutamic acid (Glutamate); Gly Glycine; His Histidine; lie Isoleucine; Leu Leucine; Lys Lysine; Met Methionine; Phe Phenylalanine; Pro Proline; Ser Serine; Thr Threonine; Trp Tryptophan; Tyr Tyrosine; Val Valine;

“Anabolism” refers to the process by which living organisms synthesize complex molecules of life from simpler ones. Anabolic processes produce peptides, proteins, polysaccharides, lipids, and nucleic acids. The energy required for anabolism is supplied by intracellular energy carriers such as adenosine triphosphate (ATP).

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

“Arable land” is land capable of being used to grow crops in the soil (e.g. not including hydroponic or other soil-less cultivation methods). The category of arable land generally excludes: desert; rocky or salty infertile soils; environments that are too cold or have too short a growing season for agriculture; heavily built urban and industrial environments; rooftops and paved roads and lots; and contaminated soils.

“Autoignition temperature” refers to the temperature at which a flammable gas mixture will combust even if there is no ignition source present.

The “biogenic carbon cycle” is the process by which plants, animals and the biosphere recycle carbon. It generally corresponds to the fast domain of the carbon cycle (the atmosphere, ocean, vegetation and soil) where carbon turnover time typically ranges from one to 100 years. The biogenic carbon cycle stands in contrast to the geological carbon cycle or the slow domain of the carbon cycle where carbon turnover times exceed 10,000 years. Soil carbon represent an intermediate case with turnover times on the order of a 10 to 500 year range.

The term “biomass” refers to a material produced by growth and/or propagation of cells (e.g., microorganism cells). Biomass may contain cells and/or intracellular contents as well as extracellular material, including, but not limited to, compounds secreted by a cell.

The term “bioreactor” or “fermenter” refers to a closed or partially closed vessel in which cells are grown and maintained. The cells may be, but are not necessarily held in liquid suspension. In some embodiments, rather than being held in liquid suspension, cells may alternatively be grown and/or maintained in contact with, on, or within another non-liquid substrate including but not limited to a solid growth support material.

The “biosphere” comprises the regions of the surface, atmosphere, and hydrosphere of the earth occupied by living organisms.

“Biostimulant” or “bio-stimulant” refers to compounds capable of stimulating the growth, proliferation and/or development of cells, when provided in the culture medium, and/or to organisms, when ingested or otherwise provided to the organism in an accessible form.

The term “carbon-fixing” reaction or pathway refers to enzymatic reactions or metabolic pathways that convert C1 carbon molecules, including forms of carbon that are gaseous under ambient conditions, including but not limited to CO₂, CO, and CH₄, into carbon-based biochemicals, including biochemical molecules that are liquid or solid under ambient conditions, or which are dissolved into, or held in suspension in, aqueous solution.

“Carbon source” refers to the types of molecules from which a microorganism derives the carbon needed for organic biosynthesis.

“Carboxydotrophic” refers to microorganisms that can tolerate or oxidize carbon monoxide. In preferred embodiments a carboxydotrophic microorganism can utilize CO as a carbon source and/or as a source of reducing electrons for biosynthesis and/or respiration.

“Chemoautotrophic” refers to the ability of an organism to obtain energy by the oxidation of chemical electron donors by chemical electron acceptors and to synthesize all the organic compounds needed by the organism to live and grow from carbon dioxide.

In the claims, as well as in the specification, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively.

The term “culturing” refers to growing and maintaining a population of cells, e.g., microbial cells or animal cells, under suitable conditions for proliferation, propagation, maintenance, development and/or differentiation, in a liquid or solid medium.

The term “derived from” encompasses the terms “originated from,” “obtained from,” “obtainable from,” “isolated from,” and “created from,” and generally indicates that one specified material finds its origin in another specified material or has features that can be described with reference to another specified material.

“Energy source” refers to either the electron donor that is oxidized by oxygen in aerobic respiration or the combination of electron donor that is oxidized and electron acceptor that is reduced in anaerobic respiration.

“Extremophile” refers to a microorganism that thrives in physically or geochemically extreme conditions (e.g., high or low temperature, pH, or high salinity) compared to conditions on the surface of the Earth or the ocean that are typically tolerated by most life forms found on or near the earth's surface.

“Fertilizer” is an organic or inorganic, natural or synthetic substance which is used to enrich the soil and to provide plants with one or more essential nutrients for ordinary vegetative growth. In certain embodiments, the term fertilizer also refers to nutrients for fungi and the production of mushrooms.

The term “gasification” refers to a generally high temperature process that converts carbon-based materials into a mixture of gases including hydrogen, carbon monoxide, and carbon dioxide called synthesis gas, syngas, or producer gas. The process generally involves partial combustion and/or the application of externally generated heat along with the controlled addition of oxygen and/or steam such that insufficient oxygen is present for complete combustion of the carbon-based material. The chemical reaction of hydrocarbons with water, oxygen, air, or any combination of these that has insufficient oxidant for complete oxidation i.e., combustion is generally referred to as gasification. It generally yields a gas mixture made up of CO and H₂ in various proportions along with carbon dioxide and, where air is used, some nitrogen. Any carbon containing feedstock having fuel value can in principle be gasified.

“Halophile” refers to a type of extremophile that thrives in environments with very high concentrations of salt.

The term “heterologous” or “exogenous,” with reference to a polynucleotide or protein, refers to a polynucleotide or protein that does not naturally occur in a specified cell, e.g., a host cell. It is intended that the term encompass proteins that are encoded by naturally occurring genes, mutated genes, and/or synthetic genes. In contrast, the term “homologous,” with reference to a polynucleotide or protein, refers to a polynucleotide or protein that occurs naturally in the cell.

“Heterotrophic” refers to a mode of growth and maintenance of an organism by taking in and metabolizing organic substances, such as plant, animal, or microorganism matter. Growth is heterotrophic when the organism does not synthesize all the organic compounds needed by the organism to live and grow from carbon dioxide and utilizes organic compounds. During heterotrophic growth, organisms cannot produce their own food and instead obtain food and energy by taking in and metabolizing organic substances, such as plant or animal matter, i.e., rather than fixing carbon from inorganic sources such as carbon dioxide.

“Hydrogen-oxidizer” or “Hydrogenotroph” refers to a microorganism that utilizes reduced H₂ as an electron donor for the production of intracellular reducing equivalents and/or in respiration.

“Hyperthermophile” refers to a type of extremophile that thrives in extremely hot environments for life, typically about 60° C. (140° F.) or higher.

The term “knallgas” refers to the mixture of molecular hydrogen and oxygen gas. A “knallgas microorganism” is a microbe that can use hydrogen as an electron donor and oxygen as an electron acceptor in respiration for the generation of intracellular energy carriers such as Adenosine-5′-triphosphate (ATP). The terms “oxyhydrogen” and “oxyhydrogen microorganism” can be used synonymously with “knallgas” and “knallgas microorganism,” respectively. Knallgas microorganisms generally use molecular hydrogen by means of hydrogenases, with some of the electrons donated from H₂ that is utilized for the reduction of NADW (and/or other intracellular reducing equivalents) and some of the electrons from H₂ that is used for aerobic respiration. Knallgas microorganisms generally fix CO₂ autotrophically, through pathways including but not limited to the Calvin Cycle or the reverse citric acid cycle [“Thermophilic bacteria”, Jakob Kristjansson, Chapter 5, Section III, CRC Press, (1992)].

The terms “lipids” refers to category of molecules that can be dissolved in nonpolar solvents (such as chloroform and/or ether) and which also have low or no solubility in water. The hydrophobic character of lipids molecules typically results from the presence of long chain hydrocarbon sections within the molecule. Lipids subsume the following molecule types: hydrocarbons, fatty acids (saturated and unsaturated), fatty alcohols, fatty aldehydes, hydroxy acids, diacids, monoglycerides, diglycerides, triglycerides, phospholipids, sphingolipids, sterols such as cholesterol and steroid hormones, fat-soluble vitamins (such as vitamins A, D, E and K), polyketides, terpenoids, and waxes.

“Life cycle scope”—“Scope 1” considers direct GHG emissions and other direct life cycle impacts such as direct land use and water use and “Scopes 2 and 3” considers indirect GHG emissions and other indirect life cycle impacts (e.g., land, water) up and/or down the supply chain. Scope 1 GHG emissions and other life cycle impacts refer to those impacts occurring on the site of a production plant or farm or resulting in the immediate location from such a production plant or farm. Scope 2 refers to life cycle impacts (e.g., GHG, land, water, etc.) associated with electricity and/or heat that is imported from another geographically removed location to a production plant or farm. Scope 3 allows for the treatment of all other indirect impacts including the extraction and production of material feedstocks used in a production plant or farm (e.g., CO₂, NH₃, inorganic mineral nutrients, etc.), the transportation of feedstocks, and, in the case of cradle-to-grave life cycle analysis (LCA), the transport, use, and disposal of sold goods, and the final waste degradation, decomposition, or sequestration processes.

“Lithoautotrophic” refers to a specific type of chemoautotrophy where the organism utilizes the oxidation of inorganic chemical electron donors by inorganic chemical electron acceptors as an energy source.

The term “lysate” refers to the liquid containing a mixture and/or a solution of cell contents that result from cell lysis. In some embodiments the lysate may be dewatered, to form a concentrated lysate, or dried to form a dry solid. In certain such embodiments the dry lysate is in a powder form. In some embodiments, the methods described herein comprise a purification of chemicals or mixture of chemicals in a cellular lysate. In some embodiments, the methods comprise a purification of amino acids and/or protein in a cellular lysate.

The term “lysis” refers to the rupture of the plasma membrane and if present, the cell wall of a cell such that a significant amount of intracellular material escapes to the extracellular space. Lysis can be performed using electrochemical, mechanical, osmotic, thermal, or viral means. In some embodiments, the methods described herein comprise performing a lysis of cells or microorganisms as described herein in order to separate a chemical or mixture of chemicals from the contents of a bioreactor. In some embodiments, the methods comprise performing a lysis of cells or microorganisms described herein in order to separate an amino acid or mixture of amino acids and/or proteins and/or peptides from the non-proteinaceous contents of a bioreactor or cellular growth medium.

“Methanogen” refers to a microorganism that generates methane as a product of anaerobic respiration.

“Methylotroph” refers to a microorganism that can use reduced one-carbon compounds including methanol as a carbon source and/or as an electron donor for their growth.

“Methanotroph” refers to a microorganism that can use reduced one-carbon compounds, such as but not limited to methane as a carbon source and/or as an electron donor for their growth.

The terms “microorganism” and “microbe” mean microscopic single celled life forms, including bacteria, yeast, microalgae, and fungi.

Minimum (or limiting) Oxygen concentration—MOC (LOC)—is refers to the limiting concentration of oxygen below which combustion is not possible, independent of the concentration of fuel. To create and sustain fire or explosion, in addition to fuel, oxygen must be present. Very broadly speaking most gaseous fuels require oxygen to be present at c. 10% v/v for combustion to take place. The main exceptions to this are carbon monoxide and hydrogen which have a MOC of c. 4.5-5.0% v/v. Adopting inertion as the basis of safety uses this phenomenon to create a safe operation—the use of an inert gas in adequate quantities lowers the volume fraction of oxygen in the gas mixture to below the MOC threshold.

The term “molecule” means any distinct or distinguishable structural unit of matter comprising one or more atoms, and includes for example hydrocarbons, lipids, polypeptides, and polynucleotides.

A “nutritionally fastidious” strain refers to an organism with complex or specific nutritional requirements, e.g., an organism that will grow only when specific nutrients are present.

The term “oleaginous” refers to something that is rich in oil or produces oil in high quantities.

“Oligopeptide” refers to a peptide that contains a relatively small number of amino-acid residues, for example, about 2 to about 20 amino acids.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

The term “organic compound” refers to any gaseous, liquid, or solid chemical compound that contains carbon atoms, with the following exceptions that are considered inorganic: carbides, carbonates, simple oxides of carbon, cyanides, and allotropes of pure carbon such as diamond and graphite.

“Peptide” refers to a compound consisting of two or more amino acids linked in a chain, the carboxyl group of each acid being joined to the amino group of the next by a bond of the type R—OC—NH—R′, and may include about 2 to about 50 amino acids.

As used herein, the term “polynucleotide” refers to a polymeric form of nucleotides of any length and any three-dimensional structure and single- or multi-stranded (e.g., single-stranded, double-stranded, triple-helical, etc.), which contain deoxyribonucleotides, ribonucleotides, and/or analogs or modified forms of deoxyribonucleotides or ribonucleotides, including modified nucleotides or bases or their analogs. Because the genetic code is degenerate, more than one codon may be used to encode a particular amino acid, and the present invention encompasses polynucleotides which encode a particular amino acid sequence. Any type of modified nucleotide or nucleotide analog may be used, so long as the polynucleotide retains the desired functionality under conditions of use, including modifications that increase nuclease resistance (e.g., deoxy, 2′-O-Me, phosphorothioates, etc.). Labels may also be incorporated for purposes of detection or capture, for example, radioactive or nonradioactive labels or anchors, e.g., biotin. The term polynucleotide also includes peptide nucleic acids (PNA). Polynucleotides may be naturally occurring or non-naturally occurring. The terms “polynucleotide,” “nucleic acid,” and “oligonucleotide” are used herein interchangeably. Polynucleotides may contain RNA, DNA, or both, and/or modified forms and/or analogs thereof. A sequence of nucleotides may be interrupted by non-nucleotide components. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR.sub.2 (“amidate”), P(O)R, P(O)OR′, CO or CH.sub.2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. Polynucleotides may be linear or circular or comprise a combination of linear and circular portions.

As used herein, “polypeptide” refers to a composition comprised of amino acids and recognized as a protein by those of skill in the art. The conventional one-letter or three-letter code for amino acid residues may be used. The terms “polypeptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also, included within the definition are, for example, polypeptides containing one or more analogs of an amino acid (including, for example, unnatural amino acids, etc.), as well as other modifications known in the art.

The term “precursor to” or “precursor of” is an intermediate towards the production of one or more of the components of a finished product.

The term “probiotic” refers to a microorganism that provides health benefits when consumed, e.g., beneficial intestinal flora.

“Producer gas” refers to a gas mixture containing various proportions of H₂, CO, and CO₂, and having heat value typically ranging between one half and one tenth that of natural gas per unit volume under standard conditions. Producer gas can be generated various ways from a variety of feedstocks, including gasification, steam reforming, or autoreforming of carbon-based feedstocks. In addition to H₂, CO, and CO₂, producer gases can contain other constituents including but not limited to methane, hydrogen sulfide, condensable gases, tars, and ash depending upon the generation process and feedstock. The proportion of N₂ in the mixture can be high or low depending on whether air is used as an oxidant in the reactor or not and if the heat for the reaction is provided by direct combustion or through indirect heat exchange.

The term “producing” includes both the production of compounds intracellularly and extracellularly, including the secretion of compounds from the cell.

“Productivity” refers to the amount of a substance produced by a microorganism per unit volume per unit time in a microbial fermentation process. For example, biomass productivity may be expressed as grams of biomass produced per liter of solution per hour.

“Psychrophile” refers to a type of extremophile capable of growth and reproduction in cold temperatures, typically about 10° C. and lower.

The term “recombinant” refers to genetic material (i.e., nucleic acids, the polypeptides they encode, and vectors and cells comprising such polynucleotides) that has been modified to alter its sequence or expression characteristics, such as by mutating the coding sequence to produce an altered polypeptide, fusing the coding sequence to that of another gene, placing a gene under the control of a different promoter, expressing a gene in a heterologous organism, expressing a gene at a decreased or elevated levels, expressing a gene conditionally or constitutively in manner different from its natural expression profile, and the like. Generally recombinant nucleic acids, polypeptides, and cells based thereon, have been manipulated by man such that they are not identical to related nucleic acids, polypeptides, and cells found in nature. A recombinant cell may also be referred to as “engineered.”

The terms “recovered,” “isolated,” “purified,” and “separated” as used herein refer to a material (e.g., a protein, nucleic acid, or cell) that is removed from at least one component with which it is naturally associated. For example, these terms may refer to a material that is substantially or essentially free from components which normally accompany it as found in its native state, such as, for example, an intact biological system.

“Reynolds number” (Re) is a dimensionless number frequently used to characterize fluid flow regimes as either being laminar or turbulent. For Reynolds numbers <2000 flow is considered to be laminar. Fully developed turbulent flow is considered to occur at Re >4000. Transition regime flow occurs 2000<Re<4000.

The phrase “substantially free” as to any given component means that such component is only present, if at all, in an amount that is a functionally insignificant amount, i.e., it does not significantly negatively impact the intended performance or function of any process or product. Typically, substantially free means less than about 1%, including less than about 0.5%, including less than about 0.1%, and also including zero percent, by weight of such component. “Substantially” means to a significant extent, such as close to 100%, or any of at least about 98%, 99%, or 99.5%.

“Sulfur-oxidizer” refers to microorganisms that utilize reduced sulfur containing compounds including but not limited to H₂S as electron donors for the production of intracellular reducing equivalents and/or in respiration.

“Syngas” or “synthesis gas” in the context of steam methane reforming (SMR) or gasification refers to a type of gas mixture, which like producer gas contains H₂ and CO, but which has been more specifically tailored in terms of H₂ and CO content and ratio and levels of impurities for the synthesis of a particular type of chemical product, such as but not limited to methanol or fischer-tropsch diesel. Syngas generally contains H₂, CO, and CO₂ as major components, and it can be generated through established methods including: steam reforming of methane, liquid petroleum gas, or biogas; or through gasification of any organic, flammable, carbon-based material, including but not limited to biomass, waste organic matter, various polymers, peat, and coal. The hydrogen component of syngas can be increased through the reaction of CO with steam in the water gas shift reaction, with a concomitant increase in CO₂ in the syngas mixture. Syngas includes cases where the H₂ faction has been highly purified and all the other carbon-containing gases (e.g., CO and CO₂) have been reduced down to very low levels or removed. Syngas in the context of the Haber-Bosch process can refer to the H₂ and N₂ inputs to the reaction, where the H₂ may be derived from SMR or gasification, or it may be derived from an alternative process such as electrolysis of water, and where the N₂ is generally sourced from air.

“Thermophile” refers to a type of extremophile that thrives at relatively high temperatures for life, typically about 45° C. to about 122° C.

“Titer” refers to amount of a substance produced by a microorganism per unit volume in a microbial culture. For example, biomass titer may be expressed as grams of biomass produced per liter of solution (e.g., culture medium).

The Upper and Lower explosion limits of a gas mixture are commonly referred to as “UEL” and “LEL,” respectively. Fires and explosions can be prevented if there is either too much fuel (i.e., fuel ‘rich’) or too little fuel (i.e., fuel ‘lean’) to support combustion.

A “vitamin” is a compound, e.g., organic compound, that is essential for growth and/or nutrition of an organism, typically required in small quantities in the diet or in a growth or culture medium.

A “vitamer” as used herein, refers to chemical analogs of a particular vitamin that are effective in functionally substituting for each other, and/or are effective in relieving a deficiency of the vitamin.

“Wild-type” refers to a microorganism as it occurs in nature.

“Yield” may refer to amount of a product produced from a feed material relative to the total amount of the substance that would be produced if all of the feed substance were converted to product. For example, amino acid yield may be expressed as % of amino acid produced relative to a theoretical yield if 100% of the feed substance were converted to amino acid. Alternatively yield may refer to the amount of product generated per substrate consumed (e.g., g biomass/g H₂). The meaning will be clarified by the units given with the yield number.

### Culturing a Microorganism

In certain embodiments, a method of the present disclosure includes culturing a microorganism, e.g., chemoautotrophic microorganism, in a bioreactor or fermenter under conditions suitable for microorganism growth and generation of a biomass (e.g., single cell protein (SCP)) that may then be converted into a protein isolate, protein concentrate, and/or protein hydrolysate composition. Any suitable methods may be used to culture the microorganisms. The microorganism may be grown under any suitable conditions, including but not limited to chemoautotrophic conditions, in an environment that is suitable for growth and production of biomass. In some embodiments, the microorganism may be grown in autotrophic culture conditions, heterotrophic culture conditions, or a combination of autotrophic and heterotrophic culture conditions. A heterotrophic culture may include a suitable source of carbon and energy, such as one or more sugar (e.g., glucose, fructose, sucrose, etc.). An autotrophic culture may include C1 chemicals such as carbon monoxide, carbon dioxide, methane, methanol, formate, and/or formic acid, and/or mixtures containing C1 chemicals, including, but not limited to various syngas compositions or various producer gas compositions, e.g., generated from low value sources of carbon and energy, such as, but not limited to, lignocellulosic energy crops, crop residues, bagasse, saw dust, forestry residue, or food, through the gasification, partial oxidation, pyrolysis, or steam reforming of said low value carbon sources, that can be used by an oxyhydrogen microorganism or hydrogen-oxidizing microorganism or carbon monoxide oxidizing microorganism as a carbon source and an energy source. Suitable ways of culturing the microorganisms and generating a biomass for use in the present methods are described, e.g., in PCT Application Nos. PCT/US2010/001402, PCT/US2011/034218, PCT/US2013/032362, PCT/US2014/029916, PCT/US2017/023110, PCT/US2018/016779, and U.S. Pat. No. 9,157,058, each of which is hereby incorporated by reference herein in its entirety. In some embodiments, the organism may be grown photosynthetically in a bioreactor, in a hydroponics system, in a greenhouse, or in a cultivated field, or may be collected from waste or natural sources.

In certain non-limiting embodiments of the present invention, syngas or producer gas feedstocks are used to produce biobased products. In certain such embodiments, the said syngas or producer gas feedstocks are produced from gasification of solid or liquid wastes including but not limited to wood, agricultural residues, forestry residues, biological or carbon-based fibers and/or polymers, plastics, diapers and/or absorptive hygiene wastes, and/or composites containing carbon-based materials. In certain embodiments of the present invention, chemoautotrophic strains utilize syngas components for their carbon and/or energy sources.

In some embodiments, the electron donors and/or electron acceptors are generated or recycled using renewable, alternative, or conventional sources of power that are low in greenhouse gas emissions. These sources of power may be selected from at least one of photovoltaics, solar thermal, wind power, hydroelectric, nuclear, geothermal, enhanced geothermal, ocean thermal, ocean wave power, and tidal power. In some embodiments, the electron donors and/or electron acceptors are generated using grid electricity during periods when electrical grid supply exceeds electrical grid demand, and wherein storage tanks buffer the generation of said electron donors and/or electron acceptor, and their consumption in the said carbon-fixing reaction.

In certain embodiments, carbon dioxide emission-free or low-carbon emission and/or renewable sources of power, including but not limited to one or more of the following: photovoltaics, solar thermal, wind power, hydroelectric, nuclear, geothermal, enhanced geothermal, ocean thermal, ocean wave power, tidal power, may be used for the production of electron donors, and particularly hydrogen gas. In certain embodiments, the hydrogen gas is generated from non-potable water or waste water or sea water or other sources of salt water or brine using the aforementioned low-carbon emission and/or renewable sources of power along with established electrolysis technologies. Certain such embodiments may further apply established reverse osmosis (RO) technologies to increase the water purity to a level that is acceptable for a given electrolyzer technology. In certain embodiments, oxyhydrogen microorganisms function as biocatalysts within the bioreactor of the present disclosure for the conversion of renewable energy and/or low or zero carbon emission energy into protein, liquid hydrocarbon fuel, or high energy density oleochemicals, or organic compounds generally, with CO₂ captured from flue gases, or from the atmosphere, or from the ocean serving as a carbon source.

In some embodiments, the electron donors comprise H₂ and/or CO and/or methane derived from a tail gas from one or more of: methane steam reforming; petroleum refining; steel production; aluminum production; manganese production; the chloralkali process; carbon black manufacture; methanol synthesis; ammonia synthesis; metallurgical processes; chemical processes; and electrochemical processes.

In some embodiments, molecular hydrogen is utilized as an electron donor. The hydrogen gas may generated via a method using at least one of the following: electrolysis of water; thermochemical splitting of water; electrolysis of brine; electrolysis and/or thermochemical splitting of hydrogen sulfide. In some embodiments, electrolysis of water for the production of hydrogen is performed using one or more of: Proton Exchange Membranes (PEM); liquid electrolytes such as KOH; alkaline electrolysis; Solid Polymer Electrolyte electrolysis; high-pressure electrolysis; and/or high temperature electrolysis of steam (HTES). In some embodiments, thermochemical splitting of water to produce hydrogen is performed using one or more of: the iron oxide cycle; cerium(IV) oxide-cerium(III) oxide cycle; zinc zinc-oxide cycle; sulfur-iodine cycle; copper-chlorine cycle; calcium-bromine-iron cycle; hybrid sulfur cycle.

Hydrogen produced by water electrolysis is utilized as an electron donor in certain embodiments of the present invention. In certain non-limiting embodiments of the present invention, ammonia, ammonium hydroxide, ammonium salt, nitrate, and/or urea may be utilized as a nitrogen source in the present invention, and may be produced via, for example the Haber-Bosch reaction, using hydrogen produced by water electrolysis. In certain embodiments of the present invention, the electron donor is hydrogen, and the nitrogen source is produced using hydrogen via the Haber-Bosch reaction, where the hydrogen in both cases is produced by electrolysis of water. In certain such embodiments, the said electrolysis is powered by low CO₂ emitting and/or renewable power.

Certain aspects of the present invention relate to synthesis gas production, feedstock pretreatment and gas generation. Certain aspects of the present invention relate to carbon monoxide conversion. Certain aspects of the present invention relate to gas purification.

H₂ and/or CO₂ utilized as a feedstock in certain embodiments of the present invention is generated from and/or increased in a syngas, producer gas, or CO containing gas mixture via the water gas shift (WGS) reaction or carbon monoxide shift conversion. In certain embodiments of the present invention, only H₂ and CO₂ are utilized as feedstocks, and CO is preferably reduced or removed from the raw synthesis gas emerging from a gasification, steam reforming, or partial oxidation process. Depending on feedstock and syngas generation technology, a raw synthesis gas may contain 10-50% carbon monoxide and varying amounts of carbon dioxide. In the water gas shift reaction, also known as the carbon monoxide shift conversion, the carbon monoxide serves as reducing agent for water to yield hydrogen and carbon dioxide. In certain embodiments of the present invention carbon monoxide is converted in this way to produce additional carbon dioxide and hydrogen. In certain such embodiments, an excess of CO₂ is produced beyond that required for the bioprocess, which can be readily separated and removed from the hydrogen stream using methods and processes well known in hydrogen purification. In certain embodiments, this said excess CO₂ emerging from WGS, SMR, and/or gasification, is also reacted through a chemoautotrophic bioprocess of the present invention by providing supplemental H₂ generated via one or more of the aforementioned electrolysis and/or thermochemical water splitting processes powered by renewable and/or low-CO₂ power sources.

In certain embodiments of the present invention, a steam reforming plant is coupled with the WGS for the production of H₂ and/or CO₂ used as feedstock in a gas bioprocess. In certain embodiments, the gas from the secondary reformer is cooled by recovering the waste-heat, which is used for raising and superheating steam. In certain embodiments, a raw synthesis gas enters a high-temperature shift (HTS) reactor loaded with an iron-chromium catalyst at 320-350° C. In certain embodiments the said raw synthesis gas has flowed out of a secondary reformer into a heat exchanger and then into the said HTS. In certain embodiments the gas temperature increases in the HTS around 50-70° C. (depending on initial CO concentration) and exits the HTS with a residual CO content of around 3%. In certain such embodiments the gas with a CO content of around 3% is cooled to around 5 to 90° C. and then input into a bioreactor according to the present invention. In other embodiments, the said gas flowing out of the HTS is cooled to around 200-210° C. for the low temperature shift (LTS), which is carried out on a copper-zinc-alumina catalyst in a downstream reaction vessel and achieves a carbon monoxide concentration of around 0.1-0.3 vol %. In certain such embodiments the gas with a CO content of around 0.1-0.3 vol % is then cooled to around 5 to 90° C. and then input into a bioreactor according to the present invention.

In certain embodiments of the present invention, only a high-temperature shift conversion is used. In certain such embodiments, this results in a carbon monoxide content of the gas after shift conversion in the range around 3-5 vol %. In certain such embodiments, the said gas is fed into a bioreactor of the present invention containing one or more of a carboxydotrophic microorganism and/or a hydrogenotrophic microorganism i.e., a consortium of microorganisms. In certain embodiments of the present invention, a partial oxidation process is followed by a high-temperature shift conversion but not a low-temperature shift conversion, In certain embodiments of the present invention, copper liquor scrubbing is used for carbon monoxide removal.

In certain embodiments bulk removal of carbon oxides from a H₂ containing gas stream is performed using a shift reaction and/or CO₂ removal. In certain such embodiments, the remaining carbon oxides, are present in the stoichiometric ratio with H₂ that is utilized by a chemoautotrophic microorganism and/or consortium and/or a carboxydotrophic microorganism and/or consortium. In certain embodiments the carbon oxides are reduced to a low level using one or more of the aforementioned methods, and then a fraction of the carbon oxides are either re-introduced back into the H₂ containing stream and fed into a bioreactor, and/or the carbon oxides are fed into a bioreactor through a separate inlet, along with the H₂-rich stream that is fed through a different inlet into the bioreactor. In certain embodiments of the present invention, following the aforementioned carbon oxide removal steps, a H₂-rich gas stream has around 0.2-0.5 vol % CO and around 0.005-0.2 vol % CO₂. In certain embodiments, the said H₂-rich gas stream with around 0.2-0.5 vol % CO and around 0.005-0.2 vol % CO₂ is pumped, diffused, or otherwise introduced to a bioreactor of the present invention. In certain embodiments of the present invention, carbon oxides are removed from a H₂-rich gas stream down to a ppm level, using methods well known and established in ammonia synthesis. In certain embodiments of the present invention, methanation is used to reduce the concentrations of the carbon oxides in a H₂-rich gas stream below 10 ppm. In certain such embodiments the H₂-rich gas stream with methane produced by methanation is fed into a bioreactor containing a consortium of microorganisms including but not limited to one or more H₂-oxidizing chemoautotrophs and one or more methanotrophs. In certain embodiments of the present invention, the Selectoxo process is utilized to reduce the CO content of a gas stream. In certain embodiments, a Selectoxo process is used in lieu of a methanation system. In certain embodiments the Selectoxo process is utilized to reduce the inert gas content as well as the CO content of a gas stream. In certain embodiments, the raw gas is mixed with the stoichiometric quantity of air or oxygen needed to convert the carbon monoxide to carbon dioxide. In certain such embodiments, the gas mixture is then passed through a precious-metal catalyst at 40-135° C. to accomplish selective oxidation. In certain embodiment, the Selectoxo process is preformed after a low-temperature shift conversion and cooling of the gas. In certain embodiments, the carbon dioxide formed by the Selectoxo reaction is directed into a bioreactor as part of a H₂-rich gas stream.

In certain embodiments of the present invention, a gas stream containing H₂ and/or CO₂ undergoes gas purification prior to being pumping into a bioreactor. In certain embodiments of the present invention, carbon dioxide, residual carbon monoxide, and/or sulfur compounds are removed from an H₂ containing gas stream.

In certain embodiments of the present invention, the gases that are used during the fermentation (i.e., bioprocess) comprise hydrogen, carbon dioxide, and oxygen. In certain such embodiments, the fermentation relies on growing a Cupriavidus microorganism, such as, for example, Cupriavidus necator or Cupriavidus metallidurans. In certain such embodiments, the said Cupriavidus, e.g., Cupriavidus necator, is grown with the aim of harvesting the cultivated biomass as a source of single cell protein (SCP).

In certain embodiments the partial pressure range of CO₂ in gas streams used as a carbon source in the present invention are around 4-7 bar. In certain such embodiments the said partial pressure of CO₂ in the range of 4-7 bar flows out of a steam reforming plant and/or WGS. In certain embodiments chemical solvents are used to capture the said CO₂ with partial pressures in the range of 4-7 bar.

In certain embodiments of the present invention, chemical solvents are used to capture and purify CO₂ taken from one or more of the following sources: fermentation off-gases (e.g., ethanol, lactic acid, etc.); biogas; industrial flue gas; WGS, SMR, and/or gasification product streams, tail gases; flared gases; geothermal or geological vent gases; and/or the atmosphere. In certain such embodiments, the said chemical solvents are based on aqueous solutions of potassium carbonate or alkanolamines containing additional activators to enhance mass transfer and, in some cases, inhibitors to limit or prevent corrosion processes. Primary and secondary amines, for example, monoethanolamine (MEA) and diethanolamine (DEA) exhibit a high mass transfer rate for carbon dioxide. In certain embodiments a primary and/or secondary amine such as but not limited to MEA and/or DEA are used to capture CO₂. However, primary and secondary amines can have a higher energy demand for regeneration than other options. In certain embodiments, tertiary amines are used to capture CO₂, such as, for example, methyldiethanolamine along with an activator. In certain embodiments, triethanolamine is used for CO₂ capture. In certain such embodiments, following CO₂ capture using triethanolamine, additional scrubbing of CO₂ is performed using a primary or secondary amine such as MEA. In certain embodiments of the present invention, a potassium carbonate process, such as but not limited to those commercially available from various licensors, is used for CO₂ capture. In certain embodiments using a potassium carbonate process for CO₂ capture, an activator and/or corrosion inhibitor are utilized in the process.

In certain embodiments of the present invention, cryogenic methods are used for the purification of H₂ containing gases. In certain embodiments cryogenic methods are used for the purification of partial oxidation gases and/or steam reforming gases. In certain embodiments, the Braun Purifier process is utilized for gas streams fed into bioreactors of the current invention. In certain embodiments a cryogenic unit is used to remove excess nitrogen gas, or most of the nitrogen gas, or all or substantially all of the nitrogen gas from an H₂ containing gas stream. In certain embodiments a cryogenic unit is used to remove excess nitrogen gas, or most of the nitrogen gas, or all or substantially all of the nitrogen gas from air. In certain embodiments a cryogenic unit is used to remove excess nitrogen gas, or most of the nitrogen gas, or all of the nitrogen gas from a gas stream flowing from a secondary reformer using air. In certain embodiments a cryogenic unit is placed downstream of a methanator. In certain embodiments, the level of inert gases and/or non-reactive gases in a gas recirculation loop are reduced through the use of a cryogenic unit. In certain such embodiments, methane and/or argon and/or nitrogen is completely and/or partially removed from a gas mixture in a gas recirculation loop. In certain embodiments, a cryogenic unit is used to completely and/or partially remove methane and/or argon and/or nitrogen from make-up gas fed into a bioreactor of the present invention. In certain embodiments, the purifier unit comprises a feed/effluent exchanger, and a rectifier column with an integrated condenser and turbo-expander. In certain embodiments, a temperature of around −185° C. is used to wash out methane and/or argon from a gas stream. In certain such embodiments, the said gas stream contains H₂. In certain embodiments, the cooling energy used in a cryogenic unit is supplied by expansion of the raw gas over a turbo-expander.

In certain embodiments, one or more purification steps are placed between a gasifier, steam reformer, partial oxidizer, and/or WGS unit, allowing the H₂/CO/CO₂ ratio to be set independent of said gasifier, steam reformer, partial oxidizer, and/or WGS unit.

In certain embodiments, one or more cooling steps are placed between: a gasifier, steam reformer, partial oxidizer, and/or WGS unit; and a bioreactor. In certain such embodiments, the gas produced by one or more of these sources is cooled via boiler feed water heating and/or raising steam.

In certain embodiments of the present invention, a liquid nitrogen wash is used for gas purification. In certain such embodiments, the said liquid nitrogen wash delivers a gas to the bioreactor and/or gas recirculation loop that is free of all impurities, including inert gases, or largely free of impurities, or removes and decreases the level of impurities. In certain embodiments, the nitrogen is liquefied in a refrigeration cycle by compression, cooling, and expansion. In certain such embodiments, it flows to the top of a wash column, where it counter currently contacts precooled synthesis gas and/or a H₂ containing gas from which most of the methane and hydrocarbons have been condensed. In certain embodiments, all the cold equipment is installed in an insulated “cold box.” In certain embodiments, the wash column temperature is about −190° C. In certain embodiments, the liquid nitrogen wash systems operate at pressures up to around 8 MPa. In certain embodiments, an air separation plant is installed in conjunction with liquid nitrogen wash for economy in operation. In certain embodiments, an air separation and nitrogen wash are closely integrated with one another so that economies can be realized in the refrigeration system.

In certain embodiments of the present invention, Pressure Swing Adsorption (PSA) is used for gas purification. In certain embodiments, PSA is used in lieu of, or in addition to, one or more of: LT shift conversion; carbon dioxide removal; methanation; and/or a secondary reformer. PSA may use molecular sieves as adsorbents in a series of vessels operated in a staggered cyclic mode changing between an adsorption phase and various stages of regeneration. In certain embodiments of the present invention a PSA is used that comprises molecular sieves as adsorbents in a series of vessels operated in a staggered cyclic mode changing between an adsorption phase and various stages of regeneration. In such a PSA system, the regeneration of the loaded adsorbent may be achieved by stepwise depressurization and by using the gas from this operation to flush other adsorbers at a different pressure level in the regeneration cycle. In certain embodiments of the present invention, a PSA is utilized where the regeneration of the loaded adsorbent may be achieved by stepwise depressurization and by using the gas from this operation to flush other adsorbers at a different pressure level in the regeneration cycle. In certain embodiments of the present invention using PSA, the hydrogen recovery from the PSA may be as high as 90%. In certain embodiments of the present invention using PSA, the number of adsorbers in one line may be as high as 10. In certain embodiments of the present invention, gas purification units are utilized that result in an output gas with about 50 ppm argon or less, and/or about 10 ppm or less of other impurities. In certain such embodiments, the said gas purification units comprise one or more PSA units.

In certain embodiments a molecular sieve is used for gas purification. In certain embodiments, a molecular sieve is used for dehydration or drying of make-up gas and/or recycled gas. In certain embodiments, the dehydration or drying of make-up and/or recycled gas occurs prior the flowing into a compressor or through a filter.

In certain embodiments, the process scheme for plants designed according to the present invention may include or consist of the production of pure or nearly pure hydrogen and capture and/or production of pure or nearly pure carbon dioxide and capture and/or production of pure or nearly pure oxygen. In certain such embodiments the hydrogen is produced via one or more of electrolysis, steam reforming, gasification, partial oxidation, and/or WGS. In certain such embodiments the carbon dioxide is captured from a flue gas stream and/or from the air and/or water and/or geological sources and/or is produced via one or more of steam reforming, gasification, partial oxidation, and/or WGS. In certain such embodiments the oxygen is captured from the air e.g., using an air separation unit and/or produced via electrolysis of water. In certain embodiments a PSA is utilized to reduce or remove nitrogen. In certain such cases the source of the nitrogen is air, in other such cases the source of the nitrogen is process air fed to a secondary reformer.

### Bioreactors

The microorganism may be cultured using any suitable bioreactor or fermenter. The bioreactors of the present disclosure may include any other suitable components for growing a microorganism culture. The bioreactor vessel may be any suitable vessel for large or small scale microbial culturing. Suitable bioreactors include but are not limited to one or more of the following: airlift reactors; biological scrubber columns; bubble columns; stirred tank reactors (STRs); continuous stirred tank reactors (CSTRs); counter-current, upflow, expanded-bed reactors; digesters and in particular digester systems such as known in the prior arts of sewage and waste water treatment or bioremediation; filters including but not limited to trickling filters, rotating biological contactor filters, rotating discs, soil filters; fluidized bed reactors; gas lift bioreactors; immobilized cell reactors; loop reactors including but not limited to stirred loops, gas lift loops; membrane biofilm reactors; pachuca tanks; packed-bed reactors; plug-flow reactors; static mixers; trickle bed reactors; and/or vertical shaft bioreactors.

In certain embodiments of the present invention, the said bioreactor is designed and constructed to contain elevated pressures and to be operated at elevated pressures such as at least around 3 bar, or at least around 5 bar, or at least around 10 bar, or at least around 20 bar, or at least around 40 bar, or at least around 80 bar, or at least around 160 bar, or at least around 320 bar, or at least around 400 bar.

In certain embodiments, one or more bioreactors are used in the present invention which have a volume of at least one m³, or at least 10 m³, or at least 50 m³, or at least 100 m³, or at least 500 m³, or at least 1,000 m³, or at least 1,500 m³.

In certain embodiments of the present invention, reactor configurations are used that improve on mass transfer by increasing pressure, gas/liquid contact time, gas/liquid contact area, and/or by improving the energy efficiency of mixing.

In certain embodiments of the present invention, the relatively severe conditions of high pressure, and/or high hydrogen partial pressures place strict requirements on the construction materials and/or design of the bioreactor. In certain non-limiting embodiments, comparatively low-alloy chromium-molybdenum steels are utilized in the construction of one or more units of the bioprocess.

Stirred or agitated vessels are ubiquitous in the chemical and process industries. Stirred or agitated vessels also have a long history in fermentation at industrial scale. They are used extensively in various area including: bio-pharmaceuticals, mammalian cell cultures, bacterial cultures, plant cell cultures, and fungal cultures, to make products including industrial enzymes, vitamins, alcohols, antibiotics, and amino-acids Often they have a single agitator motor either mounted on the bottom or top. In certain embodiments, a stirred or agitated vessel is utilized with the agitator motor mounted on the bottom, and in other embodiments, on the top. The number and type of agitation devices on the shaft can be varied. In certain embodiments of the present invention, radial type mixing devices such as Rushton turbines are utilized. In certain embodiments, a radial flow is produced, where fluid is pushed away from the turbine blades outwards to the vessel wall, where it then divides in two and deflects upwards and downwards. This establishes large circulatory vortices above and below the impeller that assist with gas bubble dispersion and increase gas hold up in the fermenter; which is desired in aerobic and gas fermentations to enhance gas mass transfer. In certain embodiments, baffles on the side of vessel walls limit vortex formation at the liquid surface. In certain embodiments, axial mixers are utilized. In certain embodiments, a combination of both radial mixers and axial mixers are utilized. In certain embodiments, the axial mixer is located towards the top of the vessel. In certain such embodiments, this is utilized to enable quick and more thorough mixing of additions into the bulk volume of the fermenter. In certain embodiments, one or more marine impellers are utilized. In certain embodiments axial type impellers are located towards the top of the mixer shaft. These impellers generally assist in promoting quicker blend/mixing times in the vessel. In certain embodiments, this is achieved by ‘down-pumping’ axial flow, and in other embodiments by ‘up-pumping’ axial flow. In certain embodiments, material is drawn downwards and in other embodiments upwards and this in turn promotes quicker mixing through the bulk of the fluid. In certain embodiments, baffles are added to the side of the vessel to prevent the creation of vortexes on the surface of the liquid at higher rotational speeds. In certain embodiments, baffles are used to prevent all the fluid rotating as a single entity in the vessel and thereby help with liquid mixing. In certain embodiments, one or more of the following impeller designs are utilized: Scaba impeller; Chemineer BT-6 impeller, Smith CD-6 impeller. In certain embodiments, the agitator flange, seal, and drive system are located at the bottom of the vessel. In certain such embodiments, suitable safety interlocks are provided to prevent gassing when the vessel is empty. In certain embodiments, such measures are taken to ensure the rotating shaft is always covered by liquids—eliminating a possible ignition source. In certain embodiments, bottom mounted magnetic mixers are used. In certain embodiments, turbulent eddy lengths on the scale as the Kolmogorov eddy length are avoided. Sanches Perez, J. A. et al.; Shear rate in stirred tank and bubble column bioreactors. Chem. Eng Journal (2006), 124, 1-5 is incorporated herein by reference in its entirety.

In certain embodiments of the present invention, one or more stirred or agitated vessels are used in a CSTR process.

In certain embodiments, jackets are used for heat transfer. As fermenters increase in scale the ratio of volume to available surface area for heat transfer becomes limiting; since heat load is proportional to the volume of biomass. To address this issue, additional surface area may be added to the fermenter. In certain embodiments, cooling coils and/or pumped external loops with heat exchangers are used for temperature control. In certain embodiments, fixed spray ball devices and/or rotary impact jet sprays are utilized for clean-in-place (CIP). In certain embodiments, these devices are combined with a fully automated CIP system so large fermenter vessels can be easily and repeatedly cleaned with minimal intervention by operations staff. In certain embodiments, sterilization and/or steaming-in-place are utilized to sterilize the vessel before and/or after a culture run. In certain embodiments, standard engineered components, sanitary piping, accessories, and control systems are integrated to create automated steam in place systems.

Paul, E., L., et al. Handbook Of Industrial Mixing, Wiley, 2004, and Perry, R., H., et al., Perry's Chemical Engineer's Handbook, 7th Ed., McGraw-Hill, 1999 are incorporated herein by reference in their entirety.

Certain aspects of the present invention relate to headspace gas analysis and design of headspace gas composition monitoring and safety systems to ensure the headspace remains safe during operation. In certain embodiments of the present invention, potential ignition sources in the headspace, and in any gas recirculation loops, are eliminated by specifying instruments that are Class 1 Div. 1 and/or Atex Zone 0 compliant. In certain embodiments the headspace, and any gas recirculation loops, and any vent lines, are maintained outside of the flammability range (i.e., fuel-rich or fuel-lean), and the instrumentation and/or design is Class 1 Div. 2. In certain embodiments where the headspace, and any gas recirculation loops or vent lines are maintained outside the flammability range, which would indicate the use of Class 1 Div. 2 instrumentation and design, Class 1 Div. 1 and/or Atex Zone 0 compliant instrumentation and design instead are utilized as an added layer of safety, In certain non-limiting embodiments, where a vessel is used for disengagement of gases, in such embodiments the disengagement vessel is designed to fully withstand the overpressure of a detonation, mitigating the safety consequences of catastrophic vessel rupture.

In certain embodiments of the present invention the mol % of O₂ in the reactor headspace is limited to mol % O₂≤5%, or mol % O₂≤4%, or mol % O₂≤3%, or mol % O₂≤3%, or mol % O₂≤2%, or mol % O₂≤1%. This is done to ensure a non-flammable, fuel-rich (e.g., H₂-rich) and/or an O₂-lean (e.g., O₂ concentration below the MOC with an added engineering margin of safety) gas mixture in the reactor headspace at a given pressure. In certain embodiments, this precaution is taken to add another layer of safety in the process beyond design and engineering to eliminate potential ignition sources.

A hollow gas entrainment impeller utilized in a STR can re-entrain headspace gases to enhance gas-to-liquid mass transfer while simultaneously providing mixing and agitation. These vessels have been developed by specialist equipment vendors primarily for gas-liquid reactions in the bulk chemical industries—e.g., hydrogenation reactions. An exemplar of the use of a hollow gas entrainment impeller utilized in a STR is the Ekato hydrogenation reactors https://www.ekato.com/products/process-plants-and-units/hydrogenation-plants-and-hydrogenation-reactors/is incorporated herein by reference in its entirety. However, the Ekato hydrogenation reactors are generally not used in a biological process. In certain non-limiting embodiments of the present invention a STR is utilized that has a hollow gas entrainment impeller. In certain such embodiments, the STR with a hollow gas entrainment impeller is a CSTR. In certain embodiments, a STR is utilized that has a hollow gas entrainment impeller with a carbon source that is CO₂ and/or other gaseous C1 feedstocks. In certain embodiments of the present invention, the culture utilized within a STR having a hollow gas entrainment impeller does not produce any CO₂ as a metabolic waste product. In certain such embodiments, the said culture does not produce any gaseous metabolic waste or co-products. In certain such embodiments, the culture grown within a stirred loop bioreactor comprises a knallgas microorganism. In certain embodiments, a STR is utilized that has a hollow gas entrainment impeller, which is augmented by an external gas recirculation loop. In certain embodiments, a specialized impeller element with highly angled blades, used to rapidly and widely distribute the feed gas into the liquid phase, is located towards the bottom of the agitator shaft. In certain such embodiments, said specialized impeller element is located near a gas sparger. In certain embodiments, further up the shaft of the agitator assembly is located an impeller element that is a “self-aspirating” device. In certain embodiments, the shaft of the agitator assembly is hollow, and this permits the self-aspirating impeller to draw in headspace gas and redistribute it into the liquid phase. In certain embodiments, a hollow gas entrainment impeller enables headspace gas to be recycled, thus greatly increasing gas utilization rates and/or feedstock conversion. In certain embodiments, a STR is utilized that does not have a hollow gas entrainment impeller, but which does have an external gas recirculation loop. Patwardhan, A. W. & Joshi, J. B. Design of gas-inducing reactors. Industrial and Engineering Chemistry Research (1999) doi:10.1021/ie970504e, and https://www.ekato.com/products/impellers/gasjet/ are incorporated herein by reference in their entirety. In certain embodiments, the bioreactor comprises a pressure vessel with a cooling jacket and/or internal cooling coils. In certain embodiments, a STR or CSTR with a hollow gas entrainment impeller is operated with a ‘fuel rich’, non-flammable headspace. In certain embodiments, a STR or CSTR with a hollow gas entrainment impeller recycles feedstock gases back from the headspace into the fermentation broth for higher utilization rates and/or more complete feedstock conversion. Certain aspects of the present invention relate to ensuring the head space always remains fuel rich/non-explosive accounting for the relative uptake rates of various feedstock gases (e.g., H₂, CO₂, and/or O₂) during the various stages of fermentation. In certain embodiments, control of input gas rates and headspace gas composition monitoring is utilized to ensure the headspace remains non-explosive during all modes of operation. In certain embodiments, the headspace and bioreactor system use Class 1 Div. 1 and/or Zone 0 Atex rated instrumentation. In certain embodiments, the headspace is maintained in a non-flammable (i.e., fuel-rich or fuel-lean) state and Class 1 Div. 2 instrumentation and/or design is utilized. In certain embodiments where the headspace, and any gas recirculation loops or vent lines are maintained outside the flammability range i.e., containing non-flammable gas mixtures, which would indicate the use of Class 1 Div. 2 instrumentation and design, Class 1 Div. 1 and/or Atex Zone 0 compliant instrumentation and design are instead utilized as an added layer of safety. In certain embodiments, a hollow gas entrainment impellor is mounted on the bottom of the vessel under the liquid, thus eliminating a potential ignition source.

A bubble column or gas lift bioreactor may improve the energy efficiency of mixing and mass transfer by relying on gas bubble buoyancy to drive turbulence and mixing and/or by relying on large hydrostatic pressures to drive dissolution of gaseous reactants into solution. Bubble columns and airlift/gas lift fermenters have a proven heritage in industrial fermentations and are generally robust and simple devices well suited for highly aerobic fermentations. These fermenters have been utilized safely and profitably at an industrial scale for decades. Airlift fermenters have been used at large industrial scale to produce single cell protein in aerobic fermentations. Notably the Quorn airlift fermenters, which have a loop geometry, are used in the production of mycoprotein for human consumption. This fermenter architecture is suitable from a sterility, shear, and cleanability context for generating large quantities of food grade, protein rich biomass.

In certain embodiments of the present invention, large volumes of gas are introduced into the bottom of a tall liquid filled column. Certain such embodiments, constitute a bubble column type or a trickle bed type bioreactor. In other non-limiting embodiments, internals ensure that the two-phase lower density fluid is segregated from a high-density single-phase liquid column which in turn sets up a circulatory flow pattern. The rate of circulation, and thus bulk mixing performance, is principally controlled by the rate of gas addition and therefore can be correlated to the superficial gas velocity in the riser section. Relatively uniform liquid velocity gradients are a characteristic that distinguishes airlift reactors from other gas liquid contact reactors. The liquid velocity is a function of several other parameters, predominantly gas superficial velocity, but also gas holdup and pressure drop along the flow path, and bioreactor geometry. Siegel et al. (Siegel, M. H., Hallaile, M. and Merchuk, J. C. (1988); Advances in Biotechnology Processes: Upstream processes, equipment and techniques (Mizrahi, A., ed) (Vol. 7) pp. 79-124, Alan R. Liss) has reviewed the mixing performance of air lift fermenters and is incorporated herein by reference in its entirety. The larger the specific power per unit volume input to a pneumatically mixed gas lift fermenter, generally the better the mixing and blending performance [Chisti, Y. and Moo-Young, M.; Communications to the Editor: Biotechnology and Bioengineering, Vol. 34, Pp. 1391-1392, (1989) is incorporated herein by reference in its entirety].

In bubble column and gas-lift bioreactors, the power input for mixing is generally achieved with no moving mechanical parts (e.g., agitators), which in turn eliminates the sterility risks associated with rotating shafts and mechanical seals directly coupled to the sterile envelope of a fermenter. The absence of rotating shafts and mechanical seals also removes a possible ignition source from inside of the fermenter where a potentially explosive gas mixture could be present. In an airlift/gaslift system the pneumatic mixing and power input/power dissipation via gas expansion is relatively homogenous across the diameter and length of the riser section. By comparison, for an agitated vessel there is a localized higher power dissipation closer to the tips of the agitator and conversely power dissipation is lower near the vessel walls. Research and a literature review indicate that airlift reactors (ALR)/gas lift fermenters (GLF) are, actually, one of the most efficient and stable fermenter types in terms of gas mass transfer performance across a wide range of transfer rates and operating conditions (Gas mass transfer performance is defined as the mass of oxygen transferred per unit power input to the fermenter). Orazem, M. E. and Erickson, L. E. (1979) Biotechnol. Bioeng. 21, pp. 69-88 is incorporated herein by reference in its entirety.

An exemplar of the use of a gas lift bioreactor is that which was used in the Imperial Chemical Industries (ICI) Pruteen single-cell protein (SCP) bioprocess. A gas lift fermenter with internal central draught tube was used by ICI to make “Pruteen”. This fermenter was operated successfully for several years at the c. 1,500 m³ scale. However, the Pruteen process did not utilize CO₂ or another gaseous C1 molecules as a carbon source. In certain non-limiting embodiments of the present invention a gas lift bioreactor is utilized. In certain such embodiments, the carbon source is CO₂ and/or other gaseous C1 feedstocks. J. A. M. van Balken, N. Open Universiteit Heerlen, and U. of Greenwich, Biotechnological Innovations in Chemical Synthesis, Chap. 4.9, ser. BIOTOL (Open universiteit). Butterworth Heinemann, 1997. [Online]. Available: https://books.google.com/books?id=uMxmP0v-iAYC is incorporated herein by reference in its entirety.

In certain embodiments, the primary electrical power demand for pneumatic agitation of the broth will be consumed by the electrical motors on the gas/air compressors. In certain embodiments, said gas/air compressors are installed and operated outside of the sterile envelope and/or located in a ‘safe’ area away from where potentially explosive gas atmospheres may be present. In certain embodiments, the gases to be used are from a piped or cryogenic supply under pressure, and in such embodiments, the energy consumption of the fermentation system could potentially be reduced by utilizing the potential energy in the pressurized gas to reduce or eliminate the need for gas compressors. In certain embodiments, H₂ gas is provided at elevated pressure from an electrolyzer. In certain embodiments, the feedstock gases are received at high pressures—e.g., from a pipeline supply, which reduces power consumption by the fermentation plant.

Most airlift fermenters and bubble columns tend to have a large disengagement zone or compartments to permit rapid and efficient desorption and degassing of: waste/toxic metabolic gases (e.g., carbon dioxide) created during conventional aerobic fermentations; unused oxygen; and inert gases e.g., residual nitrogen from air. In certain embodiments of the present invention, no waste and/or toxic metabolic gases such as carbon dioxide are produced by the culture. In certain embodiments, the introduction of inert gases in minimized by using pure input gases (e.g., oxygen instead of air). In certain embodiments comprising one or more of these features (e.g., no metabolic waste gases and/or minimal input inert gases) an airlift/gaslift fermenter or bubble column is utilized that does not have a large disengagement zone or compartments to permit rapid and efficient desorption and degassing.

Degassing of the two-phase broth in airlift fermenters causes its bulk density to increase and descend the down-comer section thus creating the circulatory liquid flow pattern. Without an effective degassing and disengagement zone these fermenter types would not operate satisfactorily in the case of traditional aerobic bioprocesses (e.g., sugar or organic carbon source). In certain embodiments, clean gases (i.e., free, or largely free from contaminants and inert gases), such as H₂, CO₂, and/or O₂ are fed into the system. In certain embodiments, the strain or strains used in the culture do not produce any gases through respiration during growth, and/or no gases are generated within the culture system. In certain embodiments, both conditions hold true, and the requirement of a degassing zone is minimal or absent. In certain such embodiments, all gases remaining unconsumed at the top of the gaslift bioreactor are recirculated within the system.

In certain embodiments, the input reactant gases (e.g., H₂, CO₂, and/or O₂) are injected at rates that meet metabolic demands of the biomass such that most or all of the gases are consumed before they reach the disengagement vessel or headspace. The said input reactant gases may, or may not include inert gases such as N₂ that pass unreacted through the working volume to the headspace. In other embodiments, the H₂, is injected at rates that meet metabolic demands of the biomass such that most or all of the H₂ is consumed before it reaches the disengagement vessel or headspace, however other gases such as CO₂, N₂, and/or O₂ still remain after passage through the working volume and coalesce into the headspace. In other embodiments, the H₂, and O₂ is injected at rates that meet metabolic demands of the biomass such that most or all of the H₂ and O₂ is consumed before they reach the disengagement vessel or headspace, however other gases such as CO₂ and/or N₂ still remain after passage through the working volume and coalesce into the headspace. In certain embodiments, where either all or most of the reactant gases are consumed during passage through the working volume, or where only the H₂ and O₂ are entirely or mostly consumed, or where only the H₂ is entirely or mostly consumed, the bioreactor type containing the said working volume may be one or more of: stirred tank; bubble column, gas lift, and/or trickle-bed reactor.

In certain embodiments utilizing a gas lift bioreactor, the consumption of gases by the culture through the riser is designed to replace or complement degassing of the two-phase broth such that its bulk density increases at the top of the column and descends the down-comer section thus creating the circulatory liquid flow pattern. In certain such embodiments, the said gas consumption allows the replacement or reduction of the gas disengagement system without compromising the system hydraulics and/or reducing the efficacy of the gaslift system.

As the bulk liquid agitation in bubble column, gas lift, and trickle bed reactors is created by the gas phase, in certain embodiments there are no ignition sources present in the headspace that are associated with top mounted mechanical agitation. In certain embodiments, the only possible sources of ignition in the headspace are instruments and/or probes. In certain such embodiments, the said instruments and/or probes are specified for operation within a potentially flammable atmosphere.

Loop reactors may provide more energy efficient gas/liquid mixing and/or mass transfer. Certain embodiments of the present invention utilize a loop reactor. A pressure cycle loop bioreactor combines features of a gas lift bioreactor with a loop geometry and liquid flow. An exemplar of the use of a pressure cycle loop bioreactor is the Quorn Foods mycoprotein bioprocess used by Marlow Foods to produce “Quorn”. Although the design and construction of the pressure cycle loop bioreactor is different than the ICI Pruteen gas lift fermenter, the operating principles are fundamentally the same. Large volumes of gas are introduced into the bottom of a tall liquid filled column. A loop ensures that the 2 phase lower density fluid is segregated from a high-density single-phase liquid column which in turn sets up a circulatory flow pattern. The physical configuration of a gas lift fermenter with an external recirculation loop generally consists of four major constituents: Riser; Downcomer; Disengagement vessel/headspace; Base—typically including some form of cooler. This technology has reportedly operated robustly at the c. 200 m³ scale at the Quorn Belasis site. However, the Quorn process does not utilize CO₂ or another gaseous C1 molecule as a carbon source. In certain non-limiting embodiments of the present invention a pressure cycle loop bioreactor is utilized. In certain such embodiments, the carbon source is CO₂ and/or other gaseous C1 feedstocks. In certain embodiments the flow regime through the loop is fully turbulent (i.e., Reynolds number is ≥4000). T. Finnigan, L. Needham, and C. Abbott, “Chapter 19—mycoprotein: A healthy new protein with a low environmental impact,” in Sustainable Protein Sources, S. R. Nadathur, J. P. D. Wanasundara, and L. Scanlin, Eds. San Diego: Academic Press, 2017, pp. 305-325. [Online]. Available: http://www.sciencedirect.com/science/article/pii/B9780128027783000196 is incorporated herein by reference in its entirety.

A mechanically stirred loop combines features of a stirred tank reactor with a loop geometry and liquid flow. Certain embodiments of the present invention utilize a mechanically stirred loop bioreactor. Examples of stirred loop reactors include the D-loop and U-loop fermenters. The terms ‘D’ and ‘U’ refer to the general orientation of the pipe that makes up the majority of the fermenter working volume. The pipe can be either oriented vertically or horizontally. If mounted vertically the fermenter is often described as a U-loop. If mounted horizontally the fermenter is described as a D-loop. Certain embodiments of the present invention that utilize a stirred loop have a U-loop type configuration, and other embodiments have a D-loop type configuration. The main components found in both types are: gas/liquid disengagement vessel—can be oriented vertically or horizontally; pipe configuration that generally creates a closed loop that originates and terminates at the disengagement vessel; often there is a pump internal to the loop (i.e. inside the sterile envelope) that provides the motive power to the broth and circulates it around the pipe loop; mounted within the loop of pipe at specific locations are static mixer elements which are used to mix and distribute the gas and liquid; upstream of the static mixers there are direct gas injection points that introduce sterile gases into the broth upstream of the mixers. The motive power to circulate the liquid phase is provided by some form of inline pump. In certain embodiments, the pump has a variable speed drive. In certain embodiments, a pump is used that can handle bubbly two-phase flow. In certain embodiments, a multi-vane, low cavitation, slowly rotating marine propeller is used for the pump—this style of pump was used historically on the Norferm/Dansk Bioprotein fermenters. In certain embodiments, the loop is in a vertical orientation (e.g., U-loop) and the pump is located at the bottom of the loop, as far as possible from the disengagement vessel (if any disengagement vessel is present). In the lower U-bend of a U-loop some separation of the liquid and gas may occur due to centrifugal forces. In certain embodiments, a pump is utilized that can handle two phase mixtures. In certain such embodiments the pump helps to redistribute the gas into the liquid phase. In certain embodiments, the loop is in a horizontal orientation (e.g., D-loop) and the pump is located relatively close to the disengagement vessel (if any disengagement vessel is present), at the bottom of the disengagement vessel. The pump must deliver enough head to overcome the frictional losses around the loop and create a superficial velocity of enough magnitude that the inline static mixers are effective at mixing gas and liquid phases. In certain embodiments a liquid velocity of at least 1-2 m/s is passed through the static mixers. In certain embodiments a liquid velocity of at least 1.25 m/s is passed through the static mixers. In certain embodiments, the superficial velocity is set at the peak mass transfer of oxygen per unit energy. In certain embodiments, that superficial liquid velocity is around 1.5-1.7 m/s. In certain embodiments, the superficial velocities are greater than 1.5 m/s. Taweel A. M., Yan J., Azizi F., Odedra D., Gomaa H. G., Using Inline static mixers to intensify gas-liquid mass transfer processes. Chemical Engineering Science 60 (2005) 6378-6390 is incorporated herein by reference in its entirety. Unlike gaslift fermenters, in stirred loop reactors the liquid circulation rate, mixing performance and gassing rate are decoupled and can be independently varied. This is because, unlike gaslift fermenter types, the circulation around the loop is created by a pump. So full and complete disengagement of the gases is not as crucial in terms of creating the desired circulatory flow pattern. In certain embodiments of the present invention, gassing rates, bulk mixing and liquid circulation rates are decoupled. In certain embodiments the flow regime through the loop is fully turbulent (i.e., Reynolds number is ≥4000).

An exemplar of the use of a stirred loop bioreactor is the U-loop bioreactor used in the UniBio SCP bioprocess. However, the UniBio process is a methanotrophic process that utilizes CH₄ as a carbon source and produces CO₂ as a gaseous metabolic waste product. In certain non-limiting embodiments of the present invention a stirred loop bioreactor is utilized. In certain embodiments of the present invention, the culture utilized within a stirred loop bioreactor does not produce any CO₂ as a metabolic waste product. In certain such embodiments, the said culture does not produce any gaseous metabolic waste or co-products. In certain such embodiments, the culture grown within a stirred loop bioreactor comprises a knallgas microorganism. In certain embodiments, the gas/liquid disengagement vessel that is typically present in stirred-loop bioreactors (e.g., U-loop or D-loop), is reduced or eliminated. In certain embodiments, there is little gas headspace or no gas headspace while the stirred loop is in operation. In such embodiments, the entire reactor volume is filled or essentially filled with working volume consisting of the liquid and gas bubble suspension. E. B. Larsen, “U-shape and/or nozzle u-loop fermenter and method of carrying out a fermentation process,” December 2002, U.S. Pat. No. 6,492,135. is incorporated herein by reference in its entirety. In certain embodiments of the present invention using a mechanically stirred-loop reactor, static mixers are used to generate turbulence. In other embodiments using a mechanically stirred-loop reactor the liquid velocity around the loop results in a Reynolds number high enough to generate turbulence. In certain such embodiments, the said turbulence generated by a high Reynolds number is sufficient to meet the mixing requirements of the bioprocess without the use of static mixers, and as such, static mixers are not utilized. In certain embodiments of the present invention using a mechanically stirred-loop reactor, both static mixers and a liquid velocity around the loop giving a Reynolds number high enough to generate turbulence are used to meet the mixing requirements of the bioprocess. In certain embodiments using a mechanically stirred-loop reactor, the liquid velocity around the loop, and particularly in the top section of the loop, acts to re-entrain back into the liquid any gas headspace that accumulates at the top of the loop. In certain embodiments having no gas/liquid disengagement vessel, the flow of the liquid around the loop results in the reduction or the elimination of any gas headspace within the loop. In certain such embodiments having a vertically oriented loop, when the stirred loop is in operation, centrifugal forces at the upper bend of the loop act to invert the relative positions of the gas and liquid phases, compared to their normal position under gravity when there is zero liquid flow velocity around the loop (i.e., gas headspace above the liquid).

Certain aspects of the present invention relate to determining the optimum loop lengths, diameters, and relative placement of static mixers.

As with air/gas lift fermenters, stirred loop bioreactors have generally had a disengagement vessel to assist with degassing broth—primarily to help remove toxic metabolic waste gases from the system thus preventing their accumulation. In certain embodiments of present invention, the bioprocess does not generate metabolic waste gases and the need/complexity of a disengagement system will be driven more by the purity of the feed gases. In certain such embodiments, pure feed gases are utilized such that the gas disengagement system can be reduced or eliminated. In certain embodiments the volume of headspace is reduced to an absolute minimum. In certain embodiments, the gases are injected at rates that meet metabolic demands of the biomass such that most or all of the gases are consumed before they reach the disengagement vessel or headspace. In other embodiments, the gases aren't entirely consumed before they reach the top of the loop, however they remain entrained in the liquid and do not degas into a headspace, but rather are forced downward on the other side of the loop by the liquid flow around the loop. In certain embodiments, the fermentation produces no waste gases—only water and biomass (e.g., suspended and dissolved organic matter). In certain such embodiments, no gas disengagement is required, and no gas disengagement vessel is needed. In certain such embodiments, the removal of the gas disengagement vessel reduces or removes the hazard associated with an explosive/flammable gas accumulation within the fermenter. Compared to an airlift fermenter the desorbing of metabolic carbon dioxide presents a challenge in stirred loop reactors as there is a tendency for the gas to remain in solution due to the action of static mixers and relatively high hydrostatic head pressures. This characteristic can prove disadvantageous in bioprocesses that produce a metabolic carbon dioxide waste product, such as heterotrophic or methanotrophic fermentations. However, in certain embodiments of the present invention, there is no net metabolic carbon dioxide waste product. In certain embodiments, carbon dioxide is instead a nutrient i.e., reactant. In such embodiments, the difficulty in desorbing carbon dioxide observed in stirred loop reactors is instead an advantage. For use with heterotrophic or methanotrophic bioprocesses producing CO₂ waste, a region in the stirred loop fermenter is often designed immediately upstream of the disengagement vessel having no static mixers, reduced head pressures, and active introduction of some form of stripping gas stream to assist with carbon dioxide removal from the liquid phase. In certain embodiments, the stirred loop bioreactor of this invention has none of these features—no absence of static mixers; no reduced head pressure; no stripping gas. In certain embodiments of the present invention, there are no gaseous products formed. In certain embodiments, elimination of a disengagement vessel and/or features upstream of the disengagement vessel in the stirred loop will greatly simplify the design and operation of a loop fermenter and/or essentially eliminate the significant hazard of potentially explosive gas accumulations.

Certain aspects of the present invention relate to the nutrient addition rates and locations in the loop. Certain aspects relate to designing nutrient addition rates and locations based upon knowledge of the loop circulation time in an attempt to limit the creation of high concentration ‘pulses’ of additions circulating around the loop. In certain embodiments, liquid additions are slowly added into the loop pipe to create uniform blends. Certain aspects of the present invention relate to pH control through understanding plug flow dynamics. In certain embodiments a form of advanced control is utilized to help achieve acceptable pH control around the loop fermenter. Certain such embodiments include gain scheduling or a characterizer function block in the control system to account for the highly non-linear nature of the pH scale, the overall process dead time, and process gain of a loop fermenter.

In certain embodiments, one or more of the following options for heat transfer in stirred loop fermenters are used: a concentric jacket heat exchanger along the length of the pipe; external heat exchanger(s); internal cooling coil(s). In certain embodiments, a concentric jacket heat exchanger is integrated along lengths of pipe. In certain such embodiments, the heat exchanger is non-invasive, and does not alter flow regimes, introduce additional pressure drops inside the loop, or introduce internal Clean In Place (CIP) challenges. In certain embodiments, external heat exchangers are used. In certain such embodiments, the said external heat exchangers comprise a side stream of broth that is extracted from the fermenter and cooled sensibly against a cooling utility stream in a suitable heat exchanger. In certain such embodiments, the cooled side stream of broth is then re-introduced into the bulk of the fermenter at a temperature lower than the desired set point, such that when it mixes with the bulk broth the resultant temperature is at a desired value. By using an external heat exchanger, the duration the broth will be out of the main fermenter is an important design and operational consideration as the broth is not oxygenated unless specific sparger or oxygen injection points are introduced into the external cooling loop. In certain embodiments, sparger or oxygen injection points are introduced into an external cooling loop. In certain embodiments, internal cooling coils are used. In certain such embodiments, thermal analysis of heat load distribution is used to help determine the location(s) and amount of heat transfer area required.

Certain aspects of the present invention relate to clean-in-place (CIP) and sterilization-in-place (SIP). In certain embodiments of the present invention, prior to and/or following a run, loop fermenters are flooded and then cleaned in place using heated water rinses and caustic washes followed by final water washes and rinses to eliminate caustic residues. In certain embodiments, a main circulation pump with a variable speed drive is used in flood filled CIP. Very generally to clean a surface using a flowing liquid the superficial velocity of the cleaning fluid must be two to four times that attained during normal operation. This ensures that a more turbulent flow regime (i.e., greater Reynolds numbers) is created during CIP and thus the wall shear stresses are greater which in turn helps to dislodge any bio-films or accumulations that have attached to the pipe wall during production. In certain embodiments, the cleaning fluid is circulated with a superficial velocity two to four times that attained during normal operation. In certain embodiments, the CIP fluid is hot and contains an appropriate cleaning agent (e.g., caustic). In certain embodiments, the combined effects of one or more of: velocity; wall shear stress; turbulence; temperature; reactive chemistry; and time, are used to physically and chemically remove biomass from suitably smooth surfaces. In certain embodiments, flood filled CIP is flowed reverse the culture flow direction around the loop to further clean the ‘downstream’ faces of internal surfaces. In certain embodiments, automated control systems and valves are used to operate the plant in a manner such that CIP fluids are directed around the extremities of the loop and any heat exchange sub-loops without causing cross contamination or creating process hazards.

For both vertical U-loops and horizontal D-loops their long, thin aspect ratios can prove difficult in ensuring steam is properly and fully introduced in their entire length whilst purging any air or non-condensable gases totally from the main loop and sub-loops if present. Certain aspects of the present invention relate to designing sizing, timing/operation and location of steam injection points, pipe slopes, drain points, and steam traps to ensure repeatable and reliable air removal and condensate removal from within the sterile envelope.

Certain loop reactors utilize forced liquid circulation with venturi eductor gas entrainment and/or re-entrainment that provides both gas to liquid mass transfer as well as mixing and agitation. These reactors have been used in the chemical industry where good gas mass transfer is required and often where hazardous gases are to be reacted with a liquid phase e.g., hydrogenation and chlorination reactions. The system generally comprises: reaction vessel; external recirculation loop with pump; top mounted gas/liquid eductor/ejector. Liquid is drawn out of the vessel into the recirculation loop via the recirculation pump. The liquid phase is then pumped through a top mounted ejector local to the vessel. The ejector is designed to accelerate the liquid such that a corresponding reduction in liquid pressure occurs (Venturi effect). A side stream of gas is located where the liquid velocity is maximized in the ejector, and thus where the liquid pressure is minimized. This low-pressure region results in gas being drawn into the flowing liquid. The resulting liquid and gas mixture is then ejected from the nozzle with downward momentum into the headspace of the reaction vessel. The high-speed jet of liquid and gas impinges on the bulk liquid surface inside the reactor resulting in further entrainment of the headspace gas into the bulk liquid as well as turbulent mixing. The net effect of this is locally high gas mass transfer rates.

The Buss ChemTech ejector loop reactor [M. Ughetti, D. Jussen, and P. Riedlberger, “The ejector loop reactor: Application for microbial fermentation and comparison with a stirred-tank bioreactor,” Engineering in Life Sciences, vol. 18, no. 5, pp. 281-286, 2018. is incorporated herein by reference in its entirety], and the venturi bioreactor described in P. Dalla-Betta and J. S. Reed, “Method and apparatus for growing microbial cultures that require gaseous electron donors, electron acceptors, carbon sources, or other nutrients,” October 2015, U.S. Pat. No. 9,157,058, which are both incorporated herein by reference in their entirety, are exemplars of loop bioreactors using the venturi effect. Thalasso, F., Naveau, H., Nyns, E., J. Design and Performance of bioreactor equipped with a Venturi injector for high gas transfer rates; The Chemical Engineering Journal, 57 (1995) B1-B5 is also incorporated herein by reference in its entirety. However, the Buss ChemTech ejector loop reactor has not been used in bioprocesses that utilize CO₂ or another gaseous C1 molecule as a carbon source, and the venturi bioreactor described in U.S. Pat. No. 9,157,058 was not designed for operation at pressures comparable to those used in chemical GTL processes e.g., hydrogenation, Fischer-Tropsch, methanol synthesis, and Haber-Bosch processes. In certain non-limiting embodiments of the present invention an ejector loop bioreactor and/or a loop bioreactor that applies forced liquid circulation and the venturi effect for gas entrainment, is utilized. In certain such embodiments of the present invention, the carbon source is CO₂ and/or other gaseous C1 feedstocks. In certain such embodiments the reactor is maintained at a pressure of at least 3 bar, or at least 5 bar, or at least 10 bar, or at least 20 bar, or at least 40 bar, or at least 80 bar, or at least 160 bar, or at least 320 bar, or at least 400 bar. In certain embodiments, the ability of this reactor to recycle headspace gas enables operating the headspace fuel rich (i.e., above the UEL for the gas mixture). In certain such embodiments, the said fuel is H₂ and the gas mixture above the UEL comprises O₂. This way expensive feedstock gases are not wasted by venting to atmosphere as is the case for fuel-rich headspaces in ‘once through’ systems. In certain embodiments, the credible sources of ignition in the headspace are instrumentation as there is no mechanical agitator located in the headspace. In certain such embodiments, these potential ignition sources are eliminated by specifying instruments that are Class 1 Div. 1 and/or Atex Zone 0 compliant. In certain embodiments the headspace is maintained outside of the flammability range (i.e., fuel-rich or fuel-lean), and the instrumentation and/or design is Class 1 Div. 2.

A membrane reactor can be used to diffuse gases such as H₂ and/or O₂ through a gas permeable/water impermeable membrane into a biofilm and/or culture medium. An exemplar of the use of a membrane reactor is the ARO Technologies denitrification membrane reactor. However, this membrane is used in a denitrification process. In certain non-limiting embodiments of the present invention a membrane bioreactor is utilized. In certain embodiments of the present invention, the cultured utilized within a membrane bioreactor utilizes O₂ as an electron acceptor. In certain such embodiments, the said culture does not produce any gaseous metabolic waste products. In certain such embodiments, the culture grown within a membrane reactor comprises a knallgas microorganism. In certain embodiments of the present invention a membrane reactor is used to produce SCP or SCP derived products. In certain embodiments of the present invention a membrane reactor is used to produce organic molecules from CO₂. In certain such embodiments, the said organic molecules are produced through anabolic biosynthetic pathways.

Bioreactors have generally been developed for bioprocesses where there are gaseous products, and/or both gaseous reactants, such as methane, carbon monoxide, and/or oxygen, and gaseous products, such as CO₂ or O₂. In certain embodiments of the present invention, the bioreactor has been specifically designed for a bioprocess that has no gaseous products. In certain embodiments of the present invention, degassing features to remove waste gases such as, but not limited to, CO₂, such as sections where the bioreactor headspace or vessel widens, or the liquid velocity or turbulence is reduced, present in typical embodiments of many of the listed bioreactors, are not present in certain embodiments of the present invention, or required to the same degree.

Certain embodiments of the present invention comprise a continuous bioprocess that uses mixtures of, CO₂, O₂, and H₂. In certain embodiments of the present invention gases are only consumed in the bioprocess, they are not produced. In certain such embodiments, only liquids and solids are produced. In certain such embodiments, the lack of gaseous product highly impacts the bioreactor and/or gas recirculation design. In certain embodiments of the present invention, bioreactors are utilized that lack the degassing features used to remove metabolic waste gases, and particularly CO₂, which are found in many aerobic bioreactors used for sugar-based and methane-based bioprocesses.

Bioreactor architectures mentioned above including but not limited to: STR; bubble column; gas lift and air lift; pressure cycle loop; stirred loop; and ejector loop; have generally been developed for bioprocesses where there are gaseous products, particularly CO₂. In certain embodiments of the present invention, one or more of the respective bioreactor architectures comprising STR; bubble column; gas lift and air lift; pressure cycle loop; stirred loop; and ejector loop; are modified to better suit a bioprocess having no gaseous products. Such modifications include, but are not limited to, reducing or eliminating degassing features for the removal of waste CO₂, such as sections where the bioreactor headspace widens, and/or the liquid velocity or turbulence is reduced. Examples of these types of features include the degassers at the top of the ICI gas lift bioreactor, the Quorn pressure cycle loop bioreactor, and the UniBio U-loop stirred loop bioreactor [see above references]. Certain embodiments of the present invention, feature either a gas lift, pressure cycle loop, and/or stirred loop bioreactor with no degasser at the top of the said bioreactor.

Certain bioreactor architectures use a downcomer, where the degassed, denser liquid circulates back down to the base of the reactor through a downcomer, such as is found in the ICI gas lift and Quorn pressure cycle loop bioreactors. In such reactors, a degasser is used at the top of the reactors to densify the liquid by removing gas bubbles as the liquid is introduced to the downcomer. In certain embodiments of the present invention, the consumption of gases by the culture, which occurs in the riser, sufficiently densifies the liquid enough by the time it reaches the top of the bioreactor, for the liquid to flow down the downcomer as designed; without any degasser at the top of the bioreactor, or with a reduced degasser compared to processes producing a CO₂ waste gas. In certain embodiments, the riser section consumption of gas is sufficiently complete such that the bulk liquid phase density increases as it ascends the riser. In certain such embodiments, this sets up circulatory motion around the fermenter without the need of a disengagement vessel.

In certain embodiments of the present invention, the water column in the reactor is on the order of at least 10 meters deep, or at least 20 meter deep, or at least 30 meters deep, or at least 40 meters deep, or over 40 meters deep. M. Albæk, K. Gernaey, M. Hansen, and S. Stocks, “Evaluation of the efficiency of alternative enzyme production technologies,” Ph.D. dissertation, 2012 is incorporated herein by reference in its entirety. In certain embodiments of the present invention, increased pressure applied on certain bioprocesses described herein are at least in part provided by hydrostatic pressure resulting from the depth of water column in bioreactors used in the present invention. In certain embodiments, an applied pressure, beyond that provided by hydrostatic pressure, is additionally used in the bioprocess. The Vertical Shaft Bioreactor is a type of airlift bioreactor used in aerobic wastewater treatment where part, or the entirety of the reactor tube is sunk underground. D. C. I. Pollock, “Means for separation of gas and solids from waste mixed liquor,” July 1981, U.S. Pat. No. 4,279,754 is incorporated herein by reference in its entirety. In certain non-limiting embodiments of the present invention, some portion of the reactor tube is sunk underground. In certain such embodiments, the material requirements and/or capital costs of the bioreactor are reduced compared to an entirely aboveground bioreactor by using the ground and/or concrete casing for additional structural support at the base of the reactor. In certain such embodiments, the said ground and/or concrete casing provide additional support where the hydrostatic pressures become high at the lower part of the water column in the bioreactor. In certain such embodiments, the said bioreactor is of a bubble column type, or gas lift type, or any other architecture where it might be feasible to implement.

In certain embodiments of the present invention, the bioreactor and/or bioprocess is designed as a “dead end” for gases, or in other words, gases flow in but they don't flow out. Certain such embodiments enable extremely high conversion of gaseous feedstocks. Certain such embodiments involve recirculation of unreacted gases from the bioreactor headspace back into the bioreactor working volume. In certain embodiments, the gaseous feedstocks are fed in only at a rate that closely matches their consumption within the bioreactor and/or bioprocess. The level of inert gases like N₂ that could build up in a dead-end type system and/or a system with gas recirculation, necessitating gas purging, are an important consideration. Certain embodiments utilize pure or nearly pure CO₂, O₂, and/or H₂ gas inputs to minimize the build-up of inert gases within the gas recirculation loop and/or dead-end bioreactor that would necessitate venting or purging. Certain embodiments minimize the introduction of inert gases into the bioreactor system. Inert gases refer to gases that cannot or are not chemically reacted or metabolized by microorganisms used in the present invention. For certain microorganism that are incapable of N₂-fixing reactions, or for which a preferred N-source is present, such as but not limited to ammonia, urea, or nitrates, N₂ is effectively inert. In certain such embodiments, the introduction of N₂ into the bioreactor system is minimized. In certain embodiments, the amount of N₂ and/or other inert gases within the system (e.g., bioreactor headspace, and any gas recirculation loop) is kept at a roughly constant level overtime through a combination of minimizing input of fresh N₂ and/or other inert gases into the system, and sufficient vent/purge stream of gases to prevent the build-up of N₂ and/or other inert gases over time.

In certain embodiments of the present invention, the bioreactor architecture is chosen on the basis of operational parameters including but not limited to one or more of the following: gas flow rates, stirring rate, effect of shear, sparging, gas addition methods, stirrer design, Oxygen Transfer Rate (OTR=k_{L}a(k_{H}*pO₂−DO)), foam formation and control, reactor dimensions, gas hold-up, operating pressure and temperature, heat duties, mass transfer requirements and limitations, heat transfer requirements and limitations, agitator requirements, operating margin relative to flammability envelope, minimized production cost for a given feedstock price, and/or safety requirements. In certain non-limiting embodiments of the present invention, the bioreactor provides an OTR sufficient to maintain a biomass productivity of at least 1 g/liter/hr or ≥2 g/L/hr or ≥3 g/L/hr or ≥5 g/L/hr or ≥10 g/L/hr or ≥20 g/L/hr or ≥30 g/L/hr or ≥40 g/L/hr or ≥50 g/L/hr or ≥70 g/L/hr or ≥90 g/L/hr or ≥100 g/L/hr. In certain non-limiting embodiments of the present invention the energy efficiency of oxygen transfer (kg O₂/kWh) is minimized for a given OTR sufficient to maintain a biomass productivity of at least 1 g/liter/hr or ≥2 g/L/hr or ≥3 g/L/hr or ≥5 g/L/hr or ≥10 g/L/hr or ≥20 g/L/hr or ≥30 g/L/hr or 40 g/L/hr or ≥50 g/L/hr or ≥70 g/L/hr or ≥90 g/L/hr or ≥100 g/L/hr.

In some embodiments, the bioreactors and methods of the present disclosure provide high-density chemoautotrophic microorganism growth. In some embodiments, the optical density at 600 nm (OD₆₀₀) of a culture grown using the bioreactors and methods of the present disclosure reaches about 100 or greater, e.g., about 150 or greater, about 200 or greater, about 250 or greater, including about 300 or greater. In some embodiments, the OD₆₀₀ is in the range of about 100 to about 400, e.g., about 150 to about 400, about 200 to about 400, including about 300 to about 400.

In some embodiments, the chemoautotrophic microorganism grows rapidly to high density. In some embodiments, the chemoautotrophic microorganism culture grows to maximum density after inoculation in about 200 hours or less, e.g., in about 190 hours or less, in about 180 hours or less, in about 170 hours or less, in about 160 hours or less, including in about 150 hours or less. In some embodiments, the chemoautotrophic microorganism culture grows to maximum density after inoculation in about 100 to about 200 hours, e.g., in about 100 to about 180 hours, in about 110 to about 170 hours, including in about 110 to about 150 hours.

In some embodiments, the bioreactors and methods of the present disclosure provide rapid growth of biomass on CO₂ as sole carbon source. In some embodiments, the biomass of the chemoautotrophic microorganism culture grows at a rate of about 0.5 g/L/hr or more, e.g., about 1 g/L/hr or more, about 1.5 g/L/hr or more, about 1.7 g/L/hr or more, about 2 g/L/hr or more, about 2.5 g/L/hr or more, including about 3 g/L/hr or more. In some embodiments, the biomass of the chemoautotrophic microorganism culture grows at a rate in the range of about 0.5 to about 3.5 g/L/hr, e.g., about 1 to 3 g/L/hr, including about 1.5 to 3 g/L/hr.

In some embodiments, the bioreactors and methods of the present disclosure provide long-term, continuous culture of a microorganism under chemoautotrophic conditions. In some embodiments, the microorganism is continuously cultured under chemoautotrophic conditions for about 100 hours or more, e.g., for about 150 hours or more, for about 200 hours or more, for about 250 hours or more, for about 300 hours or more, for about 400 hours or more, for about 500 hours or more, for about 600 hours or more, for about 800 hours or more, including for about 1,000 hours or more. In some embodiments, the microorganism is continuously cultured under chemoautotrophic conditions for about 100 to about 1000 hours, e.g., for about 150 to about 900 hours, for about 200 to about 800 hours, for about 300 hours to about 700 hours, including for about 400 hours to about 700 hours.

In certain embodiments of the present invention, any gas headspaces or accumulated gas mixtures comprising a fuel gas and an oxidizing gas and/or oxidizer within the reactor system are kept above the explosive limit for flammable gas mixtures i.e., fuel-rich with a fuel gas content above the upper explosivity limit (UEL) for the given constituents of the gas mix. In certain such embodiments, the said fuel gas comprises H₂. In certain such embodiments, the said oxidizing gas comprises O₂. In certain such embodiments, the said constituents of the gas mixture comprise H₂, O₂, and CO₂.

In certain embodiments of the present invention, any gas headspaces or accumulated gas mixtures comprising a fuel gas and an oxidizing gas and/or oxidizer within the reactor system are kept below the explosive limit for flammable gas mixtures i.e., oxidant-rich with a fuel gas content below the lower explosivity limit (LEL) for the given constituents of the gas mix. In certain such embodiments, the said fuel gas comprises H₂. In certain such embodiments, the said oxidizing gas comprises O₂. In certain such embodiments, the said constituents of the gas mixture comprise H₂, O₂, and CO₂.

In certain embodiments of the present invention, any gas headspaces or accumulated gas mixtures comprising a fuel gas and an oxidizing gas and/or oxidizer within the reactor system are kept either above the UEL or below the LEL. In certain such embodiments, the said fuel gas comprises H₂. In certain such embodiments, the said oxidizing gas comprises O₂. In certain such embodiments, the said constituents of the gas mixture comprise H₂, O₂, and CO₂.

Garcia-Ochoa, F. et al.; Bioreactor scale up and oxygen transfer rate in microbial processes: An overview. Biotechnology Advances (2009), 27, pp. 153-176 is incorporated herein by reference in its entirety.

In certain embodiments of the present invention, the headspace is operated fuel “lean” (i.e., below lower explosion limit—LEL). In certain embodiments, the headspace is operated fuel “rich” (i.e., above upper explosion limit—UEL). In certain such embodiments, headspace gases are recycled and/or re-incorporated back into the broth. In certain embodiments, the headspace is diluted such that it is outside the flammability range. In certain such embodiments, the headspace is diluted with inert gases. In certain embodiments the headspace is diluted with N₂ and/or CO₂ such that the headspace gas mixture is kept outside the flammability range.

In certain embodiments that have a head space, which is operated fuel rich i.e., above the upper explosion limit (UEL) it may be expensive (to the point where it is uneconomic to operate) and wasteful of feed gases if the fuel-rich headspace were to be vented directly to atmosphere after a single pass through the bioreactor. In certain such embodiments, the headspace gas is recycled back into the broth. Certain aspects of the present invention relate to recycling the gas within a sterile envelope without introducing credible ignition sources, and cleaning and sterilizing the external recycling loops and associated machinery needed to recycle the gas.

In certain embodiments of the present invention, either O₂ or H₂ are fed into the reactor as a limiting component. In certain embodiments were O₂ is fed into the reactor as a limiting component the reactor headspace is fuel-rich and non-flammable. In certain embodiments were H₂ is fed into the reactor as a limiting component the reactor headspace is fuel-lean and non-flammable. In certain embodiments, the least soluble gas component is fed in excess and forms the majority of the partial pressure in the headspace. In certain embodiments, the most soluble component between the electron donor (e.g., H₂) and the electron acceptor in respiration (e.g., O₂) is added as the limiting factor.

Certain embodiments of the present invention involve headspace gas composition monitoring and/or suitably designed and specified safety systems to prevent the head space becoming explosive. In certain embodiments, the headspace volume and/or disengagement vessel is minimized or eliminated. In certain embodiments, the headspace and/or disengagement vessel is designed to withstand catastrophic failure in the event of ignition and detonation. In certain such embodiments, the said headspace and/or disengagement vessel is designed to withstand the maximum blast over pressure for hydrogen/oxygen mixtures. In certain embodiments, operational procedures, interlocks, and automated sequencing are used during dynamic scenarios such as start-up, runtime, and shutdown to prevent explosive mixture formation in the headspace and ensure adherence to the intended basis of safety. Certain aspects of the present invention relate to headspace composition monitoring, safety instrumented trips, purge/inertion systems, and control/elimination of credible ignition sources from the headspace. Certain aspects of the present invention relate to a robust basis of safety for the operation of the bioreactor when it is using hydrogen, oxygen, and carbon dioxide gases and/or any other potentially flammable gas mixture.

In one aspect, a bioreactor is provided for culturing a microorganism, which includes: a reactor vessel configured to contain a culture that includes a hydrogen-oxidizing and/or carbon monoxide-oxidizing microorganism and a gas headspace overlying the culture; one or more oxygen sensor(s) configured to measure a level of dissolved oxygen in the culture, and/or a level of oxygen gas in the gas headspace; a first gas feed manifold connected to a source of oxygen gas and configured to deliver oxygen gas into the culture, wherein the gas mixture is delivered under an amount of pressure; a stirring mechanism for mixing the culture; and a gas feed controller configured to regulate, based on the measured level of dissolved oxygen in the culture and/or the measured level of oxygen gas in the gas headspace, one or more of: an extent of mixing by the stirring mechanism, a level of oxygen gas delivered to the culture via the first gas feed manifold, or the amount of pressure; a pH sensor configured to measure a pH level of the culture; a base feed manifold configured to deliver a base to the culture; a base feed controller configured to regulate an amount of the base delivered to the culture based on the measured pH level; an nutrient feed manifold configured to deliver a nutrient amendment to the culture; and a nutrient feed controller configured to regulate an amount of the nutrient amendment delivered to the culture, In certain such embodiments, the amount of nutrient amendment delivered is proportional to the amount of the base delivered.

In some embodiments, the level of oxygen gas delivered to the culture includes the partial pressure of oxygen gas and its flow rate. In some embodiments, the gas feed controller regulates a flow rate of oxygen gas delivered to the culture.

In some embodiments, the base is ammonium hydroxide or ammonia.

In some embodiments, an oxygen sensor is configured to measure a level of oxygen gas in the gas headspace, wherein the gas feed controller is configured to regulate, based on the measured level of oxygen gas in the gas headspace, one or more of: an extent of mixing by the stirring means, or a level of oxygen gas delivered to the culture via the first gas feed manifold.

In some embodiments, the bioreactor includes: a culture media feed manifold configured to deliver culture media to the culture; and a culture media feed controller configured to regulate an amount of culture media delivered to the culture.

In some embodiments, an optical density sensor is configured to measure an optical density of the culture, wherein the culture media feed controller is configured to regulate the amount of culture media delivered to the culture based on the measured optical density. The bioreactor includes the optical density sensor configured to measure the optical density in the culture, and the culture media feed controller is configured to regulate the amount of culture media delivered to the culture based on the measured optical density.

In some embodiments, a culture withdrawal manifold is configured to withdraw an amount of the culture; a liquid level sensor configured to estimate a volume of the culture; and a liquid level controller configured to regulate the amount of the culture withdrawn based on the estimated volume of the culture.

In some embodiments, a foam sensor is configured to measure a level of foaming in the vessel; an antifoam feed manifold configured to deliver an antifoaming agent to the culture; and an antifoam feed controller configured to regulate an amount of the antifoaming agent delivered to the culture based on the measured level of foaming. For example, the antifoaming agent may be or may include polypropylene glycol.

In some embodiments, the stirring mechanism may include an impeller, such as, for example, a rushton impeller or a gas-entrainment impeller.

In some embodiment, the first gas feed manifold is configured to deliver oxygen gas into the culture through a sparger, such as, for example, an air stone sparger.

In some embodiments, a second gas feed manifold is configured to deliver a gas mixture into the gas headspace, wherein the gas mixture comprises H₂ and CO₂ and may include oxygen, and wherein a partial pressure of oxygen in the gas mixture is equal to or less than a partial pressure of oxygen in the oxygen gas delivered into the culture via the first gas feed manifold.

In one aspect, a method for culturing a microorganism is provided, including: delivering oxygen gas into a culture of a hydrogen-oxidizing and/or carbon monoxide-oxidizing microorganism contained in a reactor vessel, wherein a gas headspace overlies the culture; measuring a level of oxygen gas in the headspace or a level of dissolved oxygen in the culture; regulating a rate of delivery of oxygen gas into the culture based on the measured level of oxygen gas; measuring a level of pH in the culture; and regulating a rate of delivery of a base and a nutrient amendment based on the measured level of pH, wherein the rate of delivery of the nutrient amendment is proportional to the rate of delivery of the base. In certain such embodiments culture broth is continuously withdrawn from the reactor vessel and replaced with fresh water or nutrient media, to thereby continuously culture the microorganism.

In another embodiment, a method is provided for culturing a microorganism, including: delivering a gas mixture including oxygen gas into a culture of a hydrogen-oxidizing and/or carbon monoxide-oxidizing microorganism in a vessel of a bioreactor, wherein the gas mixture is delivered under an amount of pressure; measuring a level of dissolved oxygen in the culture; and regulating the amount of pressure based on the measured level of dissolved oxygen, to thereby culture the microorganism. In certain such embodiments, the said culture is grown in a continuous process.

In another aspect, a method is provided for culturing a microorganism, including: delivering a gas mixture including oxygen gas into a culture of a hydrogen-oxidizing and/or carbon monoxide-oxidizing microorganism in a vessel of a bioreactor, wherein the gas mixture is delivered under elevated pressure; measuring a level of oxygen in the headspace; and regulating the flow on delivered oxygen gas based on the mol fraction of oxygen gas in the headspace. In certain such embodiments, H₂ and CO₂ gas are delivered under elevated pressure, as rates that match, or fall with +/−5%, or +/−10%, or +/−20% of culture demand for these gaseous nutrients, and/or at a rate that maintains a targeted pressure inside the reactor.

In another aspect, a bioreactor is provided for culturing a microorganism, including: a reactor vessel configured to contain a culture including a hydrogen-oxidizing or carbon monoxide-oxidizing microorganism; a gas feed manifold configured to deliver a gas mixture comprising oxygen gas into the culture; and a gas permeable barrier separating a first compartment fluidly connected to the culture and a second compartment including oxygen gas.

In another aspect, a method is provided for culturing a microorganism, including: delivering a gas mixture including oxygen gas into a culture of a hydrogen-oxidizing and/or carbon monoxide-oxidizing microorganism in a reactor vessel; and providing a gas permeable barrier separating a first compartment fluidly connected to the culture and a second compartment including oxygen gas; wherein a partial pressure of oxygen gas in the second compartment is greater than a partial pressure of oxygen gas in the first compartment.

The bioreactors and methods of the present disclosure provide chemoautotrophic growth conditions while reducing the risk of creating potentially dangerous gas mixtures in the bioreactor. In some embodiments, the bioreactors and methods of the present disclosure provide a sufficiently high mass transfer rate coefficient (k_{L}a) of the gaseous substrates, including oxygen gas, into the culture to sustain high productivity chemoautotrophic growth (e.g., knallgas growth) of the microorganism without accumulating a potentially explosive mixture of gases in the bioreactor. The k_{L}a may be estimated using a dynamic gassing/degassing method, the sulfite method, or via mass balance of an active culture. Chapter 8 of Cussler, E. L.; Diffusion: Mass Transfer in Fluid Systems, 3rd Ed.; Cambridge Series in Chemical Engineering is incorporated herein by reference in its entirety.

In certain embodiments of the present invention, gas (e.g., oxygen) mass transfer is improved by maximizing the gas mass transfer coefficient, k_{L}a. This coefficient is a product of the interfacial area, a, of the gas bubbles dispersed through the liquid phase and the mass transfer coefficient, k_{L}. The product, k_{L}a, is generally evaluated as it is extremely difficult to separately quantify or calculate the interfacial area of bubbles in a fermenter. Very generally for liquid/gas systems the liquid film mass transfer coefficient is rate controlling and therefore ‘k_{L}’ is multiplied with ‘a’, since the gas film mass transfer coefficient, k_{g}, can be treated as negligible. In certain embodiments, the gas mass transfer is improved by making bubbles smaller, which increases interfacial area, reduces bubble rise velocity, and increases gas holdup. In certain embodiments, the gas mass transfer is improved by reducing the film thickness between the gas bubbles and the bulk liquid. In certain embodiments, the gas mass transfer is improved both by making bubbles smaller and by reducing the film thickness between the gas bubbles and the bulk liquid. Generally greater gassing rates (more aeration) and more power input (faster agitation) result in higher gas mass transfer rates. In certain embodiments, gas mass transfer rates are increased by increasing gassing rates and/or power input e.g., agitation rates. In certain embodiments, the aeration rate and agitation rate are optimized to give the minimum total power consumption (comprising power consumption both for gas compression and for agitation) of the fermenter for a given mass transfer rate. As aeration rates increase compressor power increases, however agitation power demand decreases as the fluid density decreases locally at the impellers due to the increased volume fraction of air in the fluid. Certain aspects of the present invention relate to optimizing power consumption, oxygen transfer rate, shear effects, biomass growth, and yield on a specific substrate in terms of full cost benefit analysis.

At industrial production scale power consumption is vitally important, especially when the fermentation product is a bulk commodity of relatively low price such as crude protein. In certain embodiments, the ratio of gas mass transfer rate per unit power consumed is maximized. In certain embodiments, the distribution of the power input per unit volume is optimized.

In certain embodiments of the present invention, integrated safety measures including but not limited to one or more of: real-time gas mixture monitoring; shutdown protocols; and eliminating gas headspaces and/or gas accumulations are utilized within the reactor and bioprocess designs of the present invention. In certain embodiments of the present invention, the volume of gas headspace, and/or other gas accumulations, in the bioreactors and/or bioprocess system are reduced during operation. In certain such embodiments, the reduction in gas headspace and/or other gas accumulations during operation result, at least in part, from a liquid level rise produced by gas hold-up. In certain embodiments of the present invention, pockets of gas within the bioreactor and/or bioprocess system are minimized during operation. In certain said embodiments, minimizing said internal pockets of gas headspaces and other accumulated gases improve the intrinsic safety of the reactor and bioprocess operation. In certain embodiments of the present invention, ratio of the working volume (including gas hold-up) during operation to the total reactor volume is maximized. The bioreactor in certain embodiments, has very little, or no headspace, essentially eliminating the presence of flammable or explosive gas accumulations within the bioreactor during normal operation. In certain such embodiments, the reduction or elimination of the headspace provides for intrinsically safer operation. In certain embodiments, reduction or elimination of headspace is enabled by the fact that no gases are produced as part of the fermentation process—e.g., there is no metabolic carbon dioxide created by the culture.

In some embodiments, the microorganisms are grown and maintained in a medium suitable for chemoautotrophic growth, containing gaseous carbon and energy sources, such as but not limited to syngas, producer gas, tail gas, pyrolysis gas, or H₂ and CO₂ and/or CO gas mixtures. There is no requirement for light in chemoautotrophic CO₂ fixation, and in certain embodiments there is little light or an absence of light in the growth environment.

Provided herein are methods for growing a microorganism, e.g., a hydrogen-oxidizing or carbon monoxide-oxidizing microorganism, in a bioreactor. In some embodiments, the microorganism is cultured under conditions sufficient to support chemoautotrophic growth, including provision of sufficient electron donor (e.g., hydrogen), electron acceptor (e.g., oxygen), and carbon source (e.g., carbon dioxide) to the culture. In certain such embodiments, a safe level of oxygen (i.e., non-flammable gas mixture) is maintained in the headspace of the bioreactor vessel.

In certain embodiments of the present invention, the bioreactors describe herein are integrated with one or more H₂ and/or CO₂ sources such as but not limited to: industrial gases, tail gases, flue gases, electrolyzers, steam reformers, gasifiers, and/or water gas shift reactors. In certain such embodiments, there are one or more gas clean-up and/or purification steps that occur between the said H₂ and/or CO₂ sources and the said bioreactors. In certain non-limiting embodiments of the present invention purification steps results in a sulfur content in the gaseous feedstocks of around 0.5-1 mg S/m³ (STP) or less. In other non-limiting embodiments, the sulfur content in the gaseous feedstocks is greater than 1 mg S/m³ (STP).

In certain embodiments of the present invention production cost minimization is achieved through minimizing capital costs (e.g., materials), and/or through minimizing operational costs (e.g., electrical inputs). In certain embodiments of the present invention that utilize gaseous feedstocks including but not limited to one or more of: H₂, CO, CH₄, CO₂, or O₂, minimizing production cost strongly depends upon maximizing the conversion of the gaseous feedstock/s that are input into the bioreactor system. In certain such embodiments, H₂ is input into the bioreactor system. Certain embodiments of the present invention maximize the conversion of H₂. Key factors generally include the amount of gas venting or leakage, the gas conversion in a single pass, and the facility and efficiency in recirculating and/or re-entraining unconsumed gases back into the working volume, particularly in certain non-limiting embodiments, unconsumed H₂. In certain embodiments of the present invention gas venting or leakage is minimized and/or the gas conversion in a single pass is maximized and/or the facility and efficiency in recirculating and/or re-entraining unconsumed gases back into the working volume is enhanced. In certain such embodiments, the said gas or gases comprises H₂.

In an exemplary but nonlimiting embodiment, a bioreactor containing nutrient medium is inoculated with production cells. Generally, there will follow a lag phase prior to the cells beginning to double. After the lag phase, the cell doubling time decreases and the culture goes into the logarithmic phase. The logarithmic phase is eventually followed by an increase of the doubling time that, while not intending to be limited by theory, is thought to result from either a mass transfer limitation, depletion of nutrients including nitrogen or mineral sources, or a rise in the concentration of inhibitory chemicals, or quorum sensing by the microbes. The growth slows down and then ceases when the culture enters the stationary phase. In certain embodiments, there is an arithmetic growth phase preceding the stationary phase. In order to harvest cell mass, the culture in certain embodiments is harvested in the logarithmic phase and/or in the arithmetic phase and/or in the stationary phase.

In certain embodiments, inoculation of the culture into the bioreactor is performed by methods including, but not limited to, transfer of culture from an existing culture inhabiting another bioreactor, or incubation from a seed stock raised in an incubator. In certain embodiments, the seed stock of the strain may be transported and stored in forms including but not limited to a powder, liquid, frozen, or freeze-dried form as well as any other suitable form, which may be readily recognized by one skilled in the art. In certain non-limiting embodiments, the reserve bacterial cultures are kept in a metabolically inactive, freeze-dried state until required for restart. In certain embodiments, when establishing a culture in a very large reactor, cultures are grown and established in progressively larger intermediate scale vessels prior to inoculation of the full-scale vessel.

The growth conditions, including control of dissolved gases, such as carbon dioxide, oxygen, and/or other gases such as hydrogen, and gas pressure, as well as other dissolved nutrients, trace elements, temperature, and pH, may be controlled in the bioreactor. For certain embodiments, a protein-rich cell mass is grown to high densities and/or grown at high productivities, in liquid suspension within a bioreactor.

In certain embodiments, the chemicals used for maintenance and growth of microbial cultures as known in the art are included in the nutrient media. In certain embodiments, these chemicals may include but are not limited to one or more of the following: nitrogen sources such as ammonia, ammonium (e.g., ammonium chloride (NH₄Cl), ammonium sulfate ((NH₄)₂SO₄), ammonium nitrate (NH₄NO₃)), nitrate (e.g., potassium nitrate (KNO₃)), urea and/or an organic nitrogen source; phosphate (e.g., disodium phosphate (Na₂HPO₄), potassium phosphate (KH₂PO₄), phosphoric acid (H₃PO₄), potassium dithiophosphate (K₃PS₂O₂), potassium orthophosphate (K₃PO₄), dipotassium phosphate (K₂HPO₄)); sulfate; yeast extract; chelated iron; potassium (e.g., potassium phosphate (KH₂PO₄), potassium nitrate (KNO₃), potassium iodide (KI), potassium bromide (KBr)); and other inorganic salts, minerals, and trace nutrients (e.g., sodium chloride (NaCl), magnesium sulfate (MgSO₄ 7H₂O) or magnesium chloride (MgCl₂), calcium chloride (CaCl₂) or calcium carbonate (CaCO₃), manganese sulfate (MnSO₄ 7H₂O) or manganese chloride (MnCl₂), ferric chloride (FeCl₃), ferrous sulfate (FeSO₄ 7H₂O), ferrous chloride (FeCl₂ 4H₂O) or ferric ammonium citrate, sodium bicarbonate (NaHCO₃) or sodium carbonate (Na₂CO₃), zinc sulfate (ZnSO₄) or zinc chloride (ZnCl₂), ammonium molybdate (NH₄MoO₄) or sodium molybdate (Na₂MoO₄ 2H₂O), cuprous sulfate (CuSO₄) or copper chloride (CuCl₂ 2H₂O), cobalt chloride (CoCl₂ 6H₂O), aluminum chloride (AlCl₃·6H₂O), lithium chloride (LiCl), boric acid (H₃BO₃), nickel chloride (NiCl₂ 6H₂O), tin chloride (SnCl₂H₂O), barium chloride (BaCl₂ 2H₂O), copper selenate (CuSeO₄ 5H₂O) or sodium selenite (Na₂SeO₃), sodium metavanadate (NaVO₃), chromium salts). In certain embodiments, the mineral salts medium (MSM) formulated by Schlegel et al may be used [“Thermophilic bacteria”, Jakob Kristjansson, Chapter 5, Section III, CRC Press, (1992) is incorporated herein by reference in its entirety].

In some embodiments, the microorganism culture is provided with one or more nutrient amendments to supplement the media and promote continuous growth. In some embodiments, the nutrient amendment is provided at a rate proportional to the rate of consumption of one or more nutrients, or rate of metabolism or growth of the culture. In some embodiments, the nutrient amendment is provided at a rate proportional to the rate of consumption of a base used to maintain the culture at the appropriate pH. In certain such embodiments the base is also a nitrogen source (e.g., NH₄OH or NH₃). The pH of the culture may be measured to provide a feedback signal controlling the rate of delivery of a base. In some embodiments, the rate of delivery of a nutrient amendment to the culture is proportional to rate of delivery of the base to the culture. In some embodiments, the rate of delivery of a nutrient amendment to the culture is under pH feedback control.

The control of pH can be a challenge due to its logarithmic scale and strong dependence on process chemistry leading to highly non-linear process gain dynamics. Likewise process deadtime is strongly influenced by the chemistry of the system e.g., if pH buffers are present these generally tend to increase process deadtime and reduce/dampen process gain dynamics. Furthermore, the physics of the system impact pH control performance—long mixing times/poor mixing performance tend to increase the process deadtime and uniformity issues. Certain aspects of the present invention relate to addressing these factors influencing pH control through bioreactor and bioprocess design.

The nutrient amendment may include any suitable component of the media that may be supplemented during culture growth. The nutrient amendments may include, without limitation, one or more supplements for sodium, potassium, calcium, magnesium, zinc, manganese, iron, cobalt, copper, nickel, phosphate, chloride, sulfate, borate, and/or molybdate. In some embodiments, the nutrient amendment includes Na₂HPO₄, KH₂PO₄, MgSO₄, ferric ammonium citrate, CaCl₂), ZnSO₄, MnCl₂, H₃BO₃, COCl₂, CuCl₂, NiCl₂, and/or Na₂MoO₄.

In batch culture systems, the conditions (e.g., nutrient concentration, pH, etc.) under which the microorganism is cultivated generally change continuously throughout the period of growth. In certain non-limiting embodiments, to avoid the fluctuating conditions inherent in batch cultures, and to improve the overall productivity of the culture system, the microorganisms that are used to produce protein and/or vitamins and/or other nutrients and/or biomass and/or other biochemicals or organic molecules are grown in a continuous culture system such as a chemostat or a turbidostat. In such systems, the culture may be maintained in a perpetual exponential phase of growth by feeding it with fresh medium at a constant rate [F] while at the same time maintaining the volume [V] of the culture constant. In certain embodiments, a continuous culture system ensures that cells are cultivated under environmental conditions that remain roughly constant. In certain embodiments, the cells are maintained in a perpetual exponential phase through the use of a chemostat system. In such a case the dilution rate (D) of the culture equals the specific growth rate of the microorganism, and is given by: D=F/V. The growth rate of a microorganism in continuous culture may be changed by altering the dilution rate. In certain embodiments, the growth rate of the microorganism is changed by altering the dilution rate. In certain non-limiting embodiments, cells are grown in a chemostat or a turbidostat at a dilution rate of at least about 0.02 h⁻¹, at least about 0.05 h⁻¹, at least about 0.1 h⁻¹, at least about 0.15 h⁻¹, at least about 0.2 h⁻¹, or over 0.2 h⁻¹.

In certain embodiments, one or more of the following parameters is monitored and/or controlled in the bioreactor: waste product levels; temperature; salinity; dissolved carbon dioxide gas; liquid flow rates, pressure, gas composition, liquid level. In certain embodiments, the operating parameters affecting chemoautotrophic growth are monitored with sensors (e.g., dissolved oxygen probe or oxidation-reduction probe to gauge electron donor/acceptor concentrations), and/or are controlled either manually or automatically based upon feedback from sensors through the use of equipment including, but not limited to one or more of: actuating valves, pumps, and agitators. In certain embodiments, the temperature of the incoming culture medium as well as of incoming gases is regulated by systems such as, but not limited to, coolers, heaters, and/or heat exchangers.

In certain embodiments, the microbial culture and bioreaction is maintained using continuous influx and removal of nutrient medium and/or biomass, in steady state where the cell population and environmental parameters (e.g., cell density, pH, DO, chemical concentrations) are targeted at a constant level overtime. In certain embodiments, the constant level is an optimal level for feedstock conversion and/or production of targeted organic compounds and/or biomass. In certain embodiments, cell densities can be monitored by direct sampling, by a correlation of optical density to cell density, and/or with a particle size analyzer. In certain embodiments, the hydraulic and biomass retention times can be decoupled so as to allow independent control of both the broth chemistry and the cell density. In certain embodiments, dilution rates can be kept high enough so that the hydraulic retention time is relatively low compared to the biomass retention time, resulting in a highly replenished broth for cell growth and/or feedstock conversion and/or production of organic compounds and/or biomass. In certain embodiments, hydraulic retention time is relatively high compared to the biomass retention time through the application of a solid-liquid separation step to recover biomass followed by recycling of the separated liquid back to the bioreactor, enabling a high dilution rate with minimal water and dissolved nutrients lost from the system as wastewater and minimal input of fresh water to make-up for water losses. In certain embodiments, dilution rates are set at an optimal technoeconomic trade-off between culture broth and nutrient replenishment and/or waste product removal, and increased process costs from pumping, increased inputs, and other demands that rise with dilution rates.

In certain embodiments, dissolved oxygen (DO) is regulated and controlled by measures including but not limited to one or more of the following: aeration rates, headspace pressures, agitation rates, and/or OD via dilution rate in the case of continuous (e.g., turbidostatic) operation.

In certain embodiments, a level controller acts on a control valve that directly acts on flow into the bioreactor from one or more sterile fill lines and/or controls pneumatic top pressure of one or more upstream sterile feed vessels. In certain embodiments, dilution rates are finely balanced with harvest rates to prevent bioreactor wash-out, which occurs if dilution rates are too high relative to the specific growth rate of the organism. In certain embodiments, the phenomenon of wash out is mitigated by monitoring optical density (OD) outputs on the bioreactor as dilution rates and harvest rates are gradually increased. Coupled with these fermentation dynamic requirements is the need to regulate level at a steady value to prevent the fermenter overfilling or emptying.

In certain embodiments, the pressure and/or other variables and set to values giving optimal e.g., maximal productivity and/or yield. In certain embodiments, increasing pressure will increase productivity due to higher gas-to-liquid mass transfer rate and/or greater thermodynamic driving force from gaseous reactants to solid and/or liquid products. Temperature may have opposing effects as increasing temperature can increase gas diffusivity (e.g., k_{L}) but can also reduce gas aqueous solubility (e.g., H₂ and O₂). Optimal growth (e.g., specific growth rate) for a microorganism also generally only occurs over a limited temperature range. In certain embodiments, the bioreactor temperature is set to values giving optimal e.g., maximal productivity and/or yield. Increasing the space velocity (i.e., STP volume gas/time/working volume bioreactor) can increase the mass transfer coefficient of gas into solution (i.e., k_{L}a). Increasing the level of inert gas can lower the reaction rate for kinetic and thermodynamic reasons. There is often an optimal hydrogen/carbon dioxide ratio and hydrogen/oxygen ratio. The position of this optimum may depend on the space velocity values. In certain embodiments, a recycle rate of unconsumed gases is chose to maximize productivity while minimizing loss of unreacted H₂ from the system. In certain embodiments, the condenser temperature or water separator temperature is set to maximize water recovery from a gas recycle loop.

In certain embodiments, the number of gas bubbles is maximized and/or the distribution of gas bubbles is optimized, so as to minimize the average diffusion path length from gas phase to liquid phase, and thus increase gas to liquid mass transfer.

Certain aspects of the present invention relate to best e.g., optimal synthesis pressure at which to run the gas bioprocess. Certain aspects of the present invention involve optimization of parameters such as but not limited to feedstock price, return on investment, and site requirements. In certain embodiments the minimum amount of mechanical work needed in a synthesis loop is calculated.

In certain embodiments of the present invention the bioreactor may be internally cooled with cooling tubes running through the working volume and/or with working volume inside of tubes and the cooling medium on the shell side. In certain embodiments, the cooling medium is partially or mostly reactor feed gases, which in certain embodiments can flow counter or co-current to the gas flow in the working volume (e.g., tube-cooled converters). In certain such embodiments, to remove the heat evolved in the biosynthesis reaction (e.g., knallgas reaction), bioreactors are used with cooling tubes that run through the working volume. Using these tubes, the heat is transferred to the feed gas (e.g., O₂, CO₂, and/or H₂) to heat it to the bioreactor temperature and/or to an external cooling medium. In certain embodiments, cooling between individual working volumes is achieved by indirect heat exchange with a cooling medium. In certain such embodiments, the cooling medium may include but is not limited to cooler synthesis gas (e.g., H₂, CO₂, and/or O₂) and/or water and/or aqueous nutrient media. In certain embodiments the heat exchanger/s may be installed together with the volume/s inside one pressure shell. In certain embodiments, individual volumes are held in separate pressure vessels and use separate heat exchangers.

In certain non-limiting embodiments, the working volume maybe divided into several reactors within which the reaction proceeds adiabatically. Between the individual working volumes heat is removed by injection of colder synthesis gas and/or oxygen (quench converters) or by indirect cooling with a cooling medium (e.g., water) and/or synthesis gas and/or oxygen. In certain such embodiments, the gas flow can have an axial, cross-flow and/or radial flow pattern. In certain embodiments, cooling is achieved wholly or in part by injection of cooler, unconverted synthesis gas (cold shot) e.g., H₂ and/or CO₂ and/or O₂ between working volumes and/or into working volumes. In certain embodiments, the working volumes may be separated by static mixers. In certain embodiments, the bioprocess is designed with working volumes distributed in several sections, within one or more reactor vessels. In each working volume, the synthesis gas (e.g., H₂, CO₂, and/or O₂) reacts adiabatically, and direct or indirect cooling is provided between the working volumes for cooling the reacting mixture from a temperature above to a value below the targeted e.g., optimal temperature. In certain embodiments, the reaction profile describes a zig-zag path around the target temperature e.g., optimal temperature. In certain embodiments, space is saved within the high pressure vessel through the use of direct cooling of the working volume/s using cooled gas and/or cooled mineral nutrient inputs and/or dilution with cooled aqueous media, compared to what would be required for the equivalent cooling using heat exchangers. In certain embodiments, the different cooling methods can be combined in the same systems of bioreactors.

Certain aspects of the present invention relate to safety/safe operations of the fermenter (i.e., bioreactor), for example, with respect to accumulation of explosive gas mixtures within the fermenter. Certain aspects of the present invention relate to sterility/suitability of reactor architecture for processing biomass—e.g., consideration given to, for example, excessive shear, dead legs, and application of automated clean and sterilization sequences. Certain aspects of the present invention relate to gas mass transfer. In certain embodiments of the present invention, the fermentations are gas based therefore good mass transfer is an important design and operational consideration.

Nutrient media, as well as gases, can be added to the bioreactor as either a batch addition, or periodically, or in response to a detected depletion or programmed set point, or continuously over the period the culture is grown and maintained. For certain embodiments, the bioreactor at inoculation is filled with a starting batch of nutrient media and/or one or more gases at the beginning of growth, and no additional nutrient media and/or one or more gases are added after inoculation. For certain embodiments, nutrient media and/or one or more gases are added periodically after inoculation. For certain embodiments, nutrient media and/or one or more gases are added after inoculation in response to a detected depletion of nutrient and/or gas. For certain embodiments, nutrient media and/or one or more gases are added continuously after inoculation.

For certain embodiments, the added nutrient media does not contain any organic compounds, e.g., does not contain an organic carbon source such as sugar molecules or other organic molecules that may be metabolized by microorganisms as a carbon source.

In certain embodiments, a small amount of microorganism cells (i.e., an inoculum) is added to a set volume of culture medium; the culture is then incubated; and the cell mass passes through lag, exponential, deceleration, and stationary phases of growth.

In batch culture systems, the conditions (e.g., nutrient concentration, pH, etc.) under which the microorganism is cultivated generally change continuously throughout the period of growth. In certain non-limiting embodiments, to avoid the fluctuating conditions inherent in batch cultures, and to improve the overall productivity of the culture system, the microorganisms that are used for the production of protein and/or vitamins and/or other nutrients and/or other biochemicals are grown in a continuous culture system called a chemostat (e.g., a bioreactor or other culture vessel to which fresh medium is continuously added, while culture liquid containing left over nutrients, metabolic end products and microorganisms are continuously removed at the same rate to keep the culture volume constant). In certain embodiments the microorganisms that are used to produce protein and/or vitamins and/or other nutrients and/or other biochemicals or organic molecules are grown in a continuous culture system called a turbidostat (e.g., a continuous microbiological culture device, which has feedback between the turbidity of the culture vessel and the dilution rate).

For certain embodiments, the bioreactors have mechanisms to enable mixing of the nutrient media that include, but are not limited to, one or more of the following: spinning stir bars, blades, impellers, or turbines; spinning, rocking, or turning vessels; gas lifts, sparging; recirculation of broth from the bottom of the container to the top via a recirculation conduit, flowing the broth through a loop and/or static mixers. The culture media may be mixed continuously or intermittently.

In certain embodiments, the microorganism-containing nutrient medium may be removed from the bioreactor partially or completely, periodically or continuously, and in certain embodiments is replaced with fresh cell-free medium to maintain the cell culture in an exponential growth phase, and/or in an arithmetic growth phase, and/or to replenish the depleted nutrients in the growth medium, and/or to remove inhibitory waste products.

The ports that are standard in bioreactors may be utilized to deliver, or withdraw, gases, liquids, solids, and/or slurries, into and/or from the bioreactor vessel enclosing the microorganisms. Many bioreactors have multiple ports for different purposes (e.g., ports for media addition, gas addition, probes for pH and dissolved oxygen (DO), and sampling), and a given port may be used for various purposes during the course of a fermentation run. As an example, a port might be used to add nutrient media to the bioreactor at one point in time, and at another time might be used for sampling. In some embodiments, the multiple uses of a sampling port can be performed without introducing contamination or invasive species into the growth environment. A valve or other actuator enabling control of the sample flow or continuous sampling can be provided to a sampling port. For certain embodiments, the bioreactors are equipped with at least one port suitable for culture inoculation that can additionally serve other uses including the addition of media or gas. Bioreactor ports enable control of the gas composition and flow rate into the culture environment. For example, the ports can be used as gas inlets into the bioreactor through which gases are pumped.

Suitable ports may be utilized to deliver, or withdraw, gases, liquids, solids, and/or slurries, into and/or from the bioreactor vessel enclosing the microorganisms.

A bioreactor of the present disclosure may include any suitable gas diffuser. Suitable diffusers may include, without limitation, dome, tubular, disc, or doughnut geometries; coarse or fine bubble aerators; venturi equipment. In some embodiments, the gas diffuser is a sparger. A suitable sparger includes, without limitation, frit spargers and air-stone spargers. In some embodiments, the frit sparger is a L-frit sparger or a J-frit sparger. In some embodiments, the gas diffuser is a disk air-stone sparger.

A bioreactor of the present disclosure may include one or more inlets for introducing a gas or gas mixture into the reactor vessel. In some embodiments, the gas or gas mixture is fed into the culture medium (e.g., via the gas diffuser). In some embodiments, the bioreactor includes one inlet for feeding a gas or gas mixture into the culture, and another inlet for feeding another gas or gas mixture into the headspace.

For some embodiments, gases that may be pumped into a bioreactor include, but not are not limited to, one or more of the following: syngas, producer gas, pyrolysis gas, hydrogen gas, CO, CO₂, O₂, air, air/CO₂ mixtures, natural gas, biogas, methane, ammonia, nitrogen, noble gases, such as argon, as well as other gases. In certain embodiments of the present invention, CO₂ pumped into the system is sourced from ethanol production facilities that produce CO₂ as a byproduct. In some embodiments the CO₂ pumped into the system may come from sources including, but not limited to: CO₂ from the gasification of organic matter; CO₂ from the calcination of limestone, CaCO₃, to produce quicklime, CaO; CO₂ from methane steam reforming, such as the CO₂ byproduct from ammonia, methanol, or hydrogen production; CO₂ from combustion, incineration, or flaring; CO₂ byproduct of anaerobic or aerobic fermentation of sugar and/or any other organic carbon substrate used for fermentations; CO₂ byproduct of a methanotrophic bioprocess; CO₂ byproduct of a carboxydotrophic bioprocess; CO₂ byproduct from a heterotrophic metabolism; CO₂ from waste water treatment; CO₂ byproduct from sodium phosphate production; geologically or geothermally produced or emitted CO₂; CO₂ removed from acid gas or natural gas. In certain non-limiting embodiments, the CO₂ has been removed from an industrial flue gas, or intercepted from a geological source that would otherwise naturally emit into the atmosphere. In certain embodiments, the carbon source is CO₂ and/or bicarbonate and/or carbonate dissolved in sea water or other bodies of surface or underground water. In certain such embodiments the inorganic carbon may be introduced to the bioreactor dissolved in liquid water and/or as a solid. In certain embodiments, the carbon source is CO₂ captured from the atmosphere. In certain non-limiting embodiments, the CO₂ has been captured from a closed cabin as part of a closed-loop life support system, using equipment such as but not limited to a CO₂ removal assembly (CDRA), which is utilized, for example, on the International Space Station (ISS).

In certain non-limiting embodiments, geological features such as, but not limited to, geothermal and/or hydrothermal vents that emit high concentrations of energy sources (e.g., H₂, H₂S, CO gases) and/or carbon sources (e.g., CO₂, HCO₃⁻, CO₃²⁻) and/or other dissolved minerals may be utilized as nutrient sources for the microorganisms herein.

In certain embodiments, one or more gases in addition to carbon dioxide, or in place of carbon dioxide as an alternative carbon source, are either dissolved into solution and fed to the culture broth and/or dissolved directly into the culture broth, including but not limited to gaseous electron donors and/or carbon sources (e.g., hydrogen and/or CO and/or methane gas). In certain embodiments, input gases may include other electron donors and/or electron acceptors and/or carbon sources and/or mineral nutrients such as, but not limited to, other gas constituents and impurities of syngas (e.g., hydrocarbons); ammonia; hydrogen sulfide; and/or other sour gases; and/or O₂; and/or mineral containing particulates and ash.

In some embodiments, the microorganisms convert a fuel gas, including but not limited to syngas, producer gas, pyrolysis gas, biogas, tail gas, flue gas, CO, CO₂, H₂, natural gas, methane, and mixtures thereof. In some embodiments, the heat content of the fuel gas is at least about 100 BTU per standard cubic foot (scf). In some embodiments, a bioreactor that is used to contain and grow the microorganisms is equipped with fine-bubble diffusers and/or high-shear impellers for gas delivery.

In certain embodiments, the microorganisms grow and multiply on H₂ and CO₂ and other dissolved nutrients under microaerobic conditions. In certain embodiments, a C1 chemical such as but not limited to carbon monoxide, methane, methanol, formate, or formic acid, and/or mixtures containing C1 chemicals including but not limited to various syngas compositions generated from various gasified, pyrolyzed, or steam-reformed fixed carbon feedstocks, is biochemically converted into longer chain organic chemicals (i.e., C2 or longer and, in some embodiments, C5 or longer carbon chain molecules) under one or more of the following conditions: aerobic, microaerobic, anoxic, anaerobic, and/or facultative conditions.

The source of inorganic carbon used in the chemosynthetic reaction process steps contained within the bioreactor of certain embodiments of the present disclosure includes but is not limited to one or more of the following: a carbon dioxide-containing gas stream that may be pure or a mixture; liquefied CO₂; dry ice; dissolved carbon dioxide, carbonate ion, or bicarbonate ion in solutions including aqueous solutions such as sea water; inorganic carbon in a solid form such as a carbonate or bicarbonate minerals. Carbon dioxide and/or other forms of inorganic carbon can be introduced to the nutrient medium contained in the bioreactor either as a bolus addition, periodically, or continuously at the steps in the process where carbon-fixation occurs. Organic compounds containing only one carbon atom, which can be used in the biosynthetic reaction process steps occurring in the bioreactor of certain embodiments of the present disclosure include but are not limited to one or more of the following: carbon monoxide, methane, methanol, formate, formic acid, and/or mixtures containing C1 chemicals including but not limited to various syngas or producer compositions generated from various gasified, pyrolyzed, partially oxidized, or steam-reformed fixed carbon feedstocks or C1 containing industrial or mining or drilling tail gases, process gases, or effluent streams.

In certain embodiments, carbon dioxide containing flue gases are captured from a smokestack at temperature, pressure, and gas composition characteristic of the untreated exhaust, and directed with minimal modification into the bioreactor of the present disclosure containing a chemoautotrophic microorganism where carbon-fixation occurs. In some embodiments in which impurities harmful to chemoautotrophic organisms are not present in the flue gas, modification of the flue gas upon entering the bioreactor can be limited to the compression needed to pump the gas through the bioreactor system and/or the heat exchange needed to lower the gas temperature to one suitable for the microorganisms.

Gases in addition to carbon dioxide that are dissolved into solution and fed to the culture broth or dissolved directly into the culture broth contained in the bioreactor in certain embodiments of the present disclosure include gaseous electron donors (e.g., hydrogen gas or carbon monoxide gas), but in certain embodiments of the present disclosure, may include other electron donors such as but not limited to other gas constituents of syngas, hydrogen sulfide, and/or other sour gases.

In certain embodiments, organic compounds containing only one carbon atom (i.e. C1 compounds), such as carbon monoxide or carbon dioxide, are generated through the gasification and/or pyrolysis and/or partial oxidation and/or steam reforming of biomass and/or other organic matter (e.g., biomass and/or other organic matter from waste or low value sources), and provided as a syngas or producer gas to the culture of oxyhydrogen, or hydrogen-oxidizing, or carbon monoxide-oxidizing, or chemoautotrophic microorganisms contained in the bioreactor, where the ratio of hydrogen to carbon monoxide in the syngas may or may not be adjusted through means such as the water gas shift reaction, prior to the syngas or producer gas being delivered to the microbial culture in the bioreactor. In certain embodiments, organic compounds containing only one carbon atom are generated through methane steam reforming from methane or natural gas (e.g., stranded natural gas, or natural gas that would be otherwise flared or released to the atmosphere), or biogas, or landfill gas, and provided as a syngas or producer gas to the culture of oxyhydrogen or hydrogen-oxidizing, or carbon monoxide-oxidizing, or chemoautotrophic microorganisms in the bioreactor, where the ratio of hydrogen to carbon monoxide in the syngas may or may not be adjusted through means such as the water gas shift reaction, prior to the syngas being delivered to the microorganism culture.

In certain embodiments, hydrogen electron donors and/or C1 carbon sources for microbial growth and biosynthesis are generated from waste or low value sources of carbon and energy using methods known in to art of chemical and process engineering including but not limited to gasification, pyrolysis, partial oxidation, or steam-reforming of feedstock such as, but not limited to, municipal waste, black liquor, bagasse, agricultural waste, crop residues, wood waste, saw dust, forestry residue, food waste, stranded natural gas, biogas, landfill gas, sour gas, methane hydrates, tires, pet coke, waste carpet, sewage, manure, straw, and low value, highly lignocellulosic biomass in general.

In some embodiments, oxygen gas (independently, or in a mixture with other suitable gases) is delivered to a microorganism culture by dispersing the gas in the culture, e.g., through a sparger or other suitable gas dispersing means, to create gas bubbles that percolate through the culture. In some embodiments, the average diameter of the gas bubbles may be about 8 mm or less, e.g., about 7.5 mm or less, about 7 mm or less, about 6.5 mm or less, about 6 mm or less, about 5 mm or less, about 4 mm or less, about 3 mm or less, about 2 mm or less, including about 1 mm or less.

In some embodiments, an oxygen sensor measures the dissolved oxygen level in the culture and provides a feedback signal to a controller. The controller may regulate the rate at which oxygen gas is delivered to the culture, e.g., via a gas feed manifold and an inlet valve, based on the feedback signal. In some embodiments, an oxygen sensor measures the headspace oxygen level in the bioreactor vessel holding the culture and provides a feedback signal to the controller. The controller may respond to the feedback signal by adjusting the rate at which oxygen is delivered to the culture so as to maintain the oxygen level at the measurement site at a desirable level. In some embodiments, the feedback control of the headspace oxygen level is sufficient to maintain a safe level of oxygen (i.e., a non-flammable gas mixture) in the headspace. In some embodiments, feedback control of the headspace oxygen level prevents accumulation of an explosive mixture of gases in the headspace and reduces the risk of explosion. In some embodiments, feedback control of the headspace oxygen level targets an oxygen concentration in the headspace gas of about 5% (v/v) or lower.

In addition to oxygen, the bioreactor may be supplied with any suitable combination of gases to sustain growth of the microorganism. In some embodiments, the bioreactor is provided with a combination of gases sufficient to support chemoautotrophic growth of the microorganism, e.g., hydrogen-oxidizing or carbon monoxide-oxidizing microorganism. In some embodiments, the combination of gases includes a combination of one or more of oxygen gas, hydrogen gas, carbon dioxide gas, carbon monoxide gas, methane, nitrogen gas and air. In some embodiments, the combination of gases includes a combination of oxygen gas, hydrogen gas, carbon dioxide gas, and air. The gases may be supplied to the reactor vessel by any suitable mechanism. In some embodiments, the gases are supplied individually through separate gas feed manifolds. In some embodiments, one or more gases are mixed together before being supplied through a gas feed manifold. In some embodiments, a mixture of gases containing hydrogen gas, carbon dioxide gas and air is provided to the reactor vessel through a gas feed manifold. In some embodiments, oxygen gas is added to the mixture of gases containing hydrogen gas, carbon dioxide gas and air before being delivered to the reactor vessel.

In some embodiment, the proportion of oxygen gas in the mixture of gases delivered to the culture is under oxygen feedback control. In some embodiments, the proportion of oxygen gas in the mixture of gases delivered to the culture is about 4% (v/v) or more, e.g., about 5% (v/v) or more, about 6% (v/v) or more, about 8% (v/v) or more, about 10% (v/v) or more, about 12% (v/v) or more, including about 15% (v/v) or more. In some embodiments, the proportion of oxygen gas in the mixture of gases delivered to the culture is in the range of 0% to about 20% (v/v), e.g., about 2% (v/v) to about 18% (v/v), about 4% (v/v) to about 16% (v/v), including about 4% to 12% (v/v). In certain embodiments, O₂ gas that is essentially pure, e.g., at least 90% O₂ (v/v) or at least 95% O₂ or at least 99% O₂ (v/v) or at least 99.9% O₂ (v/v) is fed into the culture through a separate input line, along with H₂, CO₂, and/or other fuel gases that are fed into the culture through a different input line, where gas bubbles and/or dissolved gases from the said separate input lines are mixed together in the culture.

The O₂ content of a typical industrial or power plant flue gas is at least about 2% (v/v), for example, about 2% (v/v) to about 6% (v/v). In certain embodiments of the present disclosure, carbon capture from a flue gas stream is performed by a hydrogen-oxidizing microorganism contained in the bioreactor that is tolerant of gas input and bioreactor headspace oxygen levels of at least about 2% (v/v), e.g., about 2% (v/v) to about 6% (v/v). In some embodiments this hydrogen-oxidizing microorganism is an oxyhydrogen microorganism.

In some embodiments, a mixture of gases containing hydrogen gas, carbon dioxide gas, and oxygen gas or air is provided to the reactor vessel through one gas feed manifold, and oxygen gas is provided to the reactor vessel through another gas feed manifold. In some embodiments, a mixture of gases containing hydrogen gas, carbon dioxide gas, and oxygen gas or air is provided to the reactor vessel through one gas feed manifold, and another mixture of gases containing hydrogen gas, carbon dioxide gas, air and oxygen is provided to the reactor vessel through another gas feed manifold. In some embodiments, a first stream of gas is fed into the culture and a second stream of gas is fed into the headspace of the bioreactor vessel. In some embodiments, the flow of gas provided to the headspace is constant, and the flow of gas provided to the culture is regulated (e.g., under feedback control). In some embodiments, the proportion of oxygen in the flow of gas provided to the culture is regulated. In some embodiments, the proportion of oxygen in the flow of gas provided to the culture is under feedback control (e.g., oxygen feedback control).

In some embodiments, where a first stream of gas is fed into the culture and a second stream of gas is fed into the headspace of the bioreactor vessel, the oxygen partial pressure in the first stream may be raised to a level higher than would otherwise have been safe to do without the second stream. In some embodiments, the oxygen partial pressure in the first stream is higher than the oxygen partial pressure in the second stream. In some embodiments, the proportion of oxygen gas in the first stream is higher than the proportion of oxygen gas in the second stream. In some embodiments, the proportion of oxygen gas in the first stream is about 4% (v/v) or more, e.g., about 10% (v/v) or more, about 15% (v/v) or more, about 20% (v/v) or more, about 25% (v/v) or more, including about 30% (v/v) or more. In some embodiments, the proportion of oxygen gas in the first stream is in the range of 0 to about 40% (v/v), e.g., 0 to about 35% (v/v), about 4% (v/v) to about 35% (v/v), including about 5% (v/v) to about 30% (v/v).

In some embodiments, the rate of gas delivery to the culture is under oxygen feedback control. In some embodiments, the flow rate of gas delivered to the culture is under oxygen feedback control. The flow rate may be controlled by any suitable mechanism, such as, but not limited to, a valve. In some embodiments, the flow rate of oxygen gas provided to the culture is under oxygen feedback control. In some embodiments, the flow rate of a gas mixture containing oxygen gas is under oxygen feedback control. In some embodiments, the flow rate of a gas mixture containing oxygen gas and/or air, hydrogen gas, carbon dioxide gas is under oxygen feedback control. In some embodiments, the flow rate is about 0.1 vessel volume per minute (vvm) or more, e.g., about 0.2 vvm or more, about 0.3 vvm or more, about 0.4 vvm or more, about 0.5 vvm or more, about 0.8 vvm or more, including about 1 vvm or more, and in some embodiments, the flow rate is about 5 vvm or less, e.g., about 4.5 vvm or less, about 4 vvm or less, about 3.5 vvm or less, about 3 vvm or less, about 2.5 vvm or less, including about 2 vvm or less. In some embodiments, the flow rate is in the range of about 0.1 to about 5 vvm, e.g., about 0.2 to about 4.5 vvm, about 0.3 to about 4.0 vvm, about 0.4 to about 3.5 vvm, including about 0.5 to about 3 vvm.

The culture may be mixed (e.g., agitated or stirred) to promote transfer of oxygen gas and/or other gases to the culture. In some embodiments, the culture is stirred using an impeller. The impeller may be positioned at a suitable distance from a gas dispersing mechanism, such as a sparger, to promote mass transfer of the gas. The impeller may be any suitable impeller, including, but not limited to, a Rushton impeller, a gas entrainment impeller, a Rushton-style impeller with gas entrainment, or a basket impeller. The impeller may be rotated at a suitable rate and direction to promote mass transfer of oxygen gas and/or other gases to the culture.

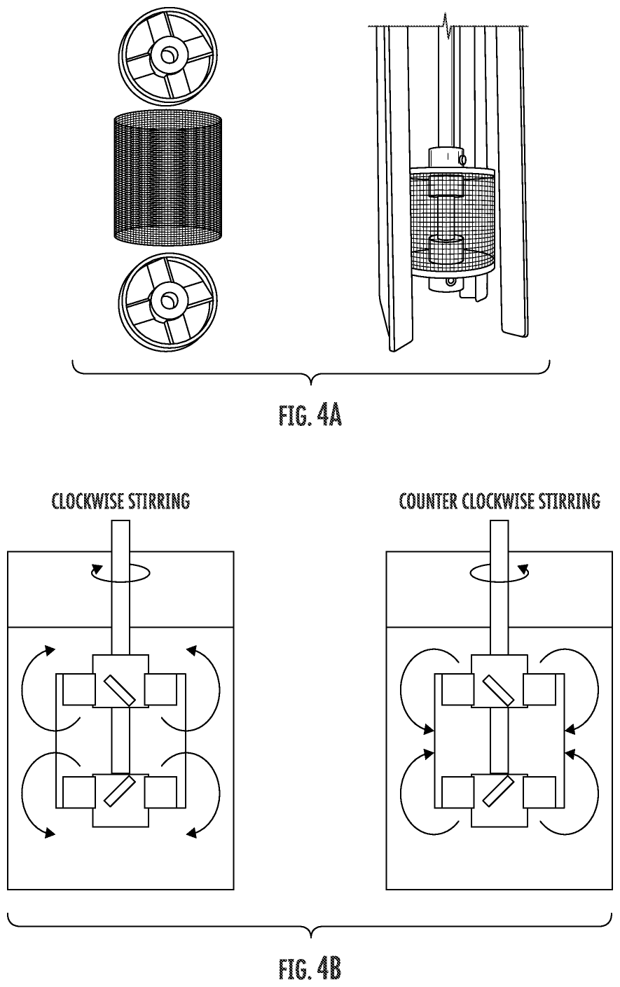

In some embodiments, where the impeller is a basket impeller, the rotation of the impeller is in a direction that minimizes foaming yet retains sufficient mass-transfer rates. To minimize foaming, the impeller may be rotated in a direction that draws liquid into the cylindrical basket from the lateral mesh surface and expels liquid from the top or bottom of the cylinder (FIG. 4B).

Any suitable number of impellers may be positioned on a stirring shaft. In some embodiments, a stirring shaft may have one, two, three or more impellers. The types of impellers on a single stirring shaft may be the same or different from each other.

In some embodiments, the rate of mass transfer of oxygen (and/or other gases) into the culture is controlled by varying the rate of mixing of the culture. In some embodiments, the rate of mixing of the culture is under oxygen feedback control. In some embodiments, the speed of rotation of an impeller is under oxygen feedback control.

In some embodiments, the culture is circulated over a gas permeable membrane that separates the culture compartment from a gas compartment containing oxygen gas, hydrogen gas, and/or other gases such that said gases (e.g., oxygen gas) diffuses from the gas compartment to the culture compartment. In some embodiments, the gas compartment is maintained at an elevated pressure. In some embodiments, the gas compartment is maintained at about 1 psig or greater, e.g., about 5 psig or greater, about 10 psig or greater, including about 15 psig or greater, and in some embodiments, the pressure is maintained at about 40 psig or less, e.g., about 30 psig or less, about 20 psig or less, about 18 psig or less, about 15 psig or less, about 12 psig or less, including about 10 psig or less. In some embodiments, the gas compartment is maintained at a pressure in the range of about 1 psig to about 40 psig, e.g., about 5 psig to about 30 psig, about 10 psig to about 20 psig, including about 15 psig to about 20 psig. In some embodiments, the gas compartment is maintained at a higher pressure than the culture pressure. In some embodiments, the gas compartment is maintained at about 1 psig or greater than the culture pressure, e.g., about 5 psig or greater, about 10 psig or greater, including about 15 psig or greater, and in some embodiments, the pressure is maintained at about 40 psig or less above the culture pressure, e.g., about 30 psig or less, about 20 psig or less, about 18 psig or less, about 15 psig or less, about 12 psig or less, including about 10 psig or less. In some embodiments, the gas compartment is maintained at a pressure in the range of about 1 psig to about 40 psig above the culture pressure, e.g., about 5 psig to about 30 psig above, or about 10 psig to about 20 psig, including about 15 psig to about 20 psig above the culture pressure.

The gas compartment may contain any suitable amount of oxygen gas to promote oxygenation of the culture across the gas permeable membrane. In some embodiments, the oxygen concentration in the gas compartment is about 20% (v/v) or more, e.g., about 30% (v/v) or more, about 40% (v/v) or more, about 50% (v/v) or more, about 60% (v/v) or more, about 70% (v/v) or more, about 80% (v/v) or more, including about 90% (v/v) or more. In some embodiments, the oxygen concentration in the gas compartment is about 100% (v/v) or less, e.g., about 95% (v/v) or less, about 90% (v/v) or less, about 85% (v/v) or less, about 80% (v/v) or less, about 75% (v/v) or less, about 70% (v/v) or less, about 60% (v/v) or less, including about 50% (v/v) or less. In some embodiments, the oxygen concentration in the gas compartment is in the range of about 20% (v/v) to about 100% (v/v), e.g., about 30% (v/v) to about 100% (v/v), about 40% (v/v) to about 100% (v/v), about 50% (v/v) to about 100% (v/v), about 60% (v/v) to about 95% (v/v), including about 70% (v/v) to about 95% (v/v). In some embodiments, the gas compartment includes a mixture of oxygen gas and nitrogen gas. In some embodiments, the gas compartment includes air.

In some embodiments, the gas permeable membrane forms a tubing. In some embodiments, the culture is passed through the lumen of the tubing and the gas compartment containing oxygen gas forms the outside of the tubing. In some embodiments, the gas compartment containing oxygen gas is in the lumen of the tubing, and the culture is circulated over the surface of the wall of the tubing.

Following passage through the reactor system holding microorganisms which uptake the gases, in certain embodiments the residual gases may either be recirculated back to the bioreactor, or burned for process heat, or flared, or injected underground, or released into the atmosphere. In certain embodiments herein utilizing H₂ as electron donor, H₂ may be fed to the culture vessel either by bubbling it through the culture medium, or by diffusing it through a hydrogen permeable-water impermeable membrane known in the art that interfaces with the liquid culture medium.

In some embodiments of the present invention centrifugal compressors proven in commercial GTL processes such as the Haber-Bosch process, are used to compress gaseous feedstocks that are pumped into the bioreactor/s. In certain embodiments of the present invention, said gaseous feedstocks include but are not limited to one or more of: H₂, CO₂, O₂, CO, CH₄, syngas, producer gas, NH₃, H₂S. In certain non-limiting embodiments, the sulfur content of the feedstock gases is around 0.5-1 mg S/m3 (STP) or less. In other non-limiting embodiments, the sulfur content of the feedstock gases is greater than 1 mg S/m3 (STP).

In certain embodiments, a C1 molecule such as but not limited to carbon dioxide, carbon monoxide, methane, methanol, formaldehyde, formate, or formic acid, and/or mixtures containing C1 molecules including but not limited to various syngas compositions generated from various gasified, pyrolyzed, or steam-reformed fixed carbon feedstocks, is utilized by the microorganism as a carbon source and is biochemically converted into longer chain organic molecules (i.e., C2 or longer and, in some embodiments, C5 or longer carbon chain molecules) under one or more of the following conditions: aerobic, microaerobic, anoxic, anaerobic, and/or facultative conditions. In some embodiments, gaseous CO₂ is utilized by the microorganism as a carbon source and is chemoautotrophically converted into longer chain organic molecules (i.e., C2 or longer and, in some embodiments, C5 or longer carbon chain molecules) under aerobic, microaerobic, anoxic, anaerobic, and/or facultative conditions. In some embodiments, H₂ is used as an electron donor and O₂ is used as an electron acceptor for the carbon fixation and conversion of the C1 carbon molecule into longer chain organic molecules. In some embodiments, H₂ is used as an electron donor and O₂ is used as an electron acceptor for chemoautotrophic carbon fixation and conversion of the CO₂ into longer chain organic molecules.

Certain aspects of the present invention relate to compression of gaseous feedstocks. Certain aspects of the present invention relate to synthesis of organic compounds from gaseous feedstocks. Certain aspects of the present invention relate to purge gas management.

In certain embodiments, an organic carbon source is used as a source of carbon and/or reducing electrons in the cell metabolism. In certain embodiments, such growth and metabolism is heterotrophic or mixotrophic.

In certain embodiments, one or more of the following parameters are monitored and/or controlled in the bioreactor: waste product levels; pH; temperature; salinity; dissolved oxygen; dissolved carbon dioxide gas; liquid flow rates; agitation rate; gas pressure. In certain embodiments, the operating parameters affecting chemoautotrophic growth are monitored with sensors (e.g., dissolved oxygen probe or oxidation-reduction probe to gauge electron donor/acceptor concentrations), and/or are controlled either manually or automatically based upon feedback from sensors through the use of equipment including but not limited to actuating valves, pumps, and agitators. In certain embodiments, the temperature of the incoming broth as well as of incoming gases is regulated by systems such as, but not limited to, coolers, heaters, and/or heat exchangers.

In some embodiments, the bioreactor includes a liquid level sensor configured to measure the liquid level of the culture in the bioreactor vessel. The liquid level sensor may provide a feedback signal to control the rate at which a portion of the culture is withdrawn from the vessel, to maintain the volume of the culture within a target range. The liquid level sensor may be any suitable sensor for determining the liquid level in the vessel. In some embodiments, the liquid level sensor is a conductance-based sensor. In some embodiments, the bioreactor may include a liquid level controller configured to regulate the rate of liquid removal from the vessel, based on a liquid level feedback signal from the liquid level sensor. The liquid level feedback control may include any suitable control loop (PI (proportional integral), PID (proportional integral derivative) or on/off). In some embodiments, the liquid level controller is configured to regulate the activity of a pump configured to remove liquid from the vessel.

In certain embodiments condensable vapors and/or gases emitted from the bioreactor are recovered. In certain such embodiments the said recovery entails cooling the off-gas to condense the said condensable vapors and/or gases. In certain such embodiments, the said condensation entails cooling and pressurizing. In certain such embodiments, the said condensable vapors and/or gases are mostly or entirely composed of water. In certain embodiments, condensable vapors and/or gases are recovered from a gas recirculation (i.e., recycle) loop by cooling the synthesis gas (e.g., H₂, CO₂, and/or O₂) to condense the condensable vapors and/or gases and separate them from the non-condensable gases (e.g., H₂, CO₂, and/or O₂). In certain such embodiments the said condensable vapors and/or gases are condensed using cooling and increased pressure. In certain embodiments, a liquid water product is separated from gas (e.g., H₂, CO₂, and/or O₂), which is recycled. In certain embodiments, liquid water is recovered from a high-pressure condenser and/or liquid broth recovered from the bioreactor is flashed to release most dissolved gases in a let-down vessel. In certain such embodiments, the liquid is flashed to around 20 bar or less. In certain such embodiments, the said released gas is recirculated back into the bioreactor and/or used as a fuel in, for example, a combustion furnace.

Certain aspects of the present invention relate to arrangement and location of the water condenser(s), recirculation compression, addition of make-up gas and extraction of purge gas. Certain aspects relate to the water condenser(s) temperature, gas pressure and location of make-up gas addition.

Certain aspects of the present invention relate to inert-gas and purge-gas management. In certain embodiments of the present invention, apart from synthesis gas, e.g., H₂, CO₂, and/or O₂, the fresh make-up gas supplied to the synthesis loop, comprising the bioreactor/s and gas recirculation loop/s, may contain small quantities of inert gases. Such inert gases may include N₂ (e.g., from process air), methane (e.g., from gas generation or gas source), argon (e.g., from process air), and helium (e.g., from the natural gas). Methane may or may not be inert depending upon if a methanotroph is included in the culture. In certain embodiments, a methanotroph is included in the culture and the methane is not an inert. In other embodiments, a methanotroph is not included in the culture and the methane is an inert. In certain embodiments, the inert gases tend to concentrate in the synthesis loop e.g., the bioreactor and/or gas recirculation loop, and must be removed to maintain the loop material balance. A high inert gas level may have various drawbacks. For example, it may decrease the bioprocess e.g., knallgas bioprocess performance by reducing the hydrogen, carbon dioxide, and/or oxygen partial pressures. The gas recycle flow may be increased by the amount of inert gas. Piping and equipment correspondingly may need to be increased in size, and the associated power consumption for gas recirculation i.e., recycle may increase. There may be an unfavorable effect on condensation of water co-product and/or of other condensable vapors or gases. Because of dilution, less water and/or other condensable vapors or gases may be condensed from the recycle synthesis gas (e.g., H₂, CO₂, and/or O₂) by less expensive cooling e.g., air and/or water cooling and/or higher temperature level refrigeration.

In certain embodiments, a portion of the inert gases dissolves in the liquid produced in the water condenser and/or in the broth harvested from the reactor. In certain embodiments, if the synthesis gas pressure is high enough, for example, around 300 bar, and/or the inert gas concentration in the synthesis loop make-up gas low enough, for example, under 0.2 vol %, then dissolution in the water co-product suffices to remove the inerts from the synthesis loop.

In certain embodiments, in addition to, or instead of, removal as dissolved gases (flash gas), inerts are removed from the gas phase by withdrawing a small purge-gas stream from the loop. Certain aspects of the present invention relate to determining the appropriate inert gas concentration and purge-gas stream via technoeconomic calculation.

In certain embodiments, one or more approaches are utilized for reducing the losses associated with a purge gas stream. In certain embodiments, the purge gas is fed to a second synthesis loop e.g., comprising one or more bioreactors and one or more gas recirculation loops operating at a slightly lower pressure. In certain such embodiments, this loop is operated at a very high inert level (e.g., 40% or more), only a very small final purge stream is necessary. In certain embodiments up to 75% of the hydrogen from the first-loop purge stream is utilized in the second loop.

In certain embodiments, hydrogen is recovered using one or more cryogenic units. In certain such embodiments, water and/or other condensable gases and/or vapors are removed from the purge gas by cooling. In certain embodiments, CO₂ and/or other water soluble gases and/or vapors are removed from the purge gas in a water wash. Certain such water washes operate at around 7.5 MPa (75 bar). In certain embodiments, molecular sieve adsorbers are utilized to eliminate moisture from the purge gas stream. In certain embodiments dry, CO₂-free purge gas flows from adsorbers to a cold box. In certain such embodiments, the said purge gas is cooled to a temperature of about −188° C. (85 K). In certain such embodiments, the said cooling involves heat exchange with cold hydrogen product from the cold box. In certain embodiments, partial condensation liquefies methane and argon as well as some of the nitrogen and helium. In certain such embodiments, these are removed in a separator, leaving a hydrogen-rich gas. In certain embodiments, the purge gas is cooled to liquid nitrogen temperatures or lower e.g., −196° C. or less. In certain such embodiments, nitrogen in the purge gas is liquified and removed via a separator, leaving a hydrogen-rich gas. In certain embodiments, liquid flows through a control valve, reducing its pressure, and into a brazed aluminum (plate-fin or core-type) heat exchanger. In certain such embodiments, hydrogen-rich gas which has already had its inert gas content reduced or removed also flows into the same exchanger through separate passages. In certain such embodiments, the vaporizing liquid and/or the hydrogen-rich gas stream are warmed by cooling the entering purge gas. In certain embodiments, liquid ammonia may be used to provide additional refrigeration. In certain embodiments, a warmed hydrogen-rich gas, from which inerts have been reduced or removed, flows back to the suction side of a synthesis gas (e.g., H₂, CO₂, and/or O₂) compressor. In certain such embodiments, the said hydrogen-rich gas flows into the second stage of a synthesis gas compressor. In certain embodiments, about 90-95% of the hydrogen in the purge gas can be recovered. In certain such embodiments, the remaining gas, with a high concentration of inerts, serves as fuel for a primary reformer and/or for heating or drying applications. In certain such embodiments, after heating in a preheater, a portion of said remaining gas serves to regenerate the molecular sieves and then flows to reformer fuel and/or heating or drying applications. Cryogenic hydrogen recovery units that may be used in certain embodiments of the present invention are supplied by firms such as Costain Engineering (formerly Petrocarbon Development), Linde, and Air Liquide, among others. In certain embodiments of the present invention, hydrogen recovery is accomplished by membrane separation.

The Monsanto Prism membrane separator system, for example, reportedly uses selective gas permeation through membranes to separate gases. This principle has been applied to separating hydrogen from other gases. The membranes are hollow fibers with diameters of about 0.5 mm. The fiber is a composite membrane consisting of an asymmetric polymer substrate and a polymer coating. The design of a single separator module (length, 3-6 m; diameter, 0.1-0.2 m) resembles a shell and tube heat exchanger. A bundle with many thousands of hollow fibers is sealed at one end and embedded in a tubesheet at the other. The entire bundle is encased in a vertical shell. In certain embodiments of the present invention, a membrane separation technology is used to separate hydrogen from other gases and in particular inert gases. In certain such embodiments, the said membrane separation technology comprises a Monsanto Prism membrane separator system, and/or Polysep Membrane System from UOP, and/or similar technologies. In certain embodiments, the purge gas is water scrubbed. In certain embodiments, the said water scrub of the purge gas is performed in a pressure range of 135-145 bar, or at less than 135 bar. In certain embodiments, the scrubbed purge gas is sent to the Prism membrane separators at a temperature of around 35° C. Trace concentrations of water vapor in the gas stream are reported to pose no problem to the membrane. In certain embodiments, a dryer system is not used to dry the purge gas stream. In certain embodiments, the purge gas stream enters the separator on the shell side, i.e., the outside of the hollow fibers. In certain such embodiments, hydrogen permeates through the wall of the fibers. In certain such embodiments, hydrogen-rich permeate gas flows down the bore of the fiber and through the tubesheet and is delivered at the bottom of the separator. In certain such embodiments, the remaining (nonpermeating) gases, including but not limited to nitrogen, methane, and argon, are concentrated on the shell side, recovered through the top and pass to the next separator module. In certain such embodiments, several separators operate in series. The rate of permeation decreases across a bank of separators as the hydrogen partial pressure differential across the membrane approaches zero. In certain such embodiments, a second bank of separators with lower pressure on the tube side is used to increase the hydrogen recovery. In certain such embodiments of the present invention, around 40-70% of the recovered hydrogen leaves the first bank of separators at around 7 MPa (70 bar). In certain such embodiments, the said recovered hydrogen is returned to the syngas (e.g., H₂, CO₂, and/or O₂) compressor. In certain such embodiments, the said recovered H₂ is returned to the second-stage suction of the syngas compressor. In certain such embodiments, the second bank permeate hydrogen is recovered at around 2.5-2.8 MPa (25-28 bar). In certain such embodiments, the said recovered hydrogen is returned to the syngas (e.g., H₂, CO₂, and/or O₂) compressor. In certain such embodiments, the said recovered H₂ is returned to the first-stage suction of the syngas compressor. In certain embodiments, in addition to, or instead of systems based on hollow fibers, membrane modules are utilized in which the membrane is in the form of a sheet wrapped around a perforated center tube using spacers to separate the layers. The raw gas flows in axial direction in the high pressure spacer and the permeate is withdrawn in the low pressure spacer. In certain embodiments, this membrane configuration is utilized to recover hydrogen from purge gas. In certain such embodiments, a Serarex module provided by Linde is utilized in such an application. In certain embodiments, the overall hydrogen recovery from the purge gas stream is around 90-95%. In certain embodiments, the remaining nonpermeate gas stream flows to primary reformer fuel.

In certain embodiments of the present invention, hydrogen recovery is accomplished using pressure swing adsorption. In certain such embodiments, pressure swing adsorption on zeolite molecular sieves (PSA) may be used for hydrogen recovery from purge gas. In certain such embodiment, the PSA process, originally developed by Union Carbide under the name HYSIV, and later marketed as Polybed PSA by UOP, is utilized for hydrogen recovery. In certain embodiments, PSA technologies offered by Linde and other companies may be utilized for H₂ recovery. In certain embodiments, PSA unit/s are operated at adsorption pressures of around 20-30 bar. In certain embodiments PSA unit/s achieve recovery rates higher than around 82% for hydrogen. In certain embodiments, carbon-based adsorbents for pressure swing adsorption are utilized. In certain such embodiments, a PSA process developed by Bergbau-Forschung and offered by Costain is utilized.

In certain embodiments of the present invention, hydrogen recovery is performed using mixed metal hydrides. In certain such embodiments, a hydride, such as LaNi₅, FeTi, or Mg₂Cu, is in the form of ballasted pellets. The ballast material serves as a heat sink to store the heat of adsorption. Subsequently, this is used to supply the heat of desorption. The ballast also is the binder for the pellets, preventing attrition. Each type of metal hydride is susceptible to certain contaminants. Therefore, selection of the metal hydride must be based on the analysis of the gas to be treated. In certain such embodiments, the system yields around 99 mol % hydrogen product at a recovery efficiency of around 90-93%.

In certain embodiments of the present invention, vented off-gas is recovered and converted into useful work and/or heat. In certain such embodiments, the said work and/or heat is used in the process. In certain embodiments the vented off-gas is from a fuel rich headspace.

In certain embodiments of the present invention, kilowatt hours per product for make-up gas compression, recycle, and refrigeration demand versus synthesis pressure are determined and an operating pressure giving minimum electrical demand per product is identified. In certain embodiments, the bioprocess is run at the pressure providing minimum electrical demand per product. In certain aspects of the present invention, the front-end pressure coming from the gas generation source can determine the entry pressure of the compressor feeding gases to the bioprocess. For example, in a plant using partial oxidation with an operating pressure of 80 bar for the produced gas, roughly less than half as much energy may be needed to compress the make-up gas to 180 bar, than, for example, in a steam reforming plant producing gas that is input at a pressure of only 25 bar.

Certain aspects of the present invention relate to selecting the best synthesis pressure based on factors including but not limited to the entire plant energy balance, the mechanical design, and the associated investment costs. In certain aspects, the costs for energy (e.g., the H₂ and/or other fuel gases) are weighed against investment.

In some embodiments the protein production and distribution of amino acid molecules produced by the microorganism is optimized through one or more of the following: control of bioreactor conditions, control of nutrient levels, and/or genetic modifications of the cells. In certain embodiments, pathways to amino acids, or proteins, or other nutrients, or whole cell products, or other biochemicals or organic acids are controlled and optimized for the production of chemical products by maintaining specific growth conditions (e.g., levels of nitrogen, oxygen, phosphorous, sulfur, trace micronutrients such as inorganic ions, and if present any regulatory molecules that might not generally be considered a nutrient or energy source). In certain embodiments, dissolved oxygen (DO) may be optimized by maintaining the broth in aerobic, microaerobic, anoxic, anaerobic, or facultative conditions, depending upon the requirements of the microorganisms. A facultative environment is considered to include aerobic upper layers and anaerobic lower layers caused by stratification of the water column, or a spatial separation of aerobic or microaerobic regions, and anaerobic regions caused by spatial separation of regions exposed to O₂ containing gases and regions that are not exposed to O₂ containing gases.

In some embodiments, the microorganisms, e.g., chemoautotrophic microorganisms, are grown under conditions conducive to accumulation of polyhydroxyalkanoate (PHA), e.g., polyhydroxybutyrate (PHB) and/or polyhydroxyvalerate (PHV), by the microorganisms. In some embodiments, the microorganisms, e.g., chemoautotrophic microorganisms are grown under limitation of one or more nutrients, such as under nitrogen or phosphorous limitation, to cause accumulation of PHA (e.g., PHB; PHV). In some embodiments, the microorganism, such as a chemoautotrophic microorganism grown on H₂/CO₂ and/or syngas, accumulates PHA, such as PHB and/or PHV, in the cell biomass. In some embodiments, PHA (e.g., PHB; PHV) is accumulated to about 50% or more of the microorganism biomass by weight, about 60% or more, or about 70% or more by weight.

In certain embodiments, the microorganisms, e.g., chemoautotrophic microorganisms are grown under conditions that promote production of vitamins, such as, but not limited to, B vitamins, e.g., one or more of vitamin B₁, vitamin B₂, and/or vitamin B₁₂, by the microorganisms. In some embodiments, the microorganisms may be grown chemoautotrophically to produce one or more vitamins, such as vitamin B₁, vitamin B₂, and/or vitamin B₁₂.

Certain aspects of the present invention relate to clean-in-place (CIP) and sterilization-in-place (SIP). Certain aspects of the present invention relate to the ability to operate pure culture fermentations, or defined and controlled consortia of microorganisms. In certain embodiments, a sterile boundary is imposed around the requisite vessels where sterile conditions are required. Certain aspects of the present invention relate to the ability to safely, repeatably and reliably create and maintain sterile envelopes to cultivate a desired concentration and purity of microbial product, such as but not limited to SCP. Certain aspects of the present invention relate to hygienic and aseptic process design principles. In certain embodiments, measures or design features including but not limited to one or more of the following are utilized in the present invention: materials in contact with the fermentation product are non-reactive, non-adsorptive, non-additive, and/or non-shedding; surfaces in contact with the fermentation product have a “smooth” surface finish, such as 0.5 μm Ra or better; welds comply with the requirements as stipulated in, for example, ASME BPE 2016; hygienic valves compatible with Clean in Place (CIP) and Sterilize in Place (SIP) are installed at sterile boundaries; self-draining hardware is utilized that prevents pooling of cleaning liquids during CIP and/or pooling of condensate during SIP; equipment inside sterile envelopes is compatible with sterilization using “clean steam” typically being held at elevated temperatures and pressures (e.g., around 133° C. at around 2.0 barg) for around 30 minutes or longer; equipment inside sterile envelopes is compatible with dilute, hot caustic solutions (e.g. around 2% w/w and around 60 to 80° C.) which are to be used for CIP; wetted elastomeric components are of hygienic design and construction and are compatible with CIP and SIP operations; piping dead legs are minimized or eliminated as far as practicable and if present are of a length that complies with good hygienic engineering practice—e.g. L/D<2—as advised in industry recognized codes of practice. In certain embodiments, at the end of steaming dry sterile air is introduced into vessels and piping. This serves three important functions: 1) to prevent vacuum formation as the steam collapses during the immediate cool down of the vessels and piping, which in turn prevents the likelihood of contaminants being drawn into the sterile envelope; 2) to help maintain sterility and integrity by preventing ingress of contaminants into the sterile envelope by using sterile air with a slight overpressure so that once the steam has collapsed and the metallic components have fully cooled, contaminants are kept out of the sterile envelope; 3) to help dry and sweep out of piping and low points in vessels residual condensate post-SIP in the sterile envelope. In certain such embodiments, the sterile air is purged and/or the vessel inerted to eliminate the potential of an explosive atmosphere being created on start up. In certain such embodiments, nitrogen is used as a purge gas. In other embodiments, particularly when the bioprocess already utilizes carbon dioxide, the carbon dioxide supply is used as a purge gas. In certain such embodiments, the carbon dioxide storage and supply system is sized for this purging and inerting duty as well as providing a constant supply of gas when the gas bioprocess is in operation.

The biomass produced by methods of the present disclosure may be separated from the liquid media by any suitable manner. Separation of cell mass from liquid suspension can be performed by methods known in the art of microbial culturing [Examples of cell mass harvesting techniques are given in International Patent Application No. WO08/00558, published Jan. 8, 1998; U.S. Pat. Nos. 5,807,722; 5,593,886 and 5,821,111], A biomass generated by the cultured microorganisms, e.g., chemoautotrophic microorganisms, may be harvested using any suitable method, and a protein hydrolysate may then be prepared from the harvested biomass. In some embodiments, the biomass is separated from the liquid media using a suitable method. Suitable methods include, without limitation, centrifugation; flocculation; flotation; filtration using a membranous, hollow fiber, spiral wound, or ceramic filter system; vacuum filtration; tangential flow filtration; clarification; settling; hydrocyclone. In certain embodiments where the microbial cell mass may be immobilized on a matrix, it may be harvested by methods including but not limited to gravity sedimentation or filtration, and separated from the growth substrate by scraping or liquid shear forces.

For certain microbial suspensions there is often a critical dry solids weight percent at which point the broth transitions from a freely flowing liquid phase to a semi-solid gel like material exhibiting visco-elastic behavior. As the dewatering process continues and dry cell weight fraction increases the material becomes highly viscous and almost impossible to pump out of vessels and along piping. Certain aspects of the present invention relate to determining the dry solids percentage at which the transition takes place and designing the final stages of the DSP train such that the wet biomass or slurry remains easy to pump. In certain such embodiments, the said wet biomass or slurry comprises single cell protein (SCP). In certain embodiments, the dewatering process is terminated before attaining this said critical dry weight percentage.

In some embodiments, biomass, e.g., protein-rich biomass, produced in the methods described herein is separated from the culture medium and used as a single cell protein (SCP). In some embodiments, the biomass is processed and/or formulated for use, e.g., processed and/or formulated for use, in one or more of the following applications: fertilizer; biostimulant; biofertilizer; fungal growth enhancer or supplement; nutrient; ingredient; animal feed or within an animal feed formulation; human food or within a human food formulation. In some embodiments, the biomass is used as a substitute, e.g., high-protein substitute, for fishmeal and/or other animal protein, and/or is used in plant fertilizer products and/or mushroom and fungal growth enhancers.

Harvested microbial cells in certain embodiments can be broken open to prepare a lysate, using well known methods including but not limited to one or more of the following: ball milling, cavitation pressure, sonication, homogenization, or mechanical shearing. In some embodiments, the cells in the biomass may be lysed by one or more freeze-thaw cycles, a lytic enzyme, detergents, solvents, or antibiotics.

The harvested biomass in some embodiments may be dried in a process step or steps. Biomass drying can be performed in certain embodiments using any suitable method, including but not limited to, one or more of the following: centrifugation, drum drying, evaporation, freeze drying, heating, spray drying, vacuum drying, and/or vacuum filtration. In certain embodiments, waste heat can be used in drying the biomass. In certain embodiments, heat waste from the industrial source of flue gas used as a carbon source can be used in drying the biomass. In certain embodiments, the heat co-product from the generation of electron donors and/or C1 carbon source can be used for drying the biomass. In certain embodiments the heat co-product from gasification, methane stream reforming, autoreforming, or partial oxidation can be used for drying the biomass. In certain embodiments, process heat generated as a co-product of syngas or producer gas generation can be used in drying the biomass. Heat waste from the industrial source of tail gas or flue gas can be used in drying the biomass, in certain embodiments. In certain embodiments, waste heat can be used in drying the biomass.

In certain embodiments of the present invention, SCP biomass is manufactured into a final product shape and size that is easy for animals to eat and digest, such as, but not limited to one or more of the following: pellets, granules, powder, and/or a slurry. In certain embodiments, a powder is made using spray drier technology. In certain embodiments, pellets are made by feeding the powder from a spray drier process or a wet slurry to a pelletizer or pellet mill.

In certain embodiments, the biomass is further processed following drying, or, without a preceding drying step, to aid the separation and production of useful biochemicals. In certain embodiments, this additional processing involves the separation of the protein or lipid content or vitamins or nucleic acids or other targeted biochemicals from the microbial biomass. In certain embodiments, the separation of the lipids can be performed by using nonpolar or polar solvents to extract the lipids, such as, but not limited to one or more of: hexane, cyclohexane, dodecane, ethyl ether, alcohol (methanol, isopropanol, ethanol, etc.), tributyl phosphate, supercritical carbon dioxide, trioctylphosphine oxide, ammonia, secondary and tertiary amines, propane, acetone, propylene carbonate, dichloromethane, or chloroform. In certain embodiments, other useful biochemicals can be extracted using solvents, including but not limited to, one or more of: chloroform, dichloromethane, acetone, ethyl acetate, propylene carbonate, and tetrachloroethylene. In certain embodiments cell lysis is performed for the separation and production of useful biochemicals.

In certain non-limiting embodiments of the present invention, a protein concentrate and/or protein isolate and water are the major bioprocess outputs (including downstream processing DSP). In certain such embodiments, the said protein concentrate and/or isolate is ≤80% protein by weight, or at least around or greater than about any of 60%, 65%, 70%, 75%, 80%, 85%, 90%, or 95% protein by weight.

Whole cell protein produced according to the present invention, may in certain embodiments be used in structured meat analogues (including fish or seafood analogues). In certain such embodiments, the said structured meat analogues have organoleptic qualities closely resembling animal meat products. In certain embodiments of the present invention, protein and/or other nutrients produced according to the present invention may be used in a broad set of nutritional products including but not limited to one or more of the following: meat alternatives, baking flours, nutritional bars, nutritional supplements, beverages, and/or animal feed products. In certain embodiments of the present invention, protein and/or other nutrients may be utilized in combination with product formulation and processing, flavors and textures, food prototyping, production, and distribution well known in the art.

Population growth and consumer demand for healthy, sustainable nutrition are expected to result in rapid market growth for these products, from current $14B/yr to $140B/yr by 2029. Current market growth is being driven by companies producing meat analogues from plant material, such as soy protein concentrates, isolates, and hydrolysates. Microbial protein produced according to the present invention has distinct sustainability advantages over the plant-based processes, particularly regarding agricultural land and water use, and a well-balanced amino acid profile comparable to animal protein, promising rapid penetration of products produced according to the present invention.

The knallgas carbon-conversion technology described in certain embodiments of the present invention is decoupled from agricultural land sources and can be farmed vertically with far lower land, water, and resource utilization than other protein sources. In certain embodiments of the present invention, the land requirement of the knallgas protein production bioprocess is lower per unit protein (m² land*yr/kg protein) than the average land requirement to produce soy protein concentrate (SPC). In certain embodiments of the present invention, the land requirement of the end-to-end knallgas protein production bioprocess is around 500 times, or around 1,000 times, or around 2,000 times, or around 2,500 times lower per unit protein (m² land*yr/kg protein) than the average land requirement for soy protein concentrate (SPC); around 5,000, or around 10,000 or around 13,000 times lower than for poultry protein; and around 100,000 times, or around 250,000 times, or around 500,000 times lower than required for beef protein [Nadathur, S., Wanasundara, J. P. D., & Scanlin, L. (2016). Sustainable Protein Sources. Elsevier Science. https://books.google.ca/books?id=ZZXBCQAAQBAJ]. Also, unlike these other animal- or plant-protein sources, there is zero requirement for arable land in certain embodiments of the present invention. In certain embodiments of the present invention, direct land usage (i.e. LCA scope 1 land footprint) of a production plant designed according to the present invention uses no arable land and has zero land footprint that corresponds to arable land. In certain embodiments of the present invention, LCA scope 1, and LCA scopes 2 and/or 3 land footprints of a production plant designed according to the present invention uses no arable land and has zero land footprint that corresponds to arable land. In certain embodiments of the present invention, direct and indirect land usage (i.e. LCA scopes 1, 2, and 3 land footprint) of a production plant designed according to the present invention uses no arable land and has zero land footprint that corresponds to arable land.

In certain embodiments of the present invention the total fresh water footprint (i.e., blue, green, and grey water in kg total water per kg protein) is lower per kg protein than the total water footprint for soy protein concentrate (SPC). In certain embodiments of the present invention the total fresh water footprint (i.e., blue, green, and grey as defined by Mekonnen et al) is around 1,000 times, or around 3,000 times, or around 5,000 times, or around 7,000 times less per kg protein than soy protein concentrate (SPC); around 10,000 times or around 20,000 times, or around 30,000 times, or around 40,000 times less than poultry protein, and around 50,000 times, or around 100,000 times, or around 140,000 times less than beef protein, according to values published by the UNESCO-IHE Institute for Water Education for these protein sources [Mekonnen, M. M. and Hoekstra, A. Y. (2010) The green, blue, and grey water footprint of crops and derived crop products, Value of Water Research Report Series No. 47, UNESCO-IHE, Delft, the Netherlands is incorporated herein by reference in its entirety].

In certain embodiments of the present invention, the cradle-to-grave GHG footprint (CO2e/kg protein assuming end-of-life that all carbon in the protein-rich biomass is metabolized back to CO₂ and emitted to the atmosphere) of the knallgas protein production bioprocess is lower than the GHG footprint per protein for SPC. In certain embodiments of the present invention, the cradle-to-grave GHG footprint of the knallgas process in certain embodiments of the present invention using biogenic CO₂ and electrolytic H₂ generated using low-CO₂ renewable power, is around 0.24 kg CO2e/kg protein or around 0.5 kg CO2e/kg protein or around 1 kg CO2e/kg protein or around 2 kg CO2e/kg protein. Certain embodiments of the present invention have a carbon negative GHG footprint cradle-to-gate e.g., around −2 kg CO2e/kg protein, or around −1 kg CO2e/kg protein, or around −0.5 kg CO2e/kg protein, or around −0.25 kg CO2e/kg protein, GHG footprint cradle-to-gate. In certain embodiments of the present invention the cradle-to-grave GHG footprint of the protein product is around 5 times lower per kg protein than that reported for SPC, or around 10 times lower, or around 20 times lower, or around 30 times lower, or around 40 times lower per kg protein than the average GHG footprint reported for SPC; around 20 times, or around 50 times, or around 75 times, or around 100 times, or around 150 times less than for poultry protein; and around 200 times, or around 400 times, or around 600 times, or around 800 times, or around 1,000 times, or around 1,200 times less than for beef protein [Global Livestock Environmental Assessment Model (GLEAM) http://www.fao.org/gleam/results/en/]. Certain embodiments of the present invention support the global need for more sustainable food production, with greater circularity in carbon flows.

In certain embodiments, the biomass is further processed following drying to complete the production of bio-based oils, oleochemicals, or biofuels or other useful chemicals through the separation of the lipid content or other targeted biochemicals from the microbial biomass. The separation of the lipids can be performed by using nonpolar solvents to extract the lipids such as, but not limited to, hexane, cyclohexane, ethyl ether, alcohol (isopropanol, ethanol, etc.), tributyl phosphate, supercritical carbon dioxide, trioctylphosphine oxide, secondary and tertiary amines, or propane. Other useful biochemicals can be extracted using solvents including but not limited to: chloroform, acetone, ethyl acetate, and tetrachloroethylene.

In certain embodiments the extracted lipid content of the biomass can be processed using any suitable method for biomass refining, including but not limited to one or more of the following—catalytic cracking and reforming; decarboxylation; hydrotreatment; isomerization—to produce hydrocarbon petroleum and petrochemical replacements, including but not limited to one or more of the following: JP-8 jet fuel, diesel, gasoline, and other alkanes, olefins, and aromatics. In some embodiments, the extracted lipid content of the biomass can be converted to ester-based fuels, such as biodiesel (fatty acid methyl ester or fatty acid ethyl ester), through processes known in the art and science of biomass refining including but not limited to transesterification and esterification.

In some embodiments the broth left over following the removal of cell mass can be pumped to a system for removal of the chemical products of chemosynthesis and/or spent nutrients which may be recycled or recovered to the extent possible and/or disposed of. Methods for processing a biomass, recovery of chemical products from the process stream and/or the removal of the waste products are described in U.S. Pat. No. 9,157,058, which is hereby incorporated by reference.

The methods and bioreactors of the present disclosure find various uses. In some embodiments, one or more amino acids, or proteins, or other nutrients, or whole cell products may be obtained by processing the biomass produced using bioreactors and methods as described herein. In some embodiments, the one or more amino acids, or proteins, or other nutrients, or whole cell products are used as an alternative or non-conventional protein and/or nutrient source. In some embodiments, the one or more amino acids, or proteins, or other nutrients, or whole cell products are components of, or precursors to, or are included within a feed or nutrient supply provided to another organism. In certain non-limiting embodiments that other type of organism consuming said nutrient supply is one or more of the following: bacteria, archaea, yeast, microalgae, seaweed, kelp, zooplankton, fungus, mushroom, plant, shellfish or other invertebrate, fish, bird, or mammal.

In certain non-limiting embodiments, proteinaceous biomass produced as described herein is used as an alternative protein source. In certain embodiments, it is used as a replacement for fish meal and/or casein and/or whey and/or soy meal. In certain non-limiting embodiments, the protein products are not deficient in lysine and/or methionine. In certain embodiments, amino acids, peptides, and/or proteins produced as described herein are used in fertilizer, biostimulant, biofertilizer, mushroom growth enhancer, feed formulations, and/or human food ingredients in place of fish meal, casein, whey, and/or soy meal and/or other plant proteins. In certain non-limiting embodiments, the protein products are not deficient in any essential amino acids. In certain non-limiting embodiments, the protein products are not deficient in lysine and/or methionine. In certain non-limiting embodiments, the proteinaceous biomass does not contain significant amounts of anti-nutritional factors. In certain embodiments, the proteinaceous biomass does not contain significant amounts of one or more of the following: gossypol, glucosinolates, saponins, or trypsin inhibitors.

In certain embodiments of the present invention, the production process and plant are compliant with stringent codes or regulations such as but not limited to the Food and Drug Administration's (FDA's) Code of Federal Regulations (CFR's) for “Current Good Manufacturing Practice” (“cGMP”), and/or the Association of American Food Control Officials CFR's. cGMP is a regulatory framework for the overall design, operation, monitoring and control of manufacturing processes as laid down and enforced by internationally recognized bodies such as the US Food and Drug Administration (FDA). Specifically, in the US cGMP guidance and regulatory requirements are stipulated in a set of Code of Federal Regulations (CFR) which interpret the US domestic Federal Drug, Food and Cosmetic act.

In certain embodiments, the process design is aligned with the requirements as laid down in Annex II (Hardware and Equipment) of the EU's Regulations for animal feed products as defined in EC Regulation 183/2005. One requirement of the said Annex II is that feed processing and storage facilities, equipment, containers, crates, vehicles, and their immediate surroundings shall be kept clean, and effective pest control programs shall be implemented. In certain embodiments, all product contact process tanks, vessels, piping, and pipe fittings are designed with the ability to be Cleaned-in-Place (CIP) either automatically or manually using hot dilute caustic, and/or be washed, flushed, and drained with potable grade water as a minimum. In certain embodiments, hardware is designed such that it can be safely dismantled and Cleaned-out-of-Place (COP) using suitable cleaning fluids/agents. In certain embodiments, facility operational personnel are trained and instructed to comply with a site quality management system including but not limited to a facility wide pest control and monitoring program. Another requirement of the said Annex II is that the lay-out, design, construction, and size of the facilities and equipment shall: (a) permit adequate cleaning and/or disinfection; (b) be such as to minimize the risk of error and to avoid contamination, cross-contamination, and any adverse effects generally on the safety and quality of the products. Machinery coming into contact with feed shall be dried following any wet cleaning process. In certain embodiments, the process comprises the ability to perform automated CIP using suitable cleaning agents for effective cleaning and disinfection of product contact vessels, piping fittings etc. In certain embodiments, where CIP is not possible, manual COP procedures are adopted and enforced to ensure hardware in contact with product is effectively cleaned and disinfected. In certain embodiments, equipment, hardware, and piping are designed and installed to be self-draining. In certain embodiments, vessels, tanks, and piping are generally closed to prevent ingress of physical and chemical contaminants from the surrounding environment. In certain embodiments, process piping, and material flows are designed to eliminate or minimize cross contamination. Another requirement of the said Annex II is that facilities and equipment to be used for mixing and/or manufacturing operations shall undergo appropriate and regular checks, in accordance with written procedures pre-established by the manufacturer for the products. In certain embodiments, the facility comprises a feed manufacturing plant and is subject to regular checks and inspections. In certain embodiments, all instrumentation associated with product quality and safety are routinely checked, maintained, and calibrated. In certain embodiments, sampling valves and/or sampling points are provided on equipment and hardware where mixing or blending is performed. In certain embodiments, on-line and/or at-line physicochemical analysis is performed on feedstocks and/or products. Another requirement of the said Annex II is that drainage facilities must be adequate for the purpose intended; they must be designed and constructed to avoid the risk of contamination of feeding stuffs. In certain embodiments, all process effluent is collected and directed into a dedicated drainage collection system. In certain embodiments, liquid effluents are stored in a liquid effluent tank and disposed of off-site by, for example, an appointed specialist waste disposal company. In certain embodiments, the aqueous effluent of the bioprocess is of high enough quality and safety that it can used for water recycling and/or beneficial uses such as crop or landscape irrigation. Another requirement of the said Annex II is that water used in feed manufacture shall be of suitable quality for animals; the conduits for water shall be of an inert nature. In certain embodiments, potable water is used in certain parts of the process—e.g., make-up of dilute caustic washing/cleaning solutions. In certain embodiments, demineralized water is used in certain parts of the process—e.g., make up of bulk liquid media or trace elements/salts solutions that are used directly in the fermentation. In certain embodiments, industrial scale stainless steel and various elastomers are used in the construction of the fermentation equipment. In certain embodiments, construction of the fermentation equipment uses materials that are widely regarded as being non-shedding, non-reactive and non-absorptive, such as but not limited to 304L, 316L grades of stainless steel and elastomers such as but not limited to viton and EPDM.

Use of chemoautotrophic microorganisms to produce proteins, amino acids and other nutrients from gaseous feedstocks comprising H₂ and/or CO₂ and/or CO and/or CH₄ is described, for example, in an International Patent application received on Mar. 18, 2017 under No. PCT/US17/231 10, and entitled MICROORGANISMS AND ARTIFICIAL ECOSYSTEMS FOR THE PRODUCTION OF PROTEIN, FOOD, AND USEFUL CO-PRODUCTS FROM C1 SUBSTRATES. This application is incorporated herein by reference in its entirety for all purposes.

In certain embodiments, a plant biostimulant and/or biofertilizer and/or organic fertilizer is produced from a biomass as described in PCT Application No. PCT/US2018/016779, filed Feb. 4, 2018, which application is incorporated herein by reference.

In some embodiments, no protein hydrolysis is performed on at least a portion of the microbial biomass, and the product is or includes a lysate of microbial cells. In some embodiments, there is no separation or drying steps applied to the lysate and/or hydrolysate, and the lysate and/or hydrolysate is a crude mixture of soluble and insoluble components. In certain such embodiments the lysate and/or hydrolysate is not clear and/or is turbid. In some embodiments there is a solid-liquid separation step applied to the lysate and/or hydrolysate, resulting in a soluble product and insoluble co-product. In certain embodiments a soluble lysate or hydrolysate product is clear and/or is not turbid. In certain embodiments, the lysate or hydrolysate is passed through ultra-filtration. In certain such embodiments, the ultra-filtration has around a 100,000 molecular weight (MW) cutoff or less, or around a 10,000 MW cut off or less. In certain embodiments, a lysate and/or hydrolysate is subjected to one or more of the following downstream processes: centrifugation; plate and frame filtration; micro filtration; ultra-filtration; nano filtration; ion exchange chromatography. In certain embodiments, the lysate and/or hydrolysate is passed through a filter that includes one or more grades of carbon. In certain such embodiments the carbon removes color from the lysate and/or hydrolysate. In certain embodiments a lysate and/or hydrolysate, and/or a filtrate of a lysate and/or hydrolysate is passed through ion exchange chromatography, e.g., to lower the salt content.

In certain embodiments, a lysate and/or hydrolysate as disclosed herein is passed through sterile filtration prior to use for growth of another cell culture.

In certain embodiments, a lysate and/or hydrolysate and/or filtrate of the same, is concentrated using one or more of the following: falling film evaporator; rising film evaporator; membrane distillation, nano filtration; reverse osmosis.

In certain embodiments one or more of a lysate and/or hydrolysate and/or extract and/or concentrate and/or isolate as described herein is used in an industrial fermentation and/or dehydrated culture media and/or cell culture application. In certain embodiments a lysate and/or hydrolysate and/or peptide composition and/or amino acid composition as described herein is subjected to ultra-filtration to remove higher molecular weight materials. In certain embodiments a cell culture grown on a medium comprising the product of such ultra-filtration outperforms a cell culture grown on the unfiltered equivalent. In certain embodiments, one or more of a lysate and/or hydrolysate as described herein is used in the growth of animal cells in a culture. In certain such embodiments, the animal cells are mammalian. In certain embodiments, a cell culture is grown using a lysate and/or hydrolysate as described herein, which produces proteins and/or tissues used to form a meat-type product. In certain such embodiments, the meat-type product is produced for human consumption.

In certain embodiments, cell cultures are grown using a lysate and/or hydrolysate as described herein, which produce one or more pharmaceutical products. In certain embodiments, a lysate and/or protein hydrolysate and/or peptide composition and/or amino acid composition and/or other nutrient or co-factor produced as described herein replaces one or more animal-derived components in media used for the growth of various natural or recombinant cells, such as prokaryotic cells, for the production of nutritionals and/or bio-pharmaceuticals. In certain embodiments, such natural or recombinant prokaryotes include, but are not limited to, one or more of the following: Bacillus subtilis; Corynebacterium ammoniagenes; Pseudomonas sp.; Streptomyces lividans. In certain embodiments, the pharmaceutical products include, but are not limited to, one or more of: antibiotics such as, but not limited to, cephalosporins and cephamycins; anti-coagulants; blood factors; vaccines; polysaccharide vaccines; recombinant vaccines; recombinant proteins; antibodies; cytokines such as, but not limited to, Interleukin-11, Human granulocyte colony-stimulating factor (hG-CSF); fusion proteins; growth factors; interferons; clotting factors; hormones such as, but not limited to, human growth hormone, insulin, gonadotropin-releasing hormone, human parathyroid hormone; monoclonal antibodies; nucleic acids; therapeutic enzymes such as, but not limited to, human tissue plasminogen activator; fibrinolytic enzymes; therapeutic proteins such as, but not limited to, Transforming Growth Factor-α-Pseudomonas Exotoxin Fusion Protein (TGF-α-PE40), Human Epidermal Growth Factor (hEGF). In certain embodiments, cell cultures are grown using a lysate and/or hydrolysate as described herein, which produce a recombinant protein. In certain embodiments, a monoclonal antibody produced using medium components (e.g., a microbial lysate and/or hydrolysate) as described herein include, but are not limited to, one or more of: Herceptin; Remicade, Rituxan, Synagis. In certain embodiments, a lysate or hydrolysate as described herein is used in a replacement for serum or serum derived components including fetal calf serum (FCS).

In certain embodiments, a whole cell biomass and/or lysate and/or protein hydrolysate and/or peptide composition and/or amino acid composition and/or PHB and/or hydroxybutyrate and/or vitamin and/or other nutrient or co-factor produced as described herein is fed to one or more other organisms or cells (e.g., one or more organisms or cells that are different than the microorganism from which the whole cell biomass and/or lysate and/or protein hydrolysate and/or peptide composition and/or amino acid composition and/or PHB and/or hydroxybutyrate and/or vitamin and/or other nutrient or co-factor is derived), including, but not limited to, one or more of the following: Actinomycetes, Aspergillus awamori, Aspergillus fumigates, Aspergillus nidulans, Aspergillus niger, Aspergillus oryzae, Aspergillus foetidus, Bacillus alkalophilus, Bacillus amyloliquefaciens, Bacillus brevis, Bacillus circulans, Bacillus clausii, Bacillus coagulans, Bacillus lentus, Bacillus licheniformis, Bacillus megaterium, Bacillus pumilis, Bacillus stearothermophilus, Bacillus subtilis, Bacillus thuringiensis, E. coli, E. coli strain B, E. coli strain C, E. coli strain K, E. coli strain W, Streptomyces lividans, Streptomyces murinus, Trichoderma atroviride, Trichoderma koningii, Trichoderma longibrachiatum, Trichoderma reesei, Trichoderma viride, Humicola insolens, Humicola lanuginose, Mucormiehei, Rhizomucor miehei, Rhodococcus opacus, 293 cells, 3T3 cells, BHK cells, CHO cells, COS cells, Cvl cells, HeLa cells, MDCK cells, P12 cells, VERO cells.

In certain embodiments, a whole cell biomass and/or lysate and/or protein hydrolysate and/or peptide composition and/or amino acid composition and/or PHB and/or hydroxybutyrate and/or vitamin and/or other nutrient or co-factor produced as described herein is fed to one or more other organisms or cells (e.g., one or more organisms or cells that are different than the microorganism from which the whole cell biomass and/or lysate and/or protein hydrolysate and/or peptide composition and/or amino acid composition and/or PHB and/or hydroxybutyrate and/or vitamin and/or other nutrient or co-factor is derived), including, but not limited to, members of one or more of the following genera: Aspergillus, Bacillus, Chrysosporium, Escherichia, Fusarium, Humicola, Kluyveromyces, Lactobacillus, Mucor, Myceliophtora, Neurospora, Penicillium, Phanerochaete, Pichia, Pleurotus, Pseudomonas, Rhizomucor, Rhodococcus, Saccharomyces, Schizosaccharomyces, Stenotrophamonas, Streptomyces, Trametes, Trichoderma, Yarrowia.

In certain embodiments, a whole cell biomass and/or lysate and/or protein hydrolysate and/or peptide composition and/or amino acid composition and/or PHB and/or hydroxybutyrate and/or vitamin and/or other nutrient or co-factor produced as described herein is fed to one or more other organisms or cells (e.g., one or more organisms or cells that are different than the microorganism from which the whole cell biomass and/or lysate and/or protein hydrolysate and/or peptide composition and/or amino acid composition and/or PHB and/or hydroxybutyrate and/or vitamin and/or other nutrient or co-factor is derived), including, but not limited to, one or more of the following: archaea cells, bacterial cells including gram-negative bacteria and/or gram-positive bacteria, filamentous fungal cells, fungus cells, insect cells, mammalian cells, animal cells, plant cells, yeast cells.

In certain embodiments, cell cultures are provided whole cell biomass and/or a lysate and/or a protein hydrolysate and/or a peptide composition and/or an amino acid composition and/or PHB and/or hydroxybutyrate and/or vitamins and/or other nutrients or co-factors produced as described herein as a nutrient source for the production of one or more microbial or chemical products such as but not limited to one or more of the following: polysaccharides, lipids, biodiesel, butanol, ethanol, propanol, isopropanol, propane, alkanes, olefins, aromatics, fatty alcohols, fatty acid esters, alcohols; 1,3-propanediol, 1,3-butadiene, 1,3-butanediol, 1,4-butanediol, 3-hydroxypropionate, 7-ADCA/cephalsporin, ε-caprolactone, γ-valerolactone, acrylate, acrylic acid, adipic acid, ascorbate, aspartate, ascorbic acid, aspartic acid, caprolactam, carotenoids, citrate, citric acid, DHA, docetaxel, erythromycin, ethylene, gamma butyrolactone, glutamate, glutamic acid, HPA, hydroxybutyrate, isopentenol, isoprene, isoprenoids, itaconate, itaconic acid, lactate, lactic acid, lanosterol, levulinic acid, lycopene, lysine, malate, malonic acid, peptides, omega-3 DHA, omega-3 EPA, omega-3 ALA, omega fatty acids, omega-7 fatty acids, omega-7 rich-oils, paclitaxel, PHA, PHB, polyketides, polyols, propylene, pyrrolidones, serine, sorbitol, statins, steroids, succinate, terephthalate, terpenes, THF, rubber, wax esters, polymers, commodity chemicals, industrial chemicals, specialty chemicals, paraffin replacements, additives, nutritional supplements, nutraceuticals, pharmaceuticals, pharmaceutical intermediates, personal care products; commercial enzymes, antibiotics, amino acids, vitamins, bioplastics, glycerol, jet fuel, diesel, gasoline, octane.

In certain embodiments, one or more defatting steps is performed on the biomass and/or lysate and/or hydrolysate. In certain embodiments the one or more defatting steps removes or decreases the content of lipopolysaccharides (LPS) in the product. In certain embodiments, one or more filtration or ultrafiltration steps are performed on the lysate and/or hydrolysate. In certain embodiments the one or more filtration or ultrafiltration steps removes or decreases the content of lipopolysaccharides (LPS) in the product. In certain embodiments, the said ultrafiltration step has a molecular weight cut-off of 100 kilodaltons (kD) or less, or 50 kD or less, or 25 kD or less, or 20 kD or less, or 10 kD or less, or 5 kD or less. In certain embodiments, the LPS removed or decreased in one or more defatting steps and/or one or more filtration or ultrafiltration steps is an endotoxin.

In some embodiments, the biomass may be processed to extract and/or purify a biodegradable polyester, prior, during, or following the production of a protein hydrolysate composition, including, but not limited to, a polyhydroxyalkanoate (PHA) polymer. In some embodiments, the biomass may be processed to extract a polymeric product that includes polyhydroxybutyrate (PHB). In some embodiments, the biomass may be processed to extract a polymeric product that includes polyhydroxyvalerate (PHV). The PHA or PHB or PHV polymer may be extracted from the biomass using any suitable method. In some embodiments, the PHA or PHB or PHV polymer may be extracted first by mixing the biomass with a solvent, such as one or more of chloroform, methanol, methylene chloride, 1,2-dichloroethane, dichloromethane, diethyl succinate, acetone, hexane, propylene carbonate, isopropanol, and ethanol. In some embodiments, the biomass is lysed (e.g., by homogenization) before mixing with the solvent. The extraction may be performed at any suitable temperature and may be performed at a temperature ranging from room temperature to 150° C., or more. In some embodiments, the extraction includes separating an aqueous phase and an organic phase after mixing the biomass with the solvent. The phase separation may be done using any suitable method, such as, but not limited to, centrifugation. In some embodiments, the extraction includes precipitation of PHA or PHB or PHV by, e.g., cooling the mixture, and/or adding an antisolvent (e.g., hexane) to the mixture. The extraction may include removing the solvent from the biomass-solvent mixture. In some embodiments, following the extraction, the extracted material is further purified by mixing the extracted material with a second solvent, such as hexane, in which non-polar lipids are soluble, but the PHA or PHB or PHV is insoluble. The second solvent may be removed after the mixing. Suitable methods for extracting a PHA or PHB or PHV polymer is described in, e.g., Fei, et al. (2016) “Effective recovery of poly-β-hydroxybutyrate (PHB) biopolymer from Cupriavidus necator using a novel and environmentally friendly solvent system” Biotechnol Prog. 32(3):678-85; Ujang, et al. (2009) “Recovery of Polyhydroxyalkanoates (PHAs) from Mixed Microbial Cultures by Simple Digestion and Saponification” Malaysia: University Teknology, Institute of Environmental and Water Resource Management, 8-15, which are incorporated herein by reference in their entireties.

In some embodiments, a biomass obtained from a microorganism, e.g., chemoautotrophic microorganism, culture may be processed to extract an organic polymer that the microorganism accumulates during growth. In some embodiments, a microorganism as described herein can grow on H₂/CO₂ and/or syngas, and the microorganism can accumulate polyhydroxyalkanoate (PHA), e.g., polyhydroxybutyrate (PHB) and/or polyhydroxyvalerate (PHV), to about 50% or more of the cell biomass by weight. In some embodiments, the microorganism has a native ability to direct a high flux of carbon through an acetyl-CoA metabolic intermediate, which can lead into fatty acid biosynthesis, along with a number of other synthetic pathways including PHA and/or PHB and/or PHV synthesis, as well as amino acids. In some embodiments, the microorganism exhibiting these traits is Cupriavidus necator (e.g., Cupriavidus necator DSM 531 or DSM 541) and/or Cupriavidus metallidurans (e.g., Cupriavidus metallidurans DSM 2839).

In certain embodiments, processing a biomass obtained from a chemoautotrophic microorganism includes extracting PHA and/or PHB and/or PHV from an insoluble hydrolysate fraction. In other embodiments, processing a biomass obtained from a chemoautotrophic microorganism includes recovering a PHA or PHB or PHV-rich solid from a soluble hydrolysate fraction. Any suitable method for extracting PHA or PHB or PHV may be used, as discussed above with respect to processing of a microorganism biomass to extract PHA or PHB or PHV.

### Microorganisms

Microorganisms utilized in the methods described herein may be natural (wild type) or engineered microorganism strains.

In some embodiments, the microorganism of the present disclosure is a chemoautotrophic microorganism. In some embodiments, microorganisms utilized in the methods described herein are chemoautotrophs. Chemoautotrophs can perform chemosynthetic reactions that fix CO₂, and/or other forms of inorganic carbon, to organic compounds, using the potential energy stored in inorganic chemicals to drive the reaction, rather than radiant energy from light as in microorganisms performing photosynthesis. In some embodiments, the microorganism of the present disclosure can perform mixotrophic growth and/or is a heterotrophic microorganism. In some embodiments, the microorganism of the present disclosure is a photosynthetic microorganism. In some embodiments, the microorganism of the present disclosure is an oxyhydrogen or knallgas strain, i.e., a microbe that can use hydrogen as an electron donor and oxygen as an electron acceptor in respiration for the generation of intracellular energy carriers such as adenosine-5′-triphosphate (ATP). Knallgas microorganisms generally use molecular hydrogen by means of hydrogenases, with some of the electrons donated from H₂ that is utilized for the reduction of NAD⁺ (and/or other intracellular reducing equivalents) and some of the electrons from H₂ used for aerobic respiration. Knallgas microorganisms generally fix CO₂ chemoautotrophically, through pathways including but not limited to the Calvin Cycle or the reverse citric acid cycle.

In some embodiments the microorganisms, or a composition comprising microorganisms, comprises one or more of the following knallgas microorganisms: Aquifex pyrophilus, Aquifex aeolicus, or other Aquifex sp.; Cupriavidus necator or Cupriavidus metallidurans or other Cupriavidus sp.; Corynebacterium autotrophicum or other Corynebacterium sp.; Gordonia desulfuricans, Gordonia polyisoprenivorans, Gordonia rubripertincta, Gordonia hydrophobica, Gordonia westfalica, or other Gordonia sp.; Nocardia autotrophica, Nocardia opaca, or other Nocardia sp.; purple non-sulfur photosynthetic bacteria, including but not limited to, Rhodobacter sphaeroides, Rhodopseudomonas palustris, Rhodopseudomonas capsulata, Rhodopseudomonas viridis, Rhodopseudomonas sulfoviridis, Rhodopseudomonas blastica, Rhodopseudomonas spheroides, Rhodopseudomonas acidophila, or other Rhodopseudomonas sp.; Rhodobacter sp.; Rhodospirillum rubrum, or other Rhodospirillum sp.; Rhodococcus opacus or other Rhodococcus sp.; Rhizobium japonicum or other Rhizobium sp.; Thiocapsa roseopersicina or other Thiocapsa sp.; Pseudomonas facilis, Pseudomonas flava, Pseudomonas putida, Pseudomonas hydrogenovora, Pseudomonas hydrogenothermophila, Pseudomonas palleronii, Pseudomonas pseudoflava, Pseudomonas saccharophila, Pseudomonas thermophile, or other Pseudomonas sp.; Hydrogenomonas pantotropha, Hydrogenomonas eutropha, Hydrogenomonas facilis, or other Hydrogenomonas sp.; Hydrogenobacter thermophilus, Hydrogenobacter halophilus, Hydrogenobacter hydrogenophilus, or other Hydrogenobacter sp.; Hydrogenophilus islandicus or other Hydrogenophilus sp.; Hydrogenovibrio marinus or other Hydrogenovibrio sp.; Hydrogenothermus marinus or other Hydrogenothermus sp.; Helicobacter pylori or other Helicobacter sp.; Xanthobacter autotrophicus, Xanthobacter flavus, or other Xanthobacter sp.; Hydrogenophaga flava, Hydrogenophaga palleronii, Hydrogenophaga pseudoflava, or other Hydrogenophaga sp.; Bradyrhizobium japonicum or other Bradyrhizobium sp.; Ralstonia eutropha or other Ralstonia sp.; Alcaligenes eutrophus, Alcaligenes facilis, Alcaligenes hydrogenophilus, Alcaligenes latus, Alcaligenes paradoxus, Alcaligenes ruhlandii, or other Alcaligenes sp.; Amycolata sp.; Aquaspirillum autotrophicum or other Aquaspirillum sp.; Arthrobacter strain 11/X, Arthrobacter methylotrophus, or other Arthrobacter sp.; Azospirillum lipoferum or other Azospirillum sp.; Variovorax paradoxus or other Variovorax sp.; Acidovorax facilis, or other Acidovorax sp.; Bacillus schlegelii, Bacillus tusciae, other Bacillus sp.; Calderobactenum hydrogenophilum or other Calderobactenum sp.; Derxia gummosa or other Derxia sp.; Flavobacterium autothermophilum or other Flavobacterium sp.; Microcyclus aquaticus or other Microcyclus sp.; Mycobacterium gordoniae or other Mycobacterium sp.; Paracoccus denitrificans or other Paracoccus sp.; Persephonella marina, Persephonella guaymasensis, or other Persephonella sp.; Renobacter vacuolatum or other Renobacter sp.; Seliberia carboxydohydrogena or other Seliberia sp., Streptomycetes coelicoflavus, Streptomycetes griseus, Streptomycetes xanthochromogenes, Streptomycetes thermocarboxydus, and other Streptomycetes sp.; Thermocrinis ruber or other Thermocrinis sp.; Wautersia sp.; cyanobacteria including but not limited to Anabaena oscillarioides, Anabaena spiroides, Anabaena cylindrica, or other Anabaena sp.; and Arthrospira platensis, Arthrospira maxima, or other Arthrospira sp.; green algae including but not limited to Scenedesmus obliquus or other Scenedesmus sp.; Chlamydomonas reinhardii or other Chlamydomonas sp.; Ankistrodesmus sp.; and Rhaphidium polymorphium or other Rhaphidium sp; as well as a consortium of microorganisms that includes oxyhydrogen microorganisms.

In some embodiments, the microorganism is selected from the genus Hydrogenobacter. In some embodiments, the microorganism is Hydrogenobacter thermophilus. In some embodiments, the microorganism contains the reverse tricarboxylic acid cycle (rTCA), also known as the reverse citric acid cycle or the reverse Krebs cycle.

In some embodiments, the microorganism is Rhodococcus opacus or Rhodococcus jostii or Rhodococcus sp. In some non-limiting embodiments, the microorganism is Rhodococcus opacus DSM 43205, DSM 43206, DSM 44193, and/or Rhodococcus sp. DSM 3346.

In some embodiments, the natural or engineered strain includes but is not limited to hydrogen utilizing microbes including but not limited to the genera Rhodococcus, Gordonia, Ralstonia or Cupriavidus. In some embodiments, the microorganism can naturally grow on H₂/CO₂ and/or syngas, wherein the microorganism can naturally accumulate lipid to about 50% or more of the cell biomass by weight. In some embodiments, the microorganisms have a native ability to send a high flux of carbon down the fatty acid biosynthesis pathway. In some embodiments, the microorganism exhibiting these traits is Rhodococcus opacus (DSM 43205 or DSM 43206 or DSM 44193).

In some embodiments, the microorganism is of the class Actinobacteria comprising no exogenous genes or one or more exogenous gene(s). In some embodiments, the microorganism is of the class Actinobacteria or the family Nocardiaceae. In some embodiments, the microorganism is a Corynebacterium, Gordonia, Rhodococcus, Mycobacterium, or Tsukamurella microorganism comprising no exogenous genes or one or more exogenous gene(s). In some embodiments, microorganism of the family Nocardiaceae comprising no exogenous genes or one or more exogenous gene(s). In some embodiments, the microorganism is of the genus Rhodococcus comprising no exogenous genes or one or more exogenous gene(s), and in some embodiments the microorganism is a strain of the species Rhodococcus sp., Rhodococcus opacus, Rhodococcus aethehvorans, Rhodococcus aurantiacus; Rhodococcus baikonurensis; Rhodococcus boritolerans; Rhodococcus equi; Rhodococcus coprophilus; Rhodococcus corynebacterioides; Nocardia corynebacterioides (synonym: Nocardia corynebacterioides); Rhodococcus erythropolis; Rhodococcus fascians; Rhodococcus globerulus; Rhodococcus gordoniae; Rhodococcus jostii; Rhodococcus koreensis; Rhodococcus kroppenstedtii; Rhodococcus maanshanensis; Rhodococcus marinonascens; Rhodococcus opacus; Rhodococcus percolatus; Rhodococcus phenolicus; Rhodococcus polyvorum; Rhodococcus pyridinivorans; Rhodococcus rhodochrous; Rhodococcus rhodnii (synonym: Nocardia rhodnii); Rhodococcus ruber (synonym: Streptothrix rubra); Rhodococcus sp. RHA1; Rhodococcus triatomae; Rhodococcus tukisamuensis; Rhodococcus wratislaviensis (synonym: Tsukamurella wratislaviensis); Rhodococcus yunnanensis; Rhodococcus zopfii. In some embodiments, a Rhodococcus microorganism is provided that is non-infectious or non-pathogenic to animals and/or plants and/or humans. In some embodiments, the microorganism is Rhodococcus equi or Rhodococcus fascians that is non-infectious to animals and/or plants. In some embodiments, the microorganism is strain Rhodococcus opacus DSM number 43205 or 43206; or Rhodococcus sp. DSM 3346. In some embodiments, the microorganism is Rhodococcus that is not a species selected from Rhodococcus equi and/or Rhodococcus fascians.

In some embodiments the microorganism is from the suborder corynebacterineae or the family burkholderiaceae. In some embodiments, the microorganism is not E. coli.

In some embodiments, the microorganisms include one or more of Cupriavidus, Rhodococcus, Hydrogenovibrio, Rhodopseudomonas, Hydrogenobacter, Gordonia, Arthrobacter, Streptomycetes, Rhodobacter, and Xanthobacter microorganisms, or any species of these genera disclosed herein.

In some non-limiting embodiments, the microorganism is a Cupriavidus species, such as Cupriavidus necator (e.g., DSM 531 or DSM 541) or Cupriavidus metallidurans.

In some embodiments, a microorganism as described herein can accumulate protein to any of about 60% or more, 65% or more, 70% or more, 75% or more, 80% or more, or 85% or more of the total cell mass by weight. In some embodiments, the microorganism exhibiting these traits is a Cupriavidus microorganism, such as Cupriavidus necator (e.g., DSM 531 or DSM 541).

In some embodiment, the microorganism can accumulate polyhydroxyalkanoate (PHA), e.g., or polyhydroxybutyrate (PHB) to at least about 50% of the cell biomass by weight. In some embodiments, the microorganism can naturally grow on H₂/CO₂ and/or syngas, and the microorganism can naturally accumulate PHA (e.g., PHB) to about 50% or more of the cell biomass by weight. In some embodiments, the microorganism has a native ability to direct a high flux of carbon through the acetyl-CoA metabolic intermediate, which can lead into fatty acid biosynthesis, along with a number of other synthetic pathways including PHA (e.g., PHB) synthesis, as well as amino acids. In some embodiments, the microorganism exhibiting these traits is a Cupriavidus microorganism, such as Cupriavidus necator (e.g., DSM 531 or DSM 541).

In some nonlimiting embodiments, the natural or engineered microorganism strain is Corynebacterium autotrophicum. In some nonlimiting embodiments, the natural or engineered microorganism is Corynebacterium autotrophicum and/or Corynebacterium glutamicum. In some embodiments, the microorganism is Hydrogenovibrio marinus. In some embodiments, the microorganism is Rhodopseudomonas capsulata, Rhodopseudomonas palustris, or Rhodobacter sphaeroides.

In some non-limiting embodiments, the microorganisms utilize chemoautotrophic metabolism to produce ATP for the support of ATP consuming biosynthetic reactions and cellular maintenance, without the co-production of methane or short chain organic acids such as acetic or butyric acid, by means of energy conserving reactions for the production of ATP, using inorganic electron donors and electron acceptors, including but not limited to the oxyhydrogen reaction.

Several different microorganisms have been characterized that can grow on carbon monoxide as an electron donor and/or carbon source (i.e., carboxydotrophic microorganisms). In some cases, carboxydotrophic microorganisms can also use H₂ as an electron donor and/or grow mixotrophically. In some cases, the carboxydotrophic microorganisms are facultative chemolithoautotrophs [Biology of the Prokaryotes, edited by J Lengeler, G. Drews, H. Schlegel, John Wiley & Sons, Jul. 10, 2009, is incorporated herein by reference in its entirety.]. In some embodiments the microorganisms or compositions comprising the microorganisms comprise one or more of the following carboxydotrophic microorganisms: Acinetobacter sp.; Alcaligenes carboxydus or other Alcaligenes sp.; Arthrobacter sp.; Azomonas sp.; Azotobacter sp.; Bacillus schlegelii or other Bacillus sp.; Hydrogenophaga pseudoflava or other Hydrogenophaga sp.; Pseudomonas carboxydohydrogena, Pseudomonas carboxydovorans, Pseudomonas compransons, Pseudomonas gazotropha, Pseudomonas thermocarboxydovorans, or other Pseudomonas sp.; Rhizobium japonicum or other Rhizobium sp.; and Streptomyces G26, Streptomyces thermoautotrophicus, or other Streptomyces sp. In certain embodiments, a carboxydotrophic microorganism is used. In certain embodiments, a carboxydotrophic microorganism that is capable of chemolithoautotrophy is used. In certain embodiments, a carboxydotrophic microorganism that is able to utilize H₂ as an electron donor in respiration and/or biosynthesis is used.

In some embodiments microorganisms are provided that can grow on syngas as the sole electron donor, source of hydrogen atoms, and carbon source.

In some embodiments the microorganisms include obligate and/or facultative chemoautotrophic microorganisms including one or more of the following: Acetoanaerobium sp.; Acetobacterium sp.; Acetogenium sp.; Achromobacter sp.; Acidianus sp.; Acinetobacter sp.; Actinomadura sp.; Aeromonas sp.; Alcaligenes sp.; Alcaligenes sp.; Aquaspirillum sp.; Arcobacter sp.; Aureobacterium sp.; Bacillus sp.; Beggiatoa sp.; Butyribacterium sp.; Carboxydothermus sp.; Clostridium sp.; Comamonas sp.; Dehalobacter sp.; Dehalococcoide sp.; Dehalospihllum sp.; Desulfobacterium sp.; Desulfomonile sp.; Desulfotomaculum sp.; Desulfovibrio sp.; Desulfurosarcina sp.; Ectothiorhodospira sp.; Enterobacter sp.; Eubacterium sp.; Ferroplasma sp.; Halothibacillus sp.; Hydrogenobacter sp.; Hydrogenomonas sp.; Leptospirillum sp.; Metallosphaera sp.; Methanobactehum sp.; Methanobrevibacter sp.; Methanococcus sp.; Methanococcoides sp.; Methanogenium sp.; Methanolobus sp.; Methanomicrobium sp.; Methanoplanus sp.; Methanosarcina sp.; Methanospirillum sp.; Methanothermus sp.; Methanothrix sp.; Micrococcus sp.; Nitrobacter sp.; Nitrobacteraceae sp., Nitrococcus sp., Nitrosococcus sp.; Nitrospina sp., Nitrospira sp., Nitrosolobus sp.; Nitrosomonas sp.; Nitrosospira sp.; Nitrosovibrio sp.; Nitrospina sp.; Oleomonas sp.; Paracoccus sp.; Peptostreptococcus sp.; Planctomycetes sp.; Pseudomonas sp.; Ralstonia sp.; Rhodobacter sp.; Rhodococcus sp.; Rhodocyclus sp.; Rhodomicrobium sp.; Rhodopseudomonas sp.; Rhodospirillum sp.; Shewanella sp.; Siderococcus sp.; Streptomyces sp.; Sulfobacillus sp.; Sulfolobus sp.; Thermothrix sp., Thiobacillus sp.; Thiomicrospira sp.; Thioploca sp.; Thiosphaera sp.; Thiothrix sp.; Thiovulum sp.; sulfur-oxidizers; hydrogen-oxidizers; iron-oxidizers; acetogens; and methanogens; consortiums of microorganisms that include chemoautotrophs; chemoautotrophs native to at least one of hydrothermal vents, geothermal vents, hot springs, cold seeps, underground aquifers, salt lakes, saline formations, mines, acid mine drainage, mine tailings, oil wells, refinery wastewater, coal seams, deep sub-surface; waste water and sewage treatment plants; geothermal power plants, sulfatara fields, and soils; and extremophiles selected from one or more of thermophiles, hyperthermophiles, acidophiles, halophiles, and psychrophiles.

In some embodiments, a consortium of microorganisms is used in the methods described herein. The consortium may include one or more of any of the microorganism species or strains or microorganisms having one or more microorganism traits described herein.

Use of knallgas microorganisms for the conversion of syngas, producer gas, or other H₂ and CO₂ and/or CO containing gas mixes into mid- to high-carbon chain number or anabolic molecules is described, for example, in a patent application filed in the United States Patent and Trademark Office on Oct. 26, 2012 under Ser. No. 13/643,872, and entitled USE OF OXYHYDROGEN MICROORGANISMS FOR NON-PHOTOSYNTHETIC CARBON CAPTURE AND CONVERSION OF INORGANIC AND/OR C1 CARBON SOURCES INTO USEFUL ORGANIC COMPOUNDS. This application is incorporated herein by reference in its entirety for all purposes.

Use of chemotrophic microorganisms for the conversion of CO₂ into useful organic chemicals is described, for example, in PCT Application No. PCT/US2010/001402, filed May 12, 2010 and entitled BIOLOGICAL AND CHEMICAL PROCESS UTILIZING CHEMOAUTOTROPHIC MICROORGANISMS FOR THE CHEMOSYTHETIC FIXATION OF CARBON DIOXIDE AND/OR OTHER INORGANIC CARBON SOURCES INTO ORGANIC COMPOUNDS, AND THE GENERATION OF ADDITIONAL USEFUL PRODUCTS. This application is incorporated herein by reference in its entirety for all purposes.

A microorganism, e.g., chemoautotrophic microorganism, of the present disclosure may be a naturally occurring strain or may be genetically engineered. The microorganism may be genetically modified to express one or more proteins that have a high nutritive value for animal cells when the hydrolysate containing the protein is provided to the cells in culture, e.g., as a culture media supplement. In some embodiments, a chemoautotrophic microorganism may be genetically modified to express a polypeptide sequence containing multiple peptide subsequences that are interspersed with protease cleavage sites. Such a polypeptide sequence may be represented schematically as: H₂N—X—C-[A-C]ₙ—Y—COOH, where “H₂N” and “COOH” represent the N- and C-terminus of the polypeptide, a respectively; “A” is a peptide subsequence; “C” is a protease cleavage site; “X” and “Y” are linker sequences that may or may not be present; and n is an integer of 1 or greater, e.g., 2 or greater, 5 or greater, 10 or greater, including 20 or greater. The peptide subsequence (A) may be designed to include a peptide sequence (A′) having a beneficial effect on animal cell growth in culture when provided in the culture medium. When the biomass obtained from the genetically modified microorganism is hydrolyzed using the appropriate protease for cutting the cleavage sites, the resulting protein hydrolysate may be enriched for the beneficial peptides.

In some embodiments, the microorganism, e.g., chemoautotrophic microorganism, may be genetically modified to disrupt expression of one or more endogenous genes involved in a biosynthetic pathway. In some embodiments, the microorganism may be genetically modified to disrupt expression of one or more gene(s) involved in the biosynthesis of a polyhydroxyalkanoate, such as polyhydroxybutyrate. Suitable gene(s) involved in the biosynthesis of a polyhydroxyalkanoate, and whose expression may be disrupted, include, without limitation, the gene encoding 3-ketothiolase, acetoacetyl-CoA reductase, and/or PHB synthase. In some embodiments, the microorganism may be genetically modified to disrupt or increase expression of one or more gene(s) involved in the biosynthesis of a vitamin, such as, but not limited to, vitamin B₁, vitamin B₂, and/or vitamin B₁₂. In some embodiments, the expression of a gene involved in the biosynthesis of vitamin B₁₂ is disrupted or increased. Expression of one or more genes may be disrupted or increased by any suitable method, e.g., by deleting or mutating all or part of the coding region or the regulatory region of the gene in the microorganism genome, or replicating the coding region or the regulatory region of the gene within the microorganism genome.

The microorganism may be genetically engineered using any suitable method. Genetic engineering of knallgas microorganisms is described, for example, in U.S. Pat. No. 9,879,290 B2, which is incorporated herein by reference in its entirety.

PCT/US20/50902 Microbial Protein Hydrolysate Compositions and Methods of Making Same 2019; PCT/US20/67555 and U.S. Ser. No. 17/363,465 High Protein Food Compositions 2019; PCT/US21/14795 and U.S. Ser. No. 17/362,998 Microorganism-derived Protein Hydrolysates, And Methods Of Preparation And Use Thereof. 2020; PCT/US2021/020147 and U.S. Ser. No. 17/362,990 Structured High-protein Meat Analogue Compositions; PCT/US2021/023949 Structured High-protein Meat Analogue Compositions With Microbial Heme Flavorants; PCT/US2018/016779 Microbial Conversion of CO₂ and Other C1 Substrates to Vegan Nutrients, Fertilizers, Biostimulants, and Systems for Accelerated Soil Carbon Sequestration 2017; PCT/US17/23110 Microorganisms And Artificial Ecosystems For The Production Of Protein, Food, And Useful Co-Products From C1 Substrates 2016; U.S. Pat. No. 9,085,785 Use Of Oxyhydrogen Microorganisms For Non-Photosynthetic Carbon Capture And Conversion Of Inorganic And/Or C1 Carbon Sources Into Useful Organic Compounds are each incorporated by reference herein in their entirety.

Embodiments of the present disclosure are further illustrated by the following Examples, which in no way should be construed as further limiting. The entire contents of all the references (including literature references, issued patents, published patent applications, and co-pending patent applications) cited throughout this application are hereby expressly incorporated by reference, in particular for the teaching that is referenced hereinabove. However, the citation of any reference is not intended to be an admission that the reference is prior art.

Other features of the invention will become apparent in the course of the following descriptions of examples. The following examples are intended to illustrate, but not limit, the invention.

### EXAMPLES

### Example 1: Effect of Pressure on Productivity

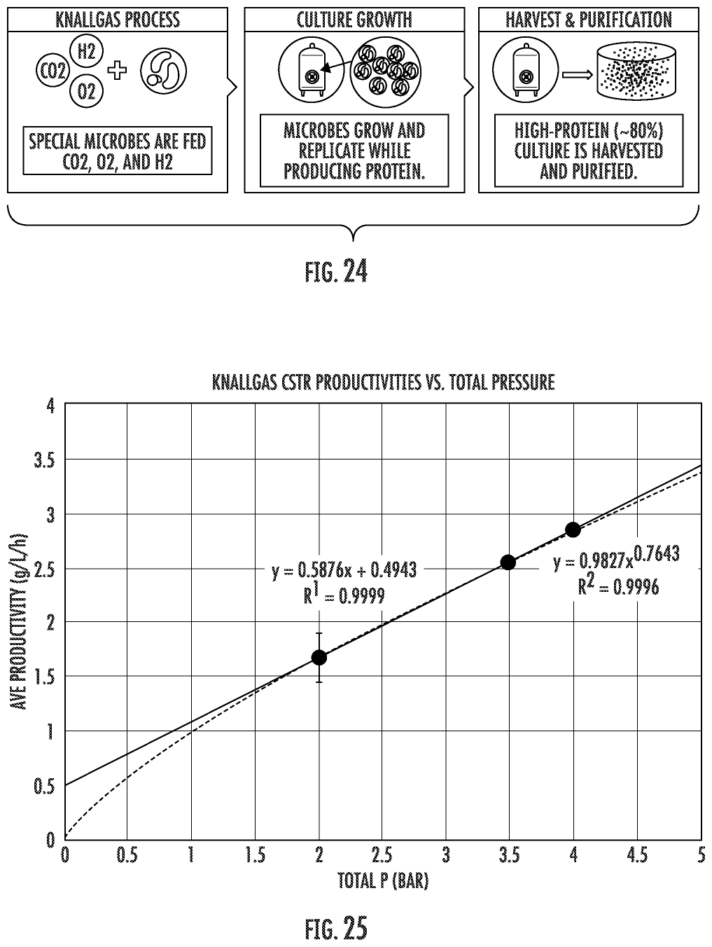

Elevated pressure has been shown to increase biomass and product yields by Cupriavidus in knallgas fermentations [Yu, J and Munasinghe, P. (2018) Gas Fermentation Enhancement for chemolithoautotrophic growth of Cupriavidus necator on carbon dioxide. Fermentation 4:63], [Garrigues, L., et al (2020) Isopropanol production from carbon dioxide in Cupriavidus necator in a pressurized bioreactor New Biotechnol 56:16-20]. In the present example the effects of pressure up to 5 bar on the productivity of C. necator has been tested in a continuous stirred tank reactor (CSTR). In an CSTR bioprocess run as a turbidostat at 4 bar pressure on CO₂ as the sole carbon source, the average biomass productivity was 2.85 g/L/h (FIG. 25). FIG. 25 also illustrates the strong positive correlation observed between pressure and CSTR productivity on CO₂.

The pressure test runs were performed in a CSTR system based on a 500 mL BioXplorer 5000P high-pressure steel stirred tank bioreactor from HEL Ltd. (www.helgroup.com). The effects of pressure up to 5 bar on the productivity of C. necator has been tested. Increasing pressure was shown to be a highly effective means of increasing productivity and optimizing yield. In addition, the ability to run at elevated pressure is important for modeling the effect of bioprocess scale-up, since scaling-up the bioreactor in the vertical direction will result in increased hydrostatic pressure. Therefore, this elevated pressure system also enabled the modeling of a major effect of scale-up.

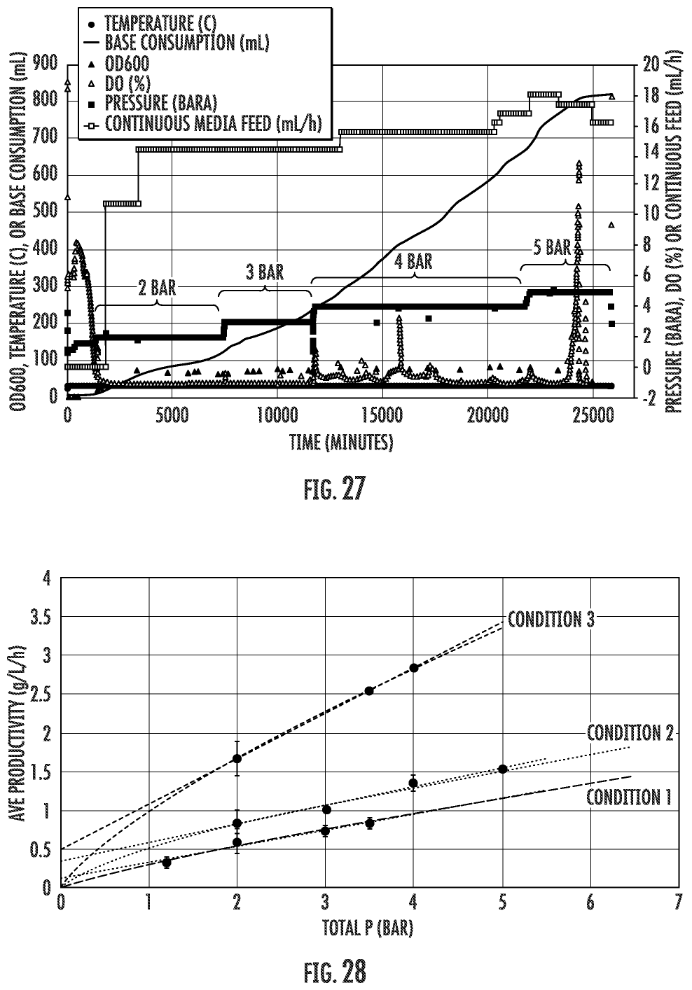

An example of some of the raw run data recorded during a pressure ramp-up experiment going from 2 to 5 bar in the CSTR is shown in FIG. 27.

The data shown in FIG. 27 include: optical density of the culture measured at 600 nm (OD₆₀₀); reactor temperature (T ° C.); base consumption (mL) required to maintain the culture at pH 7; the absolute pressure of the bioreactor in bar (bara); the dissolved O₂ in the culture broth (DO); and the rate that the continuous feed of fresh aqueous inorganic mineral media was input into the bioreactor (mL/h) during continuous operation. This input of aqueous mineral media was balanced by a continuous withdrawal of culture broth such that a roughly constant OD₆₀₀ was maintained in the bioreactor falling in the range of OD₆₀₀=50-60. In other words, the bioreactor was run approximately as a turbidostat. The liquid volume of the bioreactor (i.e., working volume) also generally remained at a roughly constant level throughout. Continuous operation in the particular pressure ramp-up experiment shown in FIG. 28 lasted for over 16 days with the reactor maintained at 2, 3, 4, and 5 bar pressure for several days each. In pressure ramp-up experiments such as this one, the pressure was varied, but all the other run parameters (e.g., agitation rate, pH, T, OD₆₀₀, input molar gas flows and % composition of H₂, CO₂, and O₂ etc.) were targeted at constant values, apart from the dilution rate (μh⁻¹), which was allowed to vary in order to maintain a targeted OD₆₀₀ (i.e., turbidostatic operation) as the pressure was increased. Culture broth was harvested continuously from the CSTR and stored in a cooled reservoir (4° C.), from which it was recovered daily, dewatered, dried, and weighed to determine productivity. The dried biomass was stored for chemical analysis and further protein purification operations. The effect of total reactor pressure (P bar) on productivity, and several other run characteristics including H₂ yield (Y_{H2} g biomass/g H₂), was determined.

### Example 2: Extending the Trend of Increasing Productivity with Increasing Pressure Out to Higher Pressures and Productivities

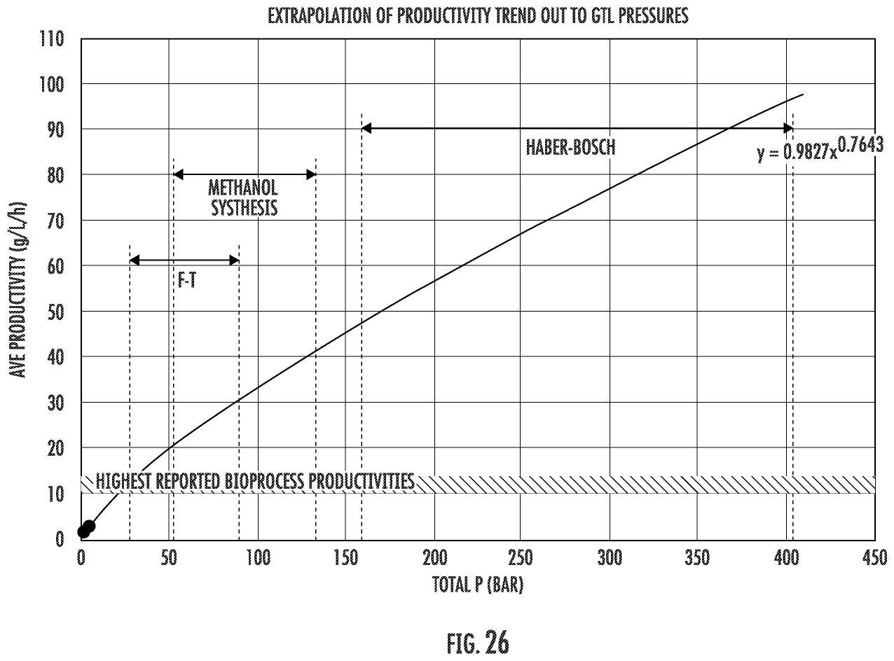

The range of operating pressures may be extended experimentally out to 50 bar, and/or above 50 bar (i.e., >50 bar) and the effects on protein production and/or the production of other biomass components and/or metabolites systematically detailed. Extrapolating the power law fit to the pressure vs. productivity correlation given in FIG. 25 out to pressure ranges used in established chemical GTL processes (FIG. 26) illustrates that ultra-high productivities on CO₂ that are unprecedented for any biological process on any substrate; heterotrophic, chemotrophic, or photosynthetic, can be achieved if the trend is continued. Reported operating pressures for the main GTL processes are 10-45 bar for Fischer-Tropsch [Botes, F. G., Dancuart, L. P., Nel, H. G., Steynberg, A. P., Vogel, A. P., Breman, B. B., & Font Freide, J. H. M. (2011). Middle distillate fuel production from synthesis gas via the Fischer-Tropsch process. In Advances in Clean Hydrocarbon Fuel Processing: Science and Technology. https://doi.org/10.1533/9780857093783.4.329], 40-120 bar for methanol synthesis [https://www.netl.doe.gov/research/coal/energy-systems/gasification/gasifipedia/methanol], and 150-400 bar for Haber-Bosch [Appl, M. (2011). Ammonia, 2. Production Processes. In Ullmann's Encyclopedia of Industrial Chemistry. https://doi.org/10.1002/14356007.o02_o11]. Thus, the bioreactor operational pressures that are tested (up to 50 bar) have ample precedent in the chemical industry for the production of low cost, high volume commodities such as ammonia, methanol, and FT-diesel. In certain embodiments of the present invention strain and/or process engineering is utilized to extend the observed trend of increasing productivity with pressure out to GTL-type pressures. In certain such embodiments the highest bioprocess productivities ever recorded on any substrate are attained. In certain such embodiments a productivity ≥10 g/L/hr is attained.

Certain embodiments of the present invention will extend the range of operating P out to 50 bar, and/or above 50 bar (i.e., >50 bar) and systematically detail the effects on protein production on and/or the production of other biomass components and/or metabolites. Extrapolating the power law fit to the pressure vs. productivity correlation out to pressure ranges used in established chemical GTL processes (FIG. 26) illustrates the potential for the present invention to deliver ultra-high productivities on CO₂ that are unprecedented for any biological process on any substrate; heterotrophic, chemotrophic, or photosynthetic. In certain embodiments of the present invention, a higher productivity (g/L/h) is attained than has ever been reported for a bioprocess on any substrate; heterotrophic, chemotrophic, or photosynthetic. In certain embodiments a productivity ≥2 g/L/hr or ≥3 g/L/hr or ≥5 g/L/hr or ≥10 g/L/hr or ≥20 g/L/hr or ≥30 g/L/hr or ≥40 g/L/hr or ≥50 g/L/hr or ≥70 g/L/hr or ≥90 g/L/hr or ≥100 g/L/hr is attained.

In certain embodiments of the present invention, strain and/or process engineering is utilized to extend the observed trend of increasing productivity with P out to GTL-type pressures (e.g., up to around 50 bar and/or ≥50 bar and/or ≥100 bar and/or ≥200 bar and/or ≥300 bar and/or ≥400 bar). In certain such embodiments, the highest bioprocess productivities ever recorded on any substrate are attained.

Using lab scale, high pressure (P) bioreactors the continuous knallgas bioprocess will be evaluated for Y_{H2} (g biomass/g H₂) and productivity (p g/L/h) in high P regimes that to our knowledge are unprecedented in bioprocesses, but which are commonplace in chemical GTL processes. This work will leverage and build upon the extensive experience the inventors have already accumulated in running continuous knallgas bioprocesses at elevated pressures with Cupriavidus necator. It will also leverage expertise that the inventors have, and expertise that is known in the art and science of designing, modeling, and constructing high P chemical reactors and in the chemical engineering of reactions involving flammable gases such as H₂ and/or syngas.

### Example 3: Optimizing Productivity at a Given Pressure

Several different pressure ramp-up experiments were performed. In these experiments a certain set of run parameters were targeted at the outset and held fixed (e.g., agitation rate, pH, T, OD₆₀₀, input molar gas flows and % gas composition of H₂, CO₂, and O₂, mineral nutrient medium composition), while the pressure was incremented upwards. This was done to elucidate the effect of total reactor pressure (P bar) on productivity. It was established that a positive correlation between total P and productivity generally exists, which extends up to at least 5 bar, for the CSTR with C. necator culture. The positive correlation of productivity with total P was demonstrated for several different CSTR run conditions (e.g., a given set of agitation rate, pH, T, OD₆₀₀, input molar gas flows and % composition of H₂, CO₂, and O₂, mineral nutrient medium composition, etc.) as shown in FIG. 28.

An increase in P was found to lead to an increase in productivity under a variety of different CSTR run conditions. Each different color trend line in FIG. 28 corresponds to a different set of CSTR run conditions, with the total P being varied and other run parameters kept fixed for a given trend line. While the productivity was generally observed to increase with P, the slope of that increase was found to vary substantially according to the set point values chosen for the various run parameters held fixed during pressure ramp-up (e.g., agitation rate, pH, T, OD₆₀₀, input molar gas flows (standard VVM) and % composition of H₂, CO₂, and O₂, mineral nutrient medium composition). Thus, a set of CSTR run conditions was identified that could nearly reach 3 g/L/h productivity at 4 bar pressure, while other sets of conditions might only give 1 to 1.5 g/L/h productivity at the same P. However, in all cases shown in FIG. 28, the mol % of O₂ in the reactor headspace was kept at mol % O₂≤5%. This was done to ensure a non-flammable, fuel-rich (i.e., H₂-rich), gas mixture in the reactor headspace, even at elevated pressures. This precaution was taken to add another layer of safety in the CSTR operation.

With the mol fraction of O₂ (f_{O2}%) constrained to f_{O2}%≤5%, elevating the total pressure (P) results in higher pO₂ at a fixed mol % given that pO₂=f_{O2}%*P (e.g., 0.05 bar O₂ at P=1 atm, 0.1 bar O₂ at P=2 atm, 0.15 bar O₂ at P=3 atm etc.). This relaxes the O₂-limitation on growth that generally exists with fuel-rich gas mixtures and enables increased productivity.

### Example 4: Increased Mol Fraction % O₂ in Non-Flammable Gas Mixtures with Increasing Pressure

It can be experimentally determined whether at elevated pressure, the mol fraction of O₂ demarking the threshold between flammable and non-flammable gas mixtures remains around 5% O₂, as is the case at ambient pressure. According to Schroeder et al 2005 [Schroder, V and Holtappels, K. (2005) “Explosion characteristics of hydrogen-air and hydrogen-oxygen mixtures at elevated pressures,” in International Conference on Hydrogen Safety, Congress Palace, Pisa, Italy, 2005, is incorporated herein by reference in its entirety], the flammability range of H₂-air gas compositions narrows as the pressure increases above ambient up to around 20 bar. Specifically, lower mol fractions of H₂ lie above the upper explosivity limit (UEL) i.e., in the fuel-rich region of the flammability diagram, as P increases, and consequently higher mol fractions of O₂ than 5% lie outside the flammability range at elevated P (i.e., on the fuel-rich non-flammable side of the flammability diagram). Thus, non-flammable fuel-rich gas mixtures can contain a higher f_{O2}% at elevated pressures than at atmospheric pressure (see FIG. 2 in Schroeder et al). This trend of increasing f_{O2}% reverses above 20 bar, however, even at 50 bar the mol fraction of O₂ demarking the boundary between flammable and non-flammable H₂-air mixtures remains higher than at ambient pressures (i.e., >5% O₂). Thus, increasing P can not only increase pO₂ in a safe H₂—CO₂—O₂ gas mixture by increasing the P term in pO₂=f_{O2}%*P, but also by enabling a safe increase in f_{O2}%. This effect can be further investigated experimentally to confirm the effect of pressure on the H₂-air UEL reported by Schroeder et al and extending the investigation to the case of H₂—CO₂—O₂ mixtures.

### Example 5: Maintaining Positive Trend of Increasing Productivity with Increasing Pressure

If the O₂ yield, k_{L}a, k_{H}, and f_{O2} remained constant, then one would expect a linear increase in productivity with pressure by the aforementioned relationship:

$$p = {Y}_{{O}_{2}} \times {k}_{L} a ( {k}_{H} {pO}_{2} - DO ) \approx {Y}_{{O}_{2}} {k}_{L} a {k}_{H} {pO}_{2} = {Y}_{{O}_{2}} {k}_{L} a {k}_{H} {f}_{{O}_{2}} P$$

However, it can be seen in FIGS. 25 and 28 that the increase in productivity with P seems to be less than linear. The best power law fit to the highest productivity trajectory with P had an exponent of 0.76 i.e., sublinear. This suggests that one or more of Y_{O2}, k_{L}a, or k_{H} were decreasing as P increased (f_{O2} was held fixed). Currently, it is suspected that a decrease in k_{L}a with increasing P is the most likely explanation and primary cause for the sublinear trend. The effect can be experimentally clarified and quantified in terms of how Y_{O2}, k_{L}a, or k_{H} are changing with P. In certain embodiments of the present invention, designs and measures are implemented to prevent or mitigate any decreases in these terms that would tend to counteract or cancel out the productivity gains expected as P is increased above 5 bar up to 50 bar and beyond (i.e., >50 bar). In certain embodiments, a particular emphasis is placed on maintaining bioreactor k_{L}a with increasing P and/or else at least minimizing any reduction that occurs with increasing P. A reduction in k_{L}a with P could counteract the effect of increasing P if the reduction became too pronounced. The O₂ yield (Y_{O2} g biomass/g O₂) is highly correlated stoichiometrically with the H₂ yield (Y_{H2} g biomass/g H₂). Therefore, the measures that are described in the Example below for maintaining or improving Y_{H2} as P is increased should, in certain embodiments of the present invention, simultaneously serve to maintain or improve Y_{O2}, and consequently positively impact productivity.

### Example 6: Increasing H₂ Yield at a Given Pressure

In another set of experiments, the dilution rate (μh⁻¹) of the CSTR was varied while the P and other run parameters were held fixed (e.g., agitation rate, pH, T, input molar gas flows and % composition of H₂, CO₂, and O₂, mineral nutrient medium composition etc.). The OD₆₀₀ was allowed to vary in response the change in dilution rate (OD₆₀₀ decreases as the dilution rate increases).

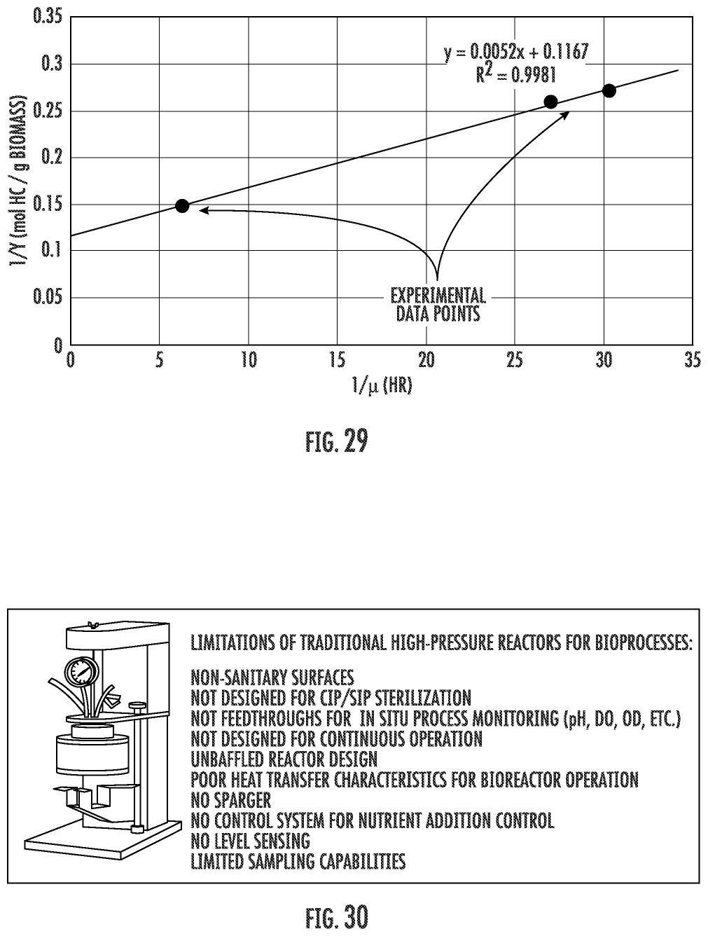

Yield data was collected from numerous turbidostat runs held at different μ. From this data a positive correlation between the inverse yield (1/Y_{H2}) and the inverse of the CSTR dilution rate (1/p) was established. In a turbidostat the dilution rate μ equals the specific growth rate of the culture (g biomass produced/h/g standing biomass). An illustration of the correlation between 1/p and 1/Y_{H2} for a set of CSTR runs, each held at a different μ, is shown in FIG. 29.

A good linear fit was observed between 1/μ and 1/Y_{H2}.

Not intending for the present invention to be limited by theory, the equation predicting this linear relationship:

$$\frac{1}{Y} = \frac{m}{μ} + \frac{1}{{Y}_{μ \_ inf}}$$

has been reported before by Bongers [Bongers, L. Energy generation and utilization in hydrogen bacteria. J. Bacteriol. (1970) doi:10.1128/jb.104.1.145-151.1970] and many other authors, where:
Y=observed yield (g biomass/mol H₂).
m=slope=maintenance energy (mol H₂/g biomass/h).
μ=specific growth rate (h⁻¹)=dilution rate in turbidostat CSTR.
Y_{μ_inf}=inverse intercept=ideal yield (g biomass/mol H₂) of cell synthesis in limit of the absence of any cell maintenance costs.

While for a given set of run parameters, the fit between 1/μ and 1/Y was consistent, a different set of fixed conditions (e.g., P, agitation rate, pH, T, input molar gas flows (standard VVM) and % composition of H₂, CO₂, and O₂, mineral nutrient medium composition etc.) generally resulted in a different slope (m) and vertical axis intercept (1/Y_{μ_inf}). Nonetheless, the expected increase in Y_{H2} with increasing μ was generally observed to hold in all cases tested. This approach of increasing the yield by increasing the dilution rate in the CSTR is one method used in certain non-limiting embodiments of the present invention to maintain and improve upon the Y_{H2} yield. In certain non-limiting embodiments of the present invention the Y_{H2}≥3.35 g biomass/g H₂.

It was generally observed that Y_{H2} increased in turbidostatic runs as P was increased. Not intending for the present invention to be limited by theory, one explanation for this is that as the productivity increases with P, the μ is increased to maintain a constant OD in the turbidostat. Therefore, by the above relationship between Y and μ, one would expect Y_{H2} to increase due to the increasing μ.

### Example 7: Experimentally Testing the Thermodynamic Impact of Increasing Pressure on H₂ Yield

Not intending to be limited by theory, another factor to consider in addition to the aforementioned effect of dilution rate, is that the thermodynamic driving force for both the knallgas respiration reaction (i.e., H₂(g)+½O₂(g)→H₂O(l)), as well as the biomass synthesis reactions (average empirical biomass reaction for C. necator found to be: 8.45H₂(g)+4.1CO₂(g)+NH₃(aq)→C_{4.1}H_{6.9}O_{1.7}N(s)+6.5H₂O(l)), will increase as the total P, and pH₂, pCO₂, and pO₂ increase. Since all the main reactants are gases, while all of the products are solids or liquids, increasing P will make both the CO₂-fixing and knallgas respiration reactions more thermodynamically favorable since they convert gases into liquids and solids. An important question that can be determined experimentally is whether the knallgas microorganism can conserve some of the increased thermodynamic gradient that is created by raising P, or if it is all lost as heat. To address this question, another aspect of the present invention is experimentally determining if and how the correlation changes between 1/p and 1/Y_{H2} over pressures ranging up to 50 bar, and beyond 50 bar (i.e., >50 bar). Not intending to be limited by theory, an ability of the knallgas microorganism to conserve and utilize the increasing thermodynamic gradient provided by increasing P should be reflected in either: the molar rate of H₂ consumed for cellular maintenance per biomass decreasing, i.e., a decrease in the slope m; and/or in an increase in Y_{μ_inf}, which would correspond to a decrease in the y-intercept (i.e., 1/Y_{μ_inf}) of a plot such as that shown in FIG. 29. In either of these scenarios, Y_{H2} would be higher at a given μ in the CSTR as the P increases. Another effect that will be experimentally investigated is whether the maximum dilution rate before culture wash-out occurs in the CSTR (μₘₐₓ) increases as P increases. An increase in μₘₐₓ would indicate an increased intrinsic specific growth rate (g biomass produced/h/g standing biomass) for the knallgas microorganism at elevated P. By the aforementioned relation between dilution rate and yield, an increased μₘₐₓ in turn should also enable higher yields by allowing the extension of the observed trend of increasing Y_{H2} with increasing μ out to higher CSTR dilution rates than are possible at lower P without culture wash out.

### Example 8: Increasing the H₂ Yield Via the Implementation of More Efficient CO₂-Fixation Pathways

The energetic cost of CO₂ fixation directly impacts the yield of protein, lipids, polysaccharides, PHB and/or PHA, and/or other biochemicals. Knallgas chemoautotrophs have evolved to use several different CO₂-fixation pathways including the reductive pentose phosphate cycle (rPP; Cupriavidus) and the reductive tricarboxylic acid cycle (rTCA; Hydrogenobacter). Genome-scale flux-balance metabolic analysis performed by the inventors indicates a >40% higher theoretical yield for protein production through the rTCA pathway. So, another aspect of the present invention is to compare a rPP organism, e.g., C. necator, against a rTCA organism, e.g., Hydrogenobacter thermophilus, in terms of biomass productivity (g/L/h) and Y_{H2} (g biomass/g H₂) in a CSTR bioprocess. In certain embodiments of the present invention, a rTCA organism is utilized in a high pressure bioreactor and/or bioprocess of the present invention. In certain such embodiments, the H₂ yield is higher for a given biomolecule produced by an rTCA organism than for a rPP organism.

In addition, we will evaluate and compare many other chemoautotrophs that use either reductive pentose phosphate (rPP) or reductive tricarboxylic acid (rTCA) CO₂ fixation cycles and/or other CO₂-fixation cycles or pathways to explore potentially favorable carbon and energy balances.

rTCA species including Hydrogenobacter thermophilus, and gram-positive knallgas species including Hydrogenibacillus schlegelii will be evaluated for CO₂ utilization efficiency under pressurized cultivation conditions and compared in terms of productivity, Y_{H2}, and protein content against C. necator under comparable conditions.

### Example 9: Adaptive Evolution to Evolve High P Performance

We will use adaptive evolution to evolve high P performance by knallgas organisms. Adaptive laboratory evolution (ALE) will be performed on C. necator for increased productivity and tolerance of elevated P. Knallgas strains will be selected that have evolved under elevated P for robust performance phenotypes as measured by enhanced biomass productivity, yield, and stress tolerance.

ALE is a powerful technology particularly amenable to evolving industrially relevant phenotypes and has been used to select for nutrient adaptation and environmental stress resistance, e.g., temperature, high salt, and P [Dragosits, M. and Mattanovich, D (2013) Adaptive laboratory evolution-principles and applications for biotechnology. Microbial Cell Fact 12:64], [Marietou, A., et al. (2015) Adaptive laboratory evolution of Escherichia coli K-12 MG1655 for growth at high hydrostatic pressure. Front Microbiol 5:749.], [Lee, S and Kim, P (2020) Current status and applications of adaptive laboratory evolution in industrial microorganisms. J Microbiol Biotechnol 30:793-803. https://doi.org/10.4014/imb.2003.03072] [González-Villanueva, M.; Galaiya, H.; Staniland, P.; Staniland, J.; Savill, I.; Wong, T. S.; Tee, K. L. Adaptive Laboratory Evolution of Cupriavidus necator H16 for Carbon Co-Utilization with Glycerol. Int. J. Mol. Sci. 2019, 20, 5737. https://doi.org/10.3390/ijms20225737]. ALE of E. coli under pressure selection conditions has evolved strains and identified gene mutations conferring tolerance to pressures up to 50 Mpa (500 bar) [Hauben, K J., et al. (1997) Escherichia coli mutants resistant to inactivation by high hydrostatic pressure. Appl. Environ. Microbiol. 63: 945-950.]. ALE does not require prior knowledge of genotype-phenotype relationships. Unlike directed mutagenesis that improves a phenotype but can also accumulate non-beneficial mutations, ALE non-intuitively finds genome-wide adaptive mutations that contribute to fitness. In the consistent environmental conditions of continuous culture, a lineage of mutations will be developed in response to selection P resulting in the selected phenotype i.e., increase tolerance and performance at elevated pressure.

Baseline pressure metrics of productivity and yield vs lower P range, from ambient-5 bar, will be established for C. necator in a BioXplorer 5000P high-pressure steel stirred tank bioreactors described in EXAMPLE 1:, and in the high P range, 5-50 bar, in the high-pressure reactor constructed in in the following example—EXAMPLE 10:. Baseline phenotypes will be established for C. necator at incrementally applied Ps between ambient-5 bar and 5-50 bar as defined by growth rate, biomass productivity and yield, and P tolerance vs time.

Adaptive ALE will be used in continuous culture format to accelerate the generation of genome-wide mutations that confer P-tolerant phenotypes. ALE in continuous culture has the advantages of consistent environmental conditions, uniform nutrient supply, constant cell density and growth rate. Initial experiments will be performed in HEL BioXplorer 5000P reactors equipped for knallgas operation and P regimes up to 5 bar. A consistent applied P will be sustained over a number of cell divisions (100-2000). The reactor will be operated at a high μ, i.e., an exponential growth regime, to select for the fastest growing mutants while washing out slow growers.

At each increment of applied P, the specific growth rate of the population will be monitored over the selection duration and populations showing stable phenotypes of increased maximum growth rate, μₘₐₓ, will be sampled for genome sequence analysis. Improved strains will in turn be exposed to a new, higher P selection round. The goal is to serially adapt the strain via the incremental selection of mutations to create the candidate strain. Five candidate strains will be selected for sustained productivity improvement relative to baseline strains at P increments in the range of ambient-5 bar. Then five more candidate strains will be selected for sustained productivity improvement relative to baseline strains at P increments in the range of 5-50 bar.

At each P selection increment whole genome resequencing will be performed for strains with improved specific growth rates for genotype-to-phenotype mapping. Mutations that are usually identified in ALE studies are single nucleotide polymorphisms (SNPs), smaller insertions and deletions (indels) and larger deletions and insertions that contribute to genetic and gene regulatory changes leading to fitness changes during the selection for improved phenotypes. Genotype to phenotype correlations will be made in candidate strains selected for performance at each incremental P, from ambient-5 bar and from 5-50 bar.

### Example 10: Reactor Design, Construction, and Operation for High Pressure to 50 Bar

We will leverage computational dynamics simulations to assist reactor design and operation in order to determine effects of elevated pressure (P) on protein production, yield, and productivity in a PHB-negative high-protein mutant C necator strain. Using lab-scale reactors operating over a range of pressures from 1-50 bar; engineering to maintain/increase k_{L}a as P increases and maximize mass transfer rate of gases into solution (α product of k_{L}a*gas partial pressure); we will assess variables of gas mass transfer, productivity (g/L/h), Y_{H2}, and protein content (wt. % protein).

The objective of this experiment will be to determine required components for ultra-high P bioreactor system based on prior experience with continuous knallgas bioprocesses with C. necator, 50 bar P requirement, and desired data collection capabilities. Commercially available options for reactors and auxiliary components will be sourced, or where no commercial sources are available, design modifications will be made and custom components acquired to meet requirements.

The high pressure reactor will integrate commercial off-the-shelf (COTS), modified off-the-shelf (MOTS), and custom components. Although there are several manufacturers of high-P reactors for use in chemistry applications such as hydrogenation processes, these reactors have numerous limitations which limit their effectiveness for bioprocesses. Examples of these drawbacks are listed in FIG. 30.

Many of these limitations can be mitigated by modifying or adding options to a typical high-P reactor. For example, a 316 stainless steel reactor body (which is widely available) and other parts in contact with the biomass can be electropolished to meet standards for sanitary surfaces (i.e., electropolishing to a Ra of 20 pin to meet basic 3-A standards). In addition, some reactor manufacturers such as Parr Instrument Company offer options for baffles and gas entrainment impellers which can improve mixing and gas-liquid contact within the reactor.

Ultra-high pressure base reactor systems (Parr Instruments) will be acquired including steel vessels capable of operation at >50 bar. The high pressure reactors will be customized for knallgas operation, e.g., delivery and entrainment of CO₂, H₂ and O₂, sensors for monitoring pH, DO, T, gas concentrations, P, and OD.

A reactor will be sourced or designed with enough ports in the headplate to accommodate new attachments, so that many additional capabilities can be added to a reactor to increase its suitability for a continuous knallgas bioprocess. However, adding components to the internal volume of the reactor greatly affects reactor characteristics such as mixing behavior and gas-liquid mass transfer.

The heat transfer properties of a reactor are also extremely important for successful operation of a bioprocess. The knallgas process with C. necator operates at 30° C. and generates heat proportional to the productivity of the organism. Some heating upon reactor startup is required to get the initial temperature up to 30° C., but when the reactor reaches high-productivity conditions, excess heat is generated that must be removed. Pressure vessels are generally designed for operation under more extreme T (up to 225° C. for standard and 500° C. for high-T versions) and P (>350 bar) conditions and are therefore manufactured with very thick walls relative to the vessel size, which creates a barrier to heat removal from the reactor via external cooling. This introduces a lag in thermal response which risks overheating the bioprocess. This problem is compounded as reactors get larger because while the heat generated in the reactor is proportional to the reactor volume, the heat transfer out is proportional to the reactor surface area. Cooling the reactor internally, via cooling coils, provides much faster heat transfer, but these coils can cause mixing dead zones within the reactor and are prone to biofouling (biomass buildup). Simpler internal cooling devices such as single loops or cooling fingers can be used to mitigate these effects. Reactor modeling will be used to determine the reactor cooling method that best balances fast heat transfer, low impact on mixing, and low biofouling.

A reactor model will be built in SOLIDWORKS and the model transferred into COMSOL for computational fluid dynamics simulations to evaluate the effects of different reactor geometries, internal components such as impellers and baffles, and inlet gas flow rates on mixing, gas-liquid contact, particle dynamics, and/or heat transfer within reactor.

Reactor modeling can be used to evaluate the effects of the previously described reactor modifications on the knallgas process and to inform reactor design decisions. Commercially available computational fluid dynamics (CFD) software packages such as COMSOL and Fluent are designed to simulate fluid flow and related behavior such as heat and mass transfer in complex environments such as those found in a stirred bioreactor with multiple phases of material present. CFD models work by breaking down the system geometry into discrete elements or volumes, iteratively solving the governing equations for each element, and then meshing the results together to allow visualization and analysis of the behavior of the entire system. An example of geometry discretization and visualizations of model results for a simulated stirred-tank bioreactor is described in Buss, A., Suleiko, A., Rugele, K., and Vanags, J. (2017) CFD Analysis of a Stirred Vessel Bioreactor with Double Pitch Blade and Rushton Type Impellers. Excerpt from the Proceedings of the 2017 COMSOL Conference, Rotterdam, which is incorporated herein by reference in its entirety.

As part of the reactor design process, an accurate, scaled model of various bioreactor options and internal components will be constructed using SOLIDWORKS, and this reactor geometry will be integrated into COMSOL. COMSOL will be utilized to visualize the predicted velocity, concentration, and temperature gradients within the reactor, identify and minimize dead zones in the reactor to achieve uniform mixing, identify shear stress concerns, optimize gas entrainment, and model gas transfer for each reactor configuration. The results of the CFD modeling will be used to guide decisions on the final reactor design.

Perform P and shakedown tests to verify functionality of reactor. Confirm safe operation of reactor at >50 bar. Design additional tests to evaluate mixing and gas-liquid transfer in the reactor. Modify reactor design based on outcome of testing.

After an initial check of equipment suitability based on modeling results, commercial equipment will be procured where suitable, materials ordered for modifications, and/or plans submitted for fabrication of custom components at an external shop. A test plan will be developed for verifying the functionality of the reactor system to ensure that the equipment functions safely and effectively at high P. Once the equipment arrives and is assembled, shakedown tests will be performed. The first testing milestone will be the verification of safe operation and leak-tightness during a P test at a minimum hydrostatic P of 50 bar.

After the reactor passes the P test, additional tests will be carried out to cross-check basic reactor performance (k_{L}a, extent of mixing, etc.) with predictions from theory and COMSOL modeling. Examples of two such tests are (1) a gassing/degassing oxygenation test to determine k_{L}a values under various operating conditions (e.g., stirrer speeds), and (2) a dye injection tracer test (performed at low P in a clear polycarbonate surrogate reactor) to visualize the uniformity of mixing within the reactor.

Data from the shakedown tests will be reviewed to propose modifications to the reactor design and/or operating conditions to improve performance. The previously developed COMSOL model will be updated as necessary to inform the design of the modifications. Additional components will be sourced as necessary to implement the modified design.

Tests will be performed in CSTR knallgas bioprocesses run at elevated P to further characterize the effects of P on productivity, Y_{H2}, Y_{H2}, and protein content as well as extend the range of P investigated to much higher values than have been tested previously. Any adjustments in gas or media compositions and/or other reaction conditions that may be required to extend the observed trend of increasing CSTR productivity with P out to much higher P (i.e., up to 50 bar) than the currently tested Ps (i.e., ambient to 5 bar), will be determined and documented. New CSTR protocols will be developed for the novel ultra-high P bioreactor (UHPB) and operating the CSTR over the 5 to 50 bar P range.

In certain embodiments of the present invention a productivity of ≥3 g/L/h or ≥5 g/L/h or ≥10 g/L/h or ≥15 g/L/h or ≥20 g/L/h on CO₂ as sole carbon source will be attained in a pressure range falling between 5 and 50 bar. In certain embodiments such embodiments an H₂ yield of Y_{H2} ∞2.5 or 3 or ≥3.2 or ≥3.3 or ≥3.4 or ≥3.5 or ≥3.6 g biomass/g H₂ consumed will be attained in a pressure range falling between 5 and 50 bar. In certain such embodiments, the said CSTR will be run continuously for at least 72 hours or at least 168 hours or at least 720 hours or at least 2,000 hours or at least 8,000 hours.

### Example 11: Characterization of Flammability Diagram for H₂—CO₂—O₂ Gas Mixtures at Elevated Pressures

Flammability testing will be performed based on ASTM E918 to determine the flammability composition limits of mixtures of H₂, O₂, and CO₂ over a range of P up to 50 bar. Obtaining this data, which is not readily available for H₂—CO₂—O₂ mixtures at elevated P in the literature, will help determine the optimal mixture of gas to feed the reactor at each pressure tested, while maintaining safe operation.

### Example 12: Bioreactor Example

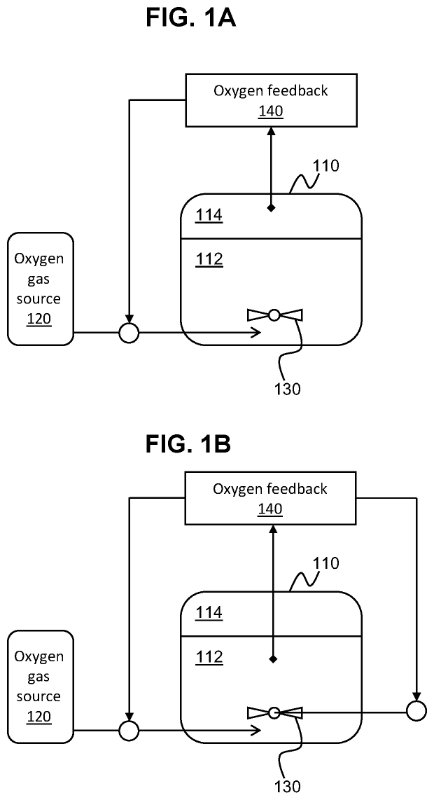

With reference to FIGS. 1A and 1B, embodiments of the bioreactor of the present disclosure are described. In some embodiments, the bioreactor includes a reactor vessel 110 that is configured to contain a culture 112 of a microorganism, e.g., a hydrogen-oxidizing or carbon monoxide-oxidizing microorganism. A gas headspace 114 may overlie the culture in the reactor vessel. The bioreactor may be configured to receive oxygen gas from an oxygen source 120. The oxygen gas may be delivered to the bioreactor as pure oxygen gas, or as part of a mixture of gases (e.g., a mixture of gases that includes hydrogen, carbon dioxide, and/or air, or any other gas mixture suitable for supporting growth of the microorganism). The oxygen gas (or mixture of gases) may be delivered to the culture, e.g., via a manifold positioned to deliver the gas into the culture. In some embodiments, the oxygen gas (or mixture of gases) is delivered to the culture through a gas diffuser (e.g., a sparger) positioned in the culture.

The bioreactor may be equipped with a mixer (e.g., a stirrer) 130 positioned in the culture and configured to mix the culture. In some embodiments, the mixer is positioned close to the gas diffuser (e.g., sparger). In some embodiments, mixer is positioned above the gas diffuser.

The bioreactor may include an oxygen sensor (or oxygen probe) that measures the level (e.g., the concentration, partial pressure, etc.) of oxygen in various compartments of the vessel. In some embodiments, the bioreactor may be configured to measure the level of oxygen in the headspace (FIG. 1A). In some embodiments, the bioreactor may be configured to measure the level of oxygen in the culture medium (FIG. 1B). In some embodiments, the bioreactor is configured to measure the level of oxygen in the headspace and the level of oxygen in the culture medium. The oxygen sensor may be any suitable sensor for measuring the level of oxygen in the headspace of the reactor vessel, or the level of dissolved oxygen in the culture medium. In some embodiments, the oxygen sensor is an optical oxygen sensor for measuring the level of dissolved oxygen in the culture medium.

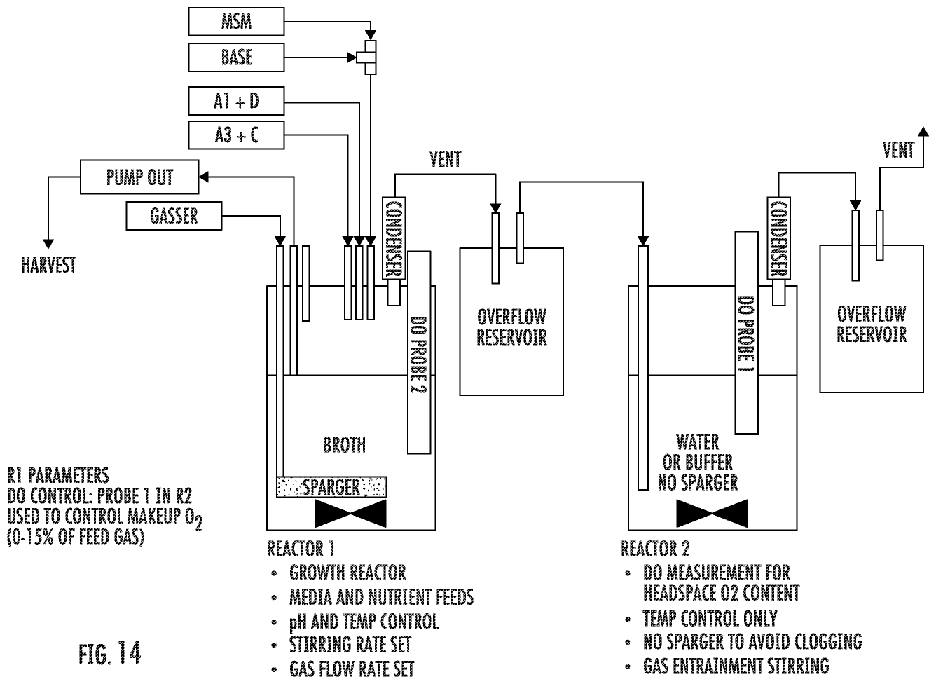

In some embodiments, the headspace oxygen level is measured using an oxygen sensor positioned in a second, “headspace” reactor (FIG. 14, Reactor 2). The headspace reactor may include a vessel containing a buffer or water, and the gas vent of the bioreactor containing the culture (Reactor 1) may be fed into the liquid compartment of the headspace reactor. The headspace reactor may include a mixer (e.g., a stirrer, for example an impeller, such as a gas entrainment impeller), and may include temperature controls to regulate the liquid temperature. An oxygen probe (e.g., an optical oxygen probe) may be positioned in the liquid compartment of the headspace reactor to measure the level of dissolved oxygen in the water or buffer. The level of dissolved oxygen measured in the headspace reactor may be proportional to the level of oxygen in the headspace gas of the reactor vessel containing the culture. In some embodiments, the dissolved oxygen probe in the headspace reactor measures the level of oxygen in the headspace of the vessel in which the microorganism culture is grown.

The bioreactor may be configured to use the measured level of oxygen in a vessel compartment (e.g., in the headspace or in the culture medium) as feedback control 140 to regulate the rate at which oxygen is delivered to the culture. The bioreactor may be configured to regulate by any suitable mechanism the rate at which oxygen is delivered to the culture. In some embodiments, the bioreactor includes a controller (e.g., a gas feed controller) configured to regulate, based on the measured level of oxygen, the rate at which oxygen is delivered to the culture. In some embodiments, the controller is configured to regulate, based on the measured level of oxygen, the flow rate of oxygen gas delivered to the culture from the oxygen gas source. In some embodiments, the controller is configured to regulate, based on the measured level of oxygen, the flow rate of a gas mixture that includes oxygen gas delivered to the culture. In some embodiments, the controller is configured to regulate, based on the measured level of oxygen, the extent of mixing (e.g., rate of stirring) by the mixer.

The gas feed controller may be configured to regulate delivery of oxygen to the bioreactor so that the level of oxygen in a bioreactor vessel compartment (e.g., the headspace or culture) is within a desired range of oxygen levels. In some embodiments, the bioreactor is configured to support chemoautotrophic growth of the microorganism (e.g., a hydrogen-oxidizing or carbon monoxide-oxidizing microorganism). In some embodiments, the gas feed controller is configured to regulate delivery of oxygen to the bioreactor so as to maintain a safe mixture of gases in the headspace. A safe mixture may include a mixture of gases (e.g., oxygen gas, hydrogen gas, carbon dioxide gas, nitrogen gas, etc.) in such proportions as to be non-flammable, including in the presence of an ignition source. In some embodiments, the gas feed controller is configured to have a target oxygen concentration in the headspace gas at about 5% (v/v) or lower.

With reference to FIG. 2, a bioreactor of the present disclosure may include a pH sensor configured to measure the pH of the culture 212 in the reactor vessel 210. The bioreactor may include a controller configured to regulate the rate at which a base 250 is delivered to the culture, based on the measured pH as feedback control 270. The controller may be configured to maintain the culture medium at a suitable pH for growing the microorganism. Any suitable control design may be used to regulate the base delivery rate based on the measured pH. In some embodiments, the controller is configured to employ a proportional integral loop to maintain the culture at a suitable pH based on the measured pH. In some embodiments, the controller is configured to maintain the microorganism culture at about pH 7.0. The base may be any suitable base, including, but not limited to, lime, sodium hydroxide, ammonia, ammonium hydroxide, caustic potash, magnesium oxide, iron oxide, and/or alkaline ash. In some embodiments, the base is ammonium hydroxide.

A bioreactor of the present disclosure may be configured to deliver to the microorganism culture nutrient amendments 260. The nutrient amendments may supplement one or more components of the culture media that are depleted as the microorganism grows. The nutrient amendments may include, without limitation, one or more supplements for sodium, potassium, calcium, magnesium, zinc, manganese, iron, cobalt, copper, nickel, phosphate, sulfate, chloride, borate, molybdate. In some embodiments, the nutrient amendment includes Na₂HPO₄, KH₂PO₄, MgSO₄, ferric ammonium citrate, CaCl₂, ZnSO₄, MnCl₂, H₃BO₃, COCl₂, CuCl₂, NiCl₂, Na₂MoO₄. In some embodiments, the bioreactor includes a controller configured to regulate the rate at which one or more nutrient amendments is delivered to the culture, based on the measured pH as feedback control. In some embodiments, the controller is configured to deliver one or more nutrient amendments to the culture at a rate proportional to the rate at which base is added to the culture.

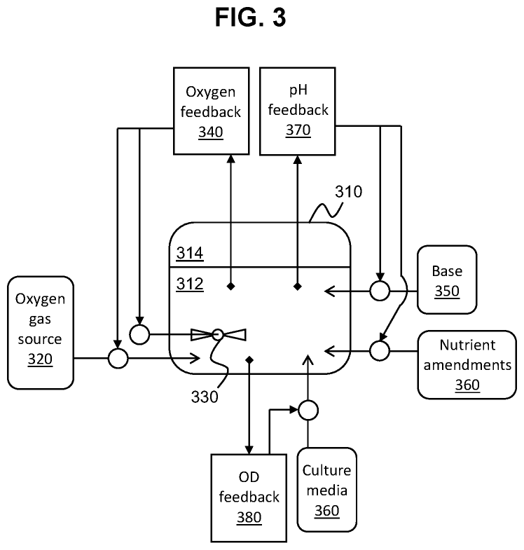

With reference to FIG. 3, in certain embodiments, a bioreactor of the present disclosure is configured to receive fresh (e.g., uninoculated) culture media 360. In some embodiments, the bioreactor is configured to receive culture media continuously from a source of fresh culture media. In some embodiments, the bioreactor may include an optical density (OD) sensor configured to measure the OD (e.g., at 600 nm) of the culture 312. In some embodiments, the bioreactor includes a controller configured to regulate the rate at which culture media is delivered to the culture. In some embodiments, the bioreactor includes a controller configured to regulate the rate at which culture media is delivered to the culture, based on the OD measurement as feedback control. In some embodiments, the controller is configured to regulate the rate of culture media addition so as to maintain the culture OD within a predetermined OD range, using the OD measurement as feedback control.

In some embodiments, the bioreactor includes a foam sensor configured to measure the foam level in the reactor vessel (e.g., at the interface of the liquid culture and the gas headspace). Any suitable sensor for detecting the foam level may be used. In some embodiments, the foam sensor is a conductance-based sensor. In some embodiments, the foam sensor provides a feedback signal to a controller configured to regulate addition of an antifoam to the culture. In some embodiments, the controller is configured to regulate the addition of antifoam such that the reactor vessel does not foam over. In some embodiments, the controller is configured to regulate the addition of antifoam such that the level of foam above the liquid level of the culture is about 5 cm or less, e.g., about 3 cm or less, about 2 cm or less, including about 1 cm or less. Any suitable antifoam may be used to reduce the amount of foam in the reactor vessel. Suitable antifoam includes, without limitation, polypropylene glycol.

The risk that foam is created can be higher in aerobic and/or gas bioprocesses growing proteinaceous material. Proteins can exhibit surfactant type properties in aqueous solutions which can lead to the formation of stable foams. (This property is sometimes deliberately exploited in the food industry, for example, whisking of egg whites to create foams to enhance texture and mouthfeel). By their nature gas bioprocesses will lead to gas bubbles rising towards the gas-liquid interface in a bioreactor headspace where stable foams can quickly accumulate ultimately leading to a ‘foam-over’ event where foam is pushed out of a vent line. This is undesirable as often vent line sterile grade filters are present and these can be quickly blinded by wet foams. In turn this can lead to rapid over-pressurization of the vessel. Either fermenter over pressure trips initiate to stop input gas flows or a pressure relief device activates. Regardless this is process upset that frequently leads to cessation of a fermentation run. In certain embodiments of the present invention, the consumption of gaseous reactants by the culture and/or the lack of any gaseous products from the culture reduces the risks of a foam-over event and/or the deleterious effects associated with it. In certain embodiments, the formation of foams can increase gas-to-liquid mass transfer, which increases the delivery of gaseous reactants into solution. In certain such embodiments, there are no gaseous waste products produced, and therefore the hinderance of degassing waste gases created by foaming, is not a problem.

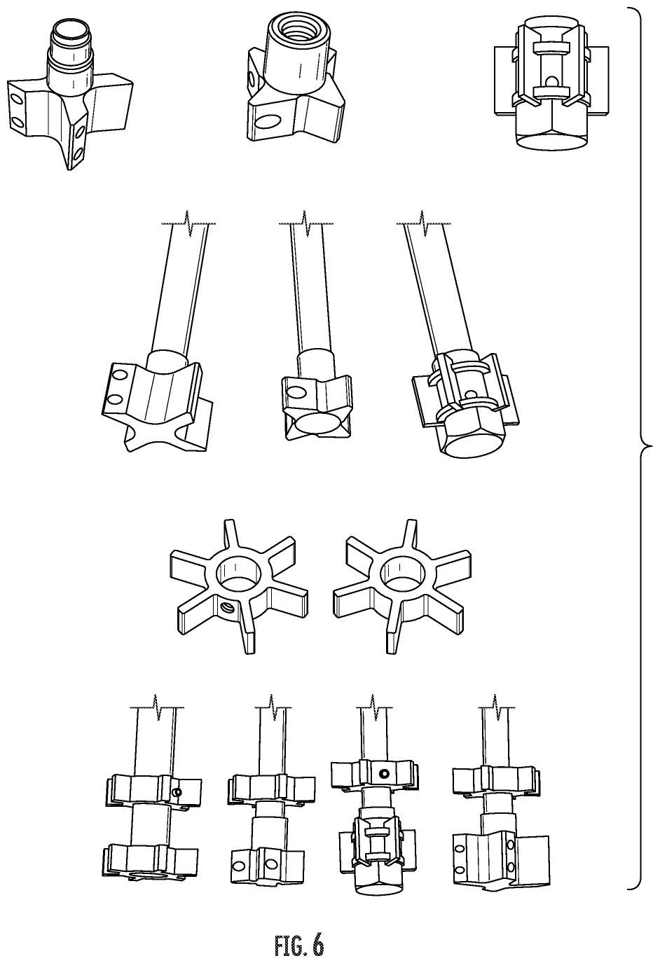

A bioreactor of the present disclosure may include any suitable mixer (e.g., stirrer) 130, 230, 330. In some embodiments, the mixer is an impeller, turbine, or hydraulic shear device. A suitable impeller includes, without limitation, a Rushton impeller, a gas entrainment impeller, a Rushton-style impeller with gas entrainment, or a basket impeller (FIGS. 4A, 5, 6). In some embodiments, the impeller is a gas entrainment impeller. In some embodiments, the bioreactor includes two or more impellers attached to the same axial shaft, such that the impellers rotate on the same axis. In some embodiments, all the impellers on a single axial shaft are the same type of impeller (e.g., a Rushton impeller). In some embodiments, the lower or lowest impeller relative to the gas headspace is a gas entrainment impeller, and the other impeller(s) are Rushton impellers.

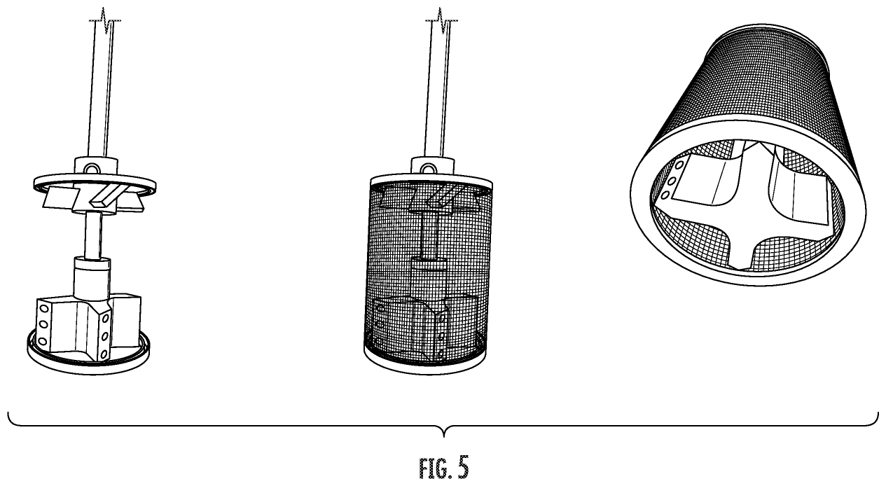

In some embodiments, the impeller is a basket impeller (FIGS. 4A and 5). In some embodiments, the basket impeller includes a mesh surface defining a lateral surface of a cylindrical basket, and top and bottom impellers capping the ends of the basket and configured to rotate around the axis of the cylindrical basket. The mesh surface may have any suitable mesh grade. In some embodiments, the top and bottom impellers are axial flow impellers (FIG. 4A). In some embodiments, the top impeller is an axial flow impeller and the bottom impeller is a gas entrainment impeller (FIG. 5).

The impeller may be rotated at a suitable rate to promote mass transfer of the gas. In some embodiments, the impeller is rotated at about 500 rpm or higher, e.g., about 600 rpm or higher, about 700 rpm or higher, about 800 rpm or higher, about 900 rpm or higher, about 1000 rpm or higher, about 1100 rpm or higher, including about 1200 rpm or higher. In some embodiments, the impeller is rotated at about 1800 rpm or lower, e.g., about 1700 rpm or lower, about 1600 rpm or lower, about 1500 rpm or lower, about 1400 rpm or lower, about 1300 rpm or lower, including about 1200 rpm or lower. In some embodiments, the impeller is rotated at a speed between about 500 rpm to about 1800 rpm, e.g., between about 600 rpm to about 1500 rpm, between about 700 rpm to about 1300 rpm, including between about 800 rpm to about 1200 rpm. In some embodiments, the speed of rotation of the impeller is varied during the incubation, based on, e.g., an oxygen feedback signal.

### Example 13: Membrane Oxygenator

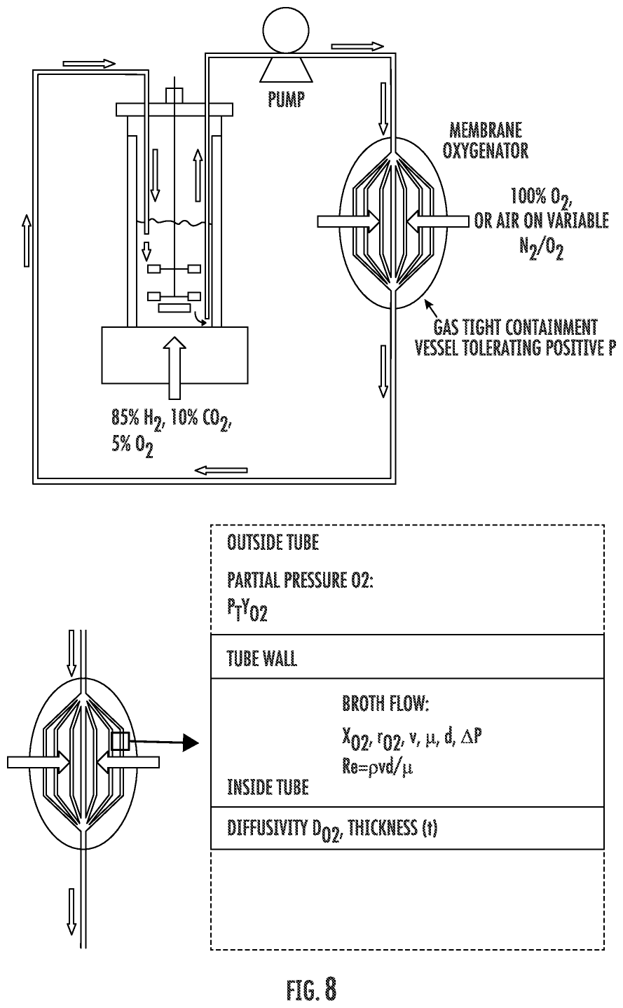

In some embodiments, a bioreactor of the present disclosure includes a membrane oxygenator that includes a gas permeable surface (e.g., a gas permeable membrane) over which the culture in the reactor vessel is circulated (FIG. 8). The gas permeable membrane separates one compartment fluidly connected with the reactor vessel, and a second compartment that contains oxygen gas. The gas permeable membrane may allow oxygen in the second compartment to diffuse across the membrane and into the culture circulating in the first compartment. The gas permeable membrane may have any suitable thickness to allow oxygen diffusion from the second compartment into the first compartment. The second compartment is not fluidly connected to the headspace of the reactor vessel, and thus the total or partial pressure of oxygen in the second compartment may be raised higher than would have otherwise been possible if the oxygen partial pressure were increased in the gas feed to the culture. Thus, in some embodiments, the total or partial pressure of oxygen in the second compartment is greater than the corresponding partial pressure of the dissolved oxygen in the culture. In some embodiments, the second compartment contains air, substantially pure oxygen, or a mixture of nitrogen and oxygen gas. In some embodiments, the second compartment is under elevated pressure.

The gas permeable membrane may be made of any suitable material that permits gas diffusion. Suitable materials include, without limitation, silicone, and polyethylene.

The gas permeable membrane may have any suitable thickness to allow diffusion of oxygen gas from the gas compartment across the membrane into the culture. The thickness may depend on the pressure inside the gas compartment and the material composition of the membrane. In some embodiments, the thickness of the membrane is about 1/32 inches or more, e.g., about 1/16 inches or more, including about ⅛ inches or more.

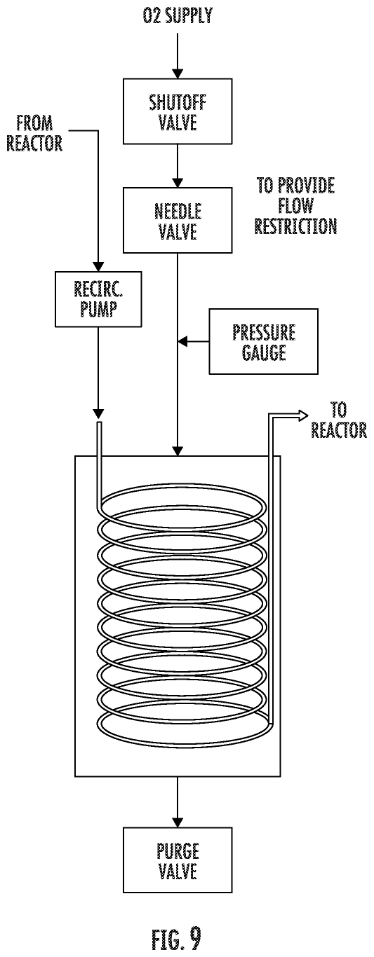

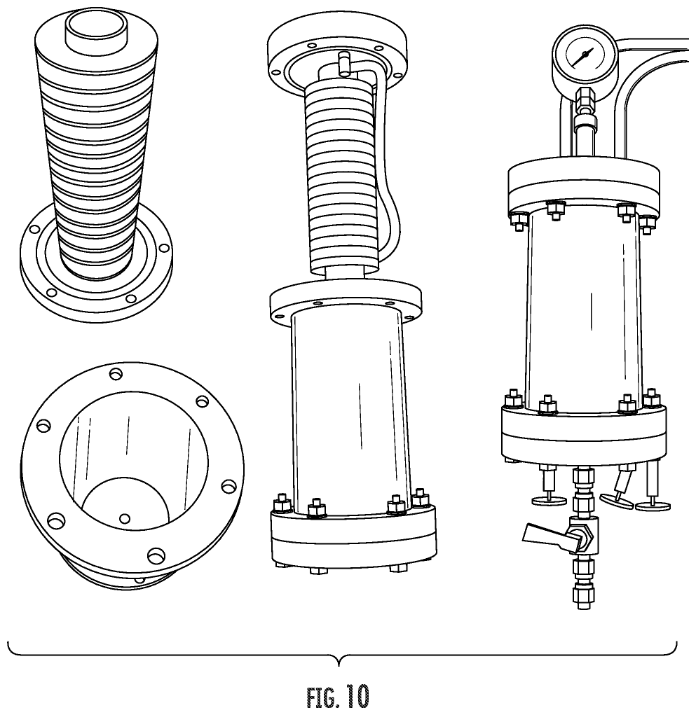

In some embodiments, the gas permeable membrane is a tubing. Any suitable dimension of tubing for achieving diffusion of oxygen into the culture may be used. The tubing may have any suitable inner diameter. In some embodiments, the tubing has an inner diameter of about 1/16 inches or more, e.g., about ⅛ inches or more, including about 3/16 inches or more. The tubing may be in any suitable configuration. In some embodiments, the tubing is coiled (FIG. 10). In some embodiments, the first compartment (through which the culture circulates) is inside the tubing, and the second compartment (containing oxygen gas) is outside of the tubing (FIGS. 8-11). A pump may be configured to circulate portions of the culture from the vessel, through the tubing, and back into the vessel.

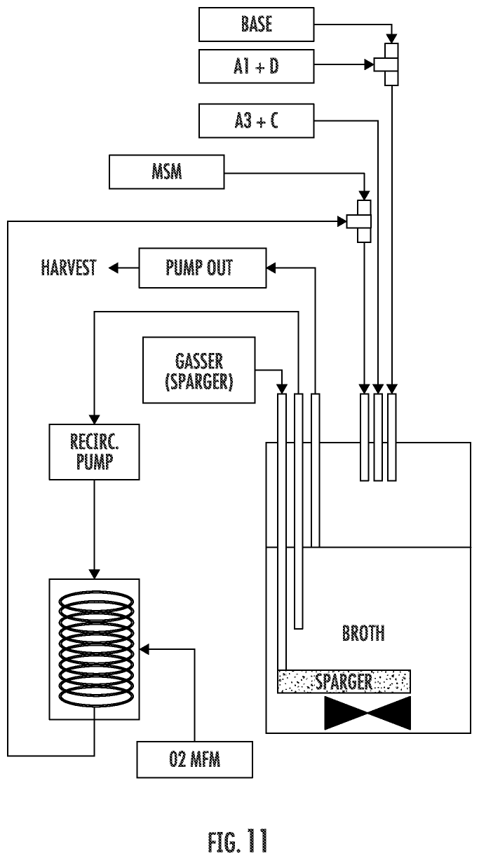

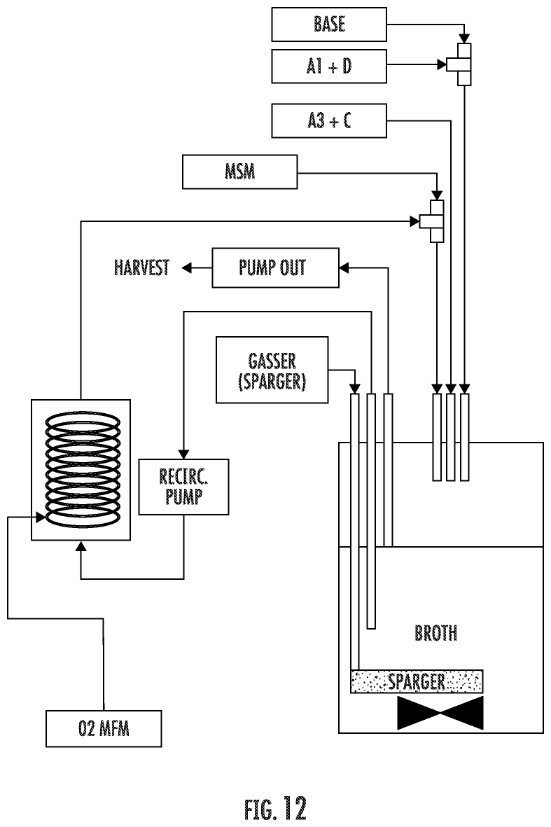

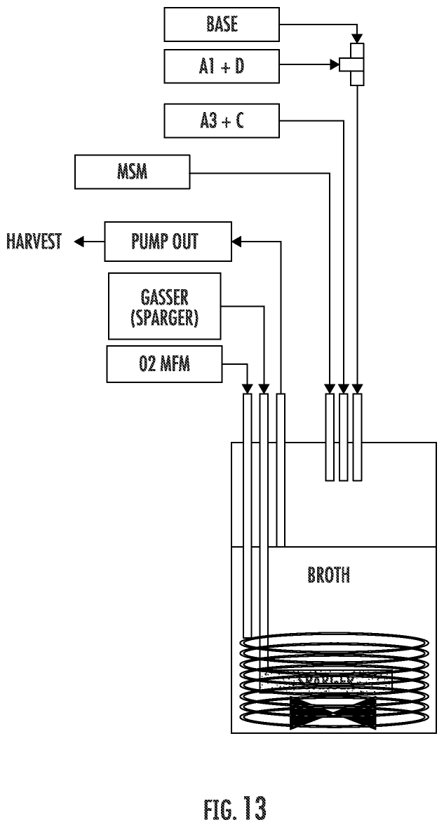

In some embodiments, the first compartment (through which the culture circulates) is outside the tubing, and the second compartment (containing oxygen gas) is inside the tubing (FIG. 12). In some embodiments, the tubing and second compartment is outside the reactor vessel (FIGS. 11 and 12). In some embodiments, a pump may be configured to circulate portions of the culture from the reactor vessel, through the first compartment, and back into the reactor vessel. In some embodiments, the tubing and second compartment is inside the reactor vessel (FIG. 13).

In some embodiments, an oxygenator includes multiple gas permeable membranes that are stacked parallel to each other to form multiple, alternating, parallel culture and oxygen gas compartments, where the culture compartments are fluidly connected to each other, and the oxygen gas compartments are fluidly connected to each other. In some embodiments, the culture circulates through at least one of the culture compartments in the oxygenator. In some embodiments, culture entering the oxygenator circulates through all the culture compartments forming a stack before exiting the oxygenator.

### Example 14: Example Bioreactor Set-Up and Protocol

The following protocol was followed in EXAMPLE 15:-EXAMPLE 18: below, unless indicated otherwise.

### Apparatus and Setup

(1) pH measurement and control (Hamilton EasyFerm Plus K8 200 pH probe)
(2) Temperature control (capable of heating and cooling to maintain 30° C.)
(3) Overhead stirring (or an alternative method of mixing) capable of generating high oxygen transfer rates (k_{L}a) nominally between 1500 and 3000 hr⁻¹
(4) Dissolved oxygen (DO) measurement and control (Hamilton VisiFerm DO 225 optical DO probe)
(5) Multitude of pumps to deliver base, nutrient amendments, and antifoam (as needed)
(6) Automatic feed of base (NH₄OH)
(7) Liquid sampling apparatus for sterile sampling
(8) Liquid withdraw for sterile withdrawal of reactor contents
(9) Sparger (or similar gas diffuser)
(10) Gas inlet with sterile filter (0.2 μm) connected to the sparger or gas diffuser
(11) Septa for sterile injection/transfer of inoculum or media using either a syringe or peristaltic pump (alternatively: configured with pumps to perform sterile transfers of inoculum and media into the reactor)
(12) Autoclavable or capable of in-place cleaning and sterilization
(13) Mass flow controllers to accurately control gas flow rate and composition, with software to adjust flowrate and composition
(14) Optical density (OD) measurement (Eppendorf BioSpectrometer for offline measurement, or BugLab BE2100 or BE3000 for continuous measurement)
(15) Condenser prior to exhaust
(16) Overflow reservoir to capture and separate foam from the exhaust line
(17) Liquid level sensor and control (conductance-based sensor, optional at smaller scales)
(18) Foam detection and control (conductance-based, optional at smaller scales)
(19) Additional pumps for continuous media feed and broth withdrawal, to be used during continuous operation.

The bioreactor may be suitable for growing a strain of C. necator, such as C. necator DSM 531 or C. necator DSM 541. The bioreactor may be configured with appropriate feedback and control methods. Suitable feedback and control loops used to monitor and control the bioreactor include:
(1) Temperature control—One or more thermocouples are used to monitor the temperature of the reactor. A combination of a process heater and process cooling water are used to maintain a temperature of 30° C. A proportional integral (PI) or proportional integral derivative (PID) control system is used to control the temperature, while the PI or PID settings will be system dependent. During typical operation, primarily cooling is required due to the exothermic nature of the organism growth. Depending on scale and equipment used, process heating may be provided by an external electric heater or through a temperature-controlled jacket while process cooling is provided by cooling water run through a jacket or internal cooling loop.

Typical Eppendorf DASGIP system settings: P=15, ti=1800.
(2) pH control—pH probe is used to monitor the pH. A proportional integral (PI) loop is used to control a pump to add base as needed to maintain a pH of 7.0. During typical operation, only base addition is needed. The PI settings are system dependent.

Typical Eppendorf DASGIP system settings: P=15, ti=45.
(3) Dissolved oxygen (DO)—A dissolved oxygen probe is used to monitor the dissolved oxygen content relative to 100% saturation for either oxygen or air as calibration. A cascade feedback loop is used to maintain a DO setpoint by adjusting the following variables: (1) stirring or mixing rate, (2) total gas flow rate, and (3) oxygen concentration in the gas mixture. Note that a 100% DO reading using air as the calibration gas will be equivalent to a 21% DO reading if oxygen is used as the calibration gas instead.

For purposes of this protocol, dissolved oxygen settings will be reported relative to oxygen.

During the high growth phase, the actual reading of the dissolved oxygen probe is found to be around 0, which indicates that the oxygen is consumed at the maximum transfer rate. This results in DO readings that do not reflect the actual concentration of oxygen in the headspace. Depending on the DO control settings for allowable oxygen concentration of the inlet gas can result in undesirable potentially explosive mixtures of gases to arise in the headspace (i.e., >5% oxygen, with the remainder hydrogen and carbon dioxide.) In order to prevent this, control of the inlet gases to achieve a maximum safe concentration of oxygen in the headspace may be used (i.e., s 5% oxygen and gas mixture on the H₂-rich side of the UEL).

Typical Eppendorf DASGIP system settings: P=0.2, ti=300.
(4) Headspace oxygen control feedback—To prevent the system from operating in a potentially explosive regime (i.e., >5% oxygen), measurement of oxygen in the headspace can be used to control the composition of oxygen in the feed gas and/or to shutoff the oxygen feed. An accurate method of oxygen measurement is needed.

For the Eppendorf DASGIP system, a second reactor with a dissolved oxygen probe is used. The second reactor is configured with temperature control, stirring with a gas entrainment impeller, and is filled with water or a buffered solution. This allows for monitoring of the headspace gas composition using an existing probe on the DASGIP system.
(5) Liquid level control—Due to the addition of base and media as well as the fact that the organism growth produces water, the liquid volume increases in the bioreactor overtime and water must either be continuously or occasionally removed to maintain the working volume. A liquid level sensor provides input for a control loop (PI, PID, or on/oft) to control a pump, which removes the excess liquid. A conductance-based sensor (or equivalent depending on process scale) is used to establish the liquid level and can be used to control the continuous or staged removal of liquid with a variable speed pump or a single speed pump, respectively. The liquid level control method will be scale- and process equipment-dependent. At small scales (<20 L), the liquid level control and liquid withdrawal can be manually performed as needed. Manual liquid withdrawal can be performed using an external peristaltic pump with tubing connected to the liquid sampling line. The reactor exterior is marked with an external liquid level scale; liquid is withdrawn using the pump until the desired level is achieved. Alternatively, a liquid withdrawal tube set at the desired liquid level height connected to a pump that is set to pump at a flowrate greater than the total of input feeds and water generation can be used to maintain the liquid level.

For the DASGIP reactor systems: the manual liquid withdrawal is used for batch operation while the liquid withdrawal tube set at the desired liquid level combined with a pump is used for continuous operation.
(6) Foam control—The growth of C. necator at high oxygen transfer conditions and high gas flow rates often results in foaming. A conductance-based sensor is used to measure the foam level either as a continuous measurement or point detection set at the maximum foam level. A basic feedback loop is used to deliver a small aliquot of antifoam as needed. Less preferred is regular manual addition of antifoam. The manual method can be used for lower-foaming system configurations, but still runs the risk of foaming over the bioreactor in between manual additions.
(7) Continuous operation—During continuous operation, the flow rate of fresh media (minimal salt media (MSM)) into the reactor is determined by optical density (OD) trends in the broth. If the OD is being measured manually, a calculation is carried out to determine the proper flow rate setting to achieve or maintain the targeted OD. Inputs to this calculation include the current OD, the trend in OD since the last measurement, the liquid volume in the reactor, and the current estimated productivity. With the integration of a continuous OD sensor such as the BugLab BE2100 or 3000, the OD control can be automatic. The BugLab system can be set up to send an analog or digital output signal which varies based on the slope of recent optical density measurements. A peristaltic pump can be controlled via the analog output on the BugLab base unit. The peristaltic pump will then use the incoming signal to set the flow rate of media into the reactor, between user-defined upper and lower flow rate limits, to maintain a constant OD.

### Media Preparation

### Minimal Salt Media (MSM):

The media used has the composition as shown in Table 1.

TABLE 1
Composition of MSM

| | | |
|---|---|---|
| Sodium phosphate dibasic | 4.5 | g/L |
| dihydrate (Na₂HPO₄•2H₂O) | | |
| Potassium phosphate | 1.5 | g/L |
| (KH₂PO₄) | | |
| Ammonium chloride | 1 | g/L |
| (NH₄Cl) | | |
| Magnesium sulfate | 0.2 | g/L |
| heptahydrate (MgSO₄•7H₂O) | | |
| Sodium bicarbonate | 0.5 | g/L |
| (NaHCO₃) | | |
| Ferric ammonium citrate | 5 | mg/L |
| (C₆H₈O₇•xFe³⁺•yNH₃) | | |
| Calcium chloride | 10 | mg/L |
| (CaCl₂•2H₂O) | | |
| Zinc sulfate | 100 | μg/L |
| (ZnSO₄•7 H₂O) | | |
| Manganese chloride | 30 | μg/L |
| tetrahydrate (MnCl₂•4_{H2O}) | | |
| Boric acid | 300 | μg/L |
| (H₃BO₃) | | |
| Cobalt chloride | 200 | μg/L |
| hexahydrate (CoCl₂•6H₂O) | | |
| Copper chloride | 10 | μg/L |
| dihydrate (CuCl₂•2 H₂O) | | |
| Nickel chloride | 20 | μg/L |
| hexahydrate (NiCl₂•6H₂O) | | |
| Sodium molybdate | 30 | μg/L |
| dihydrate (Na₂MoO₄•2H₂O) | | |

### Base Preparation (2 N Ammonium Hydroxide)

pH control is performed using 2 N NH₄OH.

2N Ammonium hydroxide is prepped from more concentrated solutions.

From 5 N NH₄OH: For every 1 L of 2N NH₄OH required, combine 400 mL of 5 M NH₄OH with 600 mL of deionized water.

From concentrated (28%) NH₄OH: For every 1 L of 2NH₄OH required, combine 137.6 mL of 28% NH₄OH with 862.4 mL deionized water.

### Antifoam

Antifoam is added to control the foaming that results from high oxygen transfer conditions. The primary antifoam used is polypropylene glycol. A variety of polypropylene glycols is available that have a range of molecular weight distributions, which result in different solubility. Polypropylene glycol P2000 from Sigma-Aldrich may be used.

### Inoculum Preparation

### Fructose Media (MSM-F) Preparation

The inoculum media is the same MSM as defined above, but with the addition of 40 g/L of D-fructose. The MSM is prepared per the protocol described above, D-fructose is added and dissolved, and the combined solution is filter sterilized. This media is referred to as MSM-F.

For a 1-L preparation: to a 1-L volumetric flask add 40 g of D-fructose, then add MSM to the 1-L mark, stir the solution until the D-fructose is dissolved, and then filter sterilize the combined solution.

Inoculum Preparation from Glycerol Stock

Inocula for the reactors are expanded in autoclaved Erlenmeyer flasks (250 or 500 mL).

Fill the autoclaved flasks with 100 ml of MSM-D inside of a biosafety cabinet and inoculate with ≥1 mL glycerol stock of C. necator. Cover the inoculated flasks and place inside of an incubator preset to 30° C. and containing a shaker plate. Once the flasks are in place, set the shaker plate to 175 rpm and leave the flasks aerating for 2-3 days, after which they will have achieved an OD of 15-30 and are ready to inoculate a bioreactor.

### Glycerol Stock

The glycerol stock can be:
expanded frozen stock culture of C. necator grown at 30° C. with shaking in tryptic soy broth or
gas-grown stock that had been prepared from pure material aseptically removed from a bioreactor during typical operation.

It was found that the use of frozen glycerol stock prepared from gas-grown organisms (number 2, above) resulted in significantly reduced lag phase (hours versus days) when inoculating bioreactors for gas-based growth.

### Preparation for Inoculating Small Bioreactors or Inoculum Train Bioreactor

Small bioreactors are inoculated using ˜50 mL of fructose grown inoculum (OD≥20) per 1 L of MSM in the bioreactor to give a starting OD of >1.

Inside of a biosafety cabinet, withdraw the inoculum from the flask using a sterile syringe outfitted with autoclaved large bore needles (12 GA×8 in). Replace the long needle with a capped and sterile needle (18G×1.5 in) for injecting through a septum into the inoculum bioreactor. Once a syringe is prepped, inoculate the bioreactor within 15-20 minutes.

### Preparation for Inoculating Larger Bioreactors

For larger bioreactors or several bioreactors in parallel, an inoculum train bioreactor is used to grow sufficient inoculum to enable starting at higher ODs. The inoculum train bioreactor is inoculated via the method described above and operated following the bioreactor operation conditions described below. The bioreactor is operated for 1-3 days with a target OD of >30. After this target is met, if the reactor is observed to be in the exponential growth phase, the inoculum train bioreactor is deemed ready to inoculate either a single larger bioreactor or multiple parallel bioreactors.

Prep the larger reactor or the parallel reactors following the appropriate procedures as described below. Following aseptic transfer procedures appropriate for the equipment used, transfer sufficient inoculum from the inoculum train bioreactor to the bioreactor(s) being inoculated such that a starting OD of 1-5 is achieved. For smaller bioreactors, syringes and the liquid withdrawal line can be used to transfer the inoculum. For larger reactors, an aseptic pump (such as a peristaltic pump) should be used to perform a sterile transfer.

### Bioreactor Operation

### Preparation

Prior to autoclaving or sterilizing-in-place: Calibrate pH probes with pH 4 and 7 standards. Assemble all reactor components as needed to ensure sterility. Prepare and sterilize all media. Calibrate mass flow controllers and pumps per their regular scheduled calibration procedure.

For the Eppendorf DASGIP reactors: Assemble the reactor with a gas entrainment impeller. Adjust the height of the impeller such that the bottom of the gas entrainment impeller is 1 7/16 inches from the bottom of the reactor baffles. Adjust the coupling between the stir shaft and the gas entrainment impeller to ensure that the gas intake hole is not blocked by the coupling. Install an airstone disk sparger at the bottom of the reactor and ensure that it does not interfere with other components. Install the DO probe, septum, liquid addition ports/lines, liquid withdrawal port (including sample vial), and condenser and check that fittings are hand-tight and O-rings are in good condition. Cover the pH probe port with a spare septum and tape over to close the port during autoclaving. Attach gas inlet tubing (with an inline sterile filter) to the sparger. Attach a vent/overflow bottle with flexible tubing to the condenser. Use cable ties to secure all connections to flexible tubing. Cover all exposed tubing ends with aluminum foil and secure with autoclave tape. Autoclave the reactors (dry) for one hour at 122° C. Separately autoclave the pH probes in buffer solution.

After autoclaving or sterilizing-in-place: Follow appropriate procedures at each step to ensure sterility. As needed, connect all process lines (gas feed, gas exhaust, liquid feeds, liquid outlet, overflow reservoir, and temperature control) to the bioreactor. Ensure all media, nutrient amendment, base, and antifoam reservoirs have sufficient volume for the run. Prefill the liquid lines as needed. Fill reactor with sterile MSM to the desired liquid volume. Turn on stirring/mixing and temperature control. Turn on cooling water to the condenser. Once the system is at 30° C., calibrate the DO sensor by: (1) starting a flow of nitrogen to the reactor until the DO reading stabilizes and set the 0% calibration and (2) start a flow of oxygen to the reactor until the DO reading stabilizes and set the calibration to 100%. Once DO is calibrated turn on the pH control and the DO control. Turn on gas delivery to the initial composition (70% hydrogen, 20% air, 10% carbon dioxide) and flowrate (0.5 VVM). Turn on liquid level control and foam control if used.

For the Eppendorf DASGIP reactors: Install the pH probes into the reactors in the biosafety cabinet. Install the reactors into the BioBlock and connect all process lines, turn on stirring, and start temperature control as described above. Use the DASGIP software to calibrate the DO probes. After calibration, start a new run in the DASGIP control software. Resume temperature control at 30° C. Start pH control at pH 7 and turn on liquid feed pumps. Set the DO controller to vary stirring rate (800-1200 rpm), gas inlet flow (0.5-2 VVM), and percentage of oxygen in the inlet gas. Note that the main gas feed to the reactors is a pre-mixed blend of H₂, air, and CO₂. To vary the percentage of oxygen in the inlet gas, a supplemental oxygen feed stream is added using the DASGIP gas mixing/delivery module. Set the DO setpoint to between 1-5% and begin stirring and gassing.

Optionally for the DASGIP reactors, to use DO control while maintaining a safe atmosphere in the reactor headspace: A two-vessel setup can be used. In this case, the first reactor is used for growing biomass, while the second reactor contains only sterile water. Both vessels are equipped with gas entrainment impellers. The vent gas from the first reactor is plumbed to the bottom of the second reactor through tubing only with no filters or spargers to avoid clogging risks. The DO probe in the second reactor is used as the input to the DO controller. In this case, the stirring and gas inlet flow rate settings are fixed, and the DO control settings vary only the oxygen percentage in the inlet gas for the first reactor. See the dissolved oxygen section below for more details.

### Inoculation

Following sterile transfer procedures appropriate for the equipment used, transfer sufficient inoculum from either a fructose grown inoculum or the inoculum train bioreactor into the bioreactor such that a starting OD of 1-5 is achieved. For smaller bioreactors, syringes and the liquid withdrawal line can be used to transfer the inoculum. For larger reactors, a sterile pump should be used to perform a sterile transfer.

### Base Addition for pH Control

Base in the form of 2N NH₄OH is added to the reactor to maintain a pH of 7.0.

If NH₄ is limiting nutrient for lipid storage, switch base to 2N NaOH when log plot of OD shows growth rate has slowed. Control pH with 2N NH₄OH until doubling time of culture increases 2-fold. At this point, switch to 2N NaOH. For Cupriavidus necator switching over to NaOH will increase synthesis of PHB.

### Nutrient Amendments

For a given amount of 2 N NH₄OH consumed for pH adjustment additional mineral nutrients from the MSM medium are proportioned in such that no mineral nutrient limitations arise.

For the fed batch protocol, the nutrient amendments are added continuously with the base addition based on ratios that prevent mineral nutrient limitations. In certain embodiments, individual pumps are used for each of the nutrient amendment solutions.

For the Eppendorf DASGIP reactors: Due to the limitation on the number of pumps available, some of the nutrient streams are combined ahead of time to enable continuous feed of nutrients. To avoid precipitation, certain compatible concentrated solutions are combined with additional deionized water. For each combined stream, mix the components, filter sterilize, and add to the stream reservoir. To avoid precipitation, the streams should not be combined prior to being fed to the reactor, nor should they be combined with the base feed prior to the reactor.

### Media Feed for Continuously Stirred Tank Reactor (CSTR) Operation

For continuous operation where the media is fed continuously to the reactor while broth is removed to maintain a set liquid volume, the media is the basal MSM described above.

### Dissolved Oxygen (DO)

Oxygen is shown to be the rate limiting nutrient and is limited by the oxygen transfer coefficient. To maintain high productivity, increased oxygen must be delivered; however, the amount of oxygen should be limited in order to avoid an explosive mixture of gases in the headspace, meaning that the gas composition in the headspace should be s 5% oxygen.

To maintain DO, a cascade feedback loop is used to adjust the following variables: (1) stirring or mixing rate, (2) total gas flow rate, and (3) oxygen concentration in the gas mixture, while keeping the ratios of hydrogen to carbon dioxide constant.

At small scales, such as with the Eppendorf DASGIP system, the bioreactor vent line is connected to a second reactor with temperature, gas entrainment stirring, and a DO probe which is in turn used to monitor the oxygen concentration of the headspace and control the oxygen concentration delivered to the bioreactor (FIG. 14). In this configuration, the stirring/mixing rate and gas flow rates are set, while only the oxygen concentration is adjusted in the feedback loop.

FIG. 14: Schematic of the configuration where a second bioreactor with a DO probe is used to monitor the headspace oxygen concentration in a bioreactor and provide feedback to control the oxygen concentration in the feed gas stream.

The stirring rate starts at 800 rpm and is allowed to increase to 1200 rpm. The total gas flow is 0.5 to 2 VVM. A premixed gas of 70% hydrogen, 20% air, and 10% carbon dioxide controlled via external mass flow controllers provides the bulk of the gas flow, while pure oxygen is added as needed to feed as much oxygen into the system as possible while maintaining a non-flammable gas headspace composition (i.e., s 5% oxygen by maintaining the DO setpoint in the said second reactor monitoring headspace oxygen concentration).

When using the headspace reactor configuration, there are some additional considerations. Before each run begins, since the target oxygen content is 5% in the headspace gas in order to maintain a non-explosive mixture, a premixed gas with 5% oxygen (composition listed in the Table 2 below) is fed into the headspace reactor before each run to calibrate the offset of the DO probe. After this check, the premixed gas feed is set back to the typical operating condition of 70% H₂, 10% CO₂, and 20% air. When using the headspace reactor configuration, the stirring rate and total gas flow in are fixed at 2.0 VVM and 1200 RPM. To achieve the DO setpoint in the headspace monitoring reactor, the DO controller varies only the supplemental oxygen fed to the cell growth reactor.

TABLE 2
Premixed gas composition containing 5% O₂

| Gas | Amount in mixture |
|---|---|
| H₂ | 66.67% |
| CO₂ | 9.52% |
| Air | 23.81% |

### Foam Control

Due to the presence of biosurfactants and high gas flow rates, foaming often occurs. To minimize foaming, antifoam can be added to the bioreactor. 0.2 mL of polypropylene glycol P2000 is added per 1 L of media once the OD is measured to be above 5-10. This often suffices to minimize the foam to be <0.5 cm above the liquid level. In cases where additional foaming occurs >1 cm above the liquid level, add 0.2 mL of antifoam as needed until the foam level drops. For small bioreactors, the antifoam is added in a sterile fashion via syringe and from an autoclaved vial of antifoam. For larger bioreactors, the antifoam should be pumped in as needed based on a conductivity-based foam sensor.

### Batch Operation

For the batch growth process, several of the manual interventions can be automated, such as base addition, nutrient amendments, DO control, foam control and OD measurement. As an independent check of the continuous OD measurement, samples can be withdrawn from the reactor at several time points over the course of a run. When taking samples, flush the line with sterile air and remove a purge sample of ˜1 mL to clear the sample tube of old un-mixed culture prior to removing a sample for OD and any other tests (DNA etc.).

### Draw-and-Fill

The draw-and-fill growth strategy is similar to batch operation during the growth portion. Once the reactor achieves a high cell density and growth has slowed (as determined by a combination of OD measurements and tracking base consumption), the bulk of the broth is removed in a sterile fashion using a pump. To the remaining broth, fresh sterile MSM is added to the operational liquid level. The remaining broth serves to inoculate the added media and growth resumes under the typical batch type operation occurs until the high cell density is once again achieved and growth has slowed, at which time another draw and fill is performed. This cycle can be repeated numerous times.

The draw-and-fill method can include performing the withdrawal while the organism is still viable in the high growth phase and leaving enough broth to have a sufficient high OD for the next “batch” growth portion of the draw and fill cycle. Draw-and-fill cycle has a growth phase start with ODs ˜20-30. The growth phase is allowed to continue until ODs around 150-200 are reached. At this point, the broth is withdrawn, leaving enough remaining in the bioreactor to start the next growth cycle with an OD of around 20-30.

A continuous OD measurement method may be used to identify the withdrawal point at high cell densities before growth slows.

### Continuously Stirred Tank Reactor (CSTR) Operation

Continuous operation is similar to fed batch operation within the reactors, with automated base addition, nutrient amendments, and DO control, and manual or automated antifoam addition and OD measurement. However, there is additional handling required for the fresh media feed and the broth withdrawal. The fresh media (MSM described above) is prepared ahead of time in a sterile container with sufficient media for several days of operation and is aseptically attached to the reactor liquid inlet line. The fresh media feed rate should be controlled either manually or automatically as described above. A pump and liquid level control are used to maintain the set liquid level. The withdrawal pump is set to operate at a faster flowrate than the fresh MSM feed due to both the nutrient amendments and water formation. The material withdrawn from the reactor is plumbed to a pre-sterilized, refrigerated (4° C.) collection reservoir. Material is harvested by pumping out of this reservoir on a regular basis, typically daily. The volume and OD of each harvest is measured, and 2 replicate 10 mL aliquots of broth may be taken to measure the cell dry weight density so that the productivity for the period encompassed by the harvested material can be calculated. The remainder of the harvest is centrifuged.

For the Eppendorf DASGIP bioreactor system: The fresh media is prepared and sterilized 6 liters at a time in 10-L carboys with attached tubing which is connected, as sterilely as possible, to the reactor liquid inlet line. A peristaltic pump with dual pump heads is used for both fresh media feed and broth withdrawal. To ensure that the reactor does not overflow, the broth withdrawal tubing is larger in diameter, so the flow rate of liquid out is greater than the flow rate of liquid in. The end of the withdrawal tubing inside the reactor is set at the desired liquid level, so no liquid is withdrawn if the broth is below the target level.

### Harvest

Material removed from the reactor is centrifuged to remove the biomass from the supernatant. Depending on scale and operational method, the material can be centrifuged in either a batch or continuous method at the equivalent of 12,000 G for 15-45 minutes at 4° C. Larger sample volumes tend to take longer to be centrifuged. Ideally, the broth is centrifuged once it is removed from the bioreactor, however if centrifuging does not occur immediately, then the broth should be refrigerated at 4° C. until centrifuging can occur. At this point additional downstream processing steps may be performed on the wet centrifuged biomass.

### Example 15: High-Productivity Growth of C. necator with Batch Operation and Draw-and-Fill Operation

An Eppendorf DASGIP bioreactor system was modified for growing C. necator (DSM 531), as described above. In each culture, the bioreactor was configured to provide nutrient amendments continuously. Some bioreactor configurations and operating parameters (including DO control setpoint, gas flow, percentage supplemental oxygen added to the feed gas, stirring rate and impeller configuration) were varied and tested as indicated in Table 3.

TABLE 3
Bioreactor configuration and operating parameters

| | DO | | Rate of | | |
|---|---|---|---|---|---|
| | Control | Gas | supplemental | Stirring | |
| | Setpoint | flow | O₂ addition | rate | Impeller |
| Run No. | (%) | (vvm) | to feed gas (%) | (rpm) | config. |
| KV0023-R5 | — | 1 | — | 1000 | GE-low, R |
| KV0036-R5 | 10 | 0.5-2 | 0-20 | 800-1000 | GE |
| KV0036-R8 | 15 | 0.5-2 | 0-25 | 800-1000 | GE |
| KV0038-R5 | 15 | 0.5-3 | 0-20 | 800-1000 | GE |
| KV0038-R7 | 20 | 0.5-3 | 0-25 | 800-1000 | GE |
| KV0041-R1 | 10 | 0.5-3 | 0-25 | 800-1000 | GE |

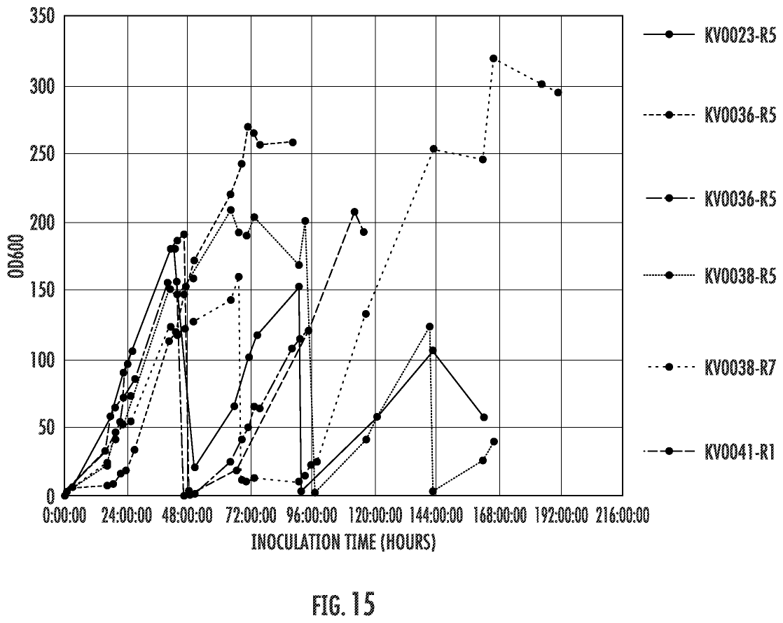

Cultures were grown in batch operation, or in draw-and-fill operation. In one culture (KV0023-R5), the oxygen level in the feed gas was held constant and was not adjusted by a DO control setpoint. In the other cultures, a DO probe in the culture provided feedback to control the oxygen level in the feed gas. The culture yield was estimated by taking optical density (OD) measurements at 600 nm. The results are shown in FIG. 15 and summarized in Table 4.

TABLE 4
Biomass yields for Batch and Draw-and-fill operations

| | Highest | Highest | Peak |
|---|---|---|---|
| | productivity | productivity | productivity |
| | over ≥72 hour | between 2 draw- | between 2 data |
| Run No. | period (g/L/h) | fills (g/L/h) | points (g/L/h) |
| KV0023-R5 | 1.12 | 1.41 | 1.94 |
| KV0036-R5 | 1.18 | 1.57 | 2.94 |
| KV0036-R8 | 1.07 | 1.53 | 2.9 |
| KV0038-R5 | 1.11 | 1.77 | 2.31 |
| KV0038-R7 | 1.34 | 1.36 | 2.42 |
| KV0041-R1 | 1.25 | 1.87 | 3.98 |

### Example 16: High-Productivity Growth of C. necator with Continuous Operation and DO Control on the Headspace

An Eppendorf DASGIP bioreactor system was adapted for growing C. necator (DSM 531 or 541), as described above. To measure the headspace oxygen level, the vent line for the bioreactor containing the culture was connected to a second reactor (“headspace reactor”) containing water or buffer (see “Bioreactor Operation” above, and FIG. 14). The DO probe in the headspace reactor was used to determine the oxygen concentration of the headspace in the bioreactor containing the culture, and to enable feedback control of the oxygen concentration delivered thereto.

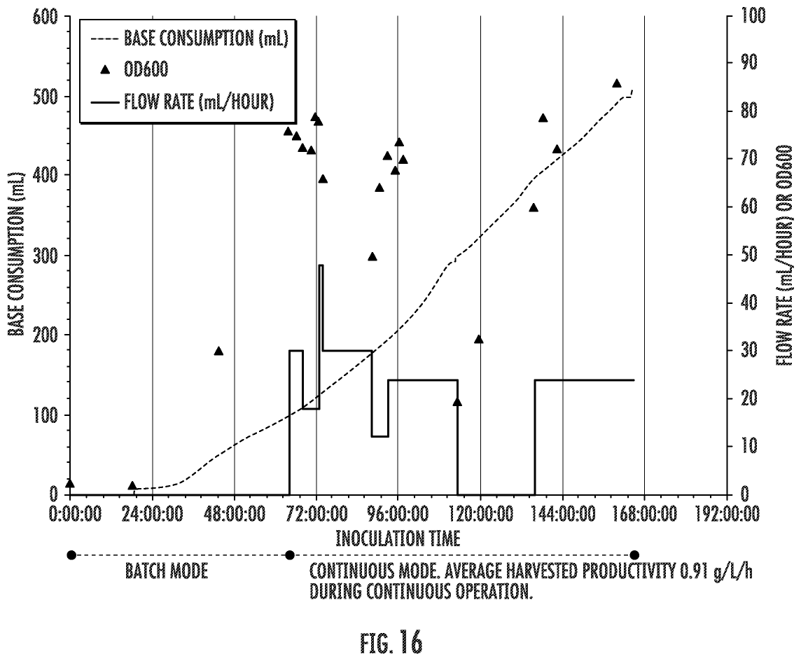

Nutrient amendments were provided continuously. The following configurations and parameters were used: gas flow: 2.5 vvm; supplemental oxygen added to the feed gas: 0-15%; stirring rate: 1200 rpm; impeller configuration: GE, R. After an initial growth in Batch operation, the bioreactors were operated continuously, with continuous addition of fresh media at flow rates adjusted as needed to maintain the culture within a target OD range, as shown in Table 5. The culture titer (g/L) was estimated by taking optical density (OD) measurements at 600 nm. The results are shown in FIG. 16 and Table 5.

FIG. 16 shows base consumption, OD₆₀₀, and fresh media flow rate for KV0051-R1.

TABLE 5
Operating parameters and biomass yield for continuous
operation with headspace DO control

| | | | DO Control | Continuous | Avg. |
|---|---|---|---|---|---|
| | | Target | Method | Run | Produc- |
| | | OD | and | Length | tivity |
| Run # | Organism | range | settings | (hours) | (g/l/hr) |
| KV0051-R1 | DSM 531 | 60-80 | Headspace | 101 | 0.91 |
| | | | O₂% | | |
| | | | at 4.2% | | |
| KV0053-R1 | DSM 541 | 60-80 | Headspace | 118 | 0.41 |
| | | | O₂% | | |
| | | | at 4.2% | | |

### Example 17: High-Productivity Growth of C. necator with Continuous Operation and Reactor DO Control

An Eppendorf DASGIP bioreactor system was adapted for growing C. necator (DSM 531 or 541), as described above. A DO probe in the culture provided feedback to control the oxygen level in the feed gas.

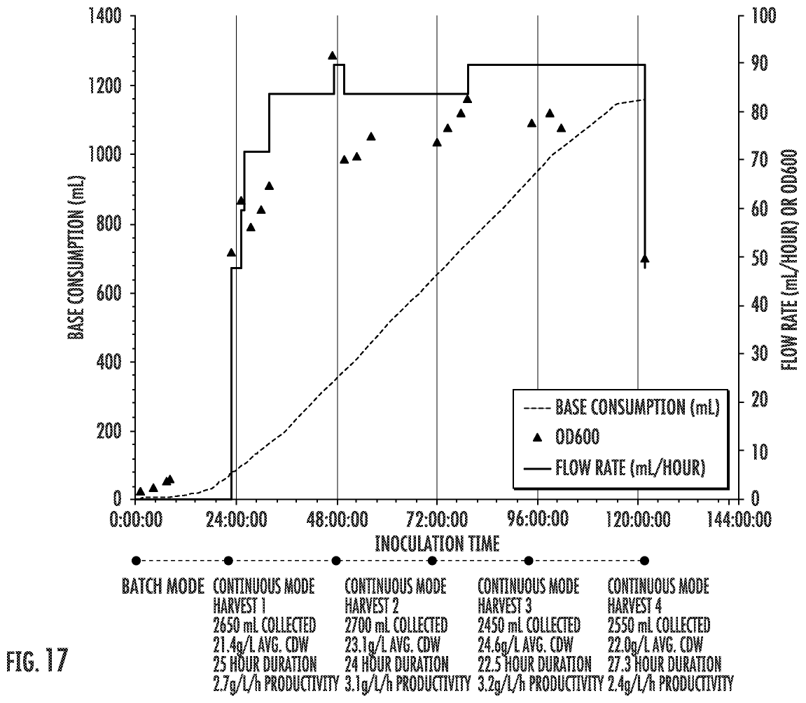

Nutrient amendments were provided continuously. The following configurations and parameters were used: DO control set point: 5%; gas flow: 2.5 vvm (KV0055-R6) or 0.5-2 vvm (KV0047-R1 and KV0056-R1); supplemental oxygen added to the feed gas: 0-15%; stirring rate: 1200 rpm (KV0055-R6) or 800-1200 rpm (KV0047-R1 and KV0056-R1); impeller configuration: GE, R. After an initial growth in Batch operation, the bioreactors were operated continuously, with continuous addition of fresh media at flow rates adjusted as needed to maintain the culture within a target OD range, as shown in Table 16. The culture yield was estimated by taking optical density (OD) measurements at 600 nm. The results are shown in FIG. 17 and Table 6. FIG. 17 shows base consumption, OD₆₀₀, and flow rate measurements for KV0047-R1.

TABLE 6
Operating parameters and biomass yield for continuous
operation with culture DO control

| | | | DO Control | Continuous | Average |
|---|---|---|---|---|---|
| | | Target | Method | Run | Produc- |
| | | OD | and | Length | tivity |
| Run # | Organism | range | settings | (hours) | (g/l/hr) |
| KV0047-R1 | DSM 531 | 60-80 | Reactor | 99 | 2.8 |
| | | | DO control* | | |
| KV0055-R6 | DSM 531 | 120-160 | Reactor | 187 | 1.5 |
| | | | DO control | | |
| KV0056-R1 | DSM 541 | 60-80 | Reactor | 144 | 1.7 |
| | | | DO control | | |

### Example 18: High-Density Growth of C. necator

An Eppendorf DASGIP bioreactor system was adapted for growing C. necator (DSM 531), as described above. In each run, the bioreactor was configured to provide nutrient amendments continuously. Some bioreactor configurations and operating parameters (including DO control setpoint, gas flow, percentage supplemental oxygen added to the feed gas, stirring rate and impeller configuration) were varied and tested as indicated in Table 7.

TABLE 7
Bioreactor configuration and operating parameters

| | DO | | Rate of | | |
|---|---|---|---|---|---|
| | Control | Gas | supplemental | Stirring | |
| | Setpoint | flow | O₂ addition | rate | Impeller |
| Run No. | (%) | (vvm) | to feed gas (%) | (rpm) | config. |
| KV0046-R5 | 10 | 0.5-3 | 0-15 | 1000 | GE, R |
| KV0033-R5 | 5 | 1 (mix | — | 800-1000 | GE, R |
| | | basis) | | | |
| KV0033-R6 | 5 | 1 (mix | — | 800-1000 | GE, R |
| | | basis) | | | |
| KV0038-R7 | 20 | 0.5-3 | 0-25 | 800-1000 | GE, R |
| KV0026-R2 | — | 1.5 | — | 1000 | R, R |

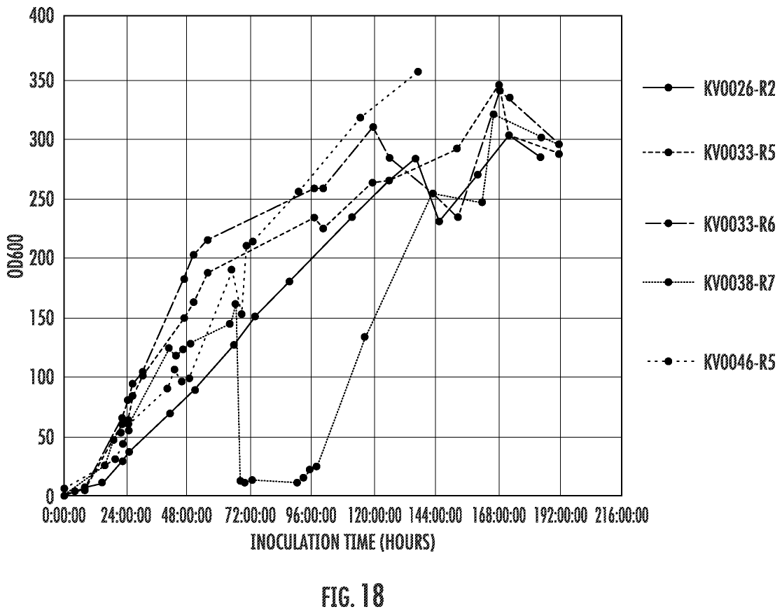

In one culture (KV0026-R2), the oxygen level in the feed gas was held constant and was not adjusted by a DO control setpoint. In the other cultures, a DO probe in the culture provided feedback to control the oxygen level in the feed gas. The culture yield was estimated by taking optical density (OD) measurements at 600 nm. The results are shown in FIG. 18 and summarized in Table 8. FIG. 18 shows OD measurements for high-density growth of C. necator.

TABLE 8
Results and biomass yields for high-density growth

| | | Time to | Est. CDW | Est. CDW |
|---|---|---|---|---|
| | | reach | at 0.25 | at 0.33 |
| | Max | max OD | g/L/OD | g/L/OD |
| Run # | OD₆₀₀ | (hrs) | (g/L) | (g/L) |
| KV0046-R5 | 357 | 137 | 89 | 118 |
| KV0033-R5 | 345 | 168 | 86 | 114 |
| KV0033-R6 | 341 | 168 | 85 | 112 |
| KV0038-R7 | 321 | 166 | 80 | 106 |
| KV0026-R2 | 303 | 172 | 76 | 100 |

Cell dry weight (CDW) estimates were based on an average cell dry weight versus measured OD (optical density) of 0.33 g/L/OD (range 0.23 to 0.42 g/L/OD, FIG. 19). FIG. 19 shows correlation between the ratio CDW density (g/L) to OD₆₀₀ to i.e., g/L/OD from measured dry cell weight of samples from cultures of C. necator (DSM 531), C. necator (DSM 541), and C. metallidurans (DSM 2839).

### Example 19: Analysis of Protein Content in Cultures

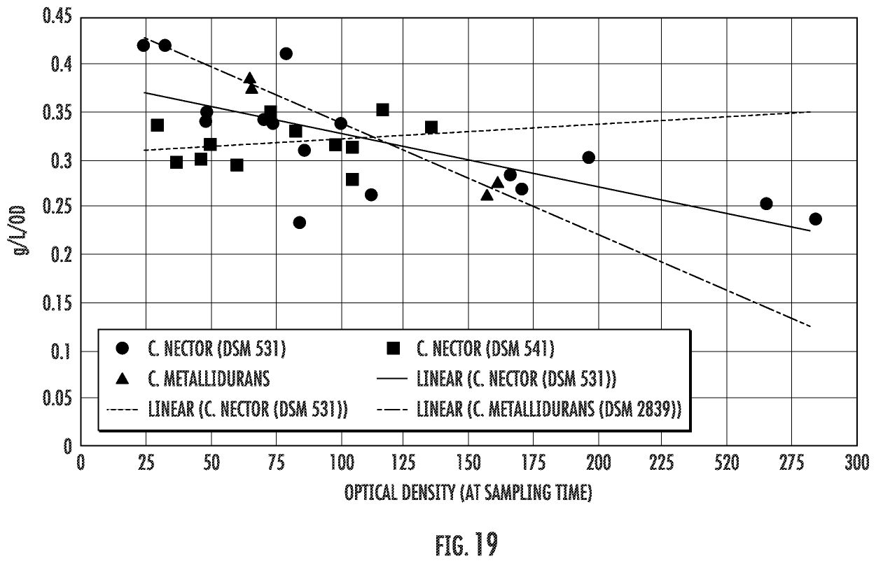

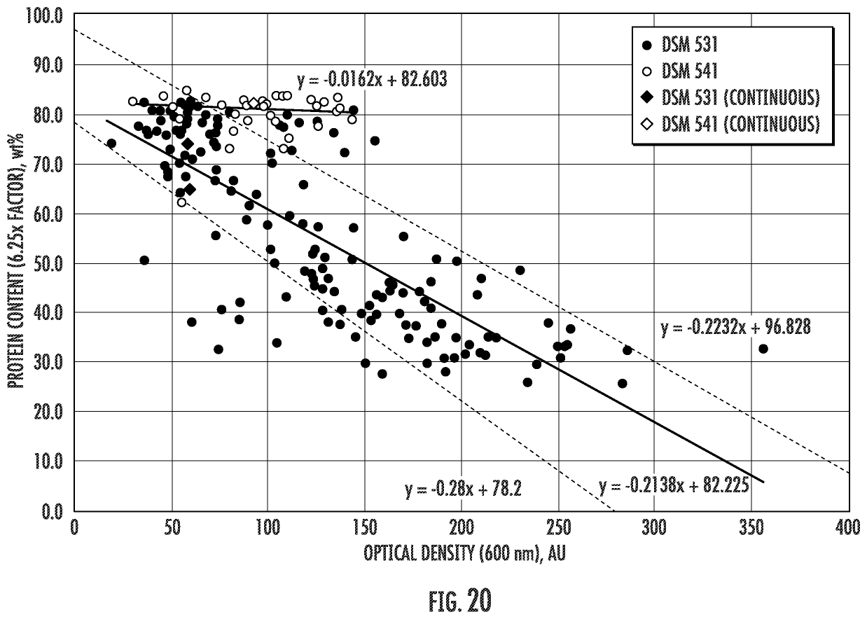

Samples taken at various ODs and time points during runs were analyzed for nitrogen content. A multiplier of 6.25 has been used to convert the % N values into % protein. The protein content vs. OD is reported in FIG. 20, for both DSM 531 and DSM 541 strains.

The trend for DSM 531 was distinctive of the samples taken to date, with a monotonically decreasing protein content with increasing OD. This is attributed to the organism entering an accumulation mode and producing more PHB instead of additional biomass. The upper and lower bounds were chosen to capture ˜90% of the data points and roughly correlate with the trendline. The outliers of these bounds may have been from sampling during upset conditions, such as immediately following a draw-and-fill, or may be correlated with some other effect. The DSM 541 strain exhibited a much flatter slope, with similar protein content at all ODs measured. However, the maximum OD reached in these sets of samples was much lower. This is attributed to the fact that DSM 541 is a non-PHB producing mutant and generates more biomass instead of accumulating PHB. However, it may be limited by cell density in the culture. From this, a desired protein content, or PHB content in the case of DSM 531, can be specified roughly based on OD for continuous operation or batch harvests, maximizing protein or PHB productivity.

The protein vs OD for the continuous run samples appeared to be consistent with the general trend for each strain.

### Example 20: High-Productivity Growth of C. necator with Continuous Operation and Split Gas Feed

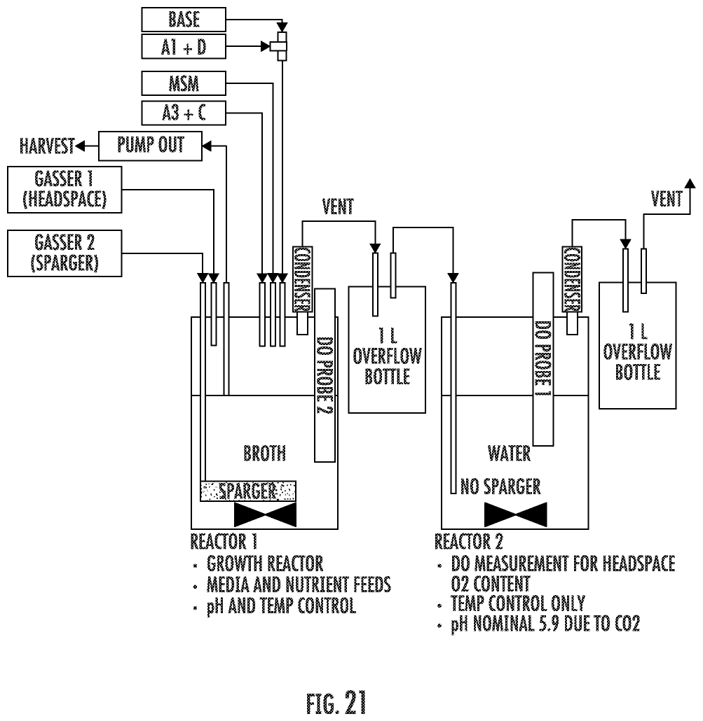

An Eppendorf DASGIP bioreactor system was adapted for growing C. necator (DSM 531 or 541), as described above. Feedback control of the oxygen gas feed was done either through DO measurement in the culture (KV0063-R6), or in a headspace reactor (KV0060-R6, KV0064-R5), as described in Example 14. Nutrient amendments were provided continuously. The bioreactor was configured to deliver the premix gas feed (70% H₂, 10% CO₂, and 20% air) to either or both the culture and the headspace of the bioreactor (FIG. 21). FIG. 21 shows a schematic showing configuration of a bioreactor for continuous operation with a split gas feed and headspace DO control. The mixed gas feed may be delivered to directly to the headspace of the culture bioreactor (“Reactor 1”) via Gasser 1, in addition to the sparger in the culture (“Broth”) via Gasser 2. The supplemental oxygen was fed through Gasser 2.

Bioreactor configuration and operating conditions are listed in Table 9.

TABLE 9
Bioreactor configuration and operating parameters

| | DO | | Rate of | | |
|---|---|---|---|---|---|
| | Control | Gas | supplemental | Stirring | |
| | Setpoint | flow | O₂ addition | rate | Impeller |
| Run No. | (%) | (vvm) | to feed gas (%) | (rpm) | config. |
| KV0060-R6^{#} | 5 (DO | 0.4-0.5 (Premix plus | 0-50 | 800-900 | Basket |
| | probe in | makeup O₂ to sparger); | | | (medium |
| | culture) | 1.5-2 (Premix to | | | mesh) |
| | | headspace) | | | |
| KV0063-R6^{§} | 5 (headspace) | 2 (Premix plus makeup | 0-100 | 1200 | GE, R |
| | | O₂ to sparger); then 2 | | | |
| | | (Premix to headspace, | | | |
| | | makeup O₂ to sparger) | | | |
| KV0064-R5^{§} | 5 (DO | 0.5-0.7 (Premix plus | 0-50 | 700 | Basket |
| | probe in | makeup O₂ to sparger); | | | (medium |
| | culture) | 1.5 (Premix to | | | mesh) |
| | | headspace) | | | |

^{#}C. necator DSM 531;
^{§}C. necator DSM 541

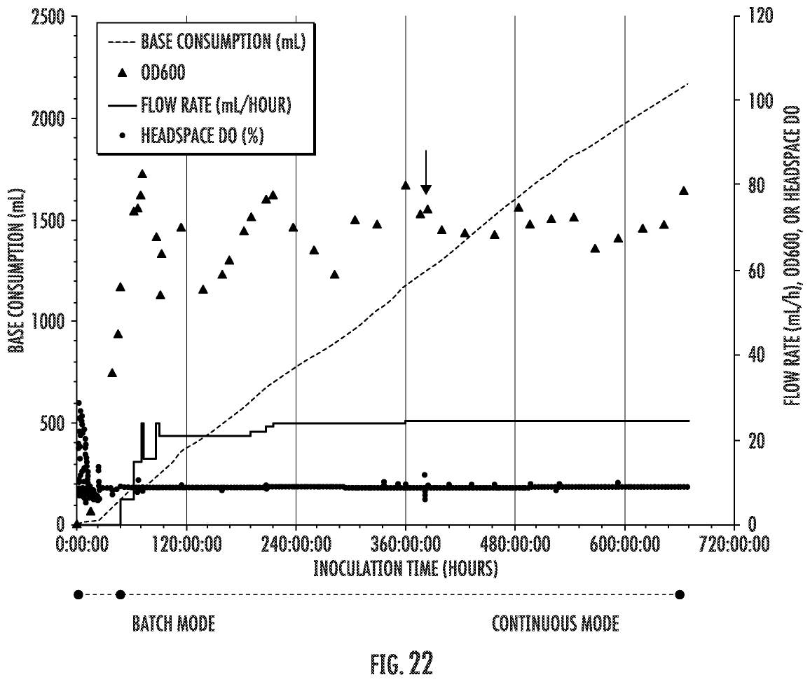

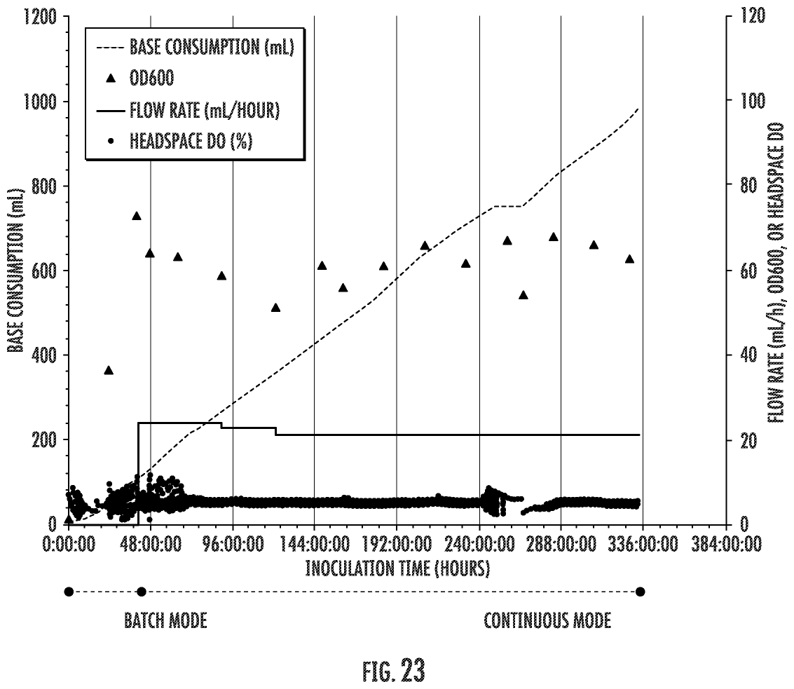

Results are shown in FIGS. 22 and 23 and Table 10. FIG. 22 shows the time course of base consumption, OD₆₀₀, media flow rate and headspace DO for KV0063-R6. The premix gas feeding was switched from the sparger to the headspace of the bioreactor at the time indicated with an arrow. The oxygen gas was fed through the sparger throughout the run. FIG. 23 shows the time course of base consumption, OD₆₀₀, media flow rate and culture DO for KV0063-R5.

TABLE 10
Run duration

| Run No. | Run duration (h) |
|---|---|
| KV0060-R6 | 314 |
| KV0063-R6 | 664 |
| KV0064-R5 | 328 |

### Example 21: Gassing/Degassing Method for Estimating k_{L}a

A bioreactor vessel was filled with 1 L water and temperature control was turned on to allow the vessel to stabilize at 30° C. Dissolved oxygen (DO) probes (Hamilton VisiFerm DO225) were calibrated. The stirring rate (“agitation”), inlet gas flow rate and pressure were set as shown in Table 11.

TABLE 11

| | | | | | | | Gassing |
|---|---|---|---|---|---|---|---|
| | | | | | | | Method |
| | | Superficial | | | | | Average |
| Air flow | | Velocity | Temp | | Impeller | Agitation | kLa |
| ( [symbol] L/h) | [symbol] M | [symbol] cm/ [symbol] | [symbol] ° C. [symbol] | Sparger | Configuration | (rpm) | ( [symbol] /hour) |
| 12 | 0.2 | 0.042 | 30 | L-large | RR | 1000 | |
| 60 | 1.0 | 0.212 | 30 | L-Large | RR | 1000 | 179 |
| 100 | 1.7 | 0.354 | 30 | L-Large | RR | 1200-1300 | 192 |
| 200 | 3.33 | 0.707 | 30 | L-Large | RR | 1200-1300 | 253 |
| 60 | 1.0 | 0.212 | 30 | Airstone [symbol] | Rotating | 1000 | |
| | | | | Disk | Basket | | |
| 120 | 2.0 | 0.424 | 30 | Airstone [symbol] | Rotating | 1200 | |
| | | | | Disk | Basket | | |
| 60 | 1.0 | 0.212 | 30 | Airstone [symbol] | GasE, R | 1200 | 174 |
| | | | | Disk | | | |
| 120 | 2.0 | 0.424 | 30 | Airstone [symbol] | GasE, R | 1200 | 182 |
| | | | | Disk | | | |
| 200 | 3.33 | 0.707 | 30 | Airstone [symbol] | GasE, R | 1200 | |
| | | | | Disk | | | |

[symbol]

indicates data missing or illegible when filed

Air was bubbled through the reactor until the DO probes read 100% and headspace was 21% O₂, at which point data recording was started.

The gas supply was switched from air to N₂ and DO vs. time data was collected during the desorption. N₂ was bubbled through the reactor until the DO probes read 0% and the headspace was 0% O₂. The gas supply was switched from N₂ to air and DO vs. time data was collected during the absorption. Air was bubbled through the reactor until the DO probes read 100% and headspace was 21% O₂.

The procedure was repeated after changing the stirring rate, inlet gas flow rate, or impeller configuration.

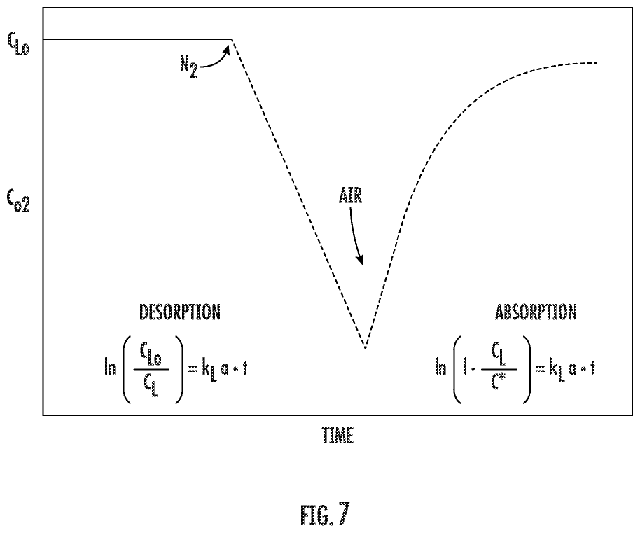

To calculate the estimated k_{L}a, f(DO) vs time for the absorption and desorption periods in the dataset were plotted. k_{L}a was equal to the slope of the linearized function for the desorption and absorption, as shown in FIG. 7, where C_{LO}=initial % DO when degassing (˜100%); C_{L}=measured % DO; and C*=% DO at saturation (100%).

FIG. 7: Schematic representation of oxygen desorption-absorption in a bioreactor, as described in Garcia-Ochoa, F., Gomez, E.; Bioreactor scale-up and oxygen transfer rate in microbial processes: an overview; Biotechnology Advances 27 (2009) 153-176, FIG. 4.

### Example 22: Protein Hydrolysate Produced from a Cupriavidus necator Culture

A Cupriavidus necator strain was cultivated chemoautotrophically in a mineral salts growth medium with CO₂ as carbon source and H₂ as electron donor. After growth, whole cell biomass was isolated from the growth medium by centrifugation and dried by lyophilization. The dried biomass was processed as follows:

Defatting the whole cell biomass: The whole cell biomass was defatted (lipids extracted out) with ammonium hydroxide and methanol (1:1:0.4, WCB: NH₄OH: MeOH) by stirring the mixture for an hour in fumehood in a tightly capped container. The mixture was vacuum filtered with Whatman 4 filter paper. The filtrate contained the extracted lipids. The retentate on the filter was the defatted biomass, which was dried at 40° C. in an incubator overnight.

Protein hydrolysis with NH₄OH and neutralization with CO₂: Solid loading of 2% of defatted dried mass into the hydrolysis step was prepared by rehydrated with the required amount of deionized (DI) water. The slurry was mixed well with a Turret Stick at 15,000 rpm for 1 min. The pH of the reaction mix was increased to 10.85 using NH₄OH 28%-30% (pre-made) solution in a fumehood. The mixture was transferred to a pressure tube (size: 120 mL) with 50 mL working volume. The mixture was then autoclaved at 110° C., 10 min, slow exhaust. The pH of the solution post autoclave was 10.82. The pH was then decreased to pH 9 by bubbling CO₂ through a cannula/18G needle inserted into the solution for 10-20 min. Enzyme digestion then was done at pH 9 with Bacterial Alkaline Protease at 55° C., 110 rpm overnight. The supernatant containing soluble hydrolyzed proteins was separated from the PHB rich crude pellet by centrifugation at 20000×g, 20 min, 5 C. The supernatant protein hydrolysate was then freeze dried. The ash content measured for this protein hydrolysate (PH) was 5%. The ash content was measured by placing a minimum of 300 mg of protein hydrolysate powder in a tared crucible and running an ash cycle in a muffle furnace. It was also independently measured by external lab analysis (SGS, North America) using AOAC method 942.05. The total amino acid content of the PH and the amino acid profile was also determined by SGS, North America using the AOAC method AOAC 994.12.

TABLE 12

| | NH₄OH/CO₂ low ash PH |
|---|---|
| % Total AA | 84.96 |
| Sample form | Dry powder |
| % Ash | 4.99 |
| Cysteine | 0.13 |
| Methionine | 1.62 |
| Tryptophan | 0.95 |
| Alanine | 7.7 |
| Arginine | 6.19 |
| Aspartic Acid | 6.1 |
| Glutamic Acid | 10.34 |
| Glycine | 5.11 |
| Histidine | 1.59 |
| Isoleucine | 3.04 |
| Leucine | 7.36 |
| Lysine | 6.28 |
| Phenylalanine | 4.95 |
| Proline | 3.77 |
| Serine | 6.1 |
| Threonine | 3.8 |
| Tyrosine | 4.41 |
| Valine | 5.52 |

### Example 23: Example of Pilot Design

The concept pilot design has the following main process sections:
Bulk gas storage
Liquid Media preparation—flexible concept design such that the fermentation can be operated on sugar as well as gases.
Bulk Liquid Media sterilization
Trace liquid additions—including alkali, acid, antifoam, and any specific trace salts/elements as required by the fermentation.
Fermentation—including seed generation.
Downstream processing—dewatering the fermentation broth and concentrating the SCP, followed by protein concentration/isolation steps.

The plant is to be designed such that the three main gas feedstocks of hydrogen, oxygen and carbon dioxide will be provided via dedicated supplies that can be varied independently of each other within pre-determined safety limits. Hydrogen gas can be either generated via electrolysis with hydrogen generators, or stored and supplied as a high-pressure gas from banks of cylinders, or stored as a cryogenic liquid. Oxygen and carbon dioxide may be stored as liquified gases in double skin cryogenic vessels and vaporized on demand using ambient aluminum fin vaporizers with integral pressure let downs stations. Alternatively, O₂ may also be sourced from an electrolyzer producing H₂. The gases will be filter sterilized using a 0.2 μm in line filter prior to entering the main fermenter. Gas flow control will be performed locally to the bioreactor using flowmeters and flow control valves.

The front end of the pilot plant can have the flexibility to handle bulk solids and liquid/solids blending to make up aqueous solutions. The non-sterile bulk media can be either sterilized in a batch mode, using a sterile media feed vessel, or via a continuous media sterilizer—allowing the plant to run either in a fed batch mode or a continuous fermentation mode.

It is planned that a single vertically oriented jacketed vessel be used for the preparation and make up of non-sterile liquid media solutions. Primarily the vessel will be used to make up bulk media solutions as required for gas fermentations. The jacketed vessel will allow the contents to be quickly heated to assist with the dissolution of powder into aqueous solutions. The present concept uses simple application of plant steam to the jacket to provide “coarse” heating functionality. The vessel shall nominally have an atmospheric working pressure and as such the maximum operating temperature will be limited to around 90° C. to prevent boil-off of aqueous solutions with steam as the heat source. An instrumented system will be used to shut off jacket steam supplies. Cooling will be provided by simple on/off cooling water flow to the jacket after the steam supply has been isolated. Jacketed heating in combination with a hygienic bottom mounted magnetic, seal-less agitator will be used for dissolving powders/solids. The agitator motor will be combined with a variable speed drive/frequency inverter to allow the rotational speed of the agitator to be varied. Bulk powders will be lifted above and then dispensed into the vessel via an overhead crane or ‘bulk bag’ hoist system. A large chute/aperture or oversized manway hatch will be provided to allow the bulk powders to fall under gravity into the vessel. Additions of solids will be measured and monitored by load cells mounted on the vessel. Not all powders and solids easily dissolve into aqueous solution. Some problematic powders can create agglomerates or clumps of solids resulting in a lumpy, inconsistent solution. In certain embodiments, a mobile, inline high shear powder blender and mixer package will be provided locally to the vessel with a recirculation line. These units are specifically designed to quickly and efficiently incorporate relatively large quantities of powders/solids into liquids with minimal manual effort and eliminate agglomerates resulting in a uniform solution consistency.

The complete media mixture will then be sterilized in downstream sterilization unit operations. The non-sterile media can be routed to either a continuous sterilizer unit or a batch sterilizer which also acts a sterile media feed vessel. In certain embodiments, the sterilization unit can consist of a sanitary heat exchanger that indirectly heats a flowing media stream against a hot utility fluid (e.g., steam) under pressure to an exit temperature of around 140-150° C. The media stream then flows into an insulated retention coil that acts as a plug flow reactor ensuring all the solution to be sterilized is kept at the elevated temperature for a defined residence time on the order of hundreds of seconds. After being held at sterilization temperatures for a specified residence time the solution will then be sensibly cooled in another hygienic heat exchanger against a cooling utility fluid and finally transferred into a bioreactor operating continuously or as a fed batch. In certain embodiments, batch sterilization under pressure is performed where a jacketed pressure vessel is charged with non-sterile media or solution and then isolated. Pressure and temperature increase will be accomplished using a combination of indirect heating by steam to the jacket and direct heating using clean steam injection. The bulk media can be agitated by the action of an agitator and use of a sparger to evenly distribute the clean steam into the bulk liquid phase to improve heat transfer and minimize batch heat-up time. In certain embodiments a bottom mounted “seal-less” agitator is used on the vessel. Once the batch temperature reaches around 130° C. it will be held at this temperature for around 30 minutes or longer to ensure an effective thermal kill of any viable biological contaminants. After the desired hold time at the sterilization temperature, clean steam flow into the vessel and to the jacket will stop. Cooling utility fluid will be applied to the vessel jacket to reduce the batch temperature such that it will not thermally shock downstream fermentation culture. To prevent vacuum formation when the clean steam flow has stopped sterile air will be introduced into the vessel. Vessel head space pressure will be controlled by the dual action of on/off flow of sterile air into the headspace combined with an outlet pressure control valve that vents sterile air from the vessel. Pneumatic sterile transfer will be used to feed the sterile media into a downstream bioreactor. A simple feedback control loop using a sanitary inline flowmeter and sanitary control valve will regulate the flow of sterile media from the vessel.

Trace solids and salt additions can also be made up and either: filter sterilized in line as a directly dosed sterile transfer to the bioreactor; or transferred to the non-sterile bulk media vessel, mixed with the bulk non-sterile media and then sterilized via the continuous media sterilizer or batch sterilized in the sterile media feed vessel.

Another dedicated bunded atmospheric tank will be used to make up aqueous solutions for trace salts and other water-soluble solids as needed during the bioprocess run. The ability to heat this tanks contents will be provided by an electric heating element. This will assist in dissolving the solids into solution and heating the solutions sufficiently so as not to thermally shock the downstream fermentation culture. In certain embodiments where it is anticipated that all the solids, once dissolved into water, remain dissolved, no vigorous agitation is provided on the tank itself to keep non-dissolved solids in suspension. In other embodiments, where precipitation of solids is considered probable, an agitator would be added to the tank. Dissolution of solids and tank circulation will be provided by a single inline powder blender and pump unit. The discharge of the unit will be connected to an eductor/jet mixer mounted inside the tank. This will provide a degree of liquid blending and tank turnover to minimize concentration and temperature gradients. Powder dispersion into the liquid phase will be provided by a powder dispersion/blending unit in an identical manner to that for the bulk media solids vessel. The trace salt/element solution can be dosed along a sterile transfer line inclusive of a sterile grade filter, thus providing a sterile addition direct to the fermenter. Alternatively, the solution can be dosed to the non-sterile bulk media vessel and blended with the bulk media for sterilization in either the continuous sterilizer or the sterile media feed vessel.

In certain embodiments the nitrogen source will comprise liquid phase ammonium hydroxide. In certain embodiments the nitrogen source will comprise gaseous, anhydrous ammonia. For embodiments utilizing ammonia, there will be refrigerated, pressurized and liquefied anhydrous ammonia double wall storage tanks. For embodiments utilizing ammonium hydroxide, there will be a bunded atmospheric, ambient temperature storage tank specified for the bulk storage of ammonium hydroxide. In certain embodiments, a dedicated tank is used for the bulk storage of ammonium hydroxide and in other embodiments an intermediate bulk container (IBC) may be used for the bulk storage of ammonium hydroxide. The ammonium will be dosed along a sterile transfer line via an inline sterilizing grade filter. The dosing pump will have a variable speed drive to permit variation of dosing rate. Dosing flow will be monitored by an inline flowmeter located outside of the sterile envelope.

The inoculation of the main production fermenter may be performed via a sterile transfer from a standard agitated vessel seed fermenter with the biomass cultivated aerobically on a sugar-based carbon source. For bacterial fermentations, the ratio between seed inoculum volume and the production volume may span the range of 1:10 to 1:1000.

pH of the fermentation will be monitored and controlled via dedicated filter sterilized acid and base additions which shall be dosed via sterile transfer lines. Fully welded piping/tubing will be specified to minimize leak points. In certain embodiments, NH₄OH or NH₃ will be used for basic adjustment. In other embodiments dilute KOH or NaOH solutions will used for pH regulation. In certain embodiments, CO₂ will be used for acidic adjustment. In other embodiments, phosphoric, nitric, or sulfuric acid will be used. Concentrations of sulfuric acid below around 20% w/w are generally regarded as being compatible with stainless steels at ambient temperatures. Dedicated bunded atmospheric storage tanks will hold the bulk alkali and any strong acids (e.g., phosphoric acid). For each chemical single dosing pumps with variable speed drives, and in certain embodiments, stroke length control, will be used to accurately dose the alkali and acid via single sterilizing grade filters into the bioreactor sterile envelope. A simple feedback control loop from the bioreactor will act on the dosing pump drive to add acid or alkali as required to maintain pH setpoint. In certain embodiments of the present invention, where a loop style bioreactor operating a plug type flow regime (e.g., radially well mixed, axially poorly mixed) is utilized, a more complex pH control system will be implemented where the circulation time of the loop will, in part, determine the rate of dosing to prevent pulses of high/low pH being established around the loop.

Temperature control functionality will be achieved by indirect heating and cooling via a pressurized tempered water system. Often on start-up and during the lag phase there is a requirement to apply heating to the broth until enough biomass is present such that it can create adequate metabolic heat to maintain constant culture temperature. Later there is a reversal from heating to cooling as the metabolic heat load increases such as during high productivity continuous runs or during the exponential phase and early stationary phase of batch or fed-batch runs. Therefore, the bioreactor will have an indirect heating and cooling tempered water system on the jacket of the fermenter. The tempered water will be circulated by a dedicated pump, and this forced circulation through the fermenter jacket will improve heat transfer and ensure minimal process deadtime. To prevent boil off the tempered water system will be pressurized. In certain embodiments, plant steam will be applied to a dedicated exchanger to heat the tempered water for controlled heat up of the bioreactor. In other embodiments, this could be supplied by an inline electric heater. When the switch over from heating to cooling occurs, plant steam flow to the heating exchanger will cease, and cooling water will be flowed through a dedicated cooling duty exchanger to begin reducing the tempered water temperature. In certain embodiments, chilling is required below the ambient wet bulb temperature, and in such embodiments, chilled water is used in addition to cooling water or instead of cooling water.

The addition of antifoam agents as needed will occur as follows. Antifoam will be sterilized and transferred along a dedicated sterile transfer line to the bioreactor. Thermal batch sterilization in a dedicated sterile vessel will be utilized. Non-sterile antifoam will be charged into this vessel. Pressure and temperature will be increased in the vessel by heating applied to the vessel in the batch thermal sterilization process. Heating will be applied both indirectly, via a jacket, and directly via clean steam injection. In certain embodiments, an agitator will be used to eliminate temperature gradients, improve heat transfer and minimize batch heat up times. Upon reaching the sterilisation temperature of around 130° C. the antifoam will be held at this value for around 30 minutes or longer to ensure a satisfactory thermal kill is achieved thus lowering bio-burden in the antifoam to acceptable limits. After the specified time at elevated temperature and pressure has elapsed, the vessel is cooled to fermentation temperatures by applying cooling utility fluid to the vessel jacket. To eliminate vacuum formation when the flow of clean steam ceases sterile air will be admitted into the vessel to create a slight positive pressure. When antifoam is required, sterile air may be used for pneumatic transfer of the antifoam along a sterile transfer line into the downstream bioreactor. An in-line flow meter and sanitary control valve will be used to accurately control antifoam flow if a prolonged, controlled rate of addition is needed.

Several instruments commonly used on bioreactors, each with their own associated control loops and configurable setpoints, will be used including but not limited to: pH, level, temperature, dissolved oxygen (DO), optical density (OD), and level. Headspace pressure monitoring and control will also be included.

The pilot plant will be equipped with an off-gas vent line, off-gas condenser, and off-gas analysis. In certain embodiments, the head space may be eliminated entirely from the fermenter as part of its basis of safety when operating gas-based fermentations. Nonetheless, the ability to purge or periodically vent gases in a controlled manner is a useful functionality to have incorporated on the fermenter.

The nitrogen source for the fermentation will be provided in the form of dosed ammonium hydroxide delivered via a dedicated sterile transfer line that is in-line sterile filtered.

Downstream process (DSP) unit operations that include but are not limited to dewatering of SCP fermentation broth will be utilized.

To address the possibility that unit operations may foul, lose efficiency, and/or need cleaning, effectively stopping the process flow, duty/standby unit operations will be installed, and/or suitably sized buffer capacity will be installed between unit operations to permit cleaning and turnaround of a unit while filling an upstream holding tank.

Fermentation broth will be harvested from the production bioreactor and held in a buffer vessel to permit de-gassing, temperature adjustment, and post fermentation additions to aid in downstream separations or processing—e.g., enzymes, flocculating agents, stabilizers etc. The buffer vessel will also provide the ability to operate downstream equipment at lower hydraulic loads or in batch mode for short periods as compared to the bioreactor harvest and dilution rate. When DSP equipment is operated as a continuous train it will have a specified combined maximum hydraulic throughput greater than the maximum bioreactor harvest rate so that accumulated buffer volume can be reduced.

Recovery of SCP biomass from aqueous broth will be achieved via a separation step involving centrifugation for initial dewatering. This dewatering may occur immediately following harvest of broth from the centrifuge or after temporary storage in a buffer tank. The centrifuge may be operated continuously or in batch mode. The recovered ‘thicks’ stream may be collected in a vessel. It may be sent through the centrifuge for second pass to further dewater the thicks.

A generic, non-sterile, agitated buffer vessel will be used with accurate temperature control capability via a pressurized tempered water system. This will permit the continued suspension of harvested biomass and allow it to be chilled, cooled, or heated. Monitoring of bulk temperature and of pH will be included on the vessel. Depending on the size of the buffer vessel and requirement for cooling/heating times, in certain embodiments a standalone temperature controller unit that is capable of both cooling and heating will used. In certain embodiments, utility fluids flowing through heat exchangers will be used. Also, of importance is the ability to fully and safely de-gas the broth, particularly if the broth has been harvested from a fuel gas containing atmosphere operating at elevated pressure. The buffer vessel will allow dissolved gases to come out of solution and be vented in a controlled and safe manner before the solution is pumped into other downstream operations. The buffer vessel will be inerted using head space blanketing via inlet and outlet pressure regulating valves. The ability to incorporate liquid additions from external containers of, for example, some form of stabilizer or pH adjustment using a local metering or dosing pump will installed.

In certain embodiments, a heating operation is performed after centrifugation. In certain embodiments, depending on broth rheology the heating operation is timed to occur before viscosity increases significantly to a point where indirect heat transfer becomes difficult to achieve. Depending on the purpose of the heating step various options exist for applying heat to the broth. In certain embodiments a simple thermal deactivation or bio-burden reduction is performed, which entails applying heat for tens of seconds at temperatures spanning 65 to 85° C.—a process like pasteurization of dairy products. In other embodiments, a more total sterilization—e.g., destruction of any spores—is performed, which entails temperatures in excess of 100° C. with corresponding pressures to prevent boiling, and exposure times on the order of tens of minutes. In certain embodiments, following heat treatment, further de-watering is performed in subsequent unit operations. This further de-watering can include, but is not limited to, further centrifugation passes and/or microfiltration and/or ultrafiltration.

The present disclosure is not limited in its application to the details of construction and the arrangement of components set forth in the description. The embodiments of the present disclosure are capable of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Although the foregoing invention has been described in some detail by way of illustration and examples for purposes of clarity of understanding, it will be apparent to those skilled in the art that certain changes and modifications may be practiced without departing from the spirit and scope of the invention, which is delineated in the appended claims. Therefore, the description should not be construed as limiting the scope of the invention.

All publications, patents, and patent applications cited herein are hereby incorporated by reference in their entireties for all purposes and to the same extent as if each individual publication, patent, or patent application were specifically and individually indicated to be so incorporated by reference.

1. A biological and chemical method for the biological conversion of inorganic and/or organic molecules comprising one or more carbon atoms into organic molecules, said method comprising:
introducing chemical reactants comprising inorganic and/or organic molecules comprising one or more carbon atom and comprising a gaseous substrate into an enclosed environment within a bioreactor that is held at an elevated pressure compared to an ambient pressure outside of the bioreactor,
wherein the enclosed environment comprises microorganism cells in a culture medium under conditions that are suitable for growing the microorganism cells and using the microorganism cells as a biocatalyst,
wherein the inorganic and/or organic molecules comprising one or more carbon atom are utilized as a carbon source by the microorganism cells for growth and/or biosynthesis of organic molecule products along with production of inorganic co-products; and converting the inorganic and/or organic molecules comprising one or more carbon atoms into the organic molecule products within the environment via at least one carbon-fixing reaction and/or at least one anabolic biosynthetic pathway contained within the microorganism cells,
wherein the carbon fixing reaction and/or anabolic biosynthetic pathway is at least partially driven by chemical and/or electrochemical energy provided by electron donors and/or electron acceptors contained within the gaseous substrate, which have been generated chemically and/or electrochemically and/or thermochemically and/or are introduced into the environment from at least one source external to the environment, and are reacted by the microorganism cells within the environment;
wherein the chemical reactants introduced into the environment comprise gaseous reactants, and wherein the organic products resulting from conversion of the carbon source and co-products of said conversion, and the products from the reaction of the electron donors and electron acceptors within the environment are all solids and/or liquids and/or dissolved solutes, and wherein none of said products or co-products from the conversion of the carbon source, and none of the products from the reaction of electron donors and electron acceptors thermodynamically favor the gas phase, and
wherein increased partial pressures of the gaseous reactants contained within the environment increase the thermodynamic driving force and kinetic rates for the conversion of the carbon source and/or the reaction of electron donors and electron acceptors.

2. The method of claim 1, wherein said elevated pressure is at least 1 bar gauge higher pressure than the ambient pressure outside of the bioreactor.

3. The method of claim 1, wherein the gaseous substrate comprises said carbon source.

4. The method of claim 1, wherein said microorganism cells are chemoautotrophic.

5. The method of claim 1, wherein said carbon source is CO₂, the electron donor is H₂, said electron acceptor is O₂. and said microorganism cells comprise knallgas microorganisms.

6. The method of claim 4, wherein said knallgas microorganisms comprise Cupriavidus necator.

7. The method of claim 4, wherein said knallgas microorganisms comprise microorganisms selected from one or more of the following genera: Cupriavidus sp., Rhodococcus sp., Hydrogenovibrio sp., Rhodopseudomonas sp., Hydrogenobacter sp., Gordonia sp., Arthrobacter sp., Streptomycetes sp. Rhodobacter sp., and/or Xanthobacter.

8. The method of claim 1, wherein said bioreactor is run in a continuous process, wherein fresh, cell-free culture medium is continually flowed into the environment, and culture broth comprising cells and/or the products of biosynthesis are continually removed from the environment.

9. The method of claim 1, wherein said bioreactor is run as a turbidostat or as a chemostat.

10. (canceled)

11. The method of claim 1, wherein
said bioreactor is connected to an external gas recirculation loop.

12. The method of claim 1, wherein said electron donor is hydrogen generated by the electrolysis of water performed using one or more of: Proton Exchange Membranes (PEM); liquid electrolytes such as KOH; alkaline electrolysis; Solid Polymer Electrolyte electrolysis; high-pressure electrolysis; and high temperature electrolysis of steam (HTES).

13. The method of claim 12, wherein said electron acceptor is oxygen that is also generated by said electrolysis of water.

14. The method claim 1, wherein said electron donors and/or electron acceptors are generated or recycled using renewable, alternative, or conventional sources of power that are low in greenhouse gas emissions, and wherein said sources of power are selected from at least one of photovoltaics, solar thermal, wind power, hydroelectric, nuclear, geothermal, enhanced geothermal, ocean thermal, ocean wave power, and tidal power.

15. The method of claim 1, wherein said electron donors and/or electron acceptors are generated using grid electricity during periods when electrical grid supply exceeds electrical grid demand, and wherein storage tanks buffer the generation of said electron donors and/or electron acceptor, and their consumption in the said carbon-fixing reaction.

16. The method of claim 1, wherein the said bioreactor is a stirred tank reactor (STR), a bubble column, a gas lift bioreactor, a trickle bed bioreactor, a pressure cycle loop bioreactor, a pressure cycle loop bioreactor, a mechanically stirred loop bioreactor, an ejector loop bioreactor, a venturi bioreactor, or a membrane bioreactor.

17. The method of claim 16, wherein said bioreactor is a STR that comprises a hollow gas entrainment impeller utilized to re-entrain headspace gases in said bioreactor.

18.-24. (canceled)

25. The method of any of claim 1, wherein the bioreactor further comprises:
a reactor vessel configured to contain a culture comprising a hydrogen-oxidizing or carbon monoxide-oxidizing microorganism and a gas headspace overlying the culture;
one or more oxygen sensor(s) configured to measure
a level of dissolved oxygen in the culture, and/or
a level of oxygen gas in the gas headspace;
a first gas feed manifold connected to a source of oxygen gas and configured to deliver oxygen gas into the culture, wherein the gas mixture is delivered under an amount of pressure;
a stirring means for mixing the culture; and
a gas feed controller configured to regulate, based on the measured level of dissolved oxygen in the culture and/or the measured level of oxygen gas in the gas headspace, one or more of:
an extent of mixing by the stirring means,
a level of oxygen gas delivered to the culture via the first gas feed manifold, or the amount of pressure;
a pH sensor configured to measure a pH level of the culture;
a base feed manifold configured to deliver a base to the culture;
a base feed controller configured to regulate an amount of the base delivered to the culture based on the measured pH level;
a nutrient feed manifold configured to deliver a nutrient amendment to the culture; and
a nutrient feed controller configured to regulate an amount of the nutrient amendment delivered to the culture,
wherein the amount of the nutrient amendment is proportional to the amount of the base.

26. The method of claim 25, wherein the level of oxygen gas delivered to the culture comprises the partial pressure of oxygen gas and its flow rate.

27. The method of claim 25, wherein the gas feed controller regulates a flow rate of oxygen gas delivered to the culture.

28. The method of claim 25, wherein the base is ammonium hydroxide.

29. The method of claim 25, wherein the bioreactor comprises an oxygen sensor configured to measure a level of oxygen gas in the gas headspace, wherein the gas feed controller is configured to regulate, based on the measured level of oxygen gas in the gas headspace, one or more of:
an extent of mixing by the stirring means, or
a level of oxygen gas delivered to the culture via the first gas feed manifold.

30. The method of claim 25, wherein the bioreactor comprises:
a culture media feed manifold configured to deliver culture media to the culture; and
a culture media feed controller configured to regulate an amount of culture media delivered to the culture.

31. The method of claim 25, wherein the bioreactor further comprises of an optical density sensor configured to measure an optical density of the culture, wherein the culture media feed controller is configured to regulate the amount of culture media delivered to the culture based on the measured optical density.

32. The method of claim 1, wherein the method further comprises:
delivering a gas mixture comprising oxygen gas into a culture of a hydrogen-oxidizing or carbon monoxide-oxidizing microorganism in a reactor vessel; and
providing a gas permeable barrier separating a first compartment fluidly connected to the culture and a second compartment comprising oxygen gas;
wherein a partial pressure of oxygen gas in the second compartment is greater than a partial pressure of oxygen gas in the gas mixture.
